(19) United States
(12) Patent Application Publication
(10) Pub. No.: US 2006/0107351 A1
(43) Pub. Date: May 18, 2006

(54) Cell visual characteristic-modifying sequences

(75) Inventors: Mirko Karan, Watsonia (AU); Filippa Brugliera, Preston (AU); John Mason, Carlton North (AU); Sophie Gwendoline Dove, St. Lucia (AU); Ian Ove Hoegh-Guldberg, St. Lucia (AU); Mark Prescott, Malvern East (AU)

Correspondence Address: SEED INTELLECTUAL PROPERTY LAW GROUP PLLC, 701 FIFTH AVE, SUITE 6300, SEATTLE, WA, 98104-7092, US

(73) Assignees: Florigene Limited, Collingwood (AU); The University of Queensland, St. Lucia (AU)

(21) Appl. No.: 11/118,112

(22) Filed: Apr. 29, 2005

Related U.S. Application Data
(63) Continuation of application No. 10/469,661, filed on Jan. 19, 2005.
(60) Provisional application No. 60/273,227, filed on Mar. 2, 2001.
(60) Provisional application No. 60/329,816, filed on Oct. 15, 2001.

(30) Foreign Application Priority Data
Mar. 21, 2001 (AU) PR3874/01

(51) Int. Cl. A01H 1/00; C12Q 1/68; C07H 21/04; C07K 14/415; C12N 1/21

(52) U.S. Cl. 800/282; 435/6; 435/419; 435/468; 435/252.3; 435/417; 530/370; 536/23.6

(57) ABSTRACT

The present invention relates generally to peptides, polypeptides or proteins having one or more amino acids or one or more amino acid sequences which exhibit color-facilitating properties, either on their own or following interaction with one or more other amino acids and to nucleic acid molecules encoding same. Such peptides, polypeptides and proteins are referred to herein as “color-facilitating molecules” or “CFMs”. The present invention further provides genetic constructs for use in genetically modifying eukaryotic or prokaryotic cells and more particularly eukaryotic tissue so as to alter their visual characteristics or capacity for exhibiting same to a human eye in the absence of excitation by an extraneous non-white light or particle emission. The present invention, therefore, extends to eukaryotic or prokaryotic cells and more particularly eukaryotic tissue, which are genetically modified to produce CFMs and which thereby exhibit altered visual characteristics in the absence of excitation by an extraneous non-white light or particle emission. In one particular embodiment, the CFMs are used to alter the visual characteristics of plants and even more particularly flower color. In another embodiment, the present invention provides gels or coatings or similar biomaterials in the form of a biomatrix comprising the CFMs such as for use as a UV sink, in a sun screen, in cosmetics, as an expression marker or other reporter molecule or for use as a photon trap to increase light intensity.

### FIELD OF THE INVENTION

The present invention relates generally to peptides, polypeptides or proteins having one or more amino acids or one or more amino acid sequences which exhibit color-facilitating properties, either on their own or following interaction with one or more other amino acids and to nucleic acid molecules encoding same. Such peptides, polypeptides and proteins are referred to herein as “color-facilitating molecules” or “CFMs”. The present invention further provides genetic constructs for use in genetically modifying eukaryotic or prokaryotic cells and more particularly eukaryotic tissue so as to alter their visual characteristics or capacity for exhibiting same to a human eye in the absence of excitation by an extraneous non-white light or particle emission. The present invention, therefore, extends to eukaryotic or prokaryotic cells and more particularly eukaryotic tissue, which are genetically modified to produce CFMs and which thereby exhibit altered visual characteristics in the absence of excitation by an extraneous non-white light or particle emission. In one particular embodiment, the CFMs are used to alter the visual characteristics of plants and even more particularly flower color. In another embodiment, the present invention provides gels or coatings or similar biomaterials in the form of a biomatrix comprising the CFMs such as for use as a UV sink, in a sun screen, in cosmetics, as an expression marker or other reporter molecule or for use as a photon trap to increase light intensity.

### BACKGROUND OF THE INVENTION

Reference to any prior art in this specification is not, and should not be taken as, an acknowledgment or any form of suggestion that this prior art forms part of the common general knowledge in Australia or any other country.

All-protein chromophores (pigments) have been isolated from the phylum Cnidaria (also known as Coelenterata). This phylum contains four classes: Scyphozoa, Cubozoa, Anthozoa and Hydrozoa. The first all-protein chromophore to be isolated, Green Fluorescent Protein (GFP), was cloned and sequenced from cDNA of the Hydrozoan Aequorea victoria, commonly called jellyfish.

Similar all-protein chromophores have been isolated from Anthozoans. Matz et al. (Nature Biotechnol. 17:969-973, 1999), used degenerative primers based on Aequorea Victoria GFP nucleotide sequence to PCR amplify cDNA isolated from four of the five orders of Anthozoa: Stolonifera, Actiniaria, Zoanthidea, and Corallimorpharia. Lukyanov et al. (Journal of Biological Chemistry 275:25879-25882, 2000) used the same methodology to isolate a non-fluorescent all-protein chromophore from Actiniaria. However, the methodology used was unable to isolate all-protein chromophores from the fifth order, Scleractinia.

The Scleractinia are corals that form architecture for coral reefs. They are otherwise known as “true” or “reef-building” corals. International Patent Publication No. WO 00/46233 and Dove et al. (Coral Reefs 19:197-204, 2000) both relate to isolation of an all-protein chromophore derived from Scleractinia pigment protein from coral tissue (PPCT).

All-protein chromophores isolated to date display a range of spectral properties which effect apparent color in specific environments. Color may be determined by absorption and/or fluorescence properties of the molecules as well as qualities of incident light. Spectral properties include absorption, excitation and emission energies, molar extinction coefficients, quantum yields and maturation parameters. In many cases, a simple amino acid substitution can have a dramatic effect on the polypeptide spectral parameters (e.g. Tsien, Ann. Rev. Biochem. 67:509, 1998; Lukyanov et al., 2000, supra). However, usefull modifications of a particular molecule are limited, as directed and random mutagenesis of specific all-protein chromophores has failed to produce desired spectral features (Tsien, 1998, supra). The result is that all-protein chromophores isolated from different sources are finding specific application niches.

One all-protein chromophores, primarily used as molecular marker, is GFP. This protein, when excited with either UV or blue light (maximally at 396 nm or 475 nm) emits green fluorescence (maximally at 500 nm) [Heim et al., Proc. Natl. Acad. Sci. USA 91:12501-12504, 1994]. GFP mutants that are altered in their maximal excitation and emission characteristics have been generated by random mutagenesis (Crameri et al., Nature Biotechnology 14:315-319, 1996). Other GFP mutants have been generated that have increased solubility and fluorescence (Davis and Vierstra, Soluble derivatives of green fluorescent protein (GFP) for use in Arabidopsis thaliana. Weeds of the World, The International Electronic Arabidopsis Newsletter ISSN 1358-6912, (Ed. Mary Anderson) vol 3ii, 1996). The fluorescence of GFP and its mutants has been exploited for non-invasive analysis and monitoring of biological samples in plants and other organisms for research purposes (Haseloff et al., Proc. Natl. Acad. Sci USA 94:2122-2127, 1997; Hu and Cheng, FEBS Letters 369:331-334, 1995; Wang and Hazelrigg, Nature 369:400-403, 1994). The use of these fluorescent GFPs, mutants and homologs as fluorescent marker pigments visible upon excitation by light of specific wavelengths is well documented (e.g. U.S. Pat. Nos. 6,027,881 and 5,958,713; Japanese Patent No. 11266883; International Patent Publication No. WO97/11094; U.S. Pat. No. 5,625,048; International Patent Application No. PCT/US99/29472 and International Patent Publication No. PCT/AU00/00056).

In contrast to other fluorescent proteins, the fluorescence of GFP is due to amino acid interaction within the molecule, generally after folding. A contiguous fluorophore-defining amino acid sequence of Ser-Tyr-Gly is modified upon folding to produce an extended aromatic system which imparts the characteristic green fluorescence to the mature protein (Cody et al., Biochemistry 32:1212-1218, 1993; Ormö et al., Science 273:1392-1395, 1996; Yang et al., Nature Biotechnol. 14:1246-1251, 1996). As stated above, GFP like molecules have been identified for nonbioluminscent Anthozoa species (Matz et al., 1999, supra) which provides evidence that GFP-like proteins are not necessarily components of bioluminescent systems but may just determine fluorescent coloration in animals (Lukyanov et al., 2000, supra). Other weakly fluorescent GFP homologs have been identified from Acropora formosa and Acropora digitifera (Dove et al., Biol. Bull. 189:288-297, 1995; Hoegh-Guldberg and Dove, 2000, supra; Salih et al., Nature 408:850-853, 2000).

All-protein chromophores are now finding application as molecular markers for monitoring polypeptide expression and localization in the fields of biochemistry, molecular and cell biology.

The present invention now describes novel all-protein chromophores (or CFMs) as well as novel and useful applications of same.

For example, the flower industry strives to develop new and different varieties of flowering plants, in particular through the manipulation of flower color. While classical breeding techniques have been used with some success to produce a wide range of colors for most of the commercial varieties of flowers, this approach has been limited by the constraints of a particular species' gene pool. For this reason, it is rare for a single species to have a full spectrum of colored varieties. The development of blue varieties of major cut flower species such as rose, chrysanthemum, tulip, lily, carnation and gerbera, for example, has proved difficult and would offer a significant opportunity in both the cut flower and ornamental markets.

Flower color is predominantly due to three types of pigment: flavonoids, carotenoids and betalains. Of the three, the flavonoids are the most common and contribute to a range of colors from yellow to red to blue. The flavonoid molecules which make the major contribution to flower color are the anthocyanins which are glycosylated derivatives of cyanidin, delphinidin, petunidin, peonidin, malvidin and pelargonidin and are localized in the vacuole. Carotenoids are natural pigments that confer yellow, orange and red colors to flowers and fruit. In plants, these pigments are localized in chromoplasts in flowers, leaves, fruit and roots.

Novel colors in ornamental plant and flowering plant species may be generated by modifying the anthocyanin pathway to produce novel anthocyanins and aurones (Davies et al., Plant Journal 13:259-266, 1998) and to alter ratios of anthocyanins to co-pigments (Holton et al., Plant Journal 4:1003-1010, 1993). Alternatively, the carotenoid biosynthetic pathway can be modified to produce novel flower colors (Mann et al., Nature Biotech. 18:888-892, 2000). The levels of anthocyanin production can also be increased by the expression of heterologous anthocyanin pathway gene regulatory factors (e.g. see Borevitz et al., Plant Cell 12:2383-2393, 2000).

These approaches have been used with some, albeit limited, success and alternative novel approaches are constantly being sought.

In work leading up to the present invention, the inventors sought, inter alia, to identify novel color-facilitating molecules (CFMs) and to use same to modify the visual characteristics of eukaryotic or prokaryotic organisms by introducing into eukaryotic or prokaryotic cells, genetic material encoding CFMs which impart a color visible to a human eye in the absence of excitation by extraneous non-white light or particle emission. In a preferred embodiment, the CFMs are proteins such as GFPs or their relatives, such as non-fluorescent GFP-homologs. The use of CFMs to modulate the color of plants or plant parts such as flowers and seeds, represents a new approach to developing plant varieties having altered color characteristics. Other uses contemplated herein for the CFMs include their use as expression markers or as general reporter molecules, as a photon trap, UV sink and in sun screen or cosmetic or may be embedded in a gel matrix and be used to convert less visible light to wavelengths which are more Visible. All such compositions are encompassed by the term “biomatrix”.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

The present invention provides peptides, polypeptides and proteins having one or more amino acid sequences which exhibit color-facilitating properties, either on their own or following interaction with one or more amino acids as well as nucleic acid molecules encoding same. Preferably, the peptides, polypeptides and proteins or their nucleic acid molecules are derived from one or more Anemonia majano, Anemonia sulcata, Clavularia sp, Zoanthus sp, Discosoma sp (e.g. Discosoma striata), Aequorea sp (e.g. Aequorea victoria), Anthozoa sp, Cassiopea sp, (e.g. Cassiopea xamachana), Millepora sp, Acropora sp (e.g. Acropora aspera and Acropora nobilis), Montipora sp, Porites murrayensis, Pocillopora damicormis, Pavona descussaca, Acanthastrea sp, Platygyra sp or Caulastrea sp. These peptides, polypeptides and proteins are referred to as “color-facilitating molecules” (CFMs) and may be in isolated form, be produced within or on a cell or may form part of a biomatrix.

Accordingly, in one aspect of the present invention, there is provided an isolated nucleic acid molecule comprising a nucleotide sequence encoding a color-facilitating molecule (CFM) which, in a cell, alone or together with one or more other molecules imparts an altered visual characteristic to said cell when visualized by a human eye in the absence of excitation by extraneous non-white light or particle emission.

The present invention also provides an isolated CFM comprising a polypeptide which, in a cell, alone or together with one or more other molecules imparts an altered visual characteristic to said cell when visualized by a human eye in the absence of excitation by extraneous non-white light or particle emission.

The preferred CFM comprises the amino-terminal end of the polypeptide set forth in SEQ ID NOs: 5, 6, 7, 8 or 9.

Particularly preferred CFMs comprise amino acid sequences selected from SEQ ID NOs:10, 11, 12, 13, 14, 15, 16, 17 or 18.

Even more preferably, the CFM is encoded by a nucleotide sequence set forth in any one of SEQ ID NOs:19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 189, 191, 193, 195, 197, 199 and 201 or a nucleotide sequence capable of hybridizing to one of the above sequences or a complementary form thereof under low stringency conditions or a nucleotide sequence having at least about 60% similarity to any one of the above sequences.

Amino acid sequences corresponding to the above nucleotide sequences correpond to SEQ ID NOs:20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 190, 192, 194, 196, 198, 200 and 202 as well as an amino acid sequence having at least about 60% similarity to any one of the above sequences.

The CFM may be in isolated form or part of a biomatrix wherein the biomatrix includes a cell, solid support, gel or bioinstrument. The CFMs are particularly useful in generating eukaryotic or prokaryotic cells exhibiting altered visual characteristics as well as biomatrices in the form of sun screen, UV traps, photon traps and illuminescent intensifiers.

In a particularly preferred embodiment, the present invention provides transgenic plants and parts thereof including flowers, roots, leaves, stems, fruit and fibers exhibiting an altered visual characteristic. dr

### BRIEF DESCRIPTION OF THE FIGURES

FIG. 1 shows a representation of multiple alignment of encoded amino acid sequences having SEQ ID NOs:20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84 and 86, representing polypeptides comprising an N-terminal SVIAK (SEQ ID NO:5) sequence.

FIG. 2 shows corresponding nucleotide sequence alignments of nucleic acid molecules, having SEQ ID NOs:19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83 and 85, encoding the polypeptides shown in FIG. 1.

FIG. 3 shows a representation of multiple alignment of encoded amino acid sequences having SEQ ID NOs:88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 and 168, for polypeptides comprising an N-terminal (M)SVIAT (SEQ ID NO:6), SGIAT (SEQ ID NO:7), SVIVT (SEQ ID NO:8) and SVSAT (SEQ ID NO:9) sequences.

FIG. 4 shows corresponding nucleotide sequence aligments of nucleic acid molecules, having SEQ ID NOs:87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165 and 167, encoding the polypeptides shown in FIGS. 3A-3D.

FIG. 5 shows a representation of an alignment of amino acid sequences having SEQ ID NOs:170, 172, 174, 176, 178 and 180, for polypeptides comprising an N-terminal SVIAK sequence (SEQ ID NO:5) and a stop codon corresponding to amino acid residue 14.

FIG. 6 shows corresponding nucleotide sequence alignments for nucleic acid molecules, having SEQ ID NOs:169, 171, 173, 175, 177 and 179, encoding the polypeptides shown in FIG. 5.

FIG. 7 is a nucleotide sequence alignment of SEQ ID NO:19 and SEQ ID NO:169, being nucleic acid sequences encoding polypeptides without and with a stop codon corresponding to amino acid residue 14, respectively.

FIG. 8 shows a representation of multiple alignment of amino acid sequences for polypeptides comprising an N-terminal SVIAK sequence (SEQ ID NO:5), including SEQ ID NOs:20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84 and 86, as well as sequences Aapat-1 (SEQ ID NO:181) and Aapat-2 (SEQ ID NO:182) which are disclosed in International Patent Publication No. WO 00/46233.

FIG. 9 shows amino acid sequence alignments of pigment polypeptides from coral tissue, grouped according to their N-terminal 5-amino acid sequence. The name and SEQ ID NO for each peptide is indicated, as well as the “Type” to which each has been assigned based on the identity of the 29 amino acids which are located within 5 Angstroms of the “QYG” fluorophore. These 29 individual, non-contiguous amino acid residues are also indicated, as are the individual non-contiguous variable amino acids residues throughout the polypeptides shown.

FIG. 10 is a diagrammatic representation of a generic bacterial expression vector based on pQE-30 (Qiagen), into which is inserted an ˜0.7 kb cDNA; depending on the source of the cDNA clone, each plasmid is designated as follows: pCGP2915—A10 clone from Acropora sp.; pCGP2916—All clone from Acropora sp.; pCGP2917—A12 clone from Acropora sp.; pCGP2918—A8 clone from Acropora sp. (SEQ ID NO:189); pCGP2920—D10 clone from Discosoma sp. (SEQ ID NO:191); pCGP2922—T3 clone from Tubastrea sp. (SEQ ID NO:195); pCGP2924—S3 clone from Sinularia sp. (SEQ ID NO:193); pCGP2919—D1 clone from Discosoma sp. (SEQ ID NO:197); pCGP2921—T1 clone from Tubastrea sp. (SEQ ID NO:201); pCGP2923—S1 clone from Sinularia sp. (SEQ ID NO:199). Abbreviations are as follows: bla=β-lactamase gene; ColElori=plasmid origin of replication. The locations of restriction endonuclease recognition sites for PstI, HindIII and BamHI are also marked. Refer to Example 3 for further details.

FIG. 11 is a graphical representation of examples of absorption scans of five “Type 1” (refer to text in Example 2 and Tables 6 and 7 for further detail) colored proteins showing extinction coefficients (ε_{λmax}) based on the method of Whitaker and Granum, 1980 (Anal. Biochem. 109:156-159) for calculating protein concentration. x-axis=relative absorption; y-axis=wavelength (run); (a) Rtms5.pep (SEQ ID NO:166), where ε₅₉₂=111,000 M⁻¹ cm⁻¹; (b) LGasv-C.pep (SEQ ID NO:44) where ε₅₉₁=53,000 M⁻¹ cm⁻¹; (c) Ce61-7sv.pep (SEQ ID NO:38) where ε_{591.5}=104,000 M⁻¹ cm⁻¹; (d) PPd57-2ms.pep (SEQ ID NO:140) where ε₅₉₃=67,000 M⁻¹ cm⁻¹; (e) Mims-C.pep (SEQ ID NO:126) where ε₅₈₉=48,000 M⁻¹ cm⁻¹.

FIG. 12 a graphical representation of examples of absorption scans of three “Type 2” (A) and two “Type 12” (B) (refer to text in Example 2 and Tables 6 and 7 for further detail) colored proteins, showing extinction coefficients (ε_{λmax}) based on the method of Whitaker and Granum (Anal. Biochem. 109:156-159, 1980) for calculating protein concentration. x-axis=relative absorption; y-axis=wavelength (nm); (A) (a) PMms-B.pep (SEQ ID NO:130) where ε_{579.5}=39,000 M⁻¹ cm⁻¹; (b) LGAsv-D.pep (SEQ ID NO:46) where ε₅₇₉=72,400 M⁻¹ cm⁻¹; (c) rtsv-2.pep (SEQ ID NO:84) where ε_{579.5}=75,000 M⁻¹ cm⁻¹; (B) (d) Misv-F.pep (SEQ ID NO:54) where ε₅₇₉=111,000 M⁻¹ cm⁻¹; (e) Acasv-C.pep (SEQ ID NO:78) where ε_{579.5}=32,300 M⁻¹ cm⁻¹.

FIG. 13 a graphical representation of examples of absorption scans of two “Type 6” (refer to text in Example 2 and Tables 6 and 7 for further detail) colored proteins, showing extinction coefficients (ε_{λmax}) based on the method of Whitaker and Granum (Anal. Biochem. 109:156-159, 1980) for calculating protein concentration. x-axis=relative absorption; y-axis=wavelength (nm); (a) LGAms-5.pep (SEQ ID NO:116) where ε_{583.5}=71,000 M⁻¹ cm⁻¹; (b)Rtms-1.pep (SEQ ID NO:162) where ε₅₈₄=44,000 M⁻¹ cm⁻¹.

FIG. 14 a graphical representation of (A) Absorption spectra and (B) Chromatogram of gel filtrated protein elution, both showing 95% confidence intervals for N=5, for raw phosphate buffer extract of two colour morphs of Acropora aspera (dark blue pigmented morph; cream morph). In (A), the estimation of blue-purple pocilloporin concentration per surface area of coral tissue is based on an extinction coefficient range of 50,000-100,000 M⁻¹ cm⁻¹. In (B), the chromatogram of gel filtrated protein elution is determined from 235 nm chromatograms and 280 nm chromatograms, applying the equation: 235nm-280 nm)/ 2.51 (Whitaker and Granum, 1980, supra). The total area under the graph represents the total soluble protein. Blue-purple pocilloporin concentration is based on the difference between areas under the blue and cream graph in the range of pocilloporin elution (24-26.5 min).

FIG. 15 is a representation of multiple alignment of encoded amino acid sequences from T1 (SEQ ID NO:202), D1 (SEQ ID NO:198), S1 (SEQ ID NO:200), T3 (SEQ ID NO:196), D10 (SEQ ID NO: 192), S3 (SEQ ID NO:194) and A8 (SEQ ID NO:190).

FIG. 16 is a representation of multiple alignment of encoded amino acid sequences from SVIAK (SEQ ID NO:5)-containing peptides T1 (SEQ ID NO:202), D1 (SEQ ID NO:198), S1 (SEQ ID NO:200), T3 (SEQ ID NO:196), D10 (SEQ ID NO:192), S3 (SEQ ID NO:194) and A8 (SEQ ID NO:190), together with the SVIAK (SEQ ID NO:5)containing peptides shown in FIG. 1, having SEQ ID NOs:20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84 and 86.

FIG. 17 is a diagrammatic representation of the yeast expression plasmid pCGP3269. The T1 cDNA (SEQ ID NO:201) cloned in a sense orientation behind the yeast glyceraldehyde 3-phosphate dehydrogenase promoter (PGAP) in the expression vector pYE22m. Abbreviations are as follows: TRP1=Trp1 gene, TGAP=terminator sequence from the yeast glyceraldehyde 3-phosphate dehydrogenase gene, IR1=inverted repeat of 2 μm plasmid, pBR322=origin of replication from E. coli. A selection of restriction enonuclase recognition sites are also marked. Refer to Example 7 for further details.

FIG. 18 is a diagrammatic representation of the yeast expression plasmid pCGP3270. The A8 cDNA (SEQ ID NO:189) cloned in a sense orientation behind the yeast glyceraldehyde 3-phosphate dehydrogenase promoter (PGAP) in the expression vector pYE22m. Abbreviations are as follows: TRP1=Trp1 gene, TGAP=terminator sequence from the yeast glyceraidehyde 3-phosphate dehydrogenase gene, IR1=inverted repeat of 2 μm plasmid, pBR322=origin of replication from E. coli. A selection of restriction enonuclase recognition sites are also marked. Refer to Example 7 for further details.

FIG. 19 is a diagrammatic representation of a plasmid, designated pCGP2756, which comprises a multiple cloning site from pNEB193 (New England Biolabs) between the CaMV (Cauliflower Mosaic Virus) 35S promoter and CaMV 35S terminator sequences. Abbreviations are as follows: Amp=ampicillin resistance gene; p35S=a promoter region from the CaMV 35S gene; t35S=a terminator fragment from the CAMV 35S gene. A selection of restriction endonuclease recognition sites are also marked. Refer to Example 9 for further details.

FIG. 20 is a diagrammatic representation of the binary plasmid pCGP2757, which comprises the CaMV35S expression cassette of pCGP2756 (FIG. 19) and a SuRB selectable marker gene. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli. Selected restriction endonuclease recognition sites are also marked. Refer to Example 9 for further details.

FIG. 21 is a diagrammatic representation of the binary plasmid pCGP2765, which comprises the A8 cDNA from Acropora sp. (SEQ ID NO:189) cloned into the binary vector pCGP2757 (FIG. 20). Abbreviations are as follows: TetR=the tetracycline resistance gene; LB=left border; RB=right border; SuRB=the coding region and terminator sequence from the acetolactate synthase gene from tobacco; p35S =a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; A8=cDNA from Acropora sp. (SEQ ID NO:189). Selected restriction endonuclease recognition sites are also marked. Refer to Example 9 for further details.

FIG. 22 is a diagrammatic representation of the binary plasmid pCGP2769, which comprises the D1 cDNA from Discosoma sp. (SEQ ID NO:197) cloned into the binary vector pCGP2757 (FIG. 20). Abbreviations are as follows: TetR=the tetracycline resistance gene; LB=left border; RB=right border, SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; D1=cDNA from Discosoma sp. (SEQ ID NO:197). Selected restriction endonuclease recognition sites are also marked. Refer to Example 9 for further details.

FIG. 23 is a diagrammatic representation of the binary plasmid pCGP2770, which comprises the S1 cDNA from Sinularia sp. (SEQ ID NO:199) cloned into the binary vector pCGP2757 (FIG. 20). Abbreviations are as follows: TetR=the tetracycline resistance gene; LB=left border, RB=right border; SuRB =the coding region and terminator sequence from the acetolactate synthase gene from tobacco; p35S =a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; S1=cDNA from Sinularia sp. (SEQ ID NO:199). Selected restriction endonuclease recognition sites are also marked. Refer to Example 9 for further details.

FIG. 24 is a diagrammatic representation of the binary plasmid pCGP2772, which comprises the T1 cDNA from Tubastrea sp. (SEQ ID NO:201) cloned into the binary vector pCGP2757 (FIG. 20). Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; T1=cDNA from Tubastrea sp. (SEQ ID NO:201). Selected restriction endonuclease recognition sites are also marked. Refer to Example 9 for further details.

FIG. 25 is a diagrammatic representation of the plasmid pCGP1116, which comprises a promoter fragment from a chalcone synthase (CHS) gene from Rosa hybrida cv. Kardinal. Abbreviations are as follows: Rose CHS=Rose chalcone synthase promoter fragment; ori =origin of replication; Amp=ampicillin resistance gene; Several restriction endonuclease recognition sites are also marked. Refer to Example 10 for further details.

FIG. 26 is a diagrammatic representation of the binary plasmid pCGP3255. The CaMV35S promoter of the 35S expression cassette of pCGP2757 (FIG. 20) has been replaced with the rose chalcone synthase promoter fragment from pCGP1116 (FIG. 25) Abbreviations are as follows: rCHS=rose chalcone synthase promoter fragment; TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene, t35S=a terminator fragment from the CaMV 35S gene; pVS1=a broad host range origin of replication from a plasmid from Pseuodomonas aeruginosa; pACYC ori=modified replicon from pACYC184 from E. coli. Refer to Example 10 for frther details.

FIG. 27 is a diagrammatic representation of the bianry plasmid pCGP2782. The T1 cDNA from Tubastrea sp. (SEQ ID NO:201) was cloned into binary vector pCGP3255 (FIG. 26) behind the rose chalcone synthase promoter fragment. Abbreviations are as follows: rCHS=rose chalcone synthase promoter fragment; TetR=the tetracycline resistance gene; LB=left border, RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; T1=cDNA from Tubastrea sp. (SEQ ID NO:201). A selection of restriction endonuclease recognition sites is also marked. Refer to Example 10 for further details.

FIG. 28 is a diagrammatic representation of the binary plasmid pCGP2773. The D1 cDNA from Discosoma sp. (SEQ ID NO:197) was cloned into binary vector pCGP3255 (FIG. 26), behind the rose chalcone synthase promoter fragment. Abbreviations are as follows: rCHS=rose chalcone synthase promoter fragment; TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CAMV 35S gene; pVS1=a broad host range origin of replication from a plasmid from Pseuodomonas aeruginosa; pACYC ori=modified replicon from pACYC184 from E. coli; D1=cDNA from Discosoma sp. (SEQ ID NO:197). A selection of restriction endonuclease recognition sites is also marked. Refer to Example 10 for further details.

FIG. 29 is a diagrammatic representation of the binary plasmid pCGP2774. The S1 cDNA from Sinularia sp. (SEQ ID NO:199) was cloned into binary vector pCGP3255 (FIG. 26), behind the rose chalcone synthase promoter fragment. Abbreviations are as follows: rCHS=rose chalcone synthase promoter fragment; TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC 184 from E. coli; S1=cDNA from Sinularia sp. (SEQ ID NO:199). A selection of restriction endonuclease recognition sites is also marked. Refer to Example 10 for further details.

FIG. 30 is a diagrammatic representation of the binary plasmid pCGP2780, which is plasmid pCGP2757 (FIG. 20) from which has been removed a ˜290 base-pair SagII fragment to allow the creation of a unique BamHI restriction endonuclease site. Abbreviations are as follows: TetR=the tetracycline resistance gene; LB=left border, RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli A selection of restriction endonuclease recognition sites is also marked. Refer to Example 11 for further details.

FIG. 31 is a diagrammatic representation of the binary plasmid pCGP2784, which is comprised of the ˜0.2 kb chloroplast transit-peptide from the small subunit of ribulose bisphosphate carboxylase gene (RBCase) from Nicotiana sylvestris, cloned into the multiple cloning site of pCGP2780 of FIG. 30. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CAMV 35S gene; pVS1=a broad host range origin of replication from a plasmid from Pseuodomonas aeruginosa; pACYC ori=modified replicon from pACYC184 from E. coli; TSSU=chloroplast transit-peptide from the small subunit of RBCase of Nicotiana sylvestris. Selected restriction endonuclease recognition sites are also marked. Refer to Example 11 for further details.

FIG. 32 is a diagrammatic representation of the binary plasmid pCGP2781, which is plasmid pCGP2772 (FIG. 24) from which has been removed a ˜290 base-pair SalI fragment to allow the creation of a unique BamHI restriction endonuclease site. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli. T1=T1 cDNA from Tubastrea sp. (SEQ ID NO:201). Selected restriction endonuclease recognition sites are also marked. Refer to Example 11 for further details.

FIG. 33 is a diagrammatic representation of the binary plasmid pCGP2785, which is comprised of the ˜0.2 kb chloroplast transit peptide from the small subunit of ribulose biphosphate carboxylase (RBCase) from Nicotiana sylvestris inserted into the CaMY 35S expression cassette of binary vector pCGP2781 (FIG. 32), upstream of the T1 cDNA. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CAMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli. T1=T1 cDNA from Tubastrea sp. (SEQ ID NO:201); TSSU=chloroplast transit peptide from the small subunit of RBCase from Nicotiana sylvestris. Selected restriction endonuclease recognition sites are also marked. Refer to Example 11 for further details.

FIG. 34 is a diagrammatic representation of the binary plasmid pCGP2787 which is comprised of the ˜0.2 kb chloroplast transit peptide from the small subunit of ribulose biphosphate carboxylase (RBCase) from Nicotiana sylvestris inserted into the Rose CHS expression cassette of binary vector pCGP2782 (FIG. 27), upstream of the T1 cDNA. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; rCHS=rose chalcone synthase promoter fragment; pVS1=a broad host range origin of replication from a plasmid from Pseuodomonas aeruginosa; pACYC ori=modified replicon from pACYC184 from E. coli. T1=T1 cDNA from Tubastrea sp. (SEQ ID NO:201); TSSU=chloroplast transit peptide from the small subunit of RBCase from Nicotiana sylvestris. Selected restriction endonuclease recognition sites are also marked. Refer to Example 11 for further details.

FIG. 35 is a diagrammatic representation of the plasmid pCGP3257, which is comprised of the basic chitinase N-terminal endoplasmic reticulum (ER) transit peptide signal sequence from Arabidopsis thaliana inserted into the CaMV 35S expression cassette of binary vector pCGP2780 (FIG. 30), downstream of the CaMV 35S promoter. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; ERT=ER transit peptide signal sequence from Arabidopsis basic chitinase gene. Selected restriction endonuclease recognition sites are also marked. Refer to Example 11 for further details.

FIG. 36 is a diagrammatic representation of the binary plasmid pCGP3259. The T1 cDNA from Tubastrea sp. (SEQ ID NO:201)with an in-frame HDEL peptide sequence at the 3′ end was cloned into the CaMV 35S expression cassette of binary vector pCGP3257 (FIG. 35), downstream of the ER transit-peptide signal sequence from Arabidopsis thaliana. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa: pACYC ori=modified replicon from pACYC 184 from E. coli; ERT:T1:HDEL=T1 cDNA clone from Tubastrea (SEQ ID NO:201) with an in-frame ER transit peptide sequence from Arabidopsis basic chitinase gene at the 5′ end and an HDEL ER retention sequence at the 3′ end. Selected restriction endonuclease recognition sites are also marked. Refer to Example 11 for further details.

FIG. 37 is a diagrammatic representation of the binary plasmid pCGP3262 which is comprised of the basic chitinase N-terminal endoplasmic reticulum (ER) transit peptide signal sequence from Arabidopsis thaliana inserted into the Rose CHS expression cassette of binary vector pCGP3255 (FIG. 26), downstream of the Rose CHS promoter. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; rCHS rose chalcone synthase promoter fragment; pVS1=a broad host range origin of replication from a plasmid from Pseuodomonas aeruginosa; pACYC ori=modified replicon from pACYC184 from E. coli; ERT=ER transit peptide signal sequence from Arabidopsis basic chitinase gene. Selected restriction endonuclease recognition sites are also marked. Refer to Example 11 for further details.

FIG. 38 is a diagrammatic representation of the binary plasmid pCGP3263. The T1 cDNA from Tubastrea sp. (SEQ ID NO:201) with an in-frame HDEL peptide sequence at the 3′ end was cloned into the Rose CHS expression cassette of binary vector pCGP3262 (FIG. 37), downstream of the ER transit-peptide signal sequence from Arabidopsis thaliana. Abbreviations are as follows: TetR=the tetracycline resistance gene; LB=left border; RB=right border; SuRB=the coding region and terminator sequence from the acetolactate synthase gene from tobacco; p35S a promoter region from the cauliflower mosaic virus (CAMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; ERT:T1:HDEL=T1 cDNA clone from Tubastrea (SEQ ID NO:201) with an in-frame ER transit peptide sequence from Arabidopsis basic chitinase gene at the 5′ end and an BDEL ER retention sequence at the 3′ end; rCHS=Rose chalcone synthase promoter fragment. Selected restriction endonuclease recognition sites are also marked. Refer to Example 11 for further details.

FIG. 39 is a diagrammatic representation of the binary plasmid pCGP3258. An in-frame fusion of the T1 coding sequence (SEQ ID NO:201) and the mgfp4 sequence was cloned into the CaMV 35S expression cassette of pCGP3257 (FIG. 35). Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; T1:mgfp4=T1 cDNA clone from Tubastrea (SEQ ID NO:201) with an in-frame fusion of the mgfp4 coding sequence. Selected restriction endonuclease recognition sites are also marked. Refer to Example 12 for further details.

FIG. 40 is a representation of an autoradiograph of an RNA blot probed with ³²P-labelled fragments of (A) a 0.7 kb BamHI/HindIII fragment of the T1 clone contained in pCGP2921 (FIG. 10) and (B) 0.8 kb HindIII fragment of SuRB contained in pCGP1651. Each lane contained a 5 to 10 μg sample of total RNA isolated from the leaves and petals of transgenic P. hybrida plants. (C) Ethidium bromide staining of the 18S rRNA is shown as an indication of RNA loading levels. Lane numbers are marked 1 to 12. The numbers above the lane numbers refer to construct pCGP numbers used in the transformation experiments. Refer to Example 15 for further details.

FIG. 41 is a representation of an autoradiograph of an RNA blot probed with 32p labelled fragments of (A) a 0.7 kb BamHI/HindIII fragment of the T1 clone contained in pCGP2921 (FIG. 10) and (B) 0.8 kb HindIII fragment of SuRB contained in pCGP1651. Each lane contained a 5 μg sample of total RNA isolated from the leaves of non-transgenic and transgenic A. thaliana plants. (C) Ethidium bromide staining of the 25S rRNA is shown as an indication of RNA loading levels. Lane numbers are marked 1 to 17. The numbers above the lane numbers refer to construct pCGP numbers used in the transformation experiments with the exception of NTG and 35Smgfp4. NTG=non transgenic; 35Smgfp4=pBIN35Smgfp4. Refer to Example 14 for further details.

FIG. 42 is a graphical representation of absorption, excitation and emission spectra for Rtms-5 (SEQ ID NO: 166) and its variants. (A) Absorption spectra for Rtms-5 (SEQ ID NO:166); (B) Absorption spectra for variants generated via site directed mutagenesis: Rtms5-H142S and Rtms-5v (SEQ ID NO:216); C Excitation (exc) and emission (em) spectra for Rtms5-H142S and Rtms-5v (SEQ ID NO:216) at wavelengths indicated.

FIG. 43 is a graphical representation of examples of excitation and emission spectra for two other colored proteins, showing extinction coefficients (ε_{λmax}) based on the method of Whitaker and Granum (1980, supra) for calculating protein concentration. x-axis=relative absorption; y-axis=wavelength (nm); (A) Aims4 (SEQ ID NO:90)-H142S, and (B) Rtms-1 (SEQ ID NO:162)-N142S; λmax for each spectrum is shown on the figure.

FIG. 44 is a diagrammatic representation of the binary plasmid pCGP2926. A ˜0.1 kb AscI/BamHI fragment (containing sequences to a prokaryotic ribosome binding site (RBS), translational initiation consensus sequence (TICS) and an RGSHHHHHH epitope) generated by ligating the primers TICS-His-FWD (SEQ ID NO:227) and TICS-His-REV (SEQ ID NO:228) was introduced into the binary plasmid pCGP2781 (FIG. 32). Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli. T1=T1 cDNA from Tubastrea sp. (SEQ ID NO:201), His=RGSHHHHHH epitope. Selected restriction endonuclease recognition sites are also marked. Refer to Example 9 for further details.

FIG. 45 is diagrammatic representation of the binary plasmid pCGP3261. An ER targeted T1:mGFP4 fusion was cloned into CaMV 35S expression cassette of the binary vector pCGP3257. Abbreviations are as follows: TetR=the tetracycline resistance gene; LB=left border, RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; ERT:T1:mGFP4:HDEL=T1 cDNA clone from Tubastrea (SEQ ID NO:201):mGFP4 in-frame fusion with an in-frame ER transit peptide sequence from Arabidopsis basic chitinase gene at the 5′ end and an HDEL ER retention sequence at the 3′ end. Selected restriction endonuclease recognition sites are also marked. Refer to Example 12 for further details.

FIG. 46 is diagrammatic representation of the binary plasmid pCGP3260. An ER targeted mGFP4 coding region was cloned into CaMV 35S expression cassette of the binary vector pCGP2780. Abbreviations are as follows: TetR=the tetracycline resistance gene; LB=left border; RB=right border; SuRB=the coding region and terminator sequence from the acetolactate synthase gene from tobacco; p35S=a promoter region from the cauliflower mosaic virus (CaMV) 35S gene; t35S=a terminator fragment from the CaMV 35S gene; pVS1=a broad host range origin of replication from a plasmid from Pseuodomonas aeruginosa; pACYC ori=modified replicon from pACYC184 from E. coli; ERT:mGFP4:HDEL=mGFP4 coding sequence with an in-frame ER transit peptide sequence from Arabidopsis basic chitinase gene at the 5′ end and an HDEL ER retention sequence at the 3′ end. Selected restriction endonuclease recognition sites are also marked. Refer to Example 12 for further details.

FIG. 47 is a photographic representation of clear nature gel electrophoresis showing separation of fluorescently labeled mitochondrial ATP synthase. 1. b-gfp fusion protein; 2. b-Rtms-5v fusion protein; 3. b-dsRed fusion protein; 4. GFP not fused to another protein.

A summary of sequence identifiers used throughout the subject specification is provided in Table 1.

TABLE 1

SUMMARY OF SEQUENCE IDENTIFIERS

SEQ
ID

| | | |
|---|---|---|
| NO. | NAME | DESCRIPTION |

| | | |
|---|---|---|
| 1 | POC FOR | oligonucleotide |
| 2 | POC 220 | oligonucleotide |
| 3 | MSVIAT FOR | oligonucleotide |
| 4 | POC 231 | oligonucleotide |
| 5 | SVIAK | N-terminal amino acid sequence of a CFM |
| 6 | (M)SVIAT | N-terminal amino acid sequence of a CFM |
| 7 | SGIAT | N-terminal amino acid sequence of a CFM |
| 8 | SVIVT | N-terminal amino acid sequence of a CFM |
| 9 | SVSAT | N-terminal amino acid sequence of a CFM |
| 10 | SVIATQMTYKVYMSGT | N-terminal amino acid sequence of a CFM |
| 11 | SVIATQMTYKVYMSPT | N-terminal amino acid sequence of a CFM |
| 12 | SVIATQVTYKVYMSGT | N-terminal amino acid sequence of a CFM |
| 13 | SGIATQMTYKVYMSGT | N-terminal amino acid sequence of a CFM |
| 14 | SVIVTQMTYKVYMSGT | N-terminal amino acid sequence of a CFM |
| 15 | SVSATQMTYKVYMSGT | N-terminal amino acid sequence of a CFM |
| 16 | SVIAKQMTYKVNMSGT | N-terminal amino acid sequence of a CFM |
| 17 | SVIAKQMTYKVYMSDT | N-terminal amino acid sequence of a CFM |
| 18 | SVIAKQMTYX₁X₂YX₃SGT | N-terminal amino acid sequence of a CFM |
| 19 | Aasv-1 | nucleotide sequence of SVIAK-type clone |
| 20 | Aasv-1.pep | translated amino acid sequence of SVIAK CFM |
| 21 | Aasv-3 | nucleotide sequence of SVIAK-type clone |
| 22 | Aasv-3.pep | translated amino acid sequence of SVIAK CFM |
| 23 | Aasv-P | nucleotide sequence of SVIAK-type clone |
| 24 | Aasv-P.pep | translated amino acid sequence of SVIAK CFM |
| 25 | Acasv-A | nucleotide sequence of SVIAK-type clone |
| 26 | Acasv-A.pep | translated amino acid sequence of SVIAK CFM |
| 27 | Acasv-C | nucleotide sequence of SVIAK-type clone |
| 28 | Acasv-C.pep | translated amino acid sequence of SVIAK CFM |
| 29 | Acasv-D | nucleotide sequence of SVIAK-type clone |
| 30 | Acasv-D.pep | translated amino acid sequence of SVIAK CFM |
| 31 | Ce61-3sv | nucleotide sequence of SVIAK-type clone |
| 32 | Ce61-3sv.pep | translated amino acid sequence of SVIAK CFM |
| 33 | Ce61-4sv | nucleotide sequence of SVIAK-type clone |
| 34 | Ce61-4sv.pep | translated amino acid sequence of SVIAK CFM |
| 35 | Ce61-5sv | nucleotide sequence of SVIAK-type clone |
| 36 | Ce61-5sv.pep | translated amino acid sequence of SVIAK CFM |
| 37 | Ce61-7sv | nucleotide sequence of SVIAK-type clone |
| 38 | Ce61-7sv.pep | translated amino acid sequence of SVIAK CFM |
| 39 | GPd58-2sv | nucleotide sequence of SVIAK-type clone |
| 40 | GPd58-2sv.pep | translated amino acid sequence of SVIAK CFM |
| 41 | LGAsv-A | nucleotide sequence of SVIAK-type clone |
| 42 | LGAsv-A.pep | translated amino acid sequence of SVIAK CFM |
| 43 | LGAsv-C | nucleotide sequence of SVIAK-type clone |
| 44 | LGAsv-C.pep | translated amino acid sequence of SVIAK CFM |
| 45 | LGAsv-D | nucleotide sequence of SVIAK-type clone |
| 46 | LGAsv-D.pep | translated amino acid sequence of SVIAK CFM |
| 47 | LGAsv-E | nucleotide sequence of SVIAK-type clone |
| 48 | LGAsv-E.pep | translated amino acid sequence of SVIAK CFM |
| 49 | Misv-A | nucleotide sequence of SVIAK-type clone |
| 50 | Misv-A.pep | translated amino acid sequence of SVIAK CFM |
| 51 | Misv-B | nucleotide sequence of SVIAK-type clone |
| 52 | Misv-B.pep | translated amino acid sequence of SVIAK CFM |
| 53 | Misv-F | nucleotide sequence of SVIAK-type clone |
| 54 | Misv-F.pep | translated amino acid sequence of SVIAK CFM |
| 55 | PM1Asv-rep | nucleotide sequence of SVIAK-type clone |
| 56 | PM1Asv-rep.pep | translated amino acid sequence of SVIAK CFM |
| 57 | PM1Csv-rep | nucleotide sequence of SVIAK-type clone |
| 58 | PM1Csv-rep.pep | translated amino acid sequence of SVIAK CFM |
| 59 | PMsv-4 | nucleotide sequence of SVIAK-type clone |
| 60 | PMsv-4.pep | translated amino acid sequence of SVIAK CFM |
| 61 | PMsv-5 | nucleotide sequence of SVIAK-type clone |
| 62 | PMsv-5.pep | translated amino acid sequence of SVIAK CFM |
| 63 | PPsv-1 | nucleotide sequence of SVIAK-type clone |
| 64 | PPsv-1.pep | translated amino acid sequence of SVIAK CFM |
| 65 | PPsv-2 | nucleotide sequence of SVIAK-type clone |
| 66 | PPsv-2.pep | translated amino acid sequence of SVIAK CFM |
| 67 | PPsv-3 | nucleotide sequence of SVIAK-type clone |
| 68 | PPsv-3.pep | translated amino acid sequence of SVIAK CFM |
| 69 | PPsv-4 | nucleotide sequence of SVIAK-type clone |
| 70 | PPsv-4.pep | translated amino acid sequence of SVIAK CFM |
| 71 | PPsv-5 | nucleotide sequence of SVIAK-type clone |
| 72 | PPsv-5.pep | translated amino acid sequence of SVIAK CFM |
| 73 | PPsv-6 | nucleotide sequence of SVIAK-type clone |
| 74 | PPsv-6.pep | translated amino acid sequence of SVIAK CFM |
| 75 | Pavsv-A | nucleotide sequence of SVIAK-type clone |
| 76 | Pavsv-A.pep | translated amino acid sequence of SVIAK CFM |
| 77 | Pavsv-B | nucleotide sequence of SVIAK-type clone |
| 78 | Pavsv-B.pep | translated amino acid sequence of SVIAK CFM |
| 79 | Pavsv-C | nucleotide sequence of SVIAK-type clone |
| 80 | Pavsv-C.pep | translated amino acid sequence of SVIAK CFM |
| 81 | RTsv-1 | nucleotide sequence of SVIAK-type clone |
| 82 | RTsv-1.pep | translated amino acid sequence of SVIAK CFM |
| 83 | RTsv-2 | nucleotide sequence of SVIAK-type clone |
| 84 | RTsv-2.pep | translated amino acid sequence of SVIAK CFM |
| 85 | RTsv-3 | nucleotide sequence of SVIAK-type clone |
| 86 | RTsv-3.pep | translated amino acid sequence of SVIAK CFM |
| 87 | Aams-2 | nucleotide sequence of (M)SVIAK-type clone |
| 88 | Aams-2.pep | translated amino acid sequence of (M)SVIAT CFM |
| 89 | Aams-4 | nucleotide sequence of (M)SVIAT-type clone |
| 90 | Aams-4.pep | translated amino acid sequence of (M)SVIAK CFM |
| 91 | Aams-5 | nucleotide sequence of SGIAT-type clone |
| 92 | Aams-5.pep | translated amino acid sequence of SGIAT CFM |
| 93 | Aams-6 | nucleotide sequence of (M)SVIAT-type clone |
| 94 | Aams-6.pep | translated amino acid sequence of (M)SVIAT CFM |
| 95 | Aams-A | nucleotide sequence of (M)SVIAT-type clone |
| 96 | Aams-A.pep | translated amino acid sequence of (M)SVIAK CFM |
| 97 | Aams-B | nucleotide sequence of (M)SVIAT-type clone |
| 98 | Aams-B.pep | translated amino acid sequence of (M)SVIA5 CFM |
| 99 | Acams-2 | nucleotide sequence of (M)SVIAT-type clone |
| 100 | Acams-2.pep | translated amino acid sequence of (M)SVIAT CFM |
| 101 | Acams-3 | nucleotide sequence of (M)SVIAT-type clone |
| 102 | Acams-3.pep | translated amino acid sequence of (M)SVIAT CFM |
| 103 | Acams-4 | nucleotide sequence of (M)SVIAT-type clone |
| 104 | Acams-4.pep | translated amino acid sequence of (M)SVIAT CFM |
| 105 | Acams-5 | nucleotide sequence of (M)SVIAT-type clone |
| 106 | Acams-5.pep | translated amino acid sequence of (M)SVIAT CFM |
| 107 | Cems-F | nucleotide sequence of (M)SVIAK-type clone |
| 108 | Cems-F.pep | translated amino acid sequence of (M)SVIAT CFM |
| 109 | Cems-G | nucleotide sequence of (M)SVIAT-type clone |
| 110 | Cems-G.pep | translated amino acid sequence of (M)SVIAT CFM |
| 111 | Cems-H | nucleotide sequence of (M)SVIAT-type clone |
| 112 | Cems-H.pep | translated amino acid sequence of (M)SVIAT CFM |
| 113 | Cems-I | nucleotide sequence of (M)SVIAT-type clone |
| 114 | Cems-I.pep | translated amino acid sequence of (M)SVIAT CFM |
| 115 | LGAms-5 | nucleotide sequence of (M)SVIAT-type clone |
| 116 | LGAms-5.pep | translated amino acid sequence of (M)SVIAT CFM |
| 117 | LGAms-6 | nucleotide sequence of (M)SVIAT-type clone |
| 118 | LGAms-6.pep | translated amino acid sequence of (M)SVIAT CFM |
| 119 | Mi68Dms | nucleotide sequence of (M)SVIAT-type clone |
| 120 | Mi68Dms.pep | translated amino acid sequence of (M)SVIAT CFM |
| 121 | Mims-A | nucleotide sequence of (M)SVIAT-type clone |
| 122 | Mims-A.pep | translated amino acid sequence of (M)SVIAK CFM |
| 123 | Mims-B | nucleotide sequence of (M)SVIAT-type clone |
| 124 | Mims-B.pep | translated amino acid sequence of (M)SVIAK CFM |
| 125 | Mims-C | nucleotide sequence of (M)SVIAT-type clone |
| 126 | Mims-C.pep | translated amino acid sequence of (M)SVIAT CFM |
| 127 | PMms-A | nucleotide sequence of (M)SVIAT-type clone |
| 128 | PMms-A.pep | translated amino acid sequence of (M)SVIAT CFM |
| 129 | PMms-B | nucleotide sequence of (M)SVIAK-type clone |
| 130 | PMms-B.pep | translated amino acid sequence of (M)SVIAT CFM |
| 131 | PMms-C | nucleotide sequence of (M)SVIAT-type clone |
| 132 | PMms-C.pep | translated amino acid sequence of (M)SVIAK CFM |
| 133 | PMms-D | nucleotide sequence of (M)SVIAT-type clone |
| 134 | PMms-D.pep | translated amino acid sequence of (M)SVIAK CFM |
| 135 | PMms-E | nucleotide sequence of (M)SVIAT-type clone |
| 136 | PMms-E.pep | translated amino acid sequence of (M)SVIAT CFM |
| 137 | PPd57-1ms | nucleotide sequence of (M)SVIAK-type clone |
| 138 | PPd57-1ms.pep | translated amino acid sequence of (M)SVIAT CFM |
| 139 | PPd57-2ms | nucleotide sequence of (M)SVIAT-type clone |
| 140 | PPd57-2ms.pep | translated amino acid sequence of (M)SVIAT CFM |
| 141 | PPd57-3 | nucleotide sequence of (M)SVIAK-type clone |
| 142 | PPd57-3.pep | translated amino acid sequence of (M)SVIAT CFM |
| 143 | PPd57-4ms | nucleotide sequence of (M)SVIAT-type clone |
| 144 | PPd57-4ms.pep | translated amino acid sequence of (M)SVIAT CFM |
| 145 | PPms-1 | nucleotide sequence of (M)SVIAK-type clone |
| 146 | PPms-1.pep | translated amino acid sequence of (M)SVIAT CFM |
| 147 | PPms-2 | nucleotide sequence of (M)SVIAK-type clone |
| 148 | PPms-2.pep | translated amino acid sequence of (M)SVIAT CFM |
| 149 | PPms-E | nucleotide sequence of (M)SVIAT-type clone |
| 150 | PPms-E.pep | translated amino acid sequence of (M)SVIAT CFM |
| 151 | PPms-G | nucleotide sequence of (M)SVIAK-type clone |
| 152 | PPms-G.pep | translated amino acid sequence of (M)SVIAK CFM |
| 153 | Pav5ms | nucleotide sequence of (M)SVIAT-type clone |
| 154 | Pav5ms.pep | translated amino acid sequence of (M)SVIAK CFM |
| 155 | Pavms-2 | nucleotide sequence of (M)SVIAT-type clone |
| 156 | Pavms-2.pep | translated amino acid sequence of (M)SVIAT CFM |
| 157 | Pavms-3 | nucleotide sequence of (M)SVIAT-type clone |
| 158 | Pavms-3.pep | translated amino acid sequence of (M)SVIAK CFM |
| 159 | Pavms-4 | nucleotide sequence of (M)SVIAT-type clone |
| 160 | Pavms-4.pep | translated amino acid sequence of (M)SVIAT CFM |
| 161 | RTms-1 | nucleotide sequence of (M)SVIAT-type clone |
| 162 | RTms-1.pep | translated amino acid sequence of (M)SVIAT CFM |
| 163 | RTms-2 | nucleotide sequence of SVSAT-type clone |
| 164 | RTms-2.pep | translated amino acid sequence of SVSAT CFM |
| 165 | RTms-5 | nucleotide sequence of (M)SVIAT-type clone |
| 166 | RTms-5.pep | translated amino acid sequence of (M)SVIAK CFM |
| 167 | RTms-6 | nucleotide sequence of SVIVT-type clone |
| 168 | RTms-6.pep | translated amino acid sequence of SVIVT CFM |
| 169 | Acasv-B | nucleotide sequence of SVIAK-type clone with a |
| | | stop codon at amino acid position 14 |
| 170 | Acasv-B.pep | translated amino acid sequence of SVIAK CFM |
| 171 | GPd58-1sv | nucleotide sequence of SVIAK-type clone with a |
| | | stop codon at amino acid position 14 |
| 172 | GPd58-1sv.pep | translated amino acid sequence of SVIAK CFM |
| 173 | GPd58-3sv | nucleotide sequence of SVIAK-type clone with a |
| | | stop codon at amino acid position 14 |
| 174 | GPd58-3sv.pep | translated amino acid sequence of SVIAK CFM |
| 175 | GPd58-4sv | nucleotide sequence of SVIAK-type clone with a |
| | | stop codon at amino acid position 14 |
| 176 | GPd58-4sv.pep | translated amino acid sequence of SVIAK CFM |
| 177 | Misv-D | nucleotide sequence of SVIAK-type clone with a |
| | | stop codon at amino acid position 14 |
| 178 | Misv-D.pep | translated amino acid sequence of SVIAK CFM |
| 179 | Pavsv-D | nucleotide sequence of SVIAK-type clone with a |
| | | stop codon at amino acid position 14 |
| 180 | Pavsv-D.pep | translated amino acid sequence of SVIAK CFM |
| 181 | Aapat-1 | amino acid sequence of coral protein disclosed in |
| | | WO00/46233 |
| 182 | Aapat-2 | amino acid sequence of coral protein disclosed in |
| | | WO00/46233 |
| 183 | dT(17)Ad2Ad1 | oligonucleotide |
| 184 | vispro-F1 | oligonucleotide |
| 185 | vispro-R1 | oligonucleotide |
| 186 | pQEprom | oligonucleotide |
| 187 | pQErev | oligonucleotide |
| 188 | Coral-R1 | oligonucleotide |
| 189 | A8 (pCGP2918) | nucleotide sequence of full-length cDNA clone |
| 190 | A8.aa | translated amino acid sequence of full-length cDNA |
| | | clone |
| 191 | D10 (pCGP2920) | nucleotide sequence of full-length cDNA clone |
| 192 | D10.aa | translated amino acid sequence of full-length cDNA |
| | | clone |
| 193 | S3 (pCGP2924) | nucleotide sequence of full-length cDNA clone |
| 194 | S3.aa | translated amino acid sequence of full-length cDNA |
| | | clone |
| 195 | T3 (pCGP2922) | nucleotide sequence of full-length cDNA clone |
| 196 | T3.aa | translated amino acid sequence of full-length cDNA |
| | | clone |
| 197 | D1 (pCGP2919) | nucleotide sequence of full-length cDNA clone |
| 198 | D1.aa | translated amino acid sequence of full-length cDNA |
| | | clone |
| 199 | S1 (pCGP2923) | nucleotide sequence of full-length cDNA clone |
| 200 | S1.aa | translated amino acid sequence of full-length cDNA |
| | | clone |
| 201 | T1 (pCGP2921) | nucleotide sequence of full-length cDNA clone |
| 202 | T1.aa | translated amino acid sequence of full-length cDNA |
| | | clone |
| 203 | Kpn.6His.F | oligonucleotide |
| 204 | T1/A8.Sal.R | oligonucleotide |
| 205 | TSSU-Fnew | oligonucleotide |
| 206 | TSSU-R | oligonucleotide |
| 207 | AscI-ER.F | oligonucleotide |
| 208 | ER-BamHI.R | oligonucleotide |
| 209 | CP-HDEL-PacI.R | oligonucleotide |
| 210 | Pst-mGFP4F | oligonucleotide |
| 211 | mGFP4-PacIR | oligonucleotide |
| 212 | visproF1-new | oligonucleotide |
| 213 | MSV-RBS | oligonucleotide |
| 214 | SVIAK-RBS | oligonucleotide |
| 215 | POC 220 H6 | oligonucleotide |
| 216 | Rtms-5v | mutated variant amino acid sequence from Rtms-5 |
| | | (SEQ ID NO:166) |
| 217 | gtCP | translated amino acid sequence of SVIAK CFM |
| 218 | poc4 | translated amino acid sequence of SVIAK CFM |
| 219 | baspoc3 | translated amino acid sequence of SVIAK CFM |
| 220 | dsFP593 | translated amino acid sequence of a CFM |
| 221 | drFP583, also known as | translated amino acid sequence of a CFM |
| | dsRed583 | |
| 222 | gfp | translated amino acid sequence of a CFM |
| 223 | MGFP-4 | nucleotide sequence from GFP-4 from Aequorea |
| | | victoria (jelly fish), mutated for plants |
| 224 | MGFP-4.pep | translated amino acid sequence of GFP-4 CFM |
| 225 | BASPOC4 | translated amino acid sequence of a CFM |
| 226 | AsFP595 | translated amino acid sequence of a CFM |
| 227 | TICS-His-FWD | oligonucleotide |
| 228 | TICS-His-REV | oligonucleotide |
| 229 | mGFP4-HDEL-PacR | oligonucleotide |
| 230 | T1.N-QN(AAT)SQ(CAG) | oligonucleotide |
| 231 | T1.S-IS(TCC) > I(ATC) | oligonucleotide |
| 232 | YGFP3UP | oligonucleotide |
| 233 | YGFP3DO | oligonucleotide |
| 234 | RFPUP1 | oligonucleotide |
| 235 | RFPDO1 | oligonucleotide |
| 236 | MSVIATUP | oligonucleotide |
| 237 | COFPDO | oligonucleotide |
| 238 | ATP4PROMUP2 | oligonucleotide |
| 239 | ATP4DO2 | oligonucleotide |
| 240 | ATP7TUP | oligonucleotide |
| 241 | ATP7TDO | oligonucleotide |
| 242 | SPPDYTLEFP | N-terminal amino acid sequence of a CFM |
| 243 | SPPDYTLERP | N-terminal amino acid sequence of a CFM |
| 244 | (D)SS(P)E | N-terminal amino acid sequence of a CFM |
| 245 | SYLPN | N-terminal amino acid sequence of a CFM |
| 246 | SYLQN | N-terminal amino acid sequence of a CFM |
| 247 | MEGIVNG-A | oligonucleotide |
| 248 | MEGIVNG-T | oligonucleotide |
| 249 | MEGIVNG-C | oligonucleotide |
| 250 | REV-MEG-T | oligonucleotide |
| 251 | REV-MEG-C | oligonucleotide |

### DETAILED DESCRIPTION OF THE PREFERRED EMBODIMENTS

The present invention is predicated on the identification of peptides, polypeptides and proteins having one or more amino acid sequences or one or more amino acid sequences which exhibit color-facilitating properties, either on their own or following interaction with one or more other amino acids and nucleic acid moleclues encoding same. Such peptides, polypeptides and proteins are referred to herein as “color-facilitating molecules” or “CFMs”. The present invention contemplates a range of uses of CFMs, including their use as color expression markers and as color intensifiers, as well as in gel-like formulations for use as photon traps and in light-filtering formulations such as topically-applied sun creens.

The present invention further contemplates the use of genetic material encoding CFMs to generate eukaryotic or prokaryotic cells or eukaryotic or prokaryotic cell tissue which, in the presence of the CFMs, exhibit altered visual characteristics to the human eye in the absence of excitation of the CFMs by extraneous non-white light or particle emission.

Such altered visual characteristics are also referred to as being altered to the naked, unaided eye. Reference to “naked” or “unaided” is not to imply that the eye may not require magnification aids such as in the form of spectacles or glasses or a magnifying glass. Reference to extraneous light or particle emission includes ultraviolet (UV) light, blue laser light, plasma irradiation, γ-irradiation, particle irradiation, single wavelength light such as 340 nm, 382 nm, 396 nm, 405 nm, 475 nm, 490 nm, 575 nm or other forms of emission or particle bombardment. It does not include white light.

Accordingly, one aspect of the present invention provides an isolated nucleic acid molecule comprising a nucleotide sequence encoding a color-facilitating molecule (CFM) which, in a cell, alone or together with one or more other molecules imparts an altered visual characteristic to said cell when visualized by a human eye in the absence of excitation by extraneous non-white light or particle emission.

Preferably, the nucleic acid molecule is derived from Anemonia majano, Anemonia sulcata, Clavulania sp, Zoanthus sp, Discosoma sp (e.g. Discosoma striata), Aequorea sp (e.g. Aequorea Victoria), Anthozoa sp, Cassiopea sp, (e.g. Cassiopea xamachana), Millepora sp, Acropora sp (e.g. Acropora aspera and Acropora nobilis), Montipora sp, Porites murrayensis, Pocillopora damicormis, Pavona descussaca, Acanthastrea sp, Platygyra sp or Caulastrea sp.

In a preferred embodiment, the nucleic acid molecule encodes a CFM with an amino acid at its N-terminal region selected from SVLAK (SEQ ID NO:5), (M)SVIAT (SEQ ID NO:6), SGIAT (SEQ ID NO:7), SVIVT (SEQ ID NO:8) or SVSAT (SEQ ID NO:9). Even more particularly, the CFM comprises an amino acid sequence selected from SVIAT QMTY KVYM SGT (SEQ ID NO:10), SVIAT QMTY KVYM PEG (SEQ ID NO:11), SVIAT QVTY KVYM SGT (SEQ ID NO:12), SGIAT QMTY KVYM SGT (SEQ ID NO:13), SVIVT QMTY KVYM SGT (SEQ ID NO:14), SVSAT QMTY KVYM SGT (SEQ ID NO:15), SVIAK QMTY KVNM SGT (SEQ ID NO:16), SVIAK QMTY KVYM SDT (SEQ ID NO:17) and SVIAK QMTY X₁X₂YX₃ SGT (SEQ ID NO:18) wherein X₁, X₂ and X₃ may be any amino acid provided that X₁ is not K; X₂ is not V; X₃ is not M.

In a particular embodiment, the present invention provides an isolated nucleic acid molecule comprising a nucleotide sequence encoding a CFM or a fragment, variant or derivative thereof, wherein said isolated nucleic acid molecule comprises a nucleotide sequence selected from the group consisting of SEQ ID NOs:19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 189, 191, 193, 195, 197, 199 and 201, or a biologically active fragment or derivative of these.

Particular preferred nucleic acid molecules comprise the nucleotide sequences set forth in SEQ ID NOs:189, 191, 193, 195, 197, 199 and 201.

The nucleic acid molecule is regarded as genetic material and generally comprises a coding region encoding a CFM optionally operably linked to a single or multiple promoters. In one embodiment, the nucleic acid molecule is a genetic construct under the control of (i.e. operably linked to) a single promoter. In another embodiment, the genetic construct is a bicistronic, tricistronic or multicistronic construct carrying the gene encoding the CFM and optionally other genes such as encoding a reporter molecule.

As used herein, the terms “nucleic acid molecule” including “genetic material” refers to any single-stranded or double-stranded nucleic acid molecule which at least comprises deoxyribonucleotides and/or ribonucleotides, including DNA (cDNA or genomic DNA), RNA, mRNA, or tRNA, amongst others. The combination of such molecules with non-nucleotide substituents derived from synthetic means or naturally-occurring sources is also contemplated by the present invention. Genetic material may also include sequences optimized for expression of codons in a particular host cell.

The present invention extends to derivatives of the nucleic acid molecules and such derivatives includes any isolated nucleic acid molecule which comprises at least 10 and preferably at least 20 contiguous nucleotides derived from the genetic sequence as described herein according to any embodiment. A derivative includes a part, fragment, portion or analog. A derivative also includes a fusion molecule between two or more genetic sequences encoding CFMs.

The present invention also comprises analogs of the nucleic acid molecules. An “analog” means any isolated nucleic acid molecule which is substantially the same as a nucleic acid molecule of the present invention or its complementary nucleotide sequence as described herein according to any embodiment, notwithstanding the occurrence of any non-nucleotide constituents not normally present in said isolated nucleic acid molecule, for example carbohydrates, radiochemicals including radionucleotides, reporter molecules such as, but not limited to, alkaline phosphatase or horseradish peroxidase, amongst others. A “homolog” is a functionally similar molecule from a different species or strain.

Generally, analogs or derivatives of the nucleic acid molecule of the invention are produced by synthetic means or alternatively, derived from naturally-occurring sources. For example, the nucleotide sequence of the present invention may be subjected to mutagenesis to produce single or multiple nucleotide substitutions, deletions and/or insertions. A derivative encompasses a nucleotide sequence modified for optimized or enhanced codon usage in a particular cell.

The genetic sequence of the present invention may comprise a sequence of nucleotides or be complementary to a sequence of nucleotides which comprise one or more of the following: a promoter sequence, a 5′ non-coding region, a cis-regulatory region such as a functional binding site for transcriptional regulatory protein or translational regulatory protein, an upstream activator sequence, an enhancer element, a silencer element, a TATA box motif, a CCAAT box motif, or an upstream open reading frame, transcriptional start site, translational start site, and/or nucleotide sequence which encodes a leader sequence. The genetic sequence also encodes the CFM.

The term “5′ non-coding region” is used herein in its broadest context to include all nucleotide sequences which are derived from the upstream region of an expressible gene, other than those sequences which encode amino acid residues which comprise the polypeptide product of said gene, wherein 5′ non-coding region confers or activates or otherwise facilitates, at least in part, expression of the gene.

The nucleic acid molecule may also be regarded as a gene. The term “gene” is used in its broadest context to include both a genomic DNA region corresponding to the gene as well as a cDNA sequence corresponding to exons or a recombinant molecule engineered to encode a functional form of a product. The term “gene” is used in its broadest sense and includes cDNA corresponding to the exons of a gene. Accordingly, reference herein to a “gene” is to be taken to include:-
(i) a classical genomic gene consisting of transcriptional and/or translational regulatory sequences and/or a coding region and/or non-translated sequences (i.e. introns, 5′- and 3′- untranslated sequences); or
(ii) mRNA or cDNA corresponding to the coding regions (i.e. exons) and 5′- and 3′-untranslated sequences of the gene.

The term “gene” is also used to describe synthetic or fusion molecules encoding all or part of a functional product.

As used herein, the term “cis-acting sequence” or “cis-regulatory region” or similar term shall be taken to mean any sequence of nucleotides which is derived from an expressible genetic sequence wherein the expression of the first genetic sequence is regulated, at least in part, by said sequence of nucleotides. Those skilled in the art will be aware that a cis-regulatory region may be capable of activating, silencing, enhancing, repressing or otherwise altering the level of expression and/or cell-type-specificity and/or developmental specificity of any structural gene sequence.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene.

In the present context, the term “promoter” is also used to describe a synthetic or fusion molecule, or derivative which confers, activates or enhances expression of a structural gene or other nucleic acid molecule, in a plant cell. Preferred promoters according to the subject invention may contain additional copies of one or more specific regulatory elements to further enhance expression in a cell, and/or to alter the timing of expression of a structural gene to which it is operably connected.

In a preferred embodiment, the nucleic acid molecules are expressed in a cell. The cell may be a eukaryotic or prokaryotic cell. Reference to a eukaryotic cell includes a mammalian animal cell, a non-mammalian animal cell or a plant cell. Insofar as the eukaryotic cell is a plant cell, the plant cell may be part of a plant callus or a whole plant. Reference to a “plant” includes ornamental or flowering plants or parts thereof such as flowers, roots, leaves, stems, seeds, fruit or fibers. Particularly preferred plant cells are those selected from rose, carnation, lisianthus, petunia, lily, tulip, pansy, gerbera or chrysanthemum.

The CFM is preferably a GFP or a derivative or homolog thereof such as a non-fluorescent GFP homolog.

Another aspect of the present invention provides an isolated color-facilitating molecule (Cow) comprising a polypeptide which, in a cell, alone or together with one or more other molecules imparts an altered visual characteristic to said cell when visualized by a human eye in the absence of excitation by extraneous non-white light or particle emission.

The CFM of the present invention is preferably a protein comprising a sequence of amino acids such that upon folding, the sequence alone or following interaction with one or more other amino acids which may be within the same molecule or in another molecule such as in a dimer, trimer or oligomer exhibits chromophore or fluorophore properties. Particularly useful proteins comprise the contiguous amino acid sequence Gln-Tyr-Gly (QYG). Even more preferably, the protein is a GFP or a homolog or derivative thereof An example of a homolog of a GFP is a non-fluorescent GFP homolog. An example of a derivative of a GFP or non-fluorescent GFP homolog is a GFP modified to cause a shift in the ratio of excitation or emission peaks. Such modifications may result in a more intense fluorescence or may exhibit altered or weaker fluorescence.

Any number of GFP or non-fluorescent GFP homologs or other derivatives may be employed as CFMs in accordance with the present invention. Examples of such molecules are from Anemonia majano, Anemonia sulcata, Clavularia sp, Zoanthus sp, Discosoma sp (e.g. Discosoma striata), Aequorea sp (e.g. Aequorea victoria), Anthozoa sp, Cassiopea sp, (e.g. Cassiopea xamachana), Millepora sp, Acropora sp (e.g. Acropora aspera and Acropora nobilis), Montipora sp, Porites murrayensis, Pocillopora damicormis, Pavona descussaca, Acanthastrea sp, Platygyra sp and Caulastrea sp.

Particularly preferred protein sequences which constitute CFMs of the present invention comprise one of the following sequences of amino acids towards the amino-terminal end of the polypeptide: “SVIAK” (SEQ ID NO:5), “(M)SVIAT” (SEQ ID NO:6), “SGIAT” (SEQ ID NO:7), “SVIVT” (SEQ ID NO:8), or “SVSAT” (SEQ ID NO:9).

Examples of such preferred protein sequences may be selected from the group consisting of:

| | |
|---|---|
| SVIAT QMTY KVYM SGT; | (SEQ ID NO:10) |
| SVIAT QMTY KVYM PGT; | (SEQ ID NO:11) |
| SVIAT QVTY KVYM SGT; | (SEQ ID NO:12) |
| SGIAT QMTY KVYM SGT; | (SEQ ID NO:13) |
| SVIVT QMTY KVYM SGT; | (SEQ ID NO:14) |
| SVSAT QMTY KVYM SGT; | (SEQ ID NO:15) |
| SVIAK QMTY KVNM SGT; | (SEQ ID NO:16) |
| SVIAK QMTY KVYM SDT; | (SEQ ID NO:17) |
| and | |
| SVIAK QMTY X₁X₂YX₃ SGT, | (SEQ ID NO:18) |

wherein X₁, X₂ and X₃ may be any amino acid provided that X₁ is not K; X₂ is not V; X₃ is not M.

Accordingly, in another aspect of the present invention there is provided an isolated polypeptide, or a biologically active fragment thereof, or a variant or derivative of these, said polypeptide comprising an amino acid sequence selected from the group consisting of SEQ ID NOs:10, 11, 12, 13, 14, 15, 16, 17 and 18, with the proviso that, in said isolated polypeptide or biologically active fragment or variant or derivative of SEQ ID NO: 18, X₁ is not lysine, X₂ is not valine, and X₃ is not methionine.

Particularly suitable molecules comprise an amino acid sequence selected from the group consisting of SEQ ID NOs:20, 22, 24, 26, 28, 30, 32, 34, 36, 38, 40, 42, 44, 46,48, 50, 52, 54, 56, 58, 60, 62, 64, 66, 68 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 190, 192, 194, 196, 198,200 and 202.

Accordingly, a preferred embodiment of the present invention provides an isolated polypeptide, or a biologically active fragment thereof, or a variant or derivative of these, said polypeptide comprising an amino acid sequence selected from the group consisting of SEQ ID NOs:20, 22, 24, 26, 28, 30; 32, 34, 36, 38, 40, 42, 44, 46, 48, 50, 52, 54, 56, 58, 60, 62, 64, 66, 68 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 190, 192, 194, 196, 198, 200 and 202 provided that, where the said biologically active fragment or variant or derivative comprises the sequence SVIAK QMTY X₁X₂YX₃ SGT, X₁ is not lysine, X₂ is not valine, and X₃ is not methionine.

Such isolated polypeptides, when present in a prokaryotic or eukaryotic cell or group of prokaryotic or eukaryotic cells such as in plant cells in the form of plant tissue or plant callus, may alone or in combination with one or more other molecules impart an altered visual characteristic to said cell or group of cells when visualized by a human eye in the absence of excitation by extraneous non-white light or particle emission.

Accordingly, another aspect of the present invention provides a prokaryotic or eukaryotic cell or group of prokaryotic or eukaryotic cells in the form of tissue wherein said cell or group of cells or their parent cells are genetically modified to enable the production of a color-facilitating molecule (CFM) which alone or together with one or more other molecules imparts an altered visual characteristic to said cell or group of cells when visualized by a human eye in the absence of excitation by extraneous non-white light or particle emission.

The CFM is as herein defined and in a preferred embodiment includes polypeptides having amino acid sequence selected from the list comprising SEQ ID NOs:20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 190, 192, 194, 196, 198, 200 and 202 provided that, where the said amino acid sequence comprises the sequence SVIAK QMTY X₁X₂YX₃ SGT, X₁ is not lysine, X₂ is not valine, and X₃ is not methionine.

A “eukaryotic” cell is regarded as any cell which is not characterized as being a “prokaryotic” cell. Particularly useful eukaryotic cells are plant cells as well as fungi and yeast. Other eukaryotic cells, however, are also contemplated such as mammalian cells, non-mammalian animal cells including insect cells as well as plant cells. A “plant” may be regarded as a monocotyledonous or dicotyledonous plant and includes ornamental and crop plants. Reference to “tissue” includes plant callus. A “prokaryotic cell” is generally a cell comprising a nucleus not surrounded by a nuclear membrane and includes bacteria and microbial cells. Such prokaryotic cells include Pseudomonas sp., E. coli, Enterobacter sp., Salmonella sp., Klebsiella sp., Acetobacter sp., Staphylocous sp., Streptococcus sp. or Bacillus sp., amongst many others.

In a preferred embodiment, the present invention provides a plant cell or group of plant cells such as in the form of plant tissue or plant callus wherein said plant cells or group of plant cells or their parent cells are genetically modified to enable production of a CFMA which alone or in combination with one or more other molecules imparts an altered visual characteristic to said cell or group of cells when visualized by a human eye in the absence of excitation by extraneous non-white light or particle emission.

Particularly preferred plants are ornamental and flowering plants. Particularly useful plants contemplated by the present invention include but are not limited to rose, carnation, lisianthus, petunia, lily, tulip, pansy, gerbera and chrysanthemum.

Reference herein to a “plant” includes parts of plants. Similarly, reference herein to “plant tissue” includes parts of plants. Examples of such plant parts, include but are not limited to, flowers, roots, leaves, stems, seeds, fruit and fibres. The term “flowers” includes parts of flowers such as petals, petioles, flower heads and flower buds. Plant tissue may also include callus material as well as embryogenic or non-embryogenic material. The term “fibre” includes cotton and hemp fibres.

Accordingly, another aspect of the present invention is directed to a plant or part of a plant including a flower, root, leaf, stem, seed, fruit or fibre or reproductive portion of said plant or cells of said plant wherein said plant or plant part comprises cells genetically modified to enable production of a CFM which alone or in combination with one or other molecules imparts an altered visual characteristic to said cells when visualized by a human eye in the absence of excitation by extraneous non-white light or particle emission.

The term “genetically modified” is used in its broadest sense and includes introducing gene(s) into cells, mutating gene(s) in cells and altering or modulating the regulation of gene(s) in cells.

A “part” of a plant includes flowers (e.g. cut or severed flowers), petals, stems, leaves and fibrous material such as cotton and vegetative, propagative and reproductive material such as cuttings, pollen, seeds and callus.

The altered visual characteristic may be exhibited by all cells in the plant or in selected tissue or plant parts such as flowers, roots, leaves, stems, seeds, fruit or fibres. The production of the CFM may, therefore, be tissue specific or tissue preferential. Furthermore, CFM production may be developmentally dependent, determined, influenced or otherwise regulated.

The CFM may be produced in the whole plant with the use of a constitutive promoter such as cauliflower mosaic virus (CaMV) 35S promoter, operably connected or operably linked to a gene or other nucleic acid molecule encoding the CFM. Alternatively, the molecule may be confined to, for example, petal tissue, epidermal cell layers of petals or to different organelles within cells. For example, a floral specific promoter such as a chalcone synthase promoter substantially limits a colored protein expression to flower petals.

The use of some gene promoters (e.g. 35S) may produce CFM accumulation in the cytoplasm of transformed cells and confer a visible color to the plant tissue. The CFM may be targeted to different organelles within the plant cell to confer a color change in the tissue visible to the naked unaided eye under white light illumination. The CFM can be targeted to plastids using a chloroplast transit peptide fused in-frame with the colored protein cDNA sequence. An example of a plasmid transit peptide that can be used is the transit peptide of the small subunit of ribulose-1,5-bisphosphate-carboxylase (e.g. InCheol et al., Molecular Breeding 5:453-461, 1999). The targeting of a CFM to plastids can dramatically increase the total amount of protein accumulated (InCheol et al., 1999, supra) and thereby increase color intensity.

Chromoplasts are numerous in the petals of some flowers, leaves and fruit. A chromoplast specific transit peptide fused in-frame with the protein cDNA sequence may be used to modify flower or other tissue color with a much reduced potential for interfering with total plant photosynthetic activity, as may occur if an constitutive promoter and a chloroplast transit peptide were used to target the CFM. The use of a chromoplast transit peptide and a floral specific promoter may be optimal for the modification of flower color.

It may be beneficial to target all CFMs to the vacuole or endoplasmic reticulum to avoid any detrimental effects to the transformed cells or plants. An example of an endoplasmic reticulum targeting peptide sequence that can be used is the amino acid sequence HDEL (Haseloff et al., 1997, supra). The CFM may also be targeted to the cell wall.

The term “operably connected” or “operably linked” in the present context means placing a structural gene (e.g. a nucleic acid molecule encoding a CFM) under the regulatory control of a promoter which then controls expression of the gene. Promoters and the like are generally positioned 5′ (upstream) to the genes which they control. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting, i.e., the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting, i.e., the genes from which it is derived.

The cells genetically modified to enable production of a CFM may be the cells into which genetic material has been introduced or they may represent progeny of genetically modified parent cells.

Accordingly, the present invention contemplates a method for generating a transgenic plant or part of a plant, wherein said plant or plant part comprises cells genetically modified to enable production of a CFM which alone or in combination with one or other molecules imparts an altered visual characteristic to said cells when visualized by a human eye in the absence of excitation by extraneous non-white light or particle emission, said method comprising introducing into said cells an isolated nucleic acid molecule encoding said CFM.

Preferably, the CFM is derived from Anemonia majano, Anemonia sulcata, Clavularia sp, Zoanthus sp, Discosoma sp (e.g. Discosoma striata), Aequorea sp (e.g. Aequorea victoria), Anthozoa sp, Cassiopea sp, (e.g. Cassiopea xamachana), Millepora sp, Acropora sp (e.g. Acropora aspera and Acropora nobilis), Montipora sp, Porites murrayensis, Pocillopora damicormis, Pavona descussaca, Acanthastrea sp, Platygyra sp or Caulastrea sp.

More preferably, the CFM comprises an amino acid sequence in its N-terminal end selected from SVIAK (SEQ ID NO:5), (M)SVIAT (SEQ ID NO:6), SGIAT (SEQ ID NO:7), SVIVT (SEQ ID NO: 8) or SVSAT (SEQ ID NO:9).

Even more preferably, the CFM comprises an amino acid sequence selected from the list comprising SVIAT QMTY KVYM SGT (SEQ ID NO:10), SVIAT QMTY KVYM PEG (SEQ ID NO: 11), SVIAT QVTY KVYM SGT (SEQ ID NO:12), SGIAT QMTY KVYM SGT (SEQ ID NO:13), SVIVT QMTY KVYM SGT (SEQ ID NO:14), SVSAT QMTY KVYM SGT (SEQ ID NO:15), SVIAK QMTY KVNM SGT (SEQ ID NO:16), SVIAK QMTY KVYM SDT (SEQ ID NO:17) and SVLAK QMTY X₁X₂YX₃ SGT (SEQ ID NO:18) wherein X₁, X₂ and X₃ may be any amino acid provided that X₁ is not K; X₂ is not V; X₃is not M.

Most preferably, the CFM is encoded by a nucleotide sequence selected from the list comprising SEQ ID NOs:19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115,117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 189, 191, 193, 195, 197, 199 and 201.

Another aspect of the present invention provides a transgenic plant wherein said plant or a part thereof such as a flower, leaf, root, stem, seed, fruit or fibre exhibits an altered visual characteristic to a human eye in the absence of extraneous non-white light or particle emission wherein cells of said transgenic plant or of a parent plant have been genetically modified to enable production of a CFM.

As stated above, the present invention extends to genetically modified mammalian cells, non-mammalian animal cells as well as plant cells.

Farmers use conventional breeding techniques to develop new colors in animals and animal products for the market, for example, colored wools and leathers or hides. Presently the main way of coloring these products toobtain novel colors is by using dyes or tints or paints or pigments on natural colored products. However, the use of the CFMs of the present invention can be employed to produce a transgenic animal which exhibits a novel color: for example, sheep with blue or red colored fleece, cows with red colored bide.

Specifically the CFM can be used in a range of agriculturally important animals such as but not limited to sheep, pigs, cattle, horses, goats, llamas, fish, ostriches, emus, ducks and chickens. Accordingly, another aspect of the present invention provides a transgenic mammalian or non-mammalian animal cell or transgenic non-human mammal or non- mammalian animal comprising said cells, said cells exhibiting an altered visual characteristic to a human eye in the absence of extraneous non-white light or particle emission wherein cells of said transgenic plant, mammal or animal or plant cells thereof have been genetically modified to enable production of a CFM.

The CFM is as herein defined. Production of the CFM may be constitutive or developmental or may be inducible in response to internal or external stimulus including stress.

Reference herein to a “color-facilitating molecule”, “CFM”, “protein”, “GFP” or “non-fluorescent GFP-homolog” includes fragments, derivatives, variants and homologs thereto. Examples of derivatives include mutants, parts, fragments and portions of these molecules including single or multiple amino acid substitutions, deletions and/or additions to the. molecules. Derivatives also include fusion molecules between two or more CFMs or between a CFM and another molecule such as a leader sequence, targeting sequence, expression-facilitating sequence and/or a reporter molecule capable of providing an identifiable signal.

As stated above a derivative also includes a modified form providing altered ratios of excitation or emission spectra. In addition, or as a consequence of the altered ratios of excitation or emission, the modified GFP or their homologs may have a more intense color-producing capacity relative to an unmodified molecule.

Furthermore, other proteins may be used in conjunction with the CFMs to alter the visual characteristics of the cells. Examples of other proteins include copper containing proteins containing one or more type 1 (CuII) motifs as found in the Fet3 protein from Saccharomyces cerevisiae (Hassett et al., Journal of Biological Chemistry 273:23274-23282, 1998) and other multinuclear copper ferroxidase enzymes such as laccase, ceruloplasmin and ascoibate oxidase (Messerschmidt and Huber, Eur. J. Biochem. 187:341-352, 1990). Similarly, the mononuclear blue or type 1 copper proteins (cupredoxins), such as plastocyanin, azurin, pseudoazurin, plantacyanin, rusticyanin, amicyanin, auracyanin and halocyanin (Nersissian et al., Protein Science 5:2184-2192, 1996). These proteins have not been associated with pigmentation in nature. However, when these proteins are concentrated an intense blue color is evident (Hassett et al., 1998, supra; Messerschmidt and Huber, 1990, supra). The over-expression of a type 1 (CuII) containing protein in flowers and other plant tissues under conditions that allow correct folding and acquisition of Cu ions can modify or impart a color visible to the naked unaided eye under white light. Reference to “in conjunction” includes reference to a fusion protein between a CFM and another protein such as a cuproprotein and well as the expression of nucleotide sequences in multicistronic form encoding a CFM and at least one other protein.

Another aspect of the present invention provides a eukaryotic or prokaryotic cell or a group of eukaryotic or prokaryotic cells in the form of a tissue wherein said cell or group of cells or their parent cells are genetically modified to produce a GFP or derivative or homolog thereof such as a non-fluorescent GFP homolog which imparts an altered visual characteristic on said cell or group of cells when visualized by a human eye in the absence of excitation by extraneous non-white light or particle emission.

Preferably, the eukaryotic cells are plant cells or plant tissue. The eukaryotic cells may, however, be mammalian cells or non-mammalian animal cells. Reference to “plant tissue” includes “callus”.

Accordingly, another aspect of the present invention is directed to a plant or part of a plant including a flower, root, leaf, stem, seed, fruit or fibre or reproductive portion of said plant or cells of said plant wherein said plant or plant part comprises cells genetically modified to enable production of a GFP or a derivative or homolog thereof such as a non-fluorescent GFP homolog which imparts an altered visual characteristic to said cells when visualized by a human eye in the absence of excitation by extraneous non-white light or particle emission.

A particularly preferred embodiment the present invention is directed to a plant or part of a plant including a flower, root, leaf, stem, seed, fruit or fibre or reproductive portion of said plant or cells of said plant wherein said plant or plant part comprises cells genetically modified to comprise a polynucleotide comprising the nucleotide sequence set forth in any one of SEQ ID NOs:19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 189, 191, 193, 195, 197, 199 or 201, or a derivative or homolog of any of these, thereby enabling production of a CFM which alone or in combination with one or more other molecules imparts an altered visual characteristic to said cell or group of cells when visualized by a human eye in the absence of excitation by extraneous non-white light or particle emission.

The present invention particularly provides, in a preferred embodiment, a genetically modified plant carrying flowers having an altered flower color relative to a non-genetically modified plant as well as cut flowers from such a plant. Reference herein to a “genetically modified plant” includes progeny of a genetically modified plant as well as hybrids and derivatives of a genetically modified plant.

The altered coloration of eukaryotic cells such as plant cells is useful not only for the ornamental plant market but also as propriety tags, for example, of seeds, root stock, flowers, crops and whole plants and plant parts. This may be particularly important for distinguishing between transgenic and non-transgenic crops, plants and other horticultural products. Furthermore, the modification of visible color in cotton fibre or hemp is a usefull means of reducing the toxicity of dye processes in color fabric manufacture. The modification of visible color in edible and/or ornamental fungal species may also be used to differentiate and enhance marketability.

The modification of visible color in fruit and vegetables may be used to differentiate and enhance their marketability. A suitable gene promoter may be used to control the expression of the CFM to signal optimal time to, for example, harvest crop plants including harvesting plant parts such as flowers or seeds. In addition, a stress-inducible promoter may be utilized to promote an early warring of water or pathogen stress, allowing for early intervention by the grower and subsequent reduction in economic loss.

Other uses for the CFM of the present invention include, for example, the production of novel colored plant extracts wherein the extract includes, for example, a flavouring or food additive or health product or beverage or juice or coloring. Beverages may include but are not limited to wines, spirits, beers, teas, coffee, milk and dairy products.

The CFM may be used to alter the color of many products such as but not limited to foods (e.g. breads and yeast products, confectionery), beverages (see above) or novelty items (e.g. toys).

A further aspect of the present invention provides a transfected or transformed cell, tissue, organ or non-cellular material which contains or is capable of producing a CFM or a functional derivative or homolog thereof. Preferably, the CFM is a protein such as GFP or a non-fluorescent GFP-homolog.

The genetic construct(s) of the present invention may be introduced into a cell by various techniques known to those skilled in the art. The technique used may vary depending on the known successful techniques for that particular organism.

Techniques for introducing recombinant DNA into cells include, but are not limited to, transformation using CaCl₂ and variations thereof, direct DNA uptake into protoplasts, PEG-mediated uptake to protoplasts, microparticle bombardment, electroporation, microinjection of DNA, microparticle bombardment of tissue explants or cells, vacuum-infiltration of tissue with nucleic acid, and T-DNA-mediated transfer from Agrobacterium to the plant tissue.

For microparticle bombardment of cells, a microparticle is propelled into a cell to produce a transformed cell. Any suitable ballistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the genetic construct may incorporate a plasmid capable of replicating in the cell to be transformed.

Examples of microparticles suitable for use in such systems include 0.1 to 10 μm and more particularly 10.5 to 5 μm tungsten or gold spheres. The DNA construct may be deposited on the microparticle by any suitable technique, such as by precipitation.

Once introduced into cells such as plant tissue, the expression of a CFM may be assayed in a transient expression system or it may be determined after selection for stable integration within for example, the plant genome. Hence, a CFM of the present invention may be useful as an expression marker. For example, genetic material encoding a CFM of the present invention, optionally operably linked to a single or multiple promoters, may be introduced into cells as a fluorescent “tag”, optionally fused with one or more other nucleic acid sequences that may encode a polypeptide or a regulatory nucleotide sequence. In this manner, a CFM fused with another polypeptide may be useful in assessing subcellular localisation of the fusion or, alternatively, as an expression marker for assessing possible activity of the regulatory nucleotide sequence in a given host cell.

Host cells may be prokaryotic cells, for example bacterial, or eukaryotic cells, for example yeast, plant, and animal cells, including human. Preferred host cells are bacterial or plant.

Plant tissue capable of subsequent clonal propagation, whether by organogenesis or embryogenesis, may be transformed with a genetic construct of the present invention and a whole plant generated therefrom. The particular tissue chosen will vary depending on the clonal propagation systems available for, and best suited to, the particular species being transformed. Exemplary tissue targets include leaf disks, pollen, embryos, cotyledons, hypocotyls, megagametopbytes, callus tissue, existing meristernatic tissue (e.g. apical meristem, axillary buds, and root meristems), and induced meristem tissue (e.g. cotyledon meristem and bypocotyl meristem).

The regenerated transformed plants may be propagated by a variety of means, such as by clonal propagation or classical breeding techniques. For example, a first generation (or T1) transformed plant may be selfed to give homozygous second generation (or T2) transformant, and the T2 plants further propagated through classical breeding techniques.

Any number of GFP or non-fluorescent GFP-homologs may be employed provided that the GFP or its homolog or other CFM imparts on a cell or group of cells an altered visual characteristic to the human eye in the absence of extraneous non-white light or particle emission. Examples of CFMs contemplated herein include but are not limited to non-fluorescent GFP-homologs such as that encoded by asFP595 (Lukyanov et al., 2000, supra) and t7SP6BASPOC3 and T7SP6BASPOC4 (Hoegh-Guldberg and Dove, 2000, supra) and fluorescent GFP variants and homologs such as described in Davis and Vierstra, 1996, supra; Haseloff et al., 1997, supra; Lukyanoy et al., 1999, supra; Matz et al., 1999, supra; Fradkov et al., FEBS Letters 479:127-130, 2000).

Accordingly, another aspect of the present invention provides a eukaryotic or prokaryotic cell or group of eukaryotic or prokaryotic cells genetically modified to comprise:
(i) a nucleotide sequence set forth in SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ED NO:189 or SEQ ED NO:191 or SEQ ID NO:193 or SEQ ID NO:195 or SEQ ID NO:197 or SEQ ID NO:199 or 201;
(ii) a nucleotide sequence having at least about 60% similarity after optimal alignment to SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or 201;
(iii) a nucleotide sequence capable of hybridizing under low stringency conditions to SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO: 89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID No:111 or SEQ ID NO:113 or SEQ ID NO :115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or 201;
(iv) a nucleotide sequence capable of encoding the amino acid sequence set forth in SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:10l or SEQ ID NO:103 or SEQ ID NO:105 or SEQ ID NO:107 or SEQ ID NO:109 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or201;
(v) a nucleotide sequence capable of encoding an amino acid sequence having at least about 60% similarity after optimal alignment to SEQ ID NO :19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ED NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ED NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ED NO:197 or SEQ ID NO:199 or 201;
(vi) a nucleotide sequence capable of hybriding under low stringency conditions to the nucleotide sequence in (iv) or (v) or its complementary form;
   wherein said nucleotide sequences encode a CFM which imparts an altered visual characterization to said cell or group of cells to a human eye in the absence of extraneous non-white light or particle emission.

More particularly, the present invention provides a eukaryotic or prokaryotic cell or group of eukaryotic or prokaryotic cells genetically modified to comprise:
(i) a nucleotide sequence set forth in SEQ ID NO:189 or SEQ ID NO:191 or SEQ ID NO:193 or SEQ ID NO:195 or SEQ ID NO:197 or SEQ ID NO:199 or SEQ ID NO:201;
(ii) a nucleotide sequence having at least about 60% similarity after optimal alignment to SEQ ID NO:189 or SEQ ID NO:191 or SEQ ID NO:193 or SEQ ID NO:195 or SEQ ID NO:197 or SEQ ID NO:199 or SEQ ID NO:201;
(iii) a nucleotide sequence capable of hybridizing under low stringency conditions to SEQ ID NO:189 or SEQ ID NO:191 or SEQ ID NO:193 or SEQ ID NO:195 or SEQ ID NO:197 or SEQ ID NO:199 or SEQ ID NO:201 or its complementary form;
(iv) a nucleotide sequence capable of encoding the amino acid sequence set forth in SEQ ID NO:190 or SEQ ID NO:192 or SEQ ID NO:194 or SEQ ID NO:196 or SEQ ID NO:198 or SEQ ID NO:200 or SEQ ID NO:202;
(v) a nucleotide sequence capable of encoding an amino acid sequence having at least about 60% similarity after optimal alignment to SEQ ID NO:190 or SEQ ID NO:192 or SEQ ID NO:194 or SEQ ID NO:196 or SEQ ID NO:198 or SEQ ID NO:200 or SEQ ID NO:202;
(vi) a nucleotide sequence capable of hybridizing under low stringency conditions to the nucleotide sequence in (iv) or (v) or its complementary form;
   wherein said nucleotide sequences encode a CFM which imparts an altered visual characterization to said cell or group of cells to a human eye in the absence of extraneous non-white light or particle emission.

Preferably, the eukaryotic cells are plant cells.

Accordingly, in another aspect of the present invention, there is provided a plant or cells of a plant or parts of a plant or progeny of a plant wherein said plant comprises cells comprising:
(i) a nucleotide sequence set forth in SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or 201;
(ii) a nucleotide sequence having at least about 60% similarity after optimal alignment to SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO;137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or 201;
(iii) a nucleotide sequence capable of hybridizing under low stringency conditions to SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or 201;
(iv) a nucleotide sequence capable of encoding the amino acid sequence set forth in SEQ ID NO: 19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO:109 or SEQ ID NO:11 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ED NO:127 or SEQ ID NO:129 or SEQ ID NO;131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ED NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or 201;
(v) a nucleotide sequence capable of encoding an amino acid sequence having at least about 60% similarity after optimal alignment to SEQ ID NO:19 or SEQ ID NO:21 or SEQ ID NO:23 or SEQ ID NO:25 or SEQ ID NO:27 or SEQ ID NO:29 or SEQ ID NO:31 or SEQ ID NO:33 or SEQ ID NO:35 or SEQ ID NO:37 or SEQ ID NO:39 or SEQ ID NO:41 or SEQ ID NO:43 or SEQ ID NO:45 or SEQ ID NO:47 or SEQ ID NO:49 or SEQ ID NO:51 or SEQ ID NO:53 or SEQ ID NO:55 or SEQ ID NO:57 or SEQ ID NO:59 or SEQ ID NO:61 or SEQ ID NO:63 or SEQ ID NO:65 or SEQ ID NO:67 or SEQ ID NO:69 or SEQ ID NO:71 or SEQ ID NO:73 or SEQ ID NO:75 or SEQ ID NO:77 or SEQ ID NO:79 or SEQ ID NO:81 or SEQ ID NO:83 or SEQ ID NO:85 or SEQ ID NO:87 or SEQ ID NO:89 or SEQ ID NO:91 or SEQ ID NO:93 or SEQ ID NO:95 or SEQ ID NO:97 or SEQ ID NO:99 or SEQ ID NO:101 or SEQ ID NO:103 or SEQ ID NO:105 or SEQ ID NO:107 or SEQ ID NO: O9 or SEQ ID NO:111 or SEQ ID NO:113 or SEQ ID NO:115 or SEQ ID NO:117 or SEQ ID NO:119 or SEQ ID NO:121 or SEQ ID NO:123 or SEQ ID NO:125 or SEQ ID NO:127 or SEQ ID NO:129 or SEQ ID NO:131 or SEQ ID NO:133 or SEQ ID NO:135 or SEQ ID NO:137 or SEQ ID NO:139 or SEQ ID NO:141 or SEQ ID NO:143 or SEQ ID NO:145 or SEQ ID NO:147 or SEQ ID NO:149 or SEQ ID NO:151 or SEQ ID NO:153 or SEQ ID NO:155 or SEQ ID NO:157 or SEQ ID NO:159 or SEQ ID NO:161 or SEQ ID NO:163 or SEQ ID NO:165 or SEQ ID NO:167 or SEQ ID NO:169 or SEQ ID NO:171 or SEQ ID NO:173 or SEQ ID NO:175 or SEQ ID NO:177 or SEQ ID NO:179 or SEQ ID NO:189 or SEQ ID NO:191 or SEQ ID NO:193 or SEQ ID NO:195 or SEQ ID NO:197 or SEQ ID NO:199 or 201;
(vi) a nucleotide sequence capable of hybriding under low stringency conditions to the nucleotide sequence in (iv) or (v) or its complementary form;
   wherein said nucleotide sequences encode a CFM which imparts an altered visual characterization to said cell or group of cells to a human eye in the absence of extraneous non-white light or particle emission.

More particularly, there is provided a plant or cells of a plant or parts of a plant or progeny of a plant wherein said plant comprises cells comprising:
(i) a nucleotide sequence set forth in SEQ ID NO:189 or SEQ ID NO: 191 or SEQ ID NO:193 or SEQ ID NO:195 or SEQ ID NO:197 or SEQ ID NO:199 or SEQ ID NO:201;
(ii) a nucleotide sequence having at least about 60% similarity after optimal alignment to SEQ ID NO:189 or SEQ ID NO:191 or SEQ ID NO:193 or SEQ ID NO:195 or SEQ ID NO:197 or SEQ ID NO:199 or SEQ ID NO:201;
(iii) a nucleotide sequence capable of hybridizing under low stringency conditions to SEQ ID NO:189 or SEQ ID NO:191 or SEQ ID NO:193 or SEQ ID NO:195 or SEQ ID NO:197 or SEQ ID NO:199 or SEQ ID NO:201 or its complementary form;
(iv) a nucleotide sequence capable of encoding the amino acid sequence set forth in SEQ ID NO:190 or SEQ ID NO:192 or SEQ ID NO:194 or SEQ ID NO:196 or SEQ ID NO:198 or SEQ ID NO:200 or SEQ ID NO:202;
(v) a nucleotide sequence capable of encoding an amino acid sequence having at least about 60% similarity after optimal alignment SEQ ID NO:190 or SEQ ID NO:192 or SEQ ID NO:194 or SEQ ID NO:196 or SEQ ID NO:198 or SEQ ID NO:200 or SEQ ID NO:202;
(vi) a nucleotide sequence capable of hybridizing under low stringency conditions to the nucleotide sequence in (iv) or (v) or its complementary form;
   wherein said nucleotide sequences encode a CFM which imparts an altered visual characterization to said plant or cells of a plant to a human eye in the absence of extraneous non-white light or particle emission.

In a particularly preferred embodiment, there is provided a use of a CFM such as but not limited to GFP or a non-fluorescent GFP-homolog in the manufacture of a plant exhibiting altered visual characteristics to all or a part of said plant or to cells of said plant to a human eye in the absence of extraneous non-white light or particle emission.

Reference herein to extraneous light is not to be read as encompassing white light or background irradiation. The altered visual characteristics are visualized in the presence of white light, for example the light as generated by an 60 W electric bulb or daylight. White light includes light that contains all the wavelengths of the visible spectrum, such as sunlight.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25:3389, 1997). A detailed discussion of sequence analysis can be found in Unit 19.3 of Ausubel et al. (Current Protocols in Molecular Biology, John Wiley & Sons Inc, 1994-1998, Chapter 15).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J. Mol. Biol. 5:109, 1962). However, the Tₘ of a duplex DNA decreases by 1° C. with every increase of 1% in the number of mismatch base pairs (Bonner and Laskey, Eur. J. Biochem. 46:83, 1974). Formamide is optional in these hybridization conditions.

Accordingly, particularly preferred levels of stringency are defined as follows: low stringency is 6×SSC buffer, 0.1% w/v SDS at 25°-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The tobacco ribosomal DNA spacer element may be used to increase the expression of CFMs or colored proteins in transgenic Arabidopsis, carnation, rose or other plant species. The tobacco ribosomal DNA spacer element can be used to increase copy number and expression levels of transgenes in plants (Borisjuk et al., Nat. Biotechnol. 18:1303-1306, 2000). The tobacco ribosomal DNA spacer element may be inserted into pCGP2772, pCGP2785, pCGP3259 or other construct used to express CFMs or colored proteins in plants.

There is a clear correlation between codon usage and gene expression levels in Arabidopsis, Caenorhabditis and Drosophila (Duret and Mouchiroud, Proc. Natl. Acad. Sci. USA 96:4482-4487,1999).

Codon usage within the open reading frames of CFM or colored proteins may be modified to increase levels of CFMs or colored protein in transgenic Arabidopsis, carnation, rose or other plant species.

A recent study by Stevens et al. (Plant Physiology 173-182, 2000) has highlighted the possibility of increasing the stability of recombinant proteins in transgenic plants by modifying protein glycosylation patterns.

Plant virus gene vectors may be used for high level gene expression of foreign genes in plants (Scholthof and Scholthof, Annu. Rev. of Phytopathol. 34:299-323, 1996; Chapman et al., Plant Journal 2:549-557, 1992).

The use of a plant virus expression system may increase levels of CFMs or colored protein in transgenic Arabidopsis, carnation, rose or other plant species. Selection of an appropriate virus type or strain may allow the expression of CFMs or colored protein in specific tissues or patterns to produce novel phenotypes. For example a CFM or colored protein gene may be incorporated into the genome of tulip breaking virus or tulip chlorotic blotch potyvirus to induce colored sector production in tulip or other flowers.

The availability of the isolated CFMs of the present invention further provides the possibility for generating antibodies, whether monoclonal or polyclonal, against any or all of these isolated sequences or derivatives or homotogs thereof.

Well-known protocols applicable to antibody production, purification and use may be found, for example, in Chapter 2 of Cologne et al. (Current Protocols in Immunology, John Wiley & Sons, N.Y., 1991-94) and Harlow and Lane, Antibodies: A Laboratory Manual, Cold Spring Harbor, Cold Spring Harbor Laboratory, 1988, which are both herein incorporated by reference.

Generally, antibodies of the invention bind to or conjugate with a polypeptide, fragment, variant or derivative thereof. For example, the antibodies may comprise polyclonal antibodies. Such antibodies may be prepared, for example, by injecting a polypeptide, fragment, variant or derivative thereof into a production species, which may include mice or rabbits, to obtain polyclonal antisera. Methods for the production of polyclonal antibodies are well known to those skilled in the art. Exemplary protocols are described in Cologan et al., 1991-1994, supra and Harlow and Lane, 1988, supra.

In lieu of polyclonal antisera obtained in a production species, monoclonal antibodies may be produced using the standard method as described by Köhler & Milstein (European Journal of Immunology 6:511-519, 1976) or by more recent modifications thereof as, for example, described in Cologne et al. (1991-1994, supra) by immortalizing spleen or other antibody-producing cells derived from a production species which has been inoculated with one or more of the polypeptides, fragments, variants or derivatives of the present invention.

The present invention also contemplates antibodies that comprise Fc or Fab fragments of the polyclonal or monoclonal antibodies referred to above. Alternatively, the antibodies may comprise single chain Fv antibodies (scfvs) against the peptides of the present invention. Such scFvs may be prepared, for example, in accordance with the methods described respectively in U.S. Pat. No. 5,091,513, European Patent No 239,400 or Winter and Milstein (Nature 349:293, 1991).

Antibodies produced in accordance with the present invention may be used for affinity chromatography in isolating natural or recombinant pigment polypeptides. For appropriate protocols, reference may be made to immunoaffinity chromatographic procedures described in Chapter 9.5 of Cologan et al. (1991-1994, supra).

Accordingly, the present invention provides an antibody specific for a CFM, said CFM comprising an amino acid sequence in its N-terminal end selected from SVIAK (SEQ ID NO:5), (M)SVIAT (SEQ ID NO:6), SGIAT (SEQ ID NO:7), SVIVT (SEQ ID NO:8) or SVSAT (SEQ ID NO:9).

Preferably, the isolated antibody is specific for a CFM comprising an amino acid sequence selected from the list comprising SVIAT QMTY KVYM SGT (SEQ ID NO:10), SVIAT QMTY KVYM PEG (SEQ ID NO:11), SVLAT QVTY KVYM SGT (SEQ ID NO:12), SGIAT QMTY KVYM SGT (SEQ ID NO:13), SVIVT QMTY KVYM SGT (SEQ ID NO:14), SVSAT QMTY KVYM SGT (SEQ ID NO:15), SVIAK QMTY KVNM SGT (SEQ ID NO:16), SVIAK QMTY KVYM SDT (SEQ ID NO:17) and/or SVIAK QMTY XX₂YX₃ SGT (SEQ ID NO:18) wherein X₁, X₂ and X₃ may be any amino acid provided that X₁ is not K; X₂ is not V; X₃ is not M.

Most preferably, the antibody is specific for a CFM comprising an amino acid sequence selected from the listing comprising SEQ ID NOs:20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154,.156, 158, 160, 162, 164, 166, 168, 170, 172, 174, 176, 178, 180, 190, 192, 194, 196, 198, 200 and 202.

Once antibodies have been produced, one or more polypeptides of the present invention may be conjugated thereto, preferably to a secondary antibody as part of an antibody staining complex, and thereby become useful as a fluorescent marker in microscopy and related procedures. Alternatively, or in addition, one or more nucleic acid sequence encoding a polypeptide of the present invention may be expressed as a recombinant polypeptide fused with a secondary antibody. These antibodies may be useful for in situ labelling procedures or in other related procedures such as fluorescence in situ hybridization (FISH).

As already described above, a fusion partner well known in the art is GFP. This fusion partner may serve as a fluorescent “tag” which facilitates the identification and/or localization, by fluorescence microscopy or by flow cytometry, of a polypeptide fused thereto. Flow cytometric methods such as fluorescence activated cell sorting (FACS) are particularly useful in this regard.

There is perpetual interest in developing high-sensitivity biochemical assays, which employ luminescence, fluorescence or visible color rather than radioisotopes, for use in research and in medicine. Interest in developing assays with visible detection systems is increasing as these often obviate the need for expensive luminescence, fluorescence or isotopic detection equipment.

Accordingly, the present invention further comprises a diagnostic assay comprising screening for the presence of CFM wherein the nucleic acid molecule encoding said CFM is expressed in a cell.

The capability of the CFMs to absorb incident light which encompasses the UV range (320-700 nm) makes them useful candidates for inclusion as components in topically-applicable sun screen formulations. The purpose of a sun screen is to block the excessive UV radiation from affecting the skin. Sun screen formulations act by deflecting and scattering the incident light that produces burning and tanning of the skin or by absorbing this light. It is known that careful selection of sun screens can offer this protection to the skin and reduce the darkening and damaging effects of the radiation.

Such a formulation would include, for example, an effective amount of one or more CFMs of the present invention, optionally admixed with a pharmaceutically acceptable vehicle such as a carrier or excipient that will not harm the skin. By “carrier” is meant a solid or liquid filler, diluent or substance that may be safely used in topical administration. These carriers may be selected from a group including powder absorbants, creams, oils, synthetic oils, phosphate buffered solutions, emulsifiers, and liquids such as emollients, propellants, solvents, humectants, thickners, isotonic saline, and pyrogen-free water. The sun screen formulation may also include other screening agents, well known in the art, such as propyl hydroxybenzoate, dimethylaminobenzoate (PABA), phenyl salicylates and/or octyl methoxycinnamate. These formulations may be prepared for topical application to the skin in the form of conventional products such as lotions, creams, ointments and aerosol products. A useful sun screen formulation and method of preparing an emulsion therefor are provided in International Patent Publication No. WO 00/46233 in Example 4.

Accordingly, the present invention provides a biomatrix comprising a CFM, said CFM comprising a polypeptide which, in a cell, alone or together with one or more other molecules imparts an altered visual characteristic to said cell when visualized by a human eye in the absence of excitation by extraneous non-white light or particle emission.

Reference to a “biomatrix” includes any composition comprising a CFM such as a cell, sun screen, a purified preparation of a CFM or any solid support onto or into which a CFM is immobilized. Reference to a biomatrix also includes a bioinstrument.

Yet another aspect of the present invention contemplates the use of a CFM in a cosmetic or light filtering composition. Cosmetics include many products that can be applied to the face or body in order to alter appearance or color. New combinations of ingredients may result in cosmetic compositions that protect against environmental stresses such as exposure to the sun. The use of a CFM in a cosmetic may provide a visible coloration that is aesthetically desirable and/or it may provide light filtering capability such as may be afforded, for example, by a sun screen.

Light filtering compositions may also be used to screen out or block UV light or different wavelengths of light within the entire spectrum. A cosmetic or light filtering composition according to the invention may also include cosmetically or pharmaceutically compatible carriers, preservatives, emusifiers, thickners, perfume, color, as well as other materials having properties which are beneficial for skin, such as moisturizers, emollients anti-ageing compounds inter alia.

Other applications of the CFMs of the present invention may also be contemplated. Since they are active in affecting the manner in which, and degree to which, various kinds of impinging light/radiation are processed and detected, the CFMs may find application in, for example, transducing or intensifying an image. For example, converting less visible wavelengths of light such as IN radiation to wavelengths that are more visible might be beneficial. A gel or similar material comprising a CFM may be located behind a membrane or selective barrier and combined with an optic fiber probe, such as an optode or micro-electrode. Changes in physical and chemical environments into which the probe is inserted may be calibrated to changes in fluorescent intensity and/or fluorescence half-life, to provide micro-scale measurements of parameters such as oxygen concentration and pH. Similar applications involving fluorescence intensity and/or half-life fluorescent imaging techniques may also incorporate a CFM of the present invention.

As stated above, each of the CFMs of the present invention and homologs thereof, has distinct excitation and emission characteristics. These may be fluorescently coupled such that captured photons can be passed successively between a plurality of CFMs, for example as many as six. This lengthens the pathway and the amount of time that a photon spends within any material comprising the CFMs and may thereby increase light intensity within these environments considerably. Such a light enhancement effect may be useful for providing additional light for growing phototrophic organisms, for example plants, algae and/or corals, by increasing the likelihood of a photon's interaction with constituent photosystems.

This embodiment of the present invention may also be useful for creating light enhancer fluids that may be used to increase light intensity within a medium above that of incident light.

Furthermore, a CFM embedded in a gel matrix or other useful material may improve image quality in situations of distorted light spectra such as, for example, under water where light is shifted to the blue end of the spectrum. A CFM rendered water-soluble may prove useful in a range of different types of liquids. Alternatively, or in addition, a derivative or homolog of polypeptide of the present invention may be synthesised by substituting amino acids or adding N- or C-terminal tags to increase their insolubility and hence make them more useful in less polar environments. In this embodiment, a CFM, or a CFM modified such as through amino acid inclusion or substitution to make it more hydrophobic, combined with a water-soluble or non-water soluble emulsion, may be used to coat materials that experience UV damage such as, for example, plastics and car upholstery.

The present invention is further described by the following non-limiting Examples.

### EXAMPLE 1

### General Methods

In general, the methods followed were as described in Sambrook et al. (Molecular Cloning: A Laboratory Manual. (2nd edition), Cold Spring Harbor Laboratory Press, USA, 1989).

The cloning vectors pBluescript and PCR script were obtained from Stratagene. pCR7 2.1 was obtained from Invitrogen.

The bacterial expression vector pQE-30 Was obtained from Qiagen.

E. coli transformation

The Escherichia coli strains used were:-

DH5αsupE44, Δ (lacZYA-ArgF)U169, (ø80lacZΔM15), hsdR17(rₖ⁻, m⁺k), recA1, endA1, gyrA96, thi-1, relA1, deoR. (Hanahan, J. Mol. Biol. 166:557 1983

XL1-Blue supE44, hsdR17(rₖ⁻, mₖ⁺), recA1, endA1, gyrA96, thi-1, relA1, lac⁻,[F′proAB, lacI^{q}, lacZΔM15, Tn10(tet^{R})] (Bullock et al., Biotechniques 5:376, 1987).

BL21-CodonPlus-RIL strain ompT hsdS(rB- mB-) dcm+ Tet^{r} gal end A Hte [argU ileY leuW Cam^{r}]M15 E. coli is derived from E.coli K12 and has the phenotype Nal^{S}, Str^{S}, Rif^{S}, Thi⁻, Ara⁺, Gal⁺, Mt1⁻; F⁻, RecA⁺, Uvr⁺, Lon⁺.

Transformation of the E. coli strains was performed according to the method of Inoue et al., (Gene 96:23-28, 1990).

Agrobacterium Tumefaciens Strains and Transformations

The disarmed Agrobacterium tumefaciens strain used was AGL0 (Lazo et al. Bio/technology 9:963-967, 1991).

Plasmid DNA was introduced into the Agrobacterium tumefaciens strain AGL0 by adding 5 μg of plasmid DNA to 100 μL of competent AGL0 cells prepared by inoculating a 50 mL LB culture and growing for 16 hours with shaking at 28° C. The cells were then pelleted and resuspended in 0.5mL of 85% v/v 100 mM CaCl₂/15% v/v) glycerol. The DNA-Agrobacterium mixture was frozen by incubation in liquid N₂ for 2 minutes and then allowed to thaw by incubation at 37° C. for 5 minutes. The DNA/bacterial mix was then placed on ice for a further 10 minutes. The cells were then mixed with 1 mL of LB (Sambrook et al., 1989 supra) media and incubated with shaking for 16 hours at 28° C. Cells of A. tumefaciens carrying the plasmid were selected on LB agar plates containing 50 μg/mL tetracycline. The confirmation of the plasmid in A. tumefaciens was done by restriction enzyme analysis of DNA isolated from the tetracycline-resistant transformants.

Saccharornyces cerevisiae strains and transformations

The yeast expression vector used was pYE22m (Tanaka et al., J. Biochem. 103:954-961, 1988).

The yeast strain G-1315 (Mat a l) (Ashikari et al., Appl. Microbiol. Biotechnol. 30: 515-520, 1989) was transformed with plasmid DNA according to Ito et al., (J. Bacteriol. 153:163-168, 1983). The transformants were selected by their ability to restore G-1315 to tryptophan prototrophy.

DNA Ligations

DNA ligations were carried out using the Amersham Ligation Kit according to procedures recommended by the manufacturer.

Isolation and Purification of DNA Fragments

Fragments were generally isolated on a 1% w/v agarose gel and purified using the QIAEX II Gel Extraction kit (Qiagen).

Reparation of Overhanging ends after Restriction Digestion

Overhanging 5′ ends were repaired using DNA polymerase (Klenow fragment) according to standard protocols (Sambrook et al., 1989 supra). Overhanging 3′ ends were repaired using T4 DNA polymerase according to standard protocols (Sambrook et al., 1989 supra).

Removal of Phosphory Groups from Nucleic Acids

Shrimp alkaline phosphatase (SAP) (USB) was typically used to remove phosphoryl groups from cloning vectors to prevent re-circularization according to the manufacturer's recommendations,

Polymerase Chain Reaction (PCR)

Unless otherwise specified, PCR conditions using plasmid DNA as template included using 2 ng plasmid, 100 ng each of primers, 2 μL 10 mM dNTP mix, 5 μL 10×PfuTurbo (registered trademark) DNA polymerase buffer (Stratageme), 0.5 μL PfuTurbo (registered trademark) DNA polymerase (2.5 units/μL) (Stratagene) in a total volume of 50 μL. Cycling conditions were an initial denaturation step of 5 min at 94° C., followed by 35 cycles of 94° C. for 20 sec, 50° C. for 30 sec and 72° C. for 1 min with a last treatment of 72° C. for 10 min and then finally storage at 40° C.

PCRs were performed in a Perkin Elmer GeneAmp PCR System 9600.

³²P-Labelling of DNA Probes

DNA fragments (50 to 100 ng were radioactively labelled with 50 μCi of [α-³²P]-dCTP using a Gigaprime kit (Geneworks). Unincorporated [α-³²P]-dCTP was removed by chromatography on a Sephadex G-50 (Fine) column.

Plasmid Isolation

Single colonies were analyzed for inserts by growing in LB broth (Sambrook et al., 1989, supra) with appropriate antibiotic selection (e.g. 100 μg/mL ampicillin or 10 to 50 μg/mL tetracycline for binary vector constructs). Plasmid DNA was purified using the alkali-lysis procedure (Sambrook et aL, 1989, supra) or using The WizardPlus SV minipreps DNA purification system (Promega) or Qiagen Plasmid Mini Kit (Qiagen). Once the presence of an insert had been determined, larger amounts of plasmid DNA were prepared from 50 mL overnight cultures using a QIAfilter Plasmid Midi kit (Qiagen).

DNA Sequence Analysis

DNA sequencing was performed using the PRISM (trademark) Ready Reaction Dye Primer Cycle Sequencing Kits from Applied Biosystems. The protocols supplied by the manufacturer were followed. The cycle sequencing reactions were performed using a Perkin Elmer PCR machine (GeneAmp PCR System 9600). Sequencing runs were performed by the Australian Genome Research Facility at The Walter and Eliza Hall Institute of Medical Research (Melbourne, Australia).

Homology searches against Genbank, SWISS-PROT and EMBL databases were performed using the FASTA and TFASTA programs (Pearson and Lipman, 1988) or BLAST programs (Altschul et al., J. Mol. Biol. 215(3):403-410, 1990). Percentage sequence similarities were obtained using LALIGN program (Huang and Miller, Adv. Appl. Math. 12:373-381, 1991) using default settings.

Multiple sequence alignments were produced using ClustalW (Thompson et al., Nucleic Acids Research 22:4673-4680, 1994).

Petunia Transformations

(a) Plant Material

Leaf tissue from mature plants of P. hybrida cv Mitchell (or Ba20 or Br140w) was treated in 1.88% w/v sodium bypochlorite for 2 minutes and then rinsed three times in sterile water. The leaf tissue was then cut into 25-50 mm² squares and precultured on MS media (Murashige and Skoog, Physiol. Plant 15:73-97, 1962) supplemented with 1.0 mg/L α-benzylaminopurine (BAP) and 0.1 mg/L α-naphthalene acetic acid (NAA) for 24 hours under white fluorescent lights.

(b) Co-cultivation of Agrobacterium and Petunia Tissue

A. tumefaciens strain AGL0 containing a binary vector were maintained at 4° C. on LB agar plates with 50 μg/mL tetracycline. A single colony was grown overnight in liquid LB medium containing 40 μg/mL tetracycline. The following morning 1-2 mL of the overnight culture was added to a fresh batch of 25 mL liquid LB medium and the culture was grown at 37° C with shaking until an absorbance reading at 650 nm (A₆₅₀) of 0.4 to 0.8 was reached. A final concentration of 5×10⁸ cells/mL was prepared by dilution in liquid MS medium containing 50 μM acetosyringone and 3% w/v sucrose B5 vitamins (Gamborg et al., Exp. Cell Res. 50:151-158, 1968). The leaf discs were dipped for 2 minutes into the inoculum and then blotted dry and placed on co-cultivation media for 5 days. The co-cultivation medium consisted of SH medium (Schenk and Hildebrandt, Can. J. Bot. 50:199-204, 1972) supplemented with 0.05 mg/L kinetin and 1.0 mg/L 2,4-D.

(c) Recovery of Transgenic Petunia Plants

After co-cultivation, the leaf discs were transferred to selection medium (MS medium supplemented with 3% w/v sucrose, 3 mg/L BAP, 0.2 mg/L IAA, 1 μg/L chlorsulfuron, 300 mg/L timentin and 0.3% w/v Gelrite Gellan Gum (Schweizerhall). Regenerating explants were transferred to fresh selection medium after 2 weeks.

Adventitious shoots which survive the chlorsulfuron selection are isolated and transferred to BPM containing 1 μg/L chlorsulfuron and 300 mg/L timentin for root induction. All cultures are maintained under a 16 hour photoperiod (60 μmol, m⁻², s⁻¹ cool white fluorescent light) at 23±2° C. When roots reach 2-3 cm in length the transgenic petunia plantlets are transferred to autoclaved Debco 51410/2 potting mix in 8 cm tubes. After 4 weeks, plants are be replanted into 15 cm pots, using the same potting mix, and maintained at 23° C. under a 14 hour photoperiod (300 μmol. m⁻², s⁻¹ mercury halide light).

Arabidopsis transformations

Arabisopsis thaliana ecotype WS-2 seeds were obtained from The University of Melbourne, Parkville, Melbourne, Australia

Plant growth conditions and transformation of A. thaliana were as essentially as described by Clough and Bent, (Plant J., 16:735-743, 1998) except that seeds from the transformed plants were selected on 100 μg/mL chlorsulfuron when binary vectors containing the SuRB selectable marker gene were used for the transformation process.

### EXAMPLE 2

### Isolation of New Colored-protein Sequences from Heron Island Coral

Coral samples were collected from Heron Island Reef flat, Queensland, Australia. These samples were viewed as whole tissue under a fluorescent microscope, as described herein.

Assessment of Fluorescence Properties

Table 2 shows taxonomic relationships of GFP isolated from the phylum Cnidaria and comparison with one amino acid sequence of the invention (Aams2-pep; SEQ ID NO:88). Fluorescent properties were analysed using an Olympus fluorescent microscope (BH2-RFL) with filter combinations, as shown in Table 3. Tables 4 and 5 show fluorescent properties of colors for different species of organisms from Anthozoa and Hydrozoa.

Total RNA Isolation

Plating corals were ground with a mortar and pestle and branching corals were scrubbed with a toothbrush directly into cold solution D, as described in Chomczynski and Sacchi, 1987, supra. Solution D-comprising tissue was homogenized using a glass homogenizer and transferred to 1.5 ml eppendorf microcentrifuge tubes. A 10% w/v 2 M sodium acetate (pH 4) solution was added prior to phenol chloroform extraction and extracted material was precipitated overnight in isopropanol at −20° C. Pellets were resuspended in solution D, and precipitated again in isopropanol. Resulting pellets were dissolved in 3 mM EDTA and 50 mM sodium acetate (pH 5) to be finally precipitated and stored at −20° C. in ethanol.

cDNA Construction

RNA isolated from collected coral tissue was used to prepare cDNA. cDNA were constructed using a directional cDNA synthesis kit from Clontech Laboratories (Palo Alto, Calif., USA) herein incorporated by reference.

5′ Forward Primers for PCR Amplification

| | |
|---|---|
| POC FOR | |
| TCC GTT ATC GCT AAA CAG ATG ACC TAC AAA | SEQ ID NO:1 |
| POC 220 | |
| GGC GAC CAC AGG TTT GCG TGT | SEQ ID NO:2 |
| MSVIAT(FOR) | |
| ATG AGT GTG ATC GCT ACA CAA | SEQ ID NO:3 |

SEQ ID NO:1 was previously designed as a 5′ (or forward primer) for PCR amplification of nucleic acids encoding coral pigment proteins. SEQ ID NO:1 was shown to anneal to nucleic acids encoding a polypeptide comprising amino acids, SWLAK (SEQ ID NO:5): Refer to Dove et al. (2001; supra) and International Patent Publication No. WO 00/46233.

SEQ ID NO:2 was originally designed as a 3′ (or reverse primer) for PCR amplification of nucleic acids encoding coral pigment polypeptides as disclosed in WO 00/46233. In addition to annealing to a 3′ region of the nucleic acid as intended, SEQ ID NO:2 also anneals to a 5′ UTR region of pocilloporin from Acropora aspera as disclosed herein.

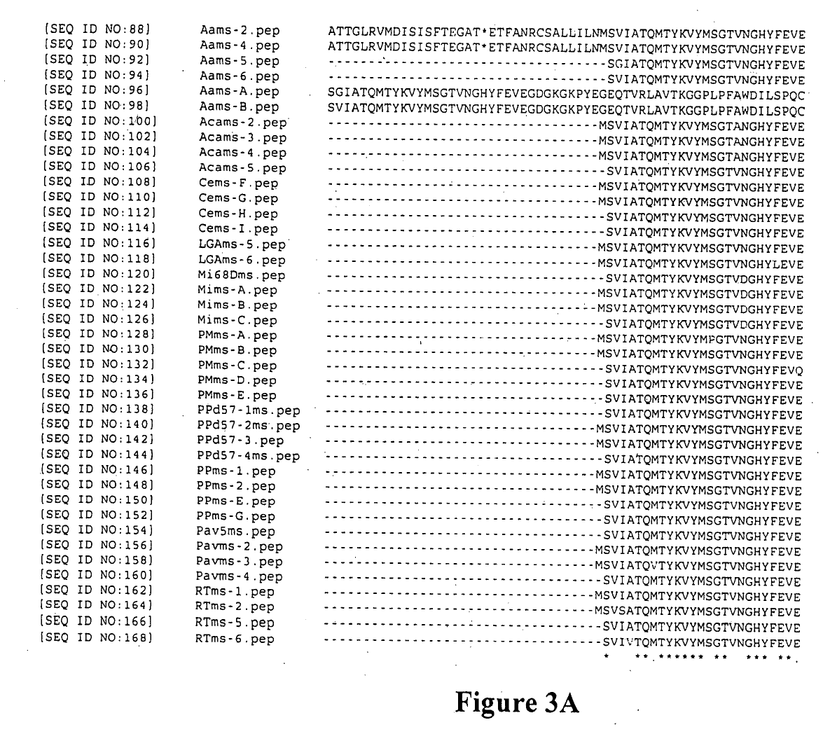

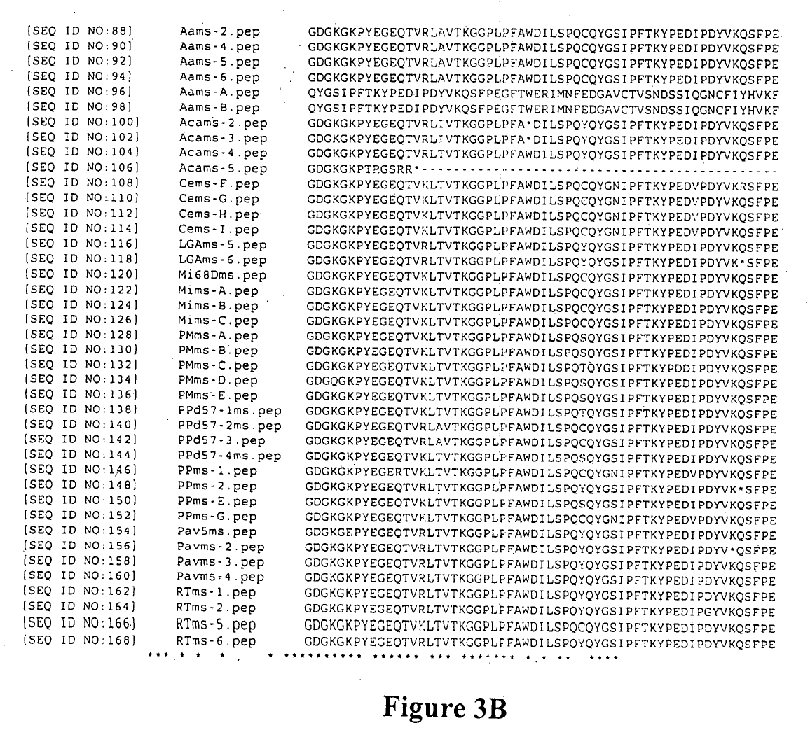

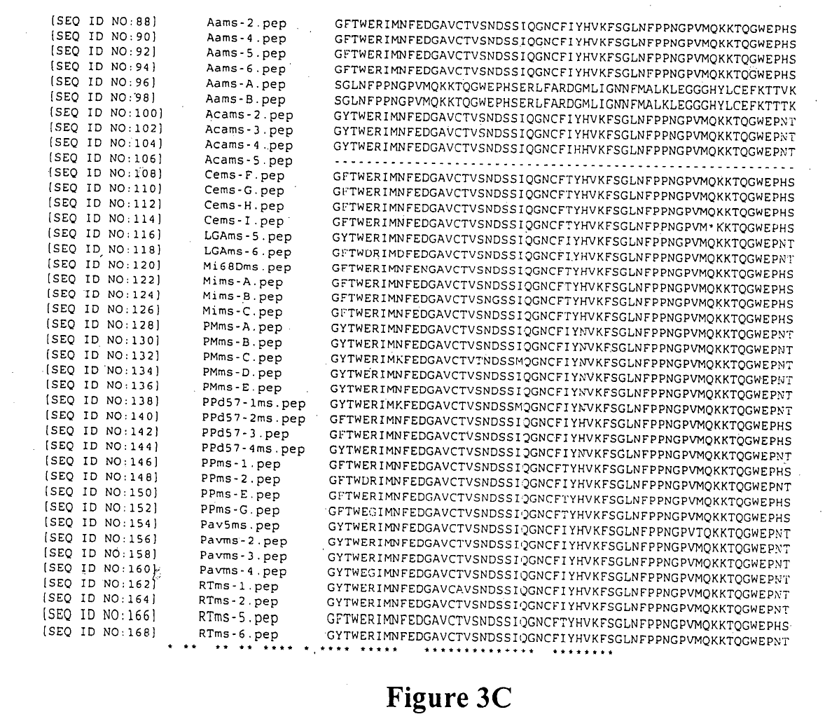

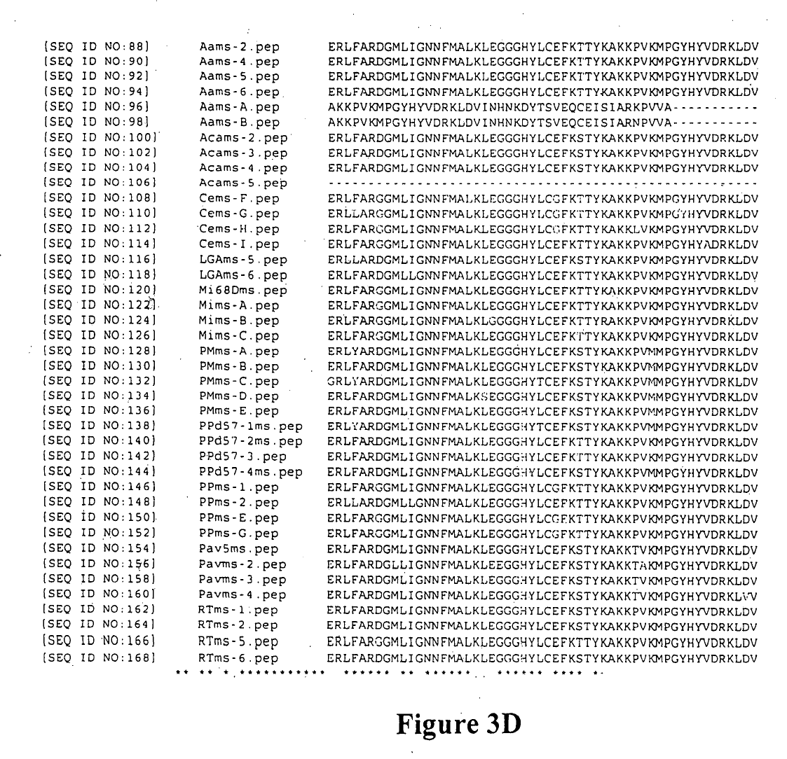

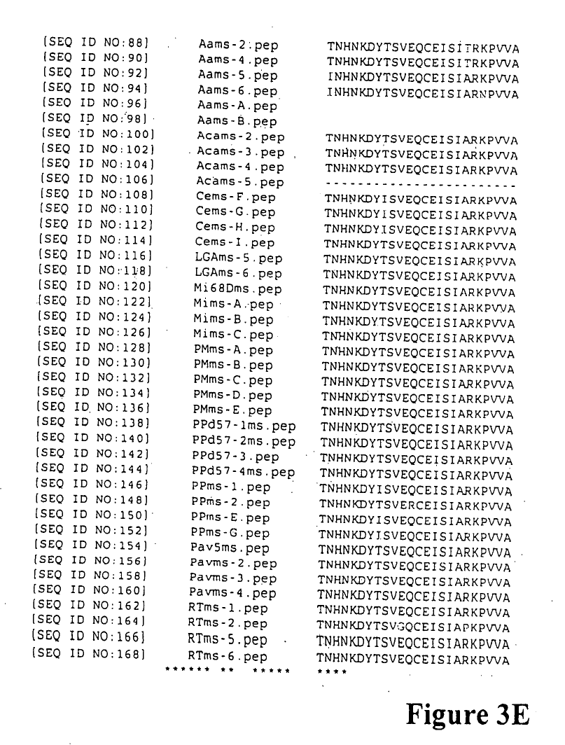

SEQ ID NO:3 is newly designed and synthesized based on sequence information from PCR amplification products using SEQ ID NO:1 and SEQ ID NO:2. The amplified products comprise 5′ UTR nucleotide sequence that includes sequence encoding a novel amino terminal end for a polypeptide similar to, but distinct from, the polypeptide disclosed in International Patent Publication No. WO 00/46233. This novel polypeptide has an amino terminal end comprising amino acids (M)SVIAT (SEQ ID NO:6; FIG. 3). Accordingly, SEQ ID NO:3 anneals to nucleic acids encoding a peptide comprising (M)SVIAT (SEQ ID NO:6). Although peptide sequences SVIAK (SEQ ID NO:5) and (M)SVIAT (SEQ ID NO:6) differ by only one amino acid, the corresponding nucleic acids only share 67% identity (12 nucleic acids of 18). Notably, SEQ ID NO:1 cannot be used to amplify sequences starting with the N-terminal peptide QM)SVIAT (SEQ ID NO:6), and SEQ ID NO:3 cannot be used to amplify sequences beginning with the SVIAK (SEQ ID SEQ ID NO:5) peptide.

3′ Reverse Primers for PCR Amplification

| | |
|---|---|
| POC 231 | |
| TTT GTG CCT TGA TTT GAC TCT | SEQ ID NO:4 |

SEQ ID NO:2 was also used as a 3′ reverse primer and is described above. SEQ ID NO:4 was designed to anneal to a 3′ end of previously isolated pocilloporin from Acropora aspera (Dove et al. [2001; supra] and International Patent Publication No. WO 00/46233).

PCR Amplification

PCR amplification was performed using a combination of the abovementioned SEQ ID NOs as described in more detail hereinafter. Hybaid PCR express (Hybaid PCR Express, Integrated Sciences, Australia) was used according to instructions provided therein. Amplification products were separated by gel electrophoresis on a 1.5% w/v agarose gel and nucleic acid bands comprising desired nucleic acids were visualized using standard methods. Agarose gel comprising the desired nucleic acids were gel purified and the gel purified nucleic acids were inserted by ligation into pGemT-vector (Promega, Madison, Wis. USA) producing a recombinant vector.

The inserted nucleic acids were sequenced using 17 and SP6 primers, which flank the inserted nucleic acid (sequencing service provided by AGRF; University of Queensland, Australia). Sequencing of the insert was performed at least twice in both forward and reverse directions until ambiguities were resolved. The following sequences were sequenced in a single direction: Ce61-7sv-rep (SEQ ID NO:37); Ce61-5sv-rep (SEQ ID NO:35); PM1Csv-rep (SEQ ID NO:57); PM1Asv-rep (SEQ ID NO:55); Mi68Dms (SEQ ID NO:119); Acams-3 (SEQ ID NO:101).

Table 6 shows amino acid sequences within 5 Angstroms of the fluorphore which encode possible spectral variants of the polypeptides of the invention comprising an amino acid (M)SVIAT (SEQ ID NO:6) at the amino terminal end. These amino acid sequences were translated from nucleic acid sequences derived by PCR using 5′ primers: SEQ ID NO:2 (5′ UTR) and SEQ ID NO:3 [(M)SVIAT]; and 3′ primers: SEQ ID NO:2 and SEQ ID NO:4.

Table 7 shows amino acid sequences within 5 Angstroms of the fluorphore which encode possible spectral variants of the polypeptides of the invention comprising an amino acid sequence SVIAK (SEQ ID NO:5) at the amino terminal end. These amino acid sequences were translated from nucleic acid sequences derived by PCR using 5′ primer SEQ ID NO:1 and 3′ primer SEQ ID NO:2, and 3′ SEQ ID NO:3.

Polypeptide Modelling

A 3-dimensional model of the polypeptides was used to predict those amino acids within 5 Angstroms of the fluorophore “QYG”. These amino acids have potential to influence spectral properties (Tsien, 1998, supra and Dove et al., 2001, supra) and are shown in Tables 6 and 7. The amino acids which are predicted to be located within 5 Angstroms of the fluorophore correspond to amino acid residues 37, 39, 56-65 (which comprises the fluorophore QYG), 86, 88, 90, 104, 106, 115, 139, 141, 143, 156, 158, 171, 192, 194, 208, 209 and 210. Amino acid residue numbers refer to numbering beginning with amino terminal amino acids S-V-I as residues 1, 2 and 3, respectively.

Information in relation to amino acid residues within 5 Angstroms of the fluorophore and details of atomic contacts for the polypeptide disclosed in Table 4 of International Patent Publication No. WO 00/46233, may be useful with the polypeptides of the present invention. In Tables 6 and 7, “Type” refers to a grouping or class of common amino acids within 5 Angstroms of the fluorophore, and “*” indicates an internal stop codon. “Name” refers to consensus sequence name from multiple repeat sequences.

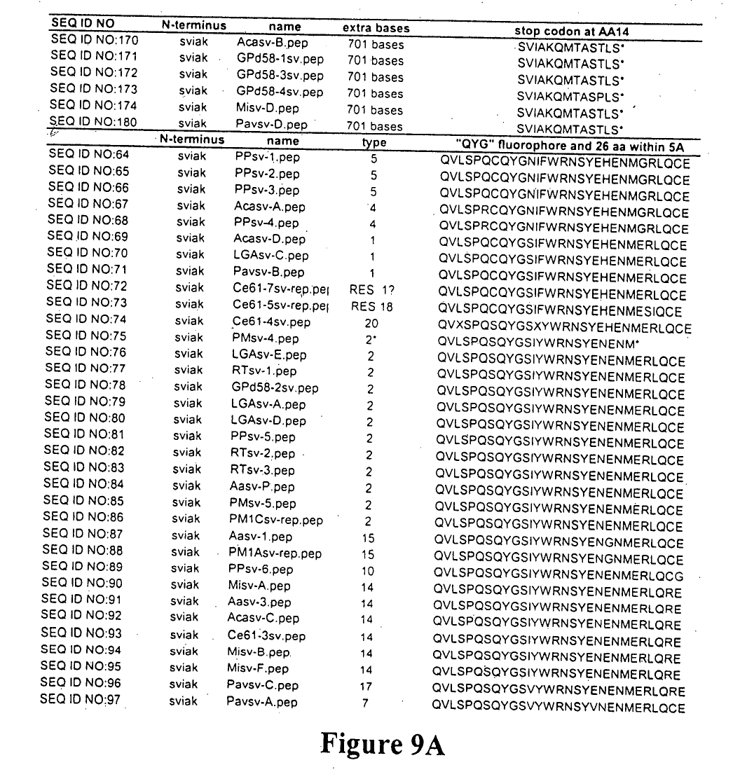

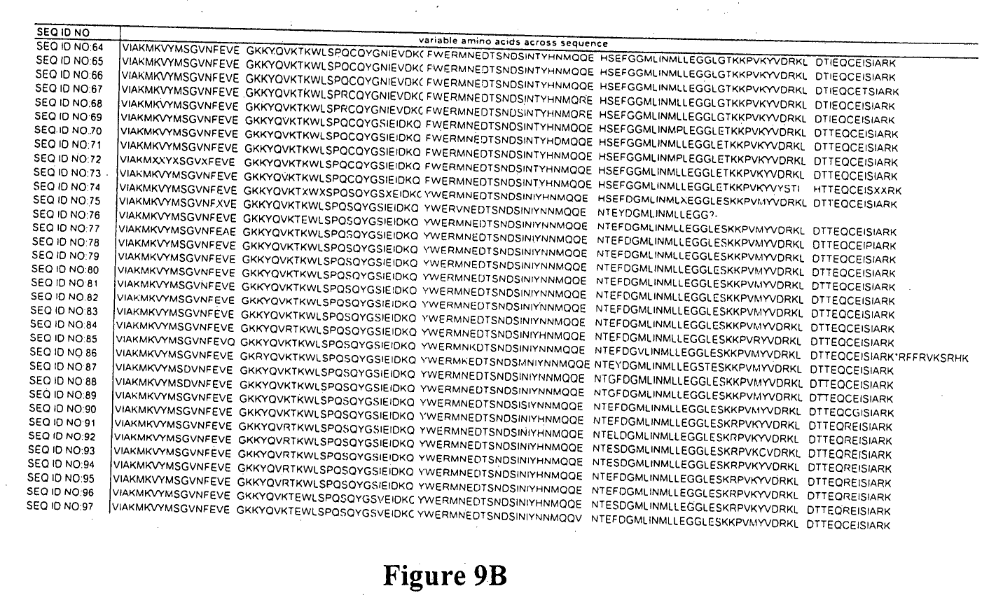

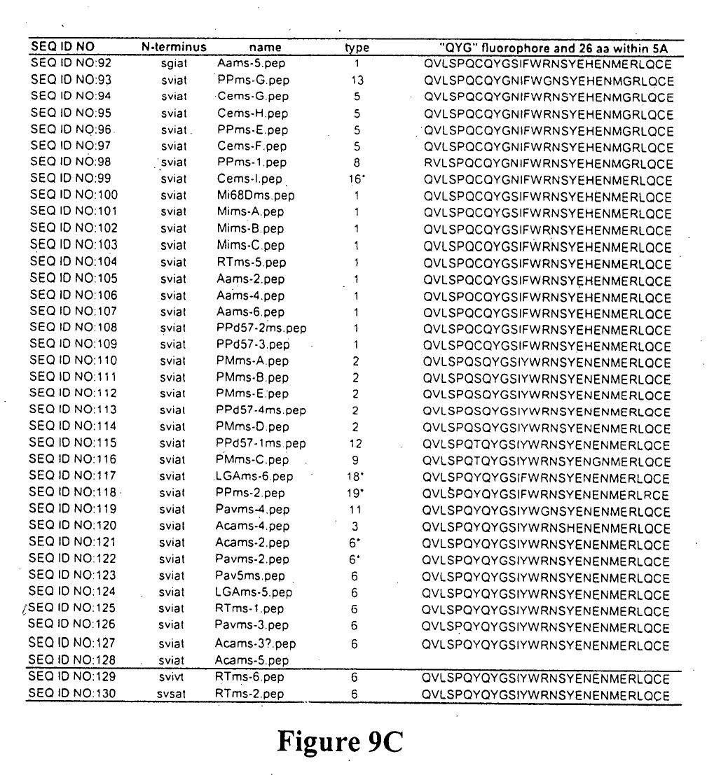

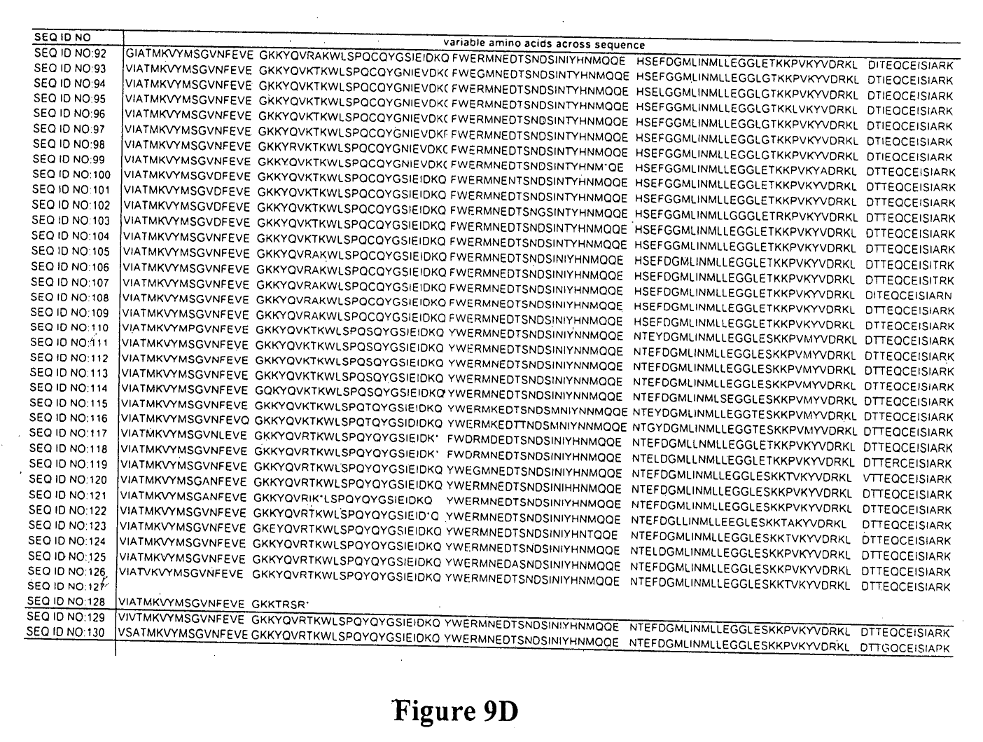

FIG. 9 lists many of the pigment polypeptides of the invention and indicates the amino acid residues that are located within 5 Angstroms of a fluorophore region of the polypeptide. In addition, those amino acids residue positions where variation is found throughout the different polypeptides are shown. Variable amino acids indicated throughout the polypeptide may be significant, as they may interfere with polypeptide folding.

Amino Acid and Nucleotide Sequence Comparisons

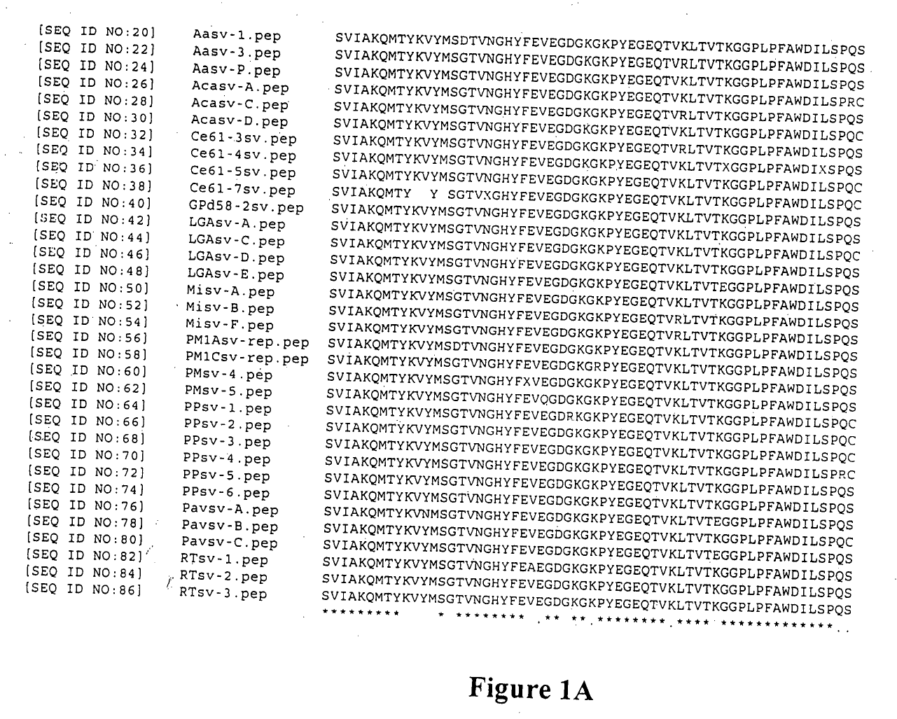

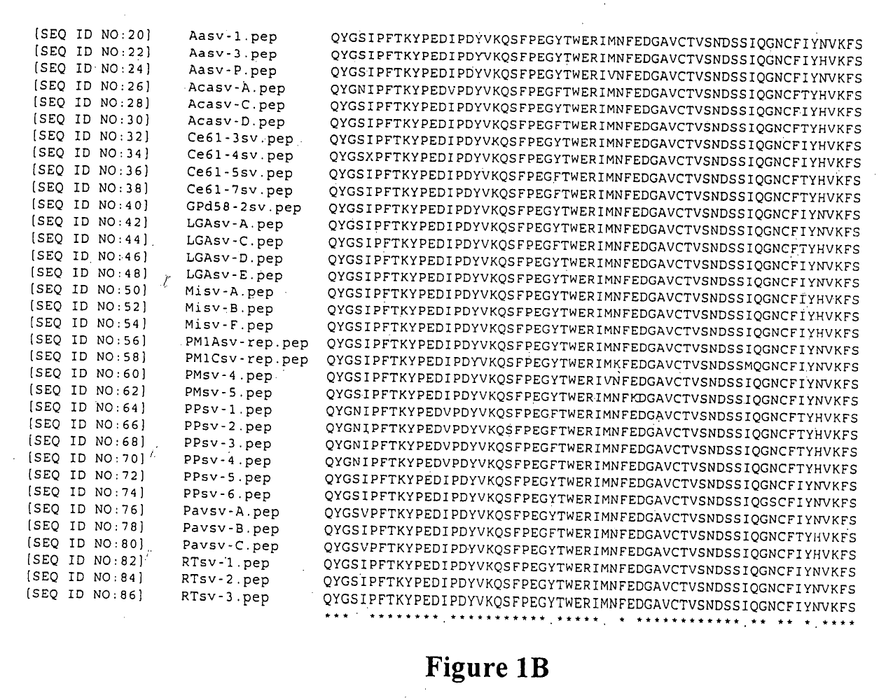

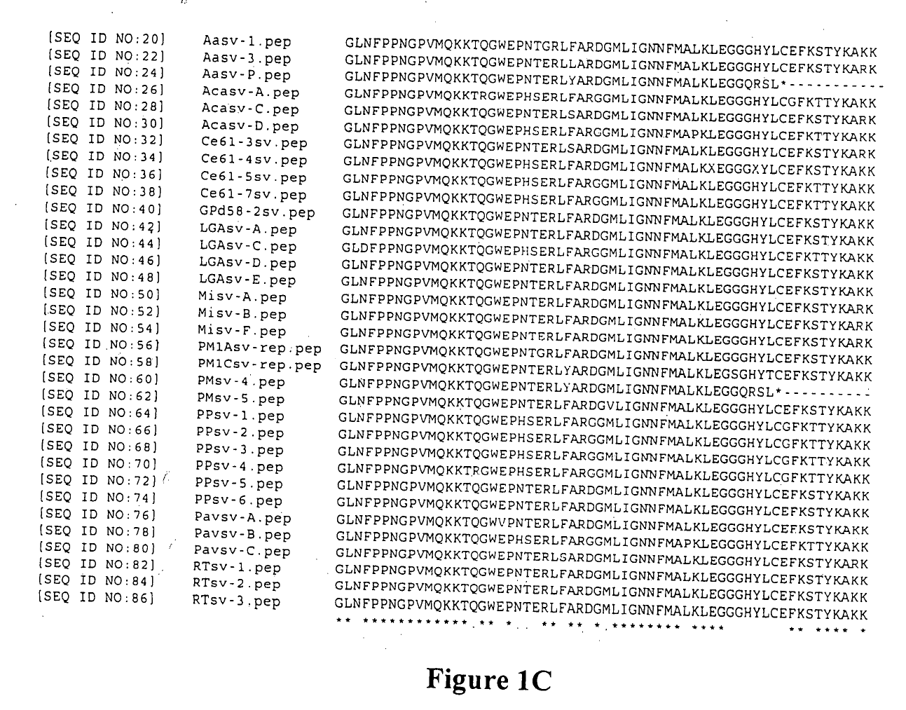

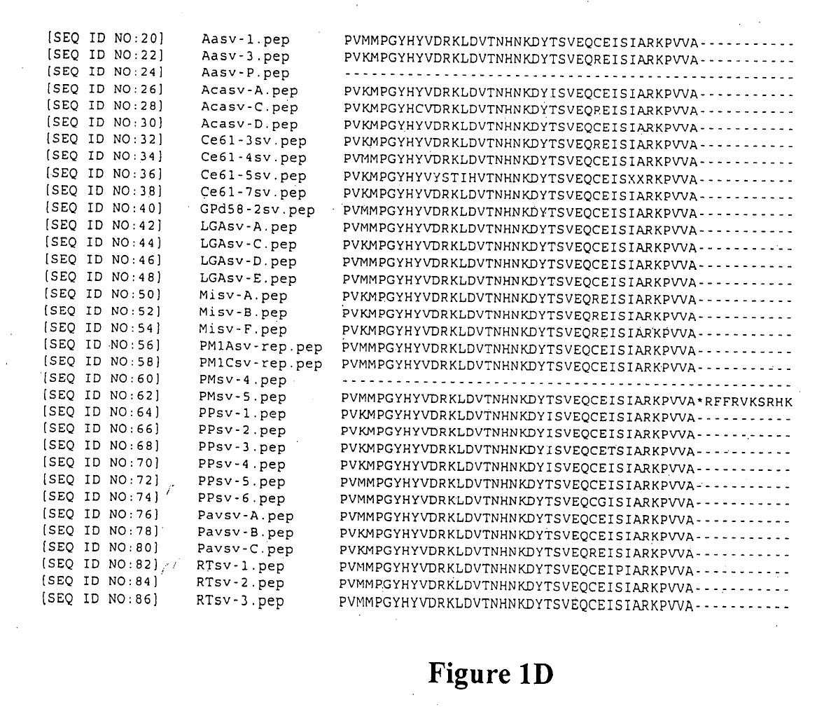

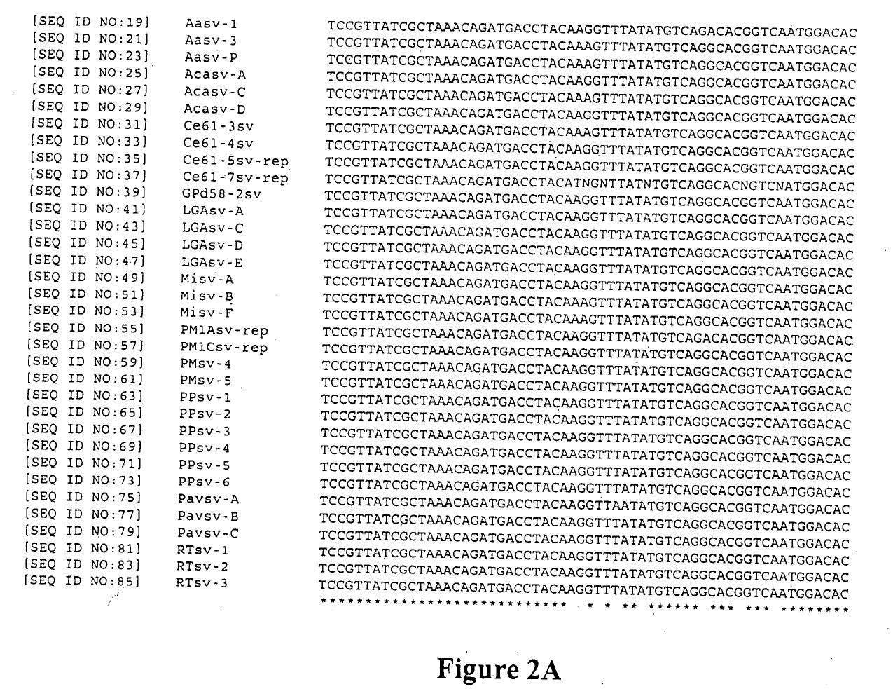

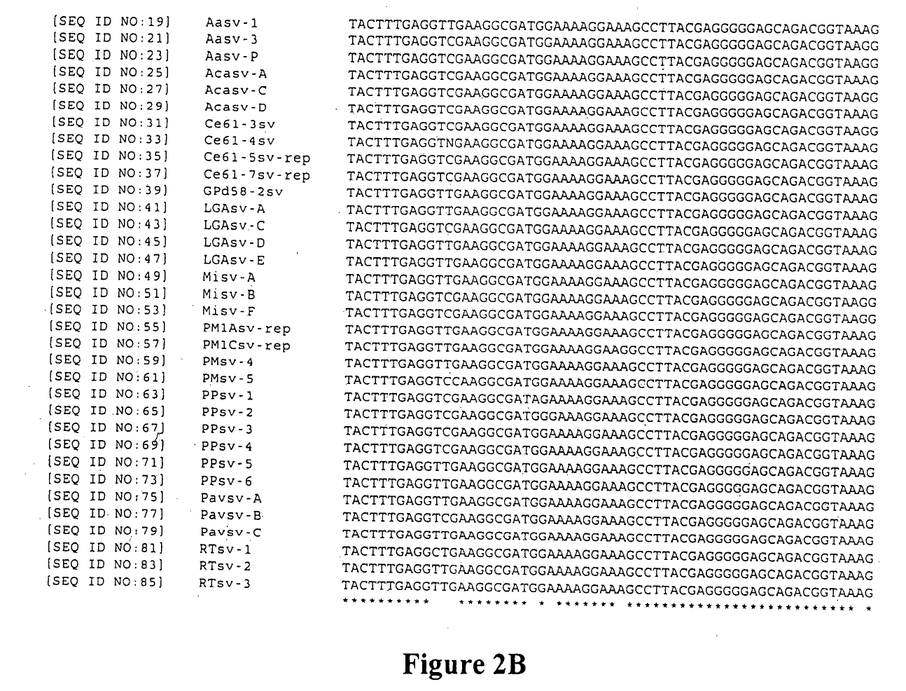

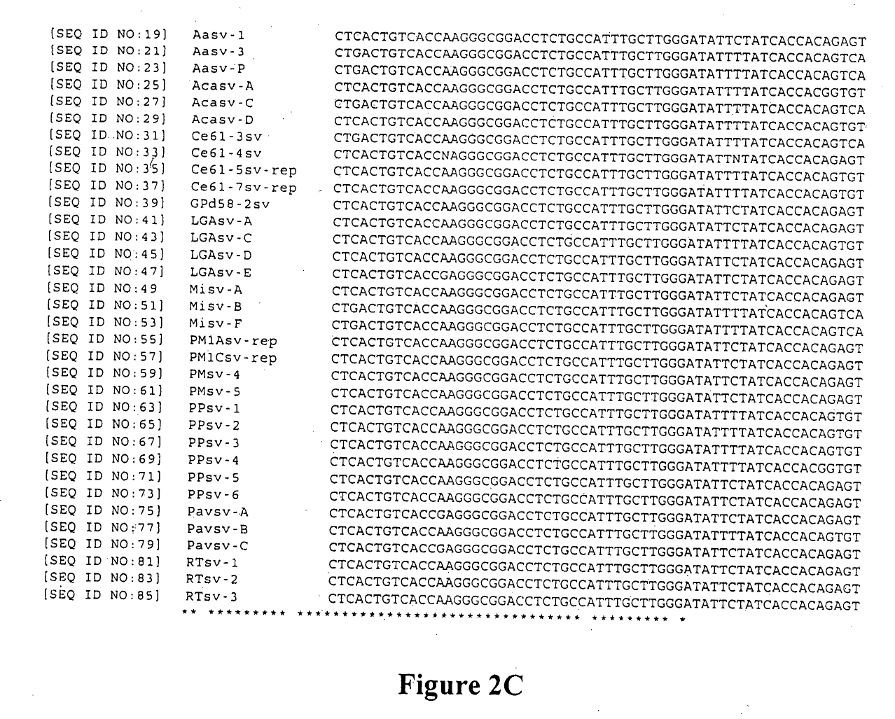

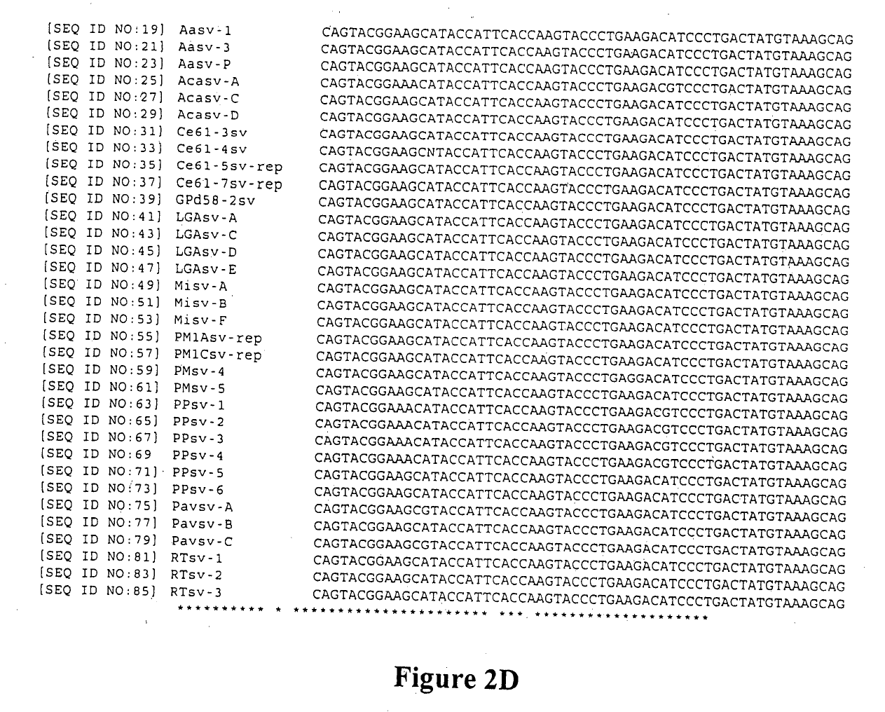

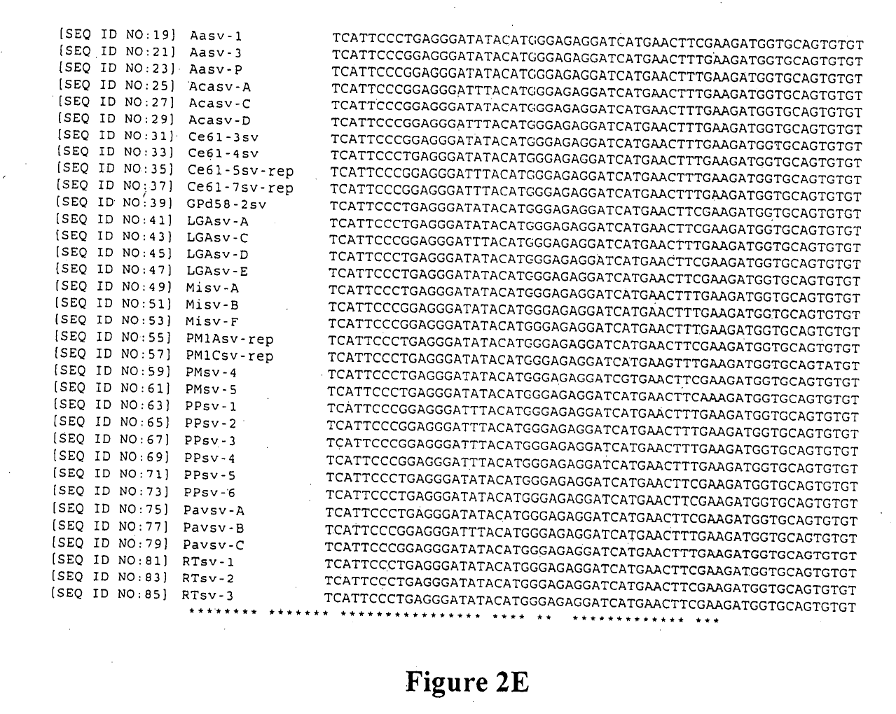

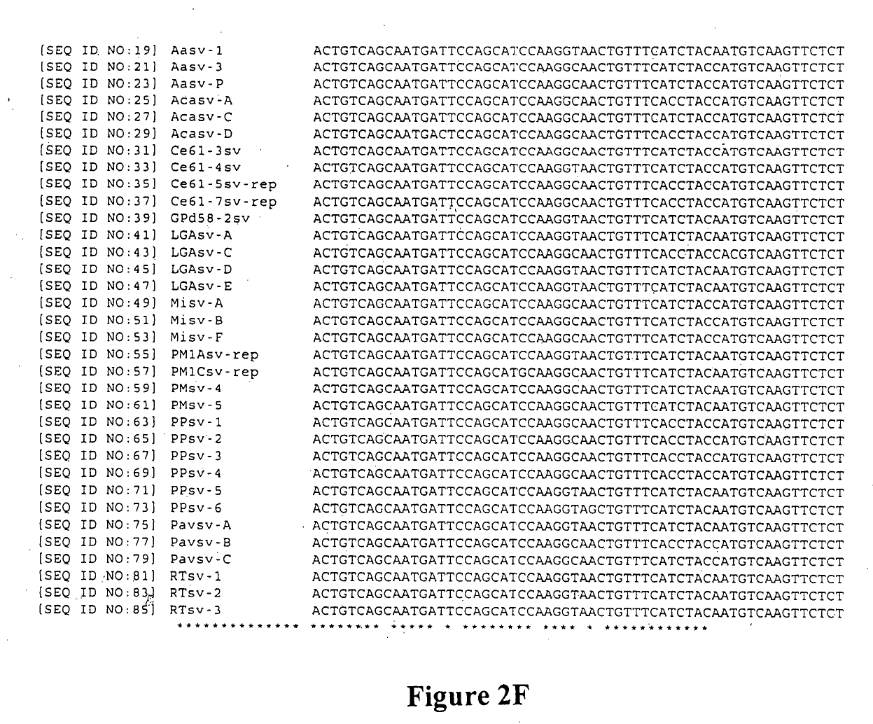

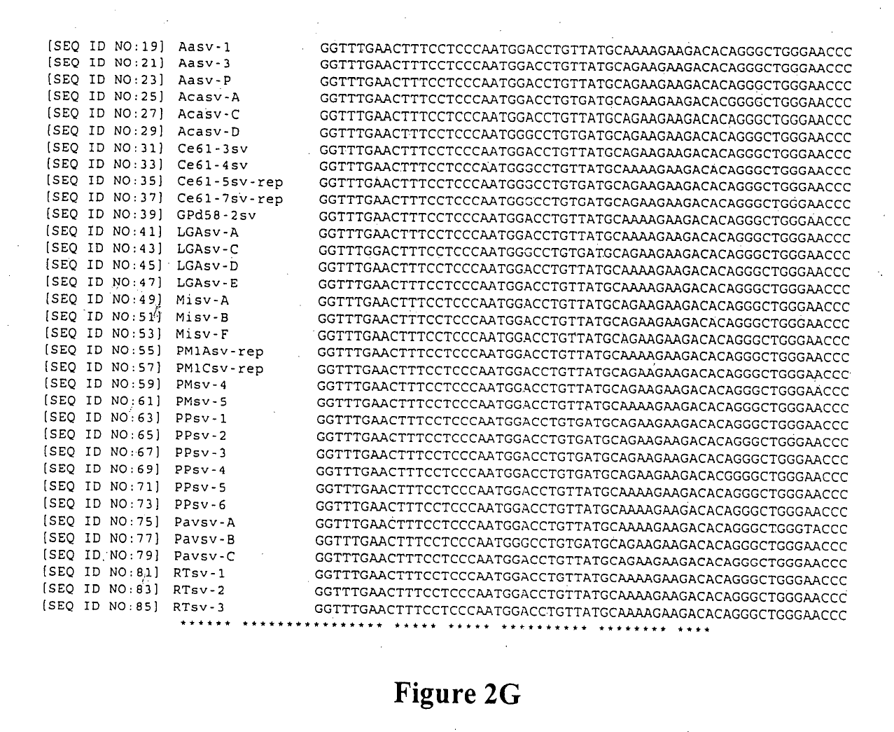

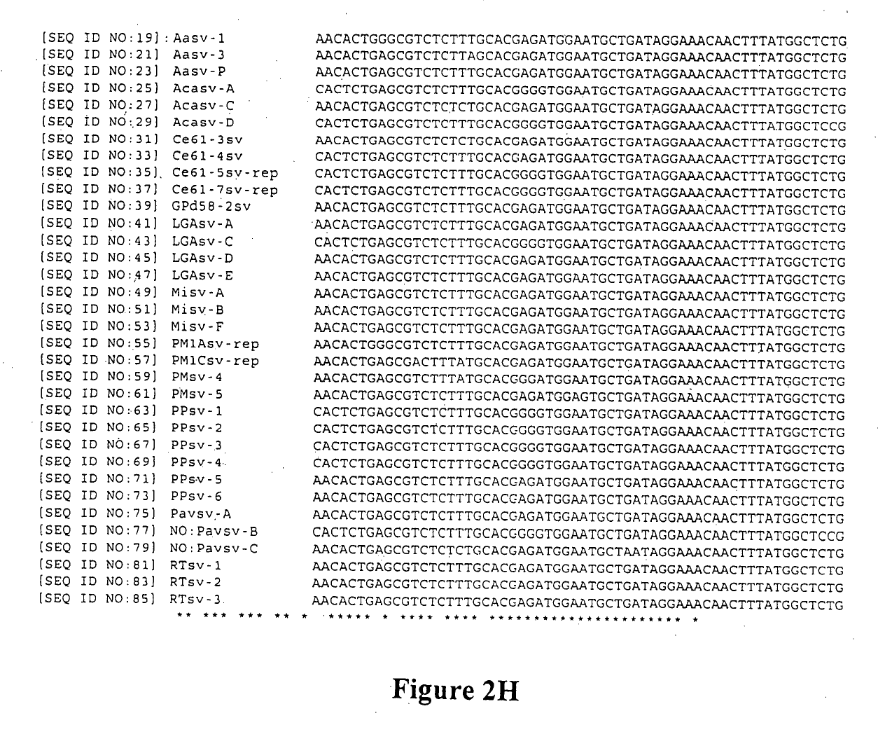

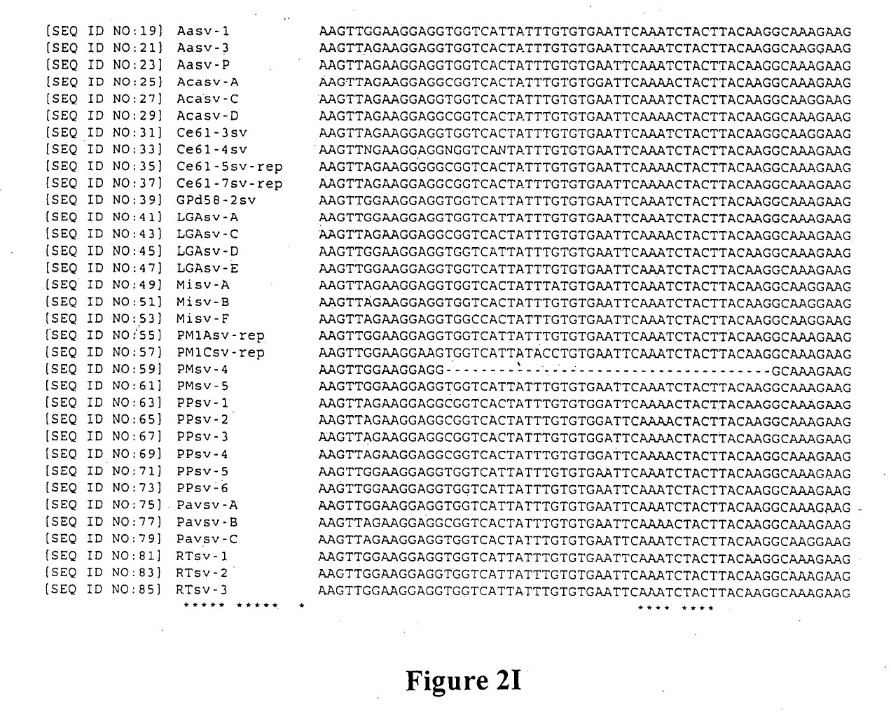

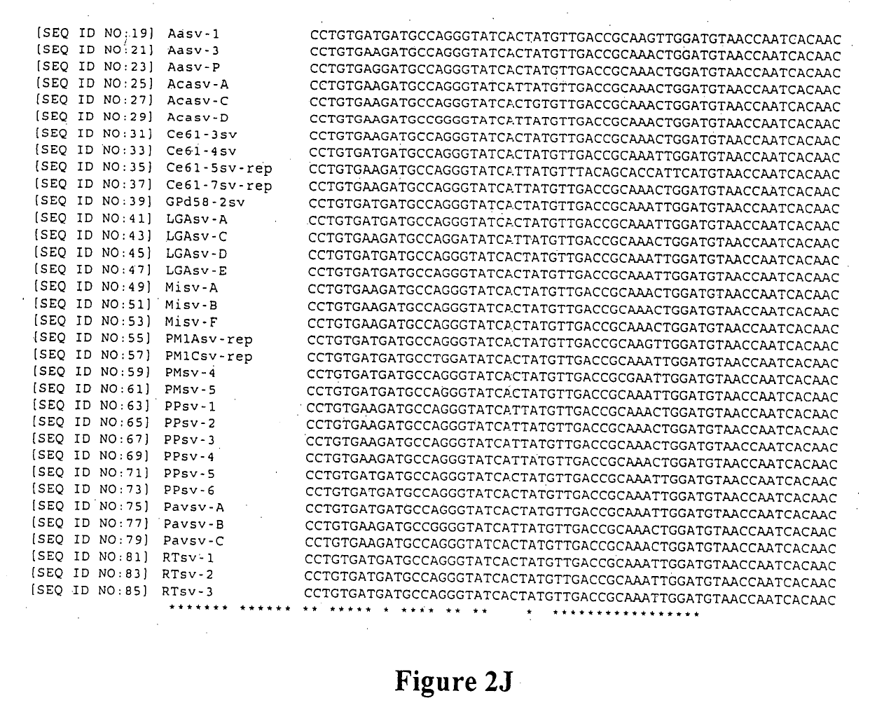

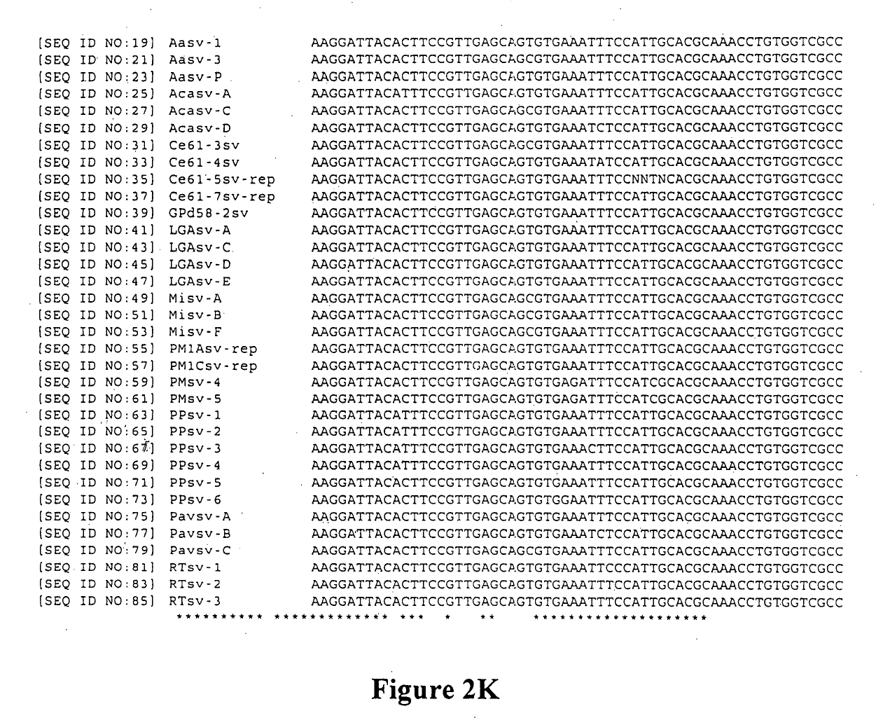

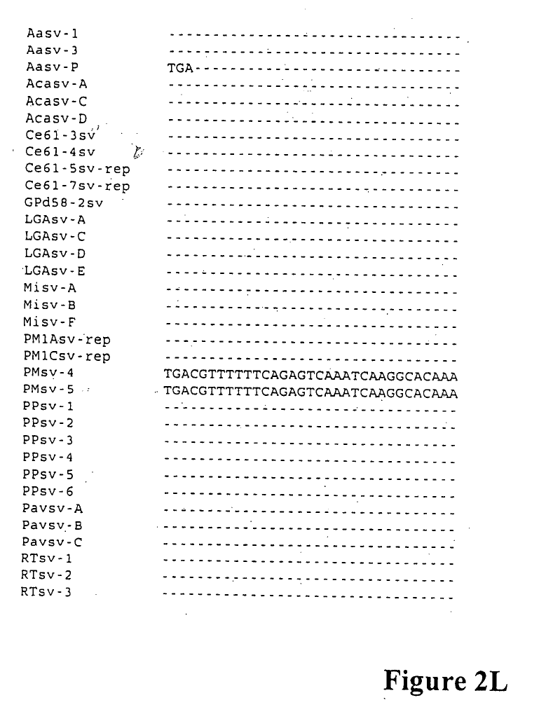

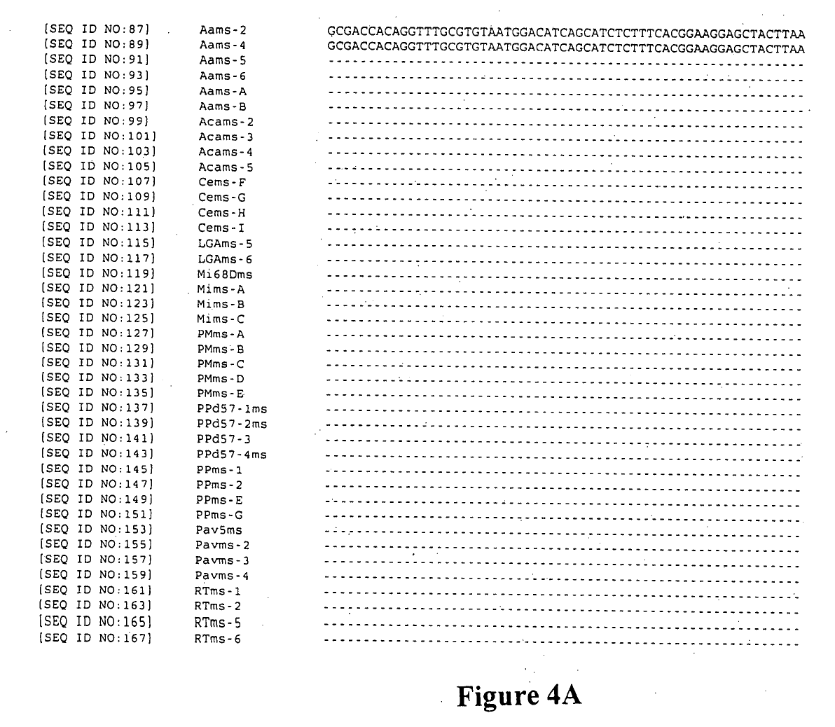

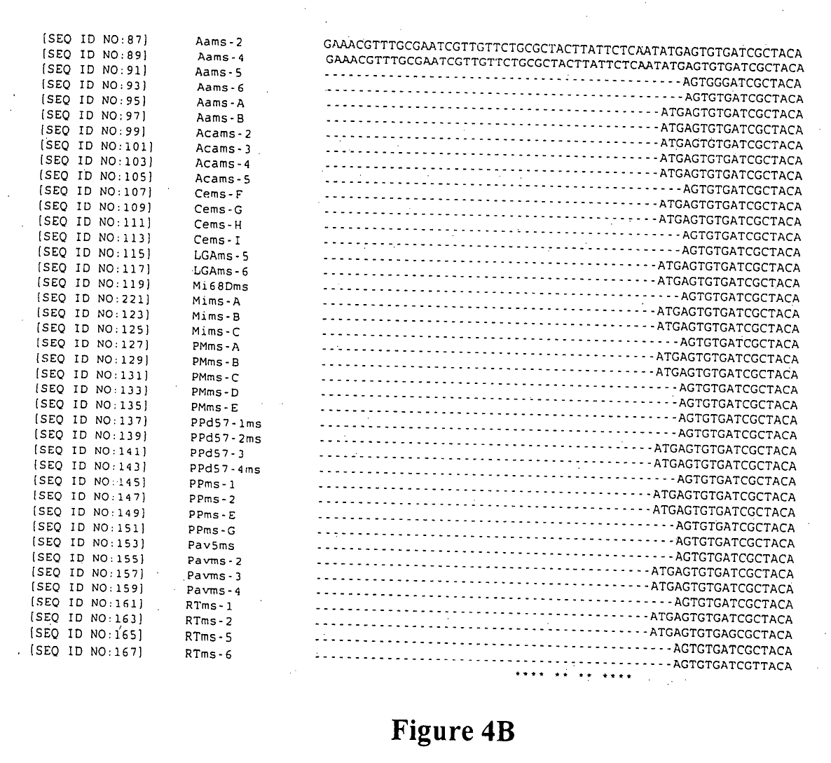

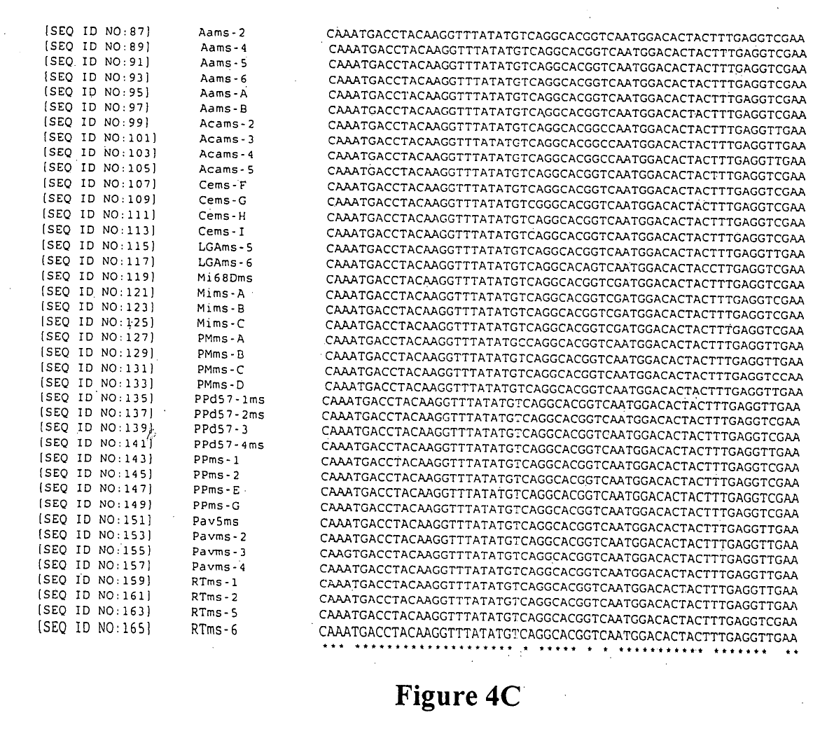

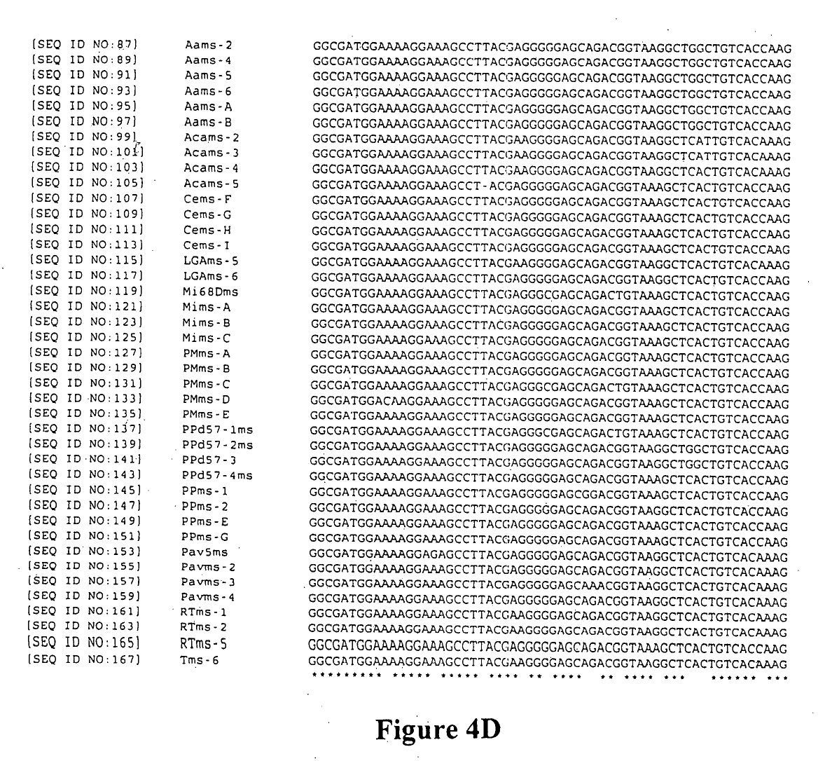

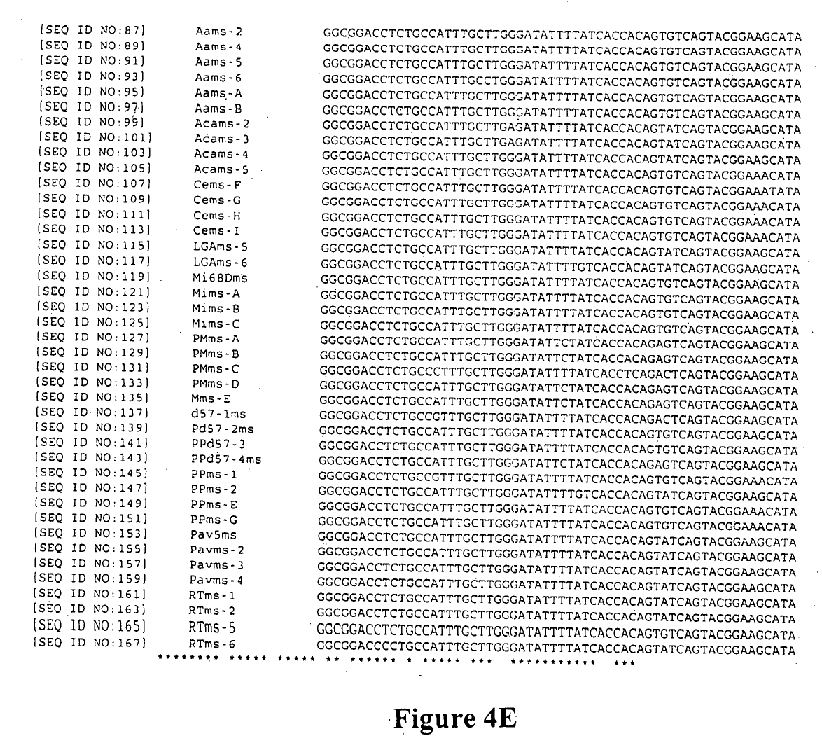

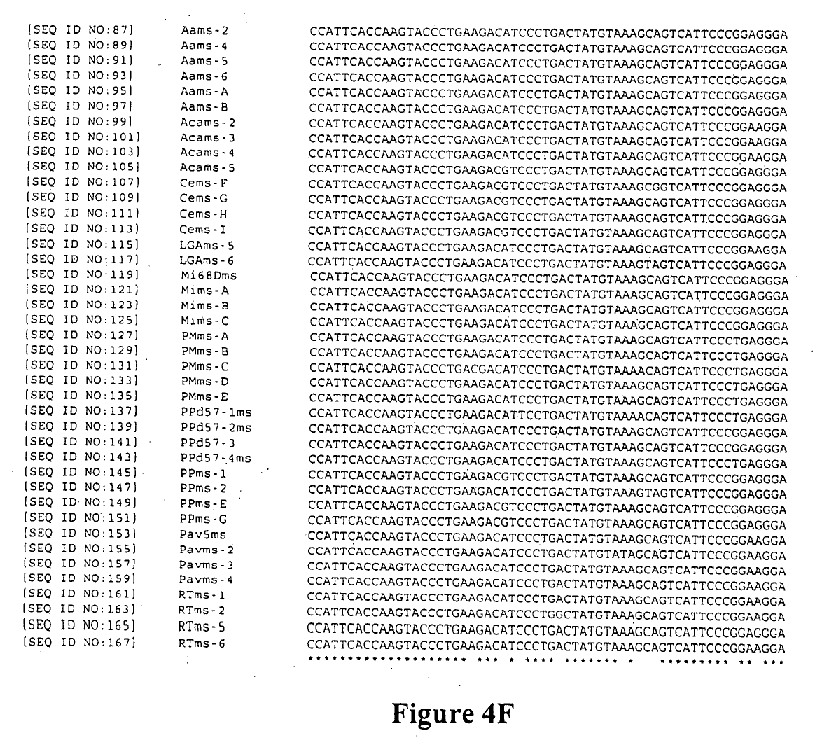

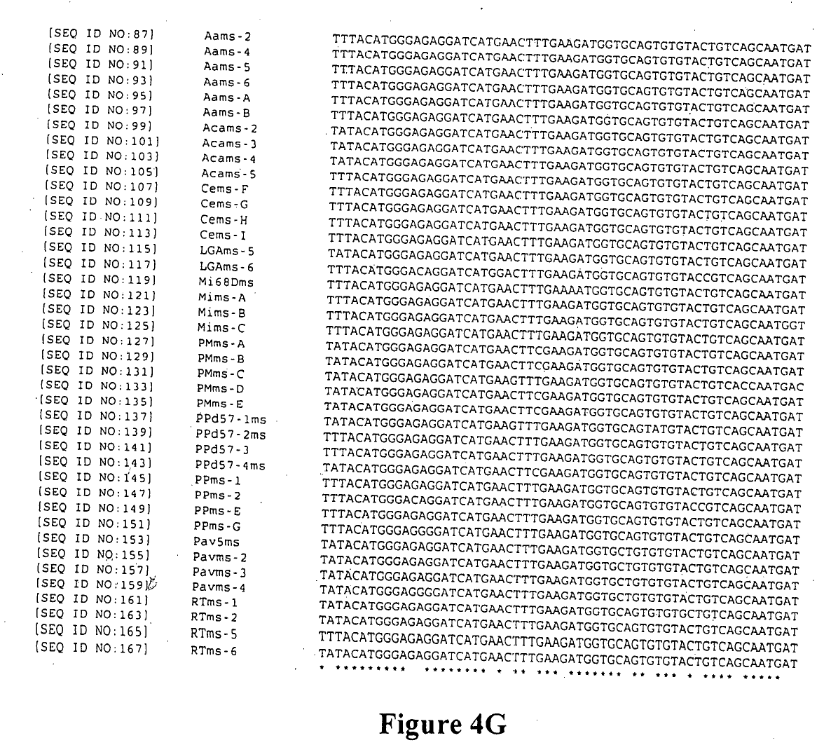

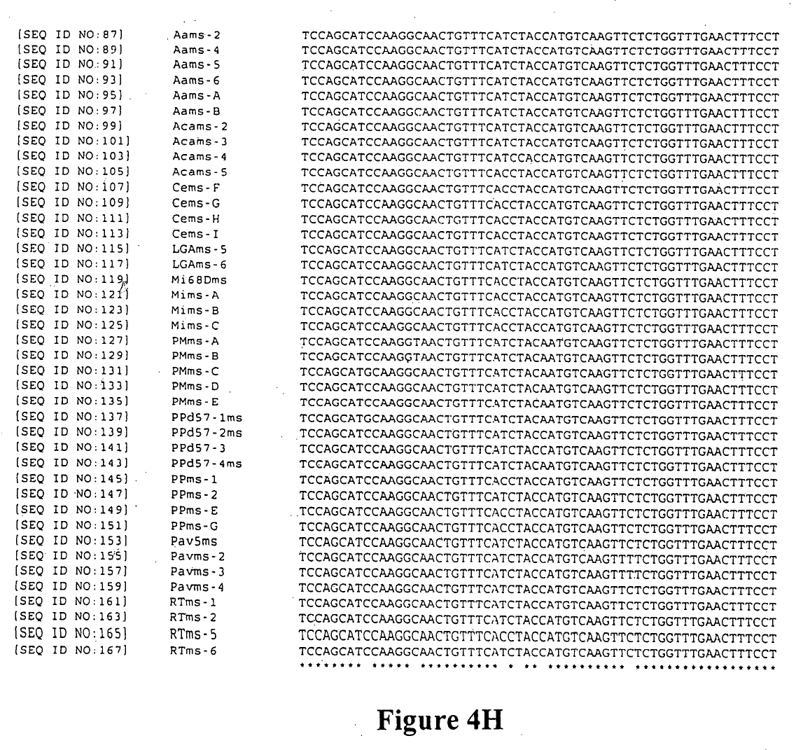

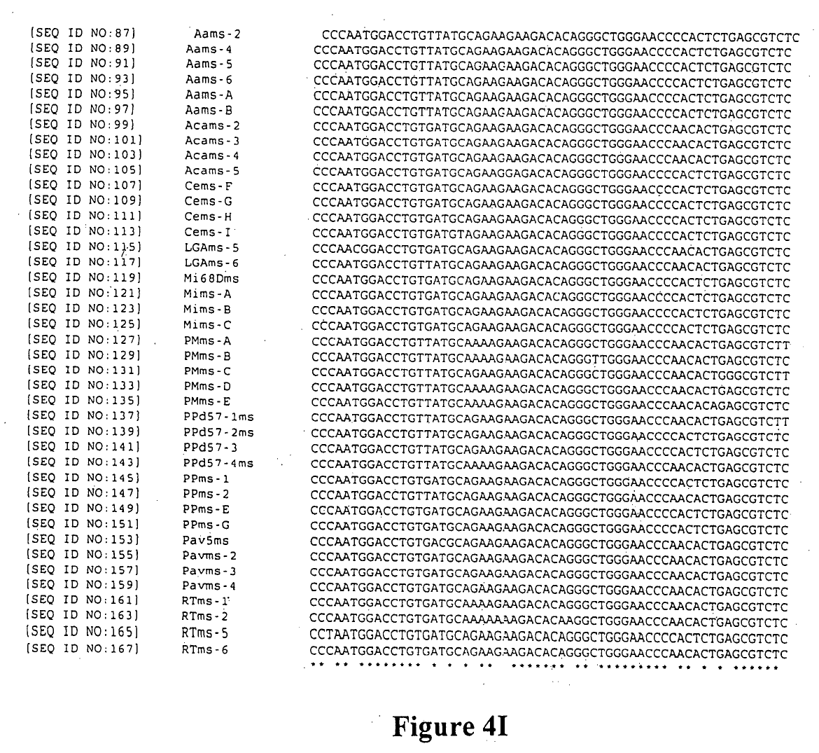

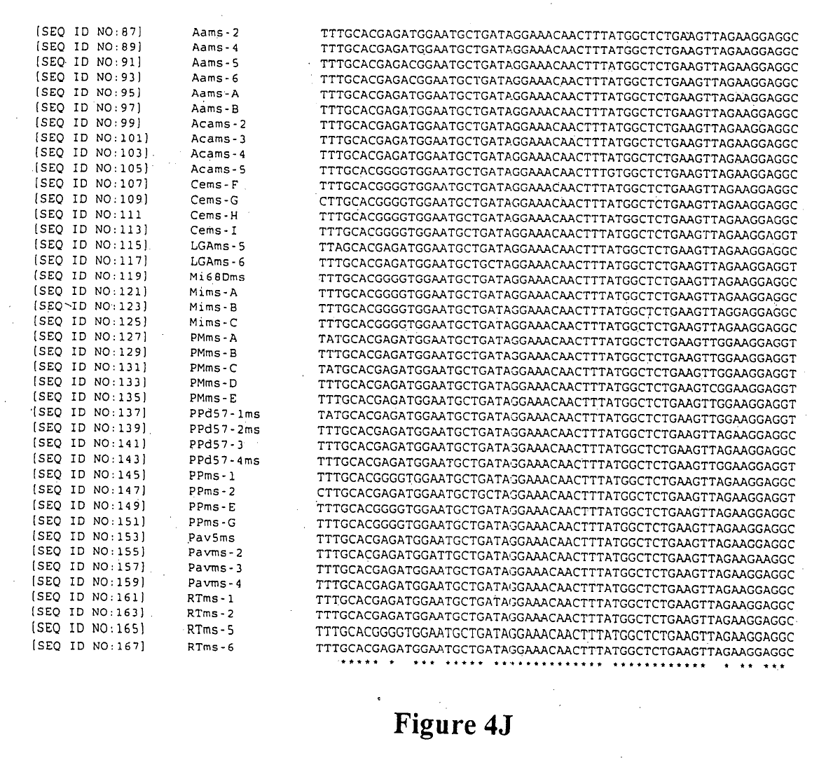

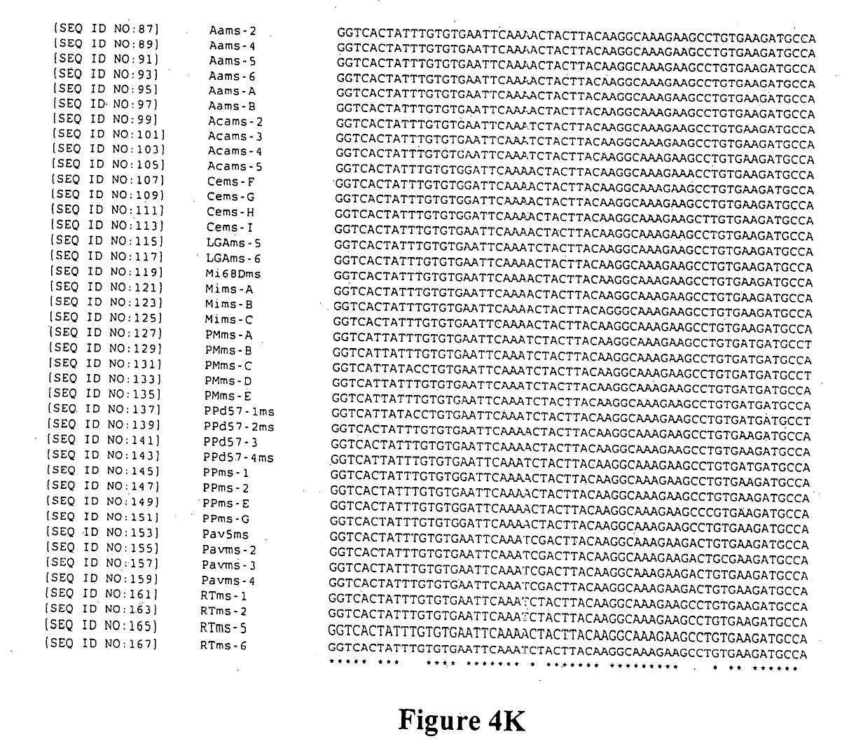

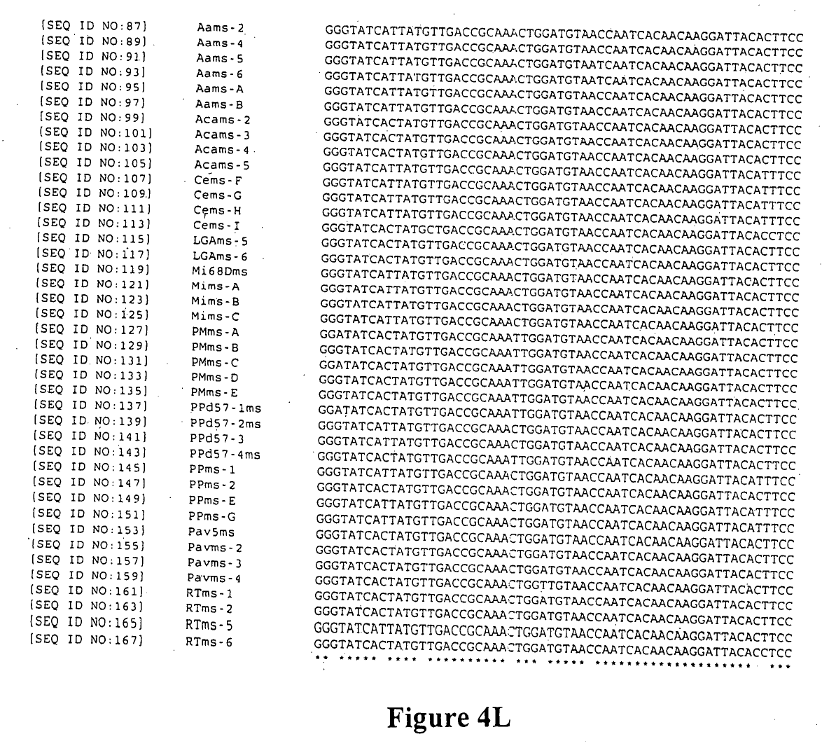

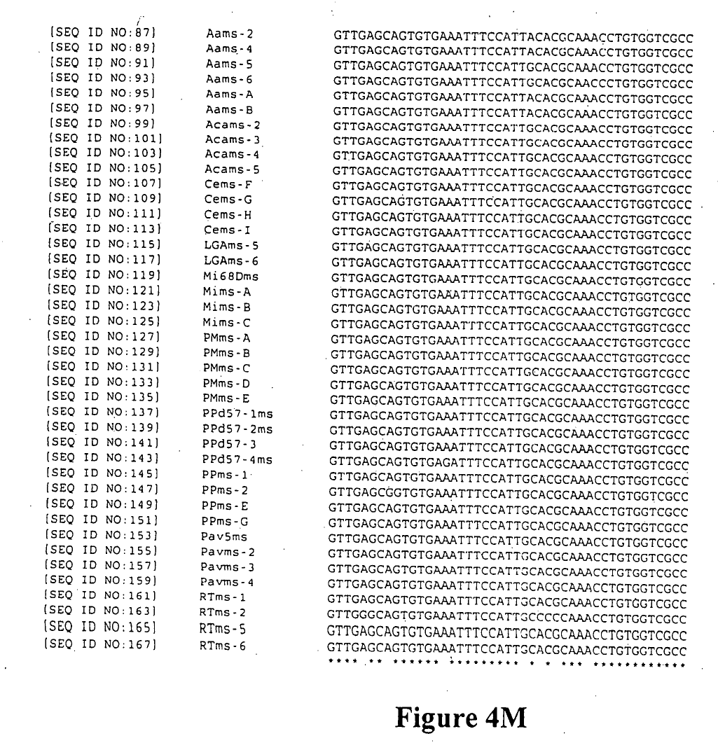

FIGS. 1 and 3 show amino acid sequences for polypeptides comprising amino terminal SVWAK (SEQ ID NO:5; FIG. 1) and comprising (M)SVIAT (SEQ ID NO:6), SGIAT (SEQ ID NO:7), SVIVT (SEQ ID NO:8) and SVSAT (SEQ ID NO:9) at or near the terminal amino end (FIG. 3). Aams-2.pep (SEQ ID NO:88) and Aams-4.pep (SEQ ID NO:90) are shown comprising additional amino acids at the amino terminal end. Alignments of the corresponding nucleotide sequences of the amino acid sequences shown in FIGS. 1 and 3 are set forth in FIGS. 2 and 4, respectively.

Polypeptides comprising five shared amino acid sequences SVIAK (SEQ ID NO:5), (M)SVIAT (SEQ ID NO:6), SGIAT (SEQ ID NO:7), SVIVT (SEQ ID NO:8) and SVSAT (SEQ ID NO:9) may be grouped accordingly. Additional common amino acids immediately adjacent to the abovementioned amino acids are shown below:

| | |
|---|---|
| SVIAK QMTY KVYM SGT; | (SEQ ID NO:10) |
| SVIAT QMTY KVYM PGT; | (SEQ ID NO:11) |
| SVIAT QVTY KVYM SGT; | (SEQ ID NO:12) |
| SGIAT QMTY KVYM SGT; | (SEQ ID NO:13) |
| SVIVT QMTY KVYM SGT; | (SEQ ID NO:14) |
| SVSAT QMTY KVYM SGT; | (SEQ ID NO:15) |
| SVIAK QMTY KVNM SGT; | (SEQ ID NO:16) |
| SVIAK QMTY KVYM SDT; | (SEQ ID NO:17) |
| and | |
| SVIAK QMTY X₁X₂YX₃ SGT, | (SEQ ID NO:18) |

wherein X₁, X₂ and X₃ may be any amino acid provided that X₁ is not K; X₂ is not V; X₃ is not M.

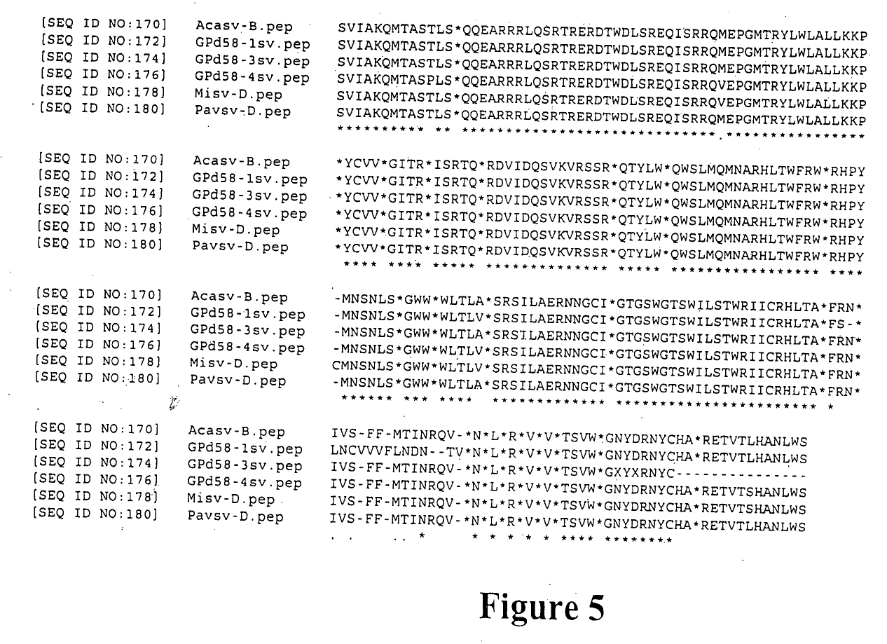

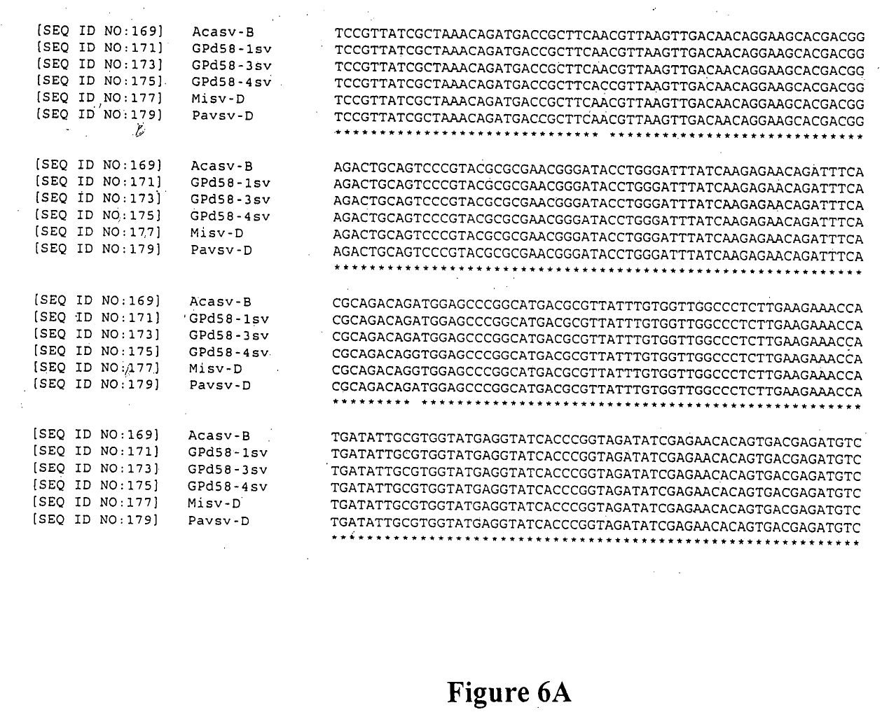

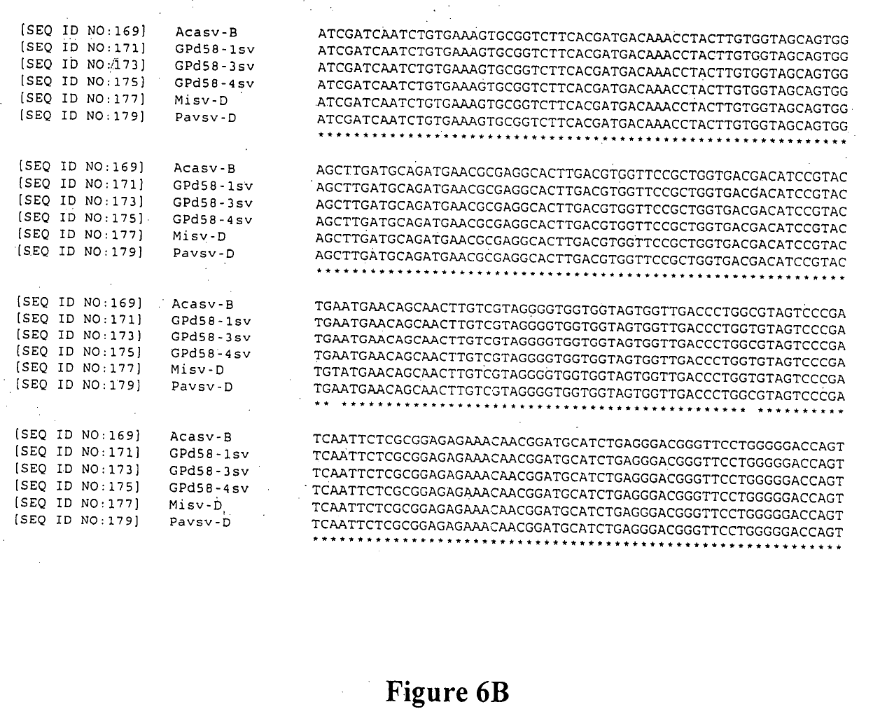

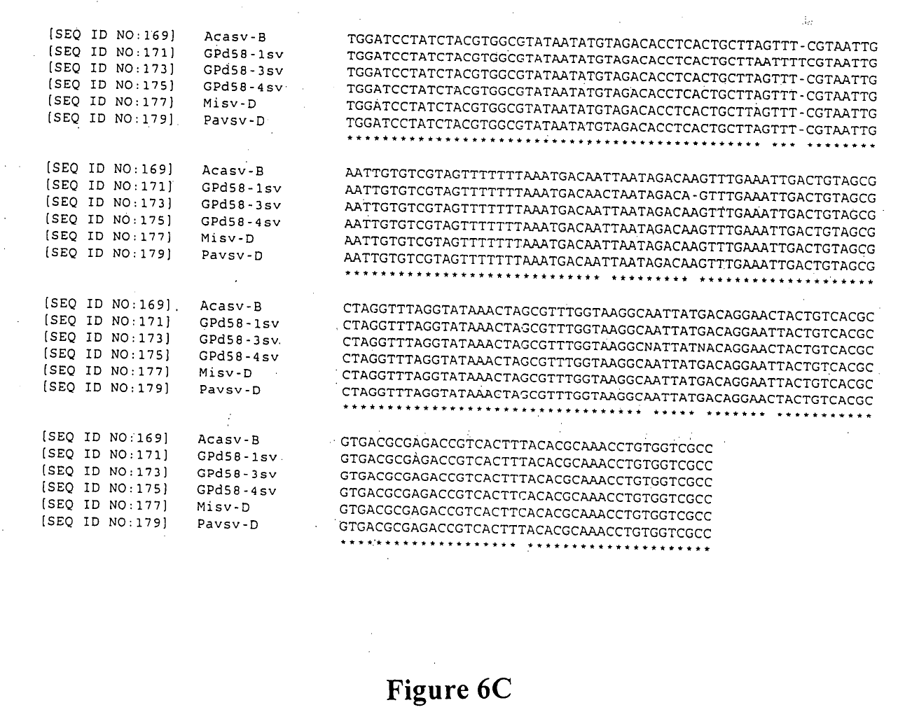

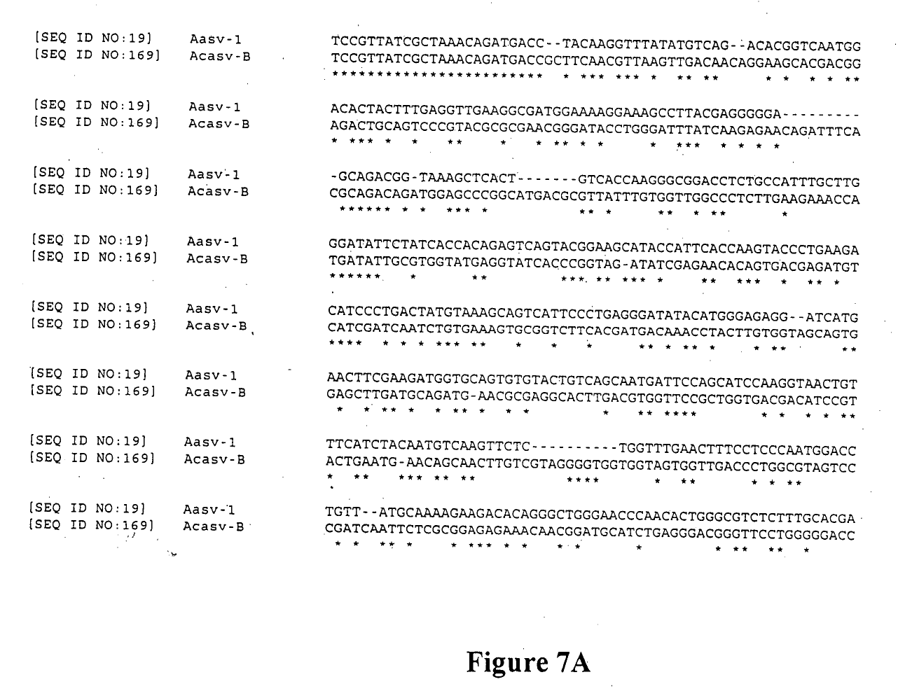

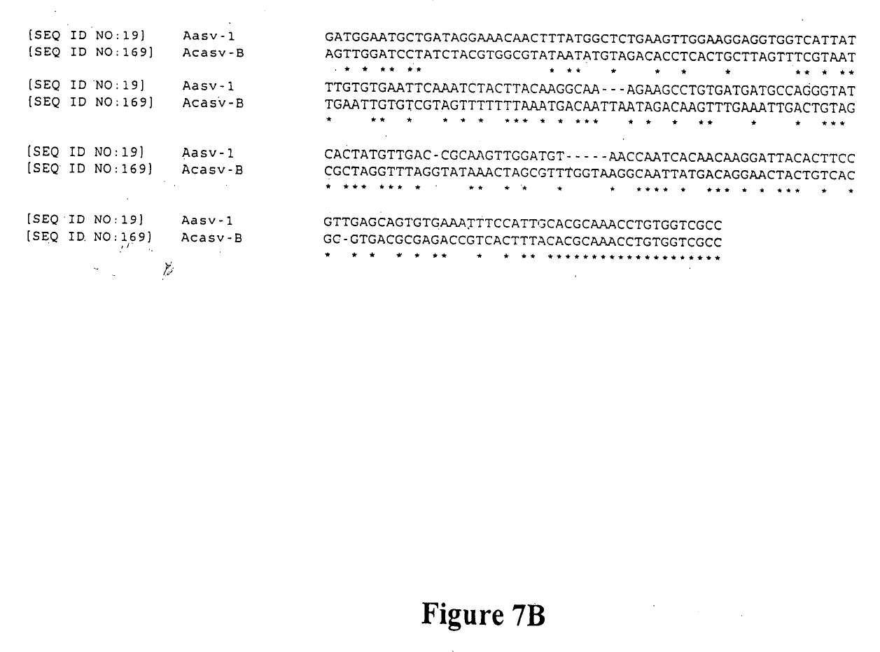

FIG. 5 shows an alignment of amino acid sequences comprising SVLAK (SEQ ID NO:5) at the amino terminus and a stop or termination codon at corresponding amino acid residue 14. The termination codon results from the addition of two nucleic acid residues. The resulting polypeptide is much different when compared with polypeptides lacking this termination codon. An alignment of the corresponding nucleic acid sequences is shown in FIG. 6. These nucleic acids are approximately 40 nucleotide bases longer than those lacking the termination codon (FIG. 6). The differences can be more redily seen by referring to FIG. 7, which shows an alignment of one nucleic acid sequence comprising the termination codon (SEQ ID NO:169) and a nucleic acid sequence lacking the termination codon (SEQ ID NO:19).

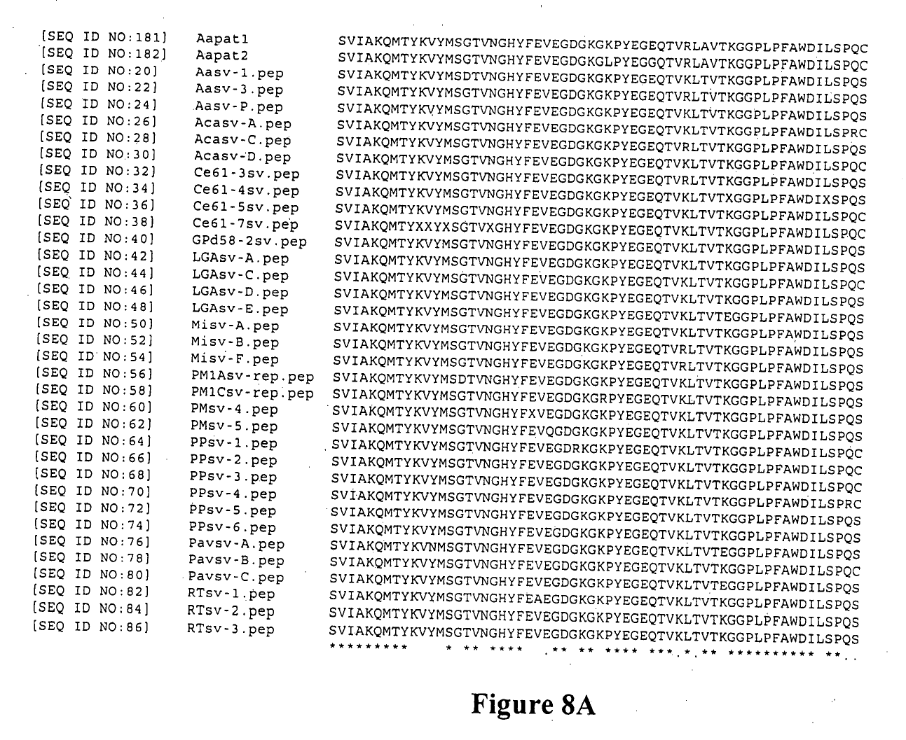

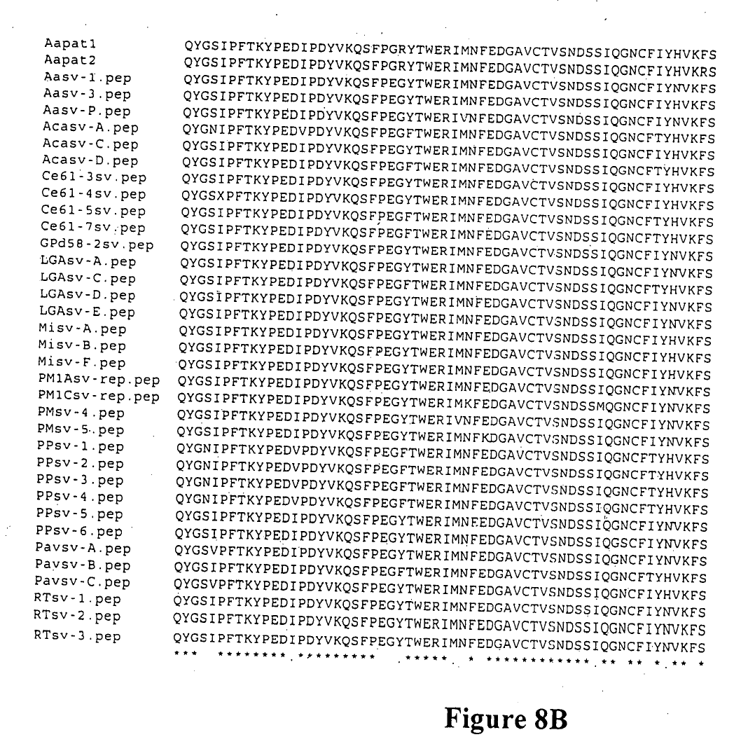

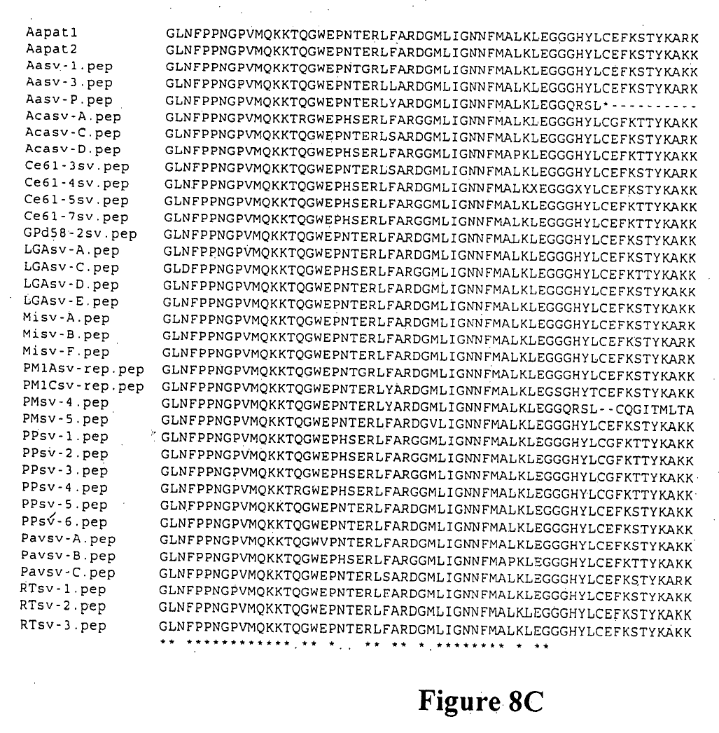

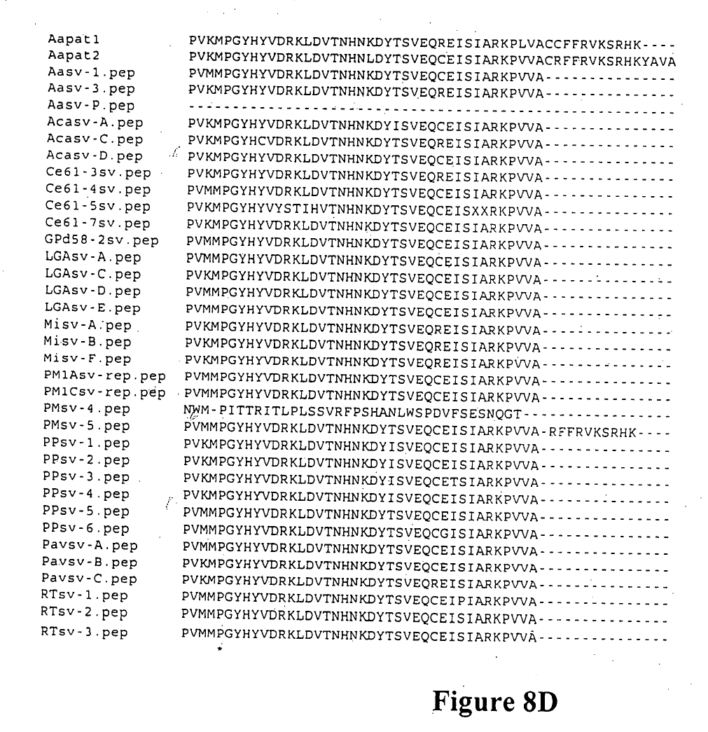

Previously-disclosed SVIAK (SEQ ID NO:5)-containing proteins Aapat-1 (SEQ ID NO:181) and Aapat-2 (SEQ ID NO:182) are also included on an amino acid sequence alignment with many of the SVIAK (SEQ ID NO:5)-containing polypeptides of the present invention, in FIG. 8. Shaded amino acid residues indicate amino acids unique to SEQ ID NO:181 and/or SEQ ID NO:182.

### EXAMPLE 3

### Isolation of New Colored-protein Sequences from Melbourne Coral

Extraction and Visualization of Colored Proteins from Coral

Samples of various coral and algae were purchased from Water World Aquarium (Melbourne, Australia) and Coburg Aquarium (Melboure, Australia). These included Goniopora sp. (“flower pot coral”) [brownish tentacles with an iridescent green centre underwater], green Acropora sp. coral (“staghom coral”), brown/light blue Porites sp. coral (“finger”), Sinularia sp. and Tubastrea sp. corals as well as deep blue and bright orange Corallimorphs (Discosoma sp.).

Small samples of each coral were incubated in 1 M sodium phosphate buffer pH 7.5 at 4° C. A sample of “purple algae” that was growing on dead coral (normally sold as “living rock”) was also collected in buffer. After 48 b the Acropora sp. extract appeared yellow-brown in color, the Porites sp. extract appeared orange in color and the purple algae extract was a clear pink color.

When the extracts were exposed to UV light the Acropora sp. extract contained orange and blue fractions, the Porites sp. extract contained pink fractions and the “purple algae” extract was a bright orange color.

Goniopora sp. coral tips were extracted in 1 M Na phosphate buffer pH 7.5. After an overnight incubation at 4° C. the extract was orange-pink under natural light and appeared orange under UV light. Fluorescent green fractions were also observed in the solid phase under UV light.

A 10 μL sample of the crude extracts described above was electrophoresed through precast SDS PAGE gels (12% w/v resolving, 4% w/v stacking gel) (Ready Gels, Biorad) in a running buffer made of 25 mM Tris-HCl, pH 8.3, 192 mM glycine, 0.1% w/v SDS at 100V for 75 min. The crude protein extracts were either denatured by boiling in 10% v/v glycerol, 3% w/v SDS, 3% P-mercaptoethanol, 0.025% w/v bromophenol blue or loaded in their native state in 5% v/v glycerol, 0.04% w/v bromophenol blue. Standards included pre-stained Low Range markers (Biorad) which contained standard protein samples of 116 kDa, 80 kDa, 51.8 kDa and 34.7 kDa.

Prior to staining with Coomassie blue (0.25% (w/v) Coomassie Brilliant Blue, 45% (v/v) methanol, 10% (v/v) acetic acid), PAGE gels were examined under a hand-held UV transilluminator (BLAK-RAY, longwave UV lamp, model B100 AP, UVP Inc). The non-denatured crude protein extract from Goniopora sp. contained orange bands (running higher than 116 kD marker protein) and blue-green bands (running between 51.8 kD and 80 kD protein markers). The non-denatured crude protein extract from Porites sp. contained two orange bands under UV light at approximately the same position as that from Goniopora sp (i.e. running higher than 116 kD marker protein). The non-denatured crude protein extract from Acropora sp. contained a single orange band under UV light at approximately the same position as that from Goniopora sp. (i.e. running higher than 116 kD marker protein) as well as a green band (running between 80 kD and 116 kD marker proteins).

These fluorescent bands were not observed in any of the denatured crude protein extracts. No protein bands were visible under natural light before Coomasie blue staining.

Isolation of RNA and Synthesis of cDNA from Coral

Total RNA was isolated from the anthozoans Acropora sp., Discosoma sp., Sinularia sp. and Tubastrea sp. using RNeasy Plant mini kit (Qiagen) or the method of Turpen and Griffith (Biotechniques 4:11-15, 1986).

Complementary DNA was synthesized using 1 μg total RNA, 1 μL DNase RQ1 RNase free (Promega), 1 μL 10×buffer (final concentration: 40 mM Tris-HCl pH 8, 10 mM NaCl, 6 mM MgCl₂, 10 mM CaCl₂). The reactions were incubated at 37° C. for 10 min then 65° C. for 10 min. One microlitre (1 μg) of primer dT(17)Ad2Ad1 (SEQ ID NO:183) was then added and the reaction was boiled for 5 min and then incubated on ice for 5 min. 4 μL 5×RT buffer, 2 μL 0.1 M DTT, 1 μL 10 mM dNTPS and 1 μL RNasin (Promega) were then added and the reaction was incubated at 50° C. for 2 min. 1 μL (200 U) Superscript II reverse trancriptase (Gibco BRL) was then added and the reaction was incubated at 50° C. for 1.5 h. The cDNA was purified using QIAquick PCR purification kit (Qiagen).

PCR of Colored Protein Sequences

Oligonucleotide primers “vispro-F1” (SEQ ID NO:184) and “vispro-R1” (SEQ ID NO:185) were designed to hybridize to the 5′ and 3′ ends of T7SP6BASPOC3 and T7SP6BASPOC4 sequences, respectively (International Patent Application No. PCT/AU00/00056). The primer “vispro-F1” (SEQ ID NO:184) contained a BamHI site for cloning into the bacterial expression vector pQE30 (Qiagen) and an AscI site with a translation initiating codon for cloning into binary vectors. The primer “vispro-R1” (SEQ ID NO:185) contains a PstI site for cloning into the bacterial expression vector pQE-30 and a PacI site with translation termination codon for cloning into binary vectors.

```
SEQ ID NO:184 vispro-F1 (5′ to 3′)
            AscI BamHI
CAG GGCGCGCC ATG GGA TCC GTT ATC GCT AAA CAG ATG ACC
                            M G S V I A K Q M T
SEQ ID NO:185 vispro-R1 (5′ to 3′)
              PacI PstI
GGG TTA ATT AAG CTG CAG GGC GAC CAC AGG TTT GCG TG
        stop N L Q L A V V P K R
```

Polymerase chain reactions were set up using 20 pmole vispro-F1 (SEQ ID NO:184) and 20 pmole vispro-R1 (SEQ ID NO:185) primers and 5 μL cDNA synthesized from coral RNA as template, 2.5 units HotStarTaq (trademark) DNA polymerase (Qiagen), 200 μM dNTP mix and 1 X PCR buffer (Qiagen) in a 50 μL reaction.

PCR conditions included a denaturation step at 95° C. for 15 min, followed by 35 cycles of 94° C. for 30 sec, 50° C. for 30 sec and 72° C. for 1 min with a final treatment at 72° C. for 10 min followed by storage at 4° C.

PCR products were electrophoresed through a 1% w/v agarose gel. Products of 700 bp were excised from the gel and purified using QIAEX II Gel Extraction Kit (Qiagen). Purified DNA was digested with BamHI and PstI restriction enzymes and re-purified using a QIAquick PCR purification Kit (Qiagen). The purified DNA was ligated with BamHI/PstI ends of the bacterial expression vector pQE-30 (Qiagen). Ligated DNA was transformed into Eschericia coli BL21-RIL, M15 (containing pREP4 (Qiagen)) or XL1-blue competent cells and plated onto Luria Broth (LB) agar plates containing 100 μg/mL ampicillin. After overnight incubation at 37° C a colony lift on nylon membrane (DuPon/NEN) was taken an d placed colony side up onto LB agar containing 100 μg/mL ampicillin and 1 mM IPTG. The plates were incubated overnight at 37° C. or alternatively at room temperature for 2 nights. Blue and purple colored colonies that were visible under natural light were obtained from products originating from Acropora sp., Discosoma sp., Sinularia sp. and Tubastrea sp.

Cultures of the purple and blue colonies were initiated and incubated overnight at 37° C. Plasmid DNA was isolated and analyzed by restriction endonuclease digestion. Plasmid DNA isolated from purple colonies included pCGP2915 (A10 clone from Acropora sp.), pCGP2916 (A11 clone from Acropora sp.), pCGP2917 (A12 clone from Acropora sp.), pCGP2918 (A8 clone from Acropora sp.), pCGP2920 (D10 clone from Discosoma sp.), pCGP2922 (T3 clone from Tubastrea sp.), pCGP2924 (S3 clone from Sinularia sp.).

Plasmid DNA isolated from blue colonies included pCGP2919 (D1 clone from Discosoma sp.), pCGP2921 (T1 clone from Tubastrea sp.), pCGP2923 (S1 clone from Sinularia sp.).

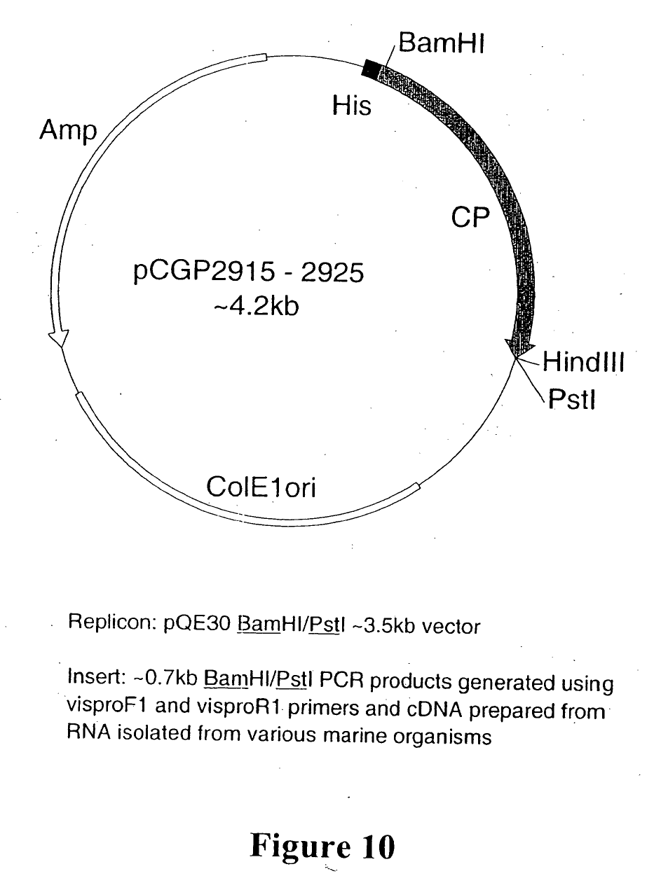

See FIG. 10 for all schematics of above mentioned plasmids.

Sequence Analysis of CDNA Clones

Complete sequence analysis of the cDNA clones contained in the pQE-30 vectors was generated using pQEprom (Qiagen) (SEQ ID NO:186), pQErev (Qiagen) (SEQ ID NO:187), Coral-R1 (SEQ ID NO:188), vispro-F1 (SEQ ID NO:184) and vispro-R1 (SEQ ID NO:185) as sequencing primers.

| | |
|---|---|
| pQEprom | |
| CCC GAA AAG TGC CAC CTG | SEQ ID NO:186 |
| pQErev | |
| GTT CTG AGG TCA TTA CTG G | SEQ ID NO:187 |
| Coral-R1 | |
| TCA GGG TAC TTG GTC AAT GG | SEQ ID NO:188 |

Complete nucleotide sequences were generated from the:-

A8 cDNA clone from Acropora sp. contained in pCGP2918 (SEQ ID NO:189);

D10 cDNA clone from Discosoma sp contained in pCGP2920 (SEQ ID NO:191);

S3 cDNA clone from Sinularia sp contained in pCGP2924 (SEQ ID NO:193);

T3 cDNA clone from Tubastrea sp. contained in pCGP2922 (SEQ ID NO:195);

D1 cDNA clone from Discosoma sp. contained inpCGP2919 (SEQ ID NO:197);

S1 cDNA clone from Sinularia sp. contained in pCGP2923 (SEQ ID NO:199); and

T1 cDNA clone from Tubastrea sp. contained in pCGP2921 (SEQ ID NO:201).

The A8 nucleotide sequence contained a putative open reading frame of 669 bases which encodes a putative polypeptide of 223 amino acids (SEQ ID NO:190).

The D10 nucleotide sequence contained a putative open reading frame of 669 bases which encodes aputative polypeptide of 223 amino acids (SEQ ID NO:192).

The S3 nucleotide sequence contained a putative open reading frame of 669 bases which encodes a putative polypeptide of 223 amino acids (SEQ ID NO:194).

The T3 nucleotide sequence contained a putative open reading frame of 669 bases which encodes a putative polypeptide of 223 amino acids (SEQ ID NO:196).

The D1 nucleotide sequence contained a putative open reading frame of 669 bases which encodes a putative polypeptide of 223 amino acids (SEQ ID NO:198).

The S1 nucleotide sequence contained a putative open reading frame of 669 bases which encodes a putative polypeptide of 223 amino acids (SEQ ID NO:200).

The T1 nucleotide sequence contained a putative open reading frame of 669 bases which encodes a putative polypeptide of 223 amino acids (SEQ ID NO:202).

Nucleotide and amino acid sequence similarities were determined using LALIGN (Huang and Miller, 1991, supra). The sequences isolated from the four species of coral share high nucleic acid and amino acid sequence similarities (Table 8 and Table 9).

### EXAMPLE 4

### Colored Protein Expression from Heron Island Coral cDNAs

For expression in bacteria, nucleotide sequences encoding CFMs were retrieved from pGEM-T cloning vector using a forward oligonucleotide primer consisting of the NotI restriction binding site, a ribosomal binding site, a spacer and 15 bases encoding the N-terminus of the protein and a reverse oligonucleotide primer encoding H6-tag (POC220-H6; POC220 is SEQ ID NO:2). PCR product was gel purified and diluted (×10) prior to cloning into PCRII-TOPO and transformed into Top 10 cells (Invitogen). Cells were induced with 0.5 mM IPTG, and protein was purified on Ni-columns (Pro-Bond, Invitrogen) eluting with 50 mM, 200 mM, 350 mM and 500 mM Inidazole in PBS pH 6.0, prior to overnight dialysis against 50 mM Potassium Phosphate pH 6.65.

Expression of Examples of Type 1 Peptides

Results of expressing sequences of type 1 (as defined in Tables 6 and 7 and in FIG. 9) in bacteria are set forth in Table 10. Only non-identical sequences are shown. Several additional sequences, which are identical to those shown in the Table, are indicated at the top of the Table (i.e.: Acasv-D=PavsvB, etc.). Sequence alignment is taken from International Patent Publication No. WO 00/46233 and Dove et al. (2001; supra). Horizontal bars above the amino acid sequence indicate β-strands from GFP structure. The chromophore “QYG” is shown in white type on black background. Amino acid differences in the sequences are grey-shaded.

The majority of type 1 sequences are deep blue with λₘₐₓ ranging from 589 nm to 593 nm. Naturally-occurring amino acid substitution L161P, as seen in RTms5 (SEQ ID NO:166) compared with Acasv-D (SEQ ID NO:30) leads to clear bacteria that no longer absorb within 520-600 nm range. Reverse substitution of P161L re-establishes the ability to absorb in this range. The alignment shows amino acids that appear to affect colour of protein and those that do not.

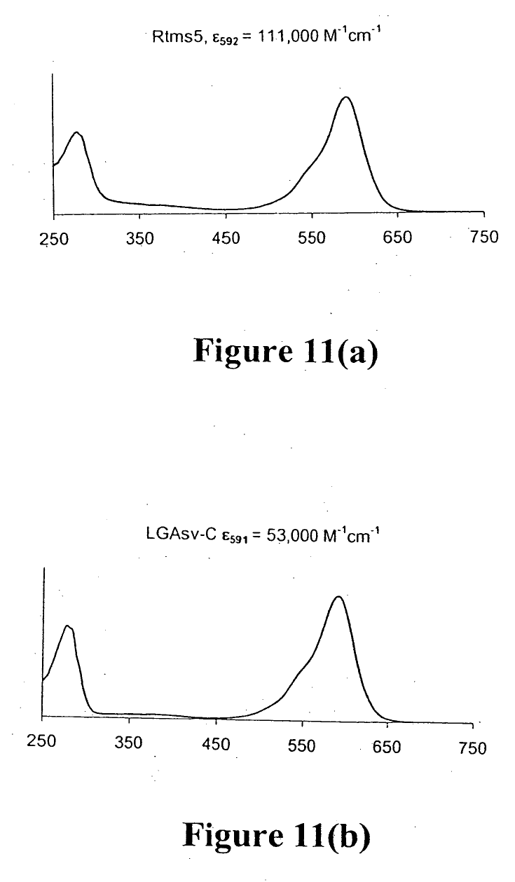

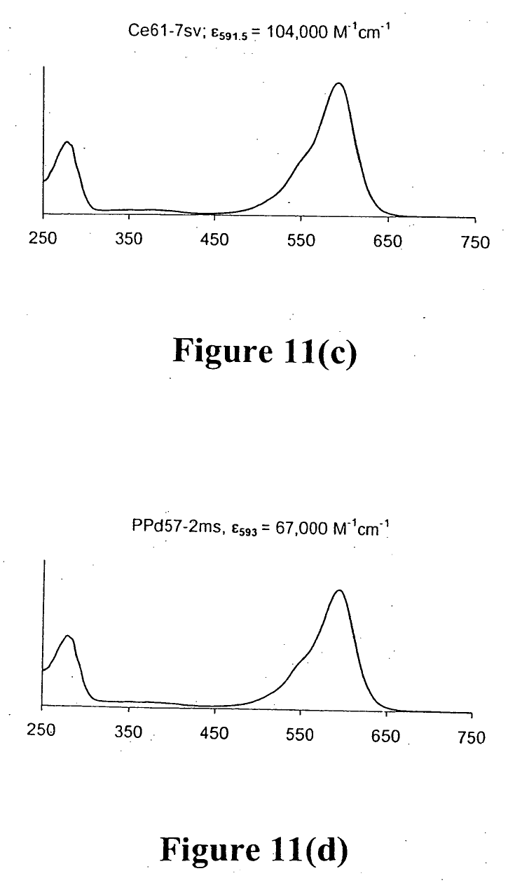

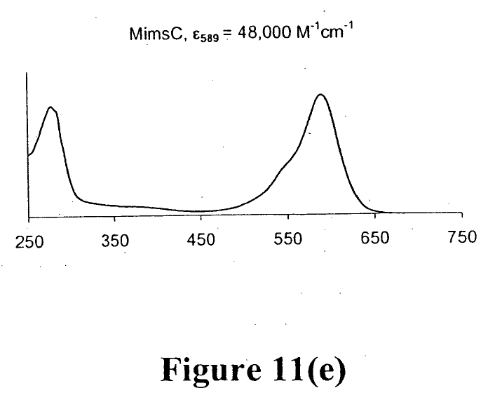

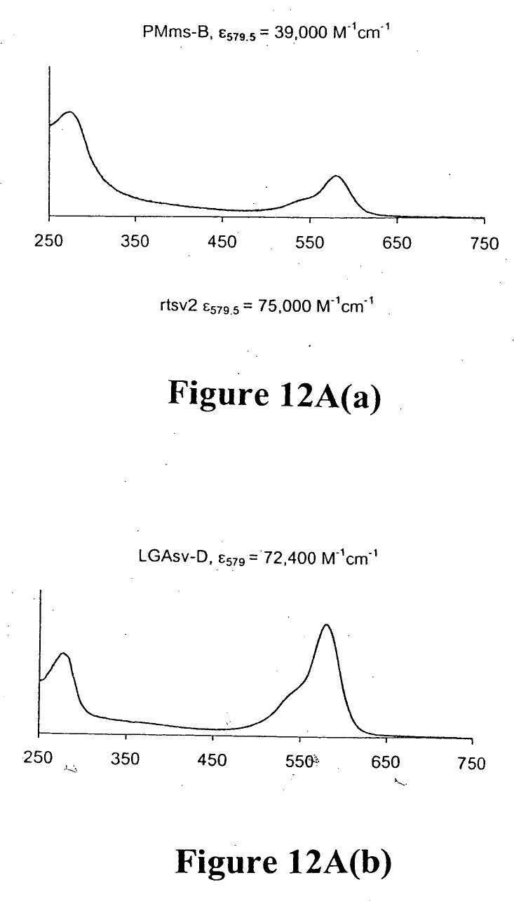

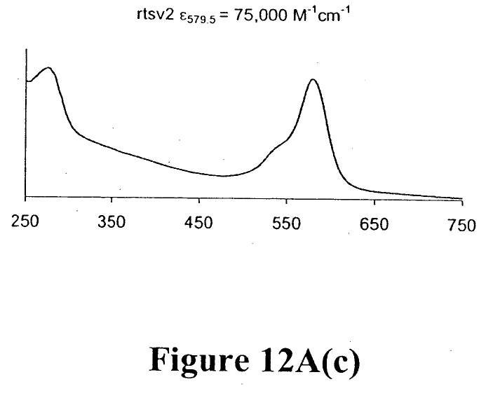

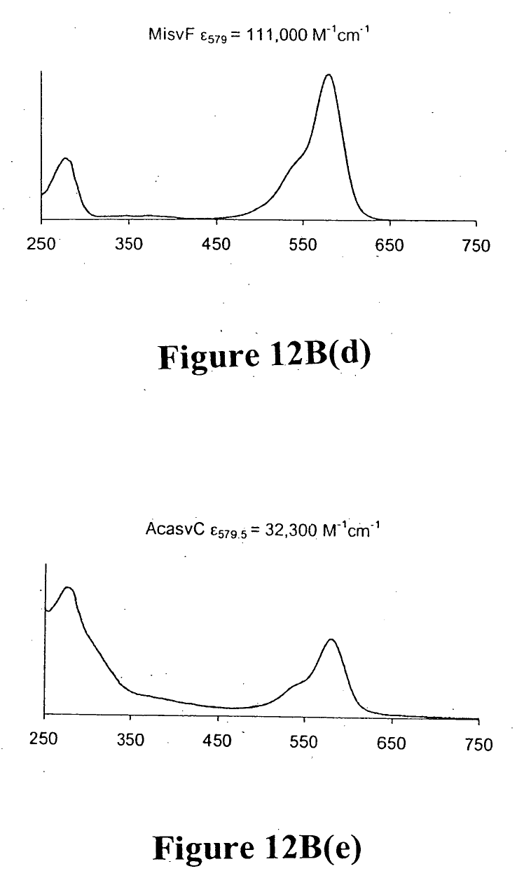

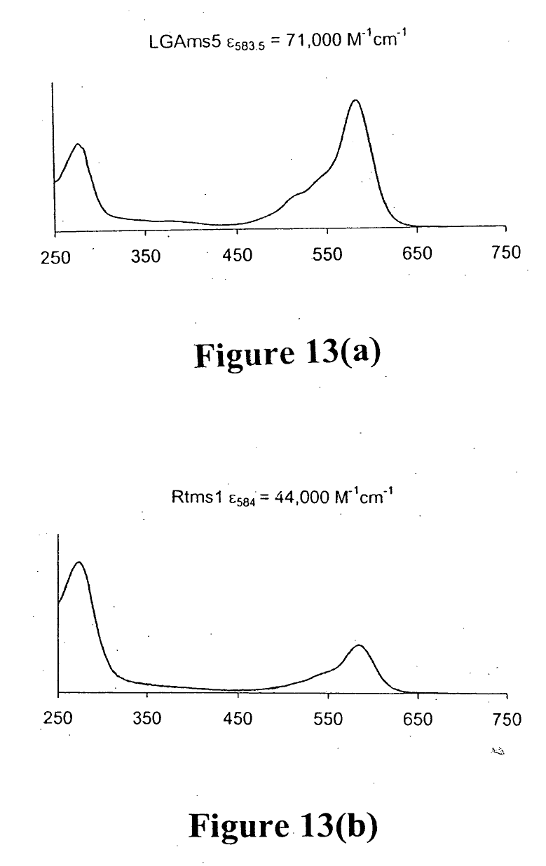

Absorption scans for examples of expressed type 1 sequences are shown in FIG. 11. Extinction coefficients at λₘₐₓ, as shown in this and in subsequent FIGS. 12 and 13, are based on the method of Whitaker and Granum (1980, Yupra) for protein detection. Extinction coefficient variability is partly due to the state of protein maturation; similar variability has been demonstrated for DsRed (Baird et al., Proc. Natl. Acad. Sci. USA 97:11984-11989,2000).

Expression of Examples of Types 2 and 14 Peptides

Results of expressing sequences of type 2 in bacteria are shown in Table 11. Again, only non-identical sequences are shown. Additional sequences, identical to those shown in the Table, are indicated at the top of the Table (i.e.: PMms-B=Pms-E=PPd57-4ms, etc.). The majority of type 2 sequences are pinky-purple with λₘₐₓ ranging from 579 nm to 580 nm. Naturally-occurring amino acid substitution P15S leads to clear bacteria that no longer absorb within 520-600 nm range. Alignment shows amino acids that do not affect the colour of protein, although it was noted that some of these proteins had a greater tendency to aggregate and precipitate than did others.

Analogous results, following expressing of type 14 sequences in bacteria, are shown in Table 12. Only non-identical sequences are shown. Table formatting is the same as in Tables 10 and 11. The majority of type 14 sequences are pinky-purple with λₘₐₓ ranging from 579 nm to 579.5 nm. Alignment shows amino acids that do not affect the colour of protein. It was noted, however, that MisvF and MisvA, with AA147=F, was more soluble at higher concentrations than at others.

The spectral properties of Type 2 and Type 14 sequences are similar. This may be driven by AA61, which is Ser in both of these cases as opposed to Cys in type 1 and Thr in type 6 sequences. FIGS. 12A and B show absorption scans for examples of expressed type 2 and type 14 sequences. As described above for type 1 sequences, observed extinction coefficient variability is partly due to the state of protein maturation.

Expression of Examples of Type 6 Peptides

Examples of Type 6 sequences were similarly expressed in bacteria. Again, only non-identical sequences are shown. In this case, the majority of sequences are blue-purple, with λₘₐₓ ranging from 583.5 nm to 585.5 nm. Alignment shows that naturally occurring amino acid substitutions V8M and/or T182P lead to colourless bacteria, as does G238E, and that substitutions at AA101 and AA147 have slight effect on λₘₐₓ. Results are shown in Table 13 (see over). The format is the same as for Tables 10, 11 and 12.

FIG. 13 shows absorption scans for examples of expressed type 6 sequences. As already stated above, extinction coefficient variability is partly due to the state of protein maturation and similar variability has been demonstrated for DsRed (Baird et al. 2000).

Expression of Examples of Peptides of other Types

Results of bacterial expression of sequence types other than the major types 1, 2, 6 and 14, are shown in Table 14 (see over). Many of the sequences that failed to express blue-purple or pink proteins were isolated from cDNA in which this was not the predominant GF? homolog present.

### EXAMPLE 5

### Estimation of Amount of Total Soluble Protein for Colored Proteins

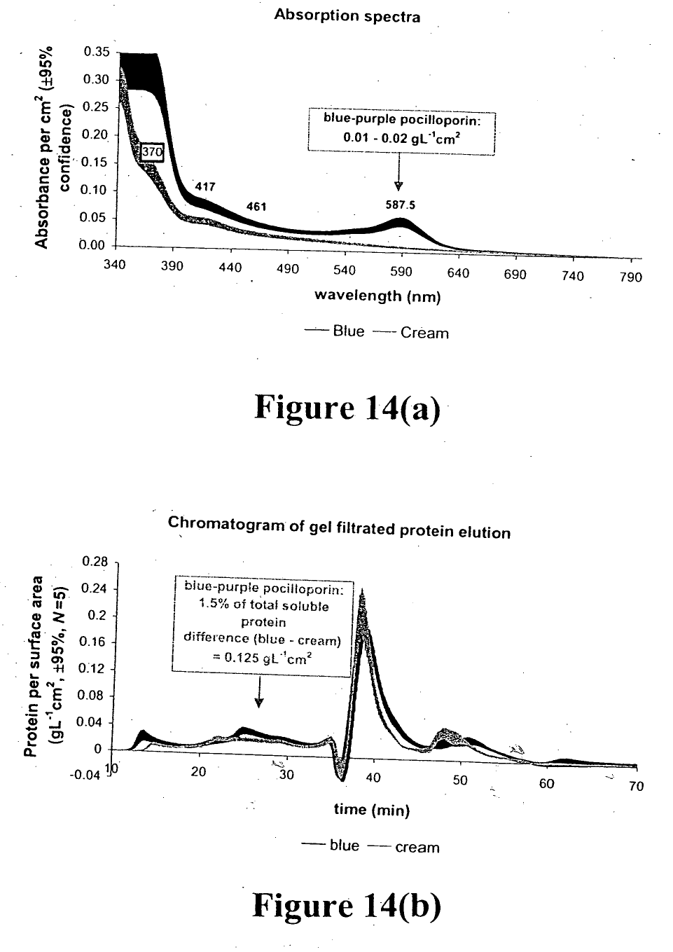

Raw phosphate buffer extracts of two colour morphs of Acropora aspera (a dark blue pigmented morph and a cream morph) were used in the determination of the colored protein proportion of total soluble protein. Two separate estimations were made—by absorption spectroscopy and by gel filtration (n=5; 95% confidence intervals, in each case). Results are set forth in FIGS. 14A/B.

FIG. 14A shows an absorption scan of the two Acropora aspera morphs. Estimation of blue-purple pocilloporin concentration (Dove et al., 1995, supra; Dove et al., 2001, supra) per surface area of coral tissue is based on an extinction coefficient range of 50,000-100,000 M⁻¹cm⁻¹. FIG. 14B shows the results for chromatograms of gel filtrated protein elution, determined from 235 nm and 280 nm chromatograms, applying the equation (235 nm −280 nm)/2.51 (Whitaker and Granum, 1980, supra). The total area under the graph provides a measure of the total soluble protein. Blue-purple pocilloporin concentration is based on the difference between areas under the blue and cream graphs in the range of pocilloporin elution (24-26.5 min). Notably the independent methods for blue-purple pocilloporin concentration give similar results.

### EXAMPLE 6

### Colored Protein Expression from Melbourne Coral cDNAs

Colonies of coral CDNA clones isolated from Discosoma sp. (D2 (pCGP2925 (blue in color)), Sinulatia sp. (S1, pCGP2923) and Tubastrea sp. (T1, pCGP2921, T3, pCGP2922) were grown overnight with shaking at 37° C. in 2 mL LB media containing 100 μg/mL ampicillin. One mL of the overnight culture was then used to inoculate 25 mL LB media containing 100 μg/mL ampicillin. This culture was then incubated at 37° C. with shaking until the OD₆₀₀ was around 0.5. IPTG was added to a final concentration of 1 mM and the cultures were grown overnight with shaking at 37° C. Cells (10 mL) of the incubated cultures were pelleted by centrifugation at 2000 rpm for 10 min. The bacterial pellets and supernatant of the D2 (pCGP2925), S1 (pCGP2923) and T1 (pCGP2921) were blue those of T3 (pCGP2922) were purple under natural light. Bacterial pellets were stored at −20° C.

Proteins contained in the supernatant of the cultures were concentrated using Centricon 30 spin columns (Amicon) according to the manufacturer's instructions. The final volume of each of the concentrated protein extract was ˜200 μL.

Aliquots (8 μL) of the concentrated proteins derived from the supernatants of the cultures were electrophoresced through precast SDS PAGE gels (12% w/v resolving, 4% w/v stacking gel) (Ready Gels, BIORAD) in a running buffer made of 25 mM Tris-HCl, Ph 8.3, 192 mM glycine, 0.1% w/v SDS at 100V for 75 min. Standards included Biorad Pre-stained Broad Range markers which contained standard protein samples of 206 kDa, 119 kDa, 91 kDa, 51.4 kDa, 34.7 kDa, 28.1 kDa, 20.4 kDa and 7.2k Da.

Samples were either denatured by boiling in 10% v/v glycerol, 3% w/v SDS, 3% β-mercaptoethanol (BME), 0.025% w/v bromophenol blue or denatured by boiling in 10% v/v glycerol, 3% w/v SDS, 0.025% w/v bromophenol blue or loaded in their native state in 5% v/v glycerol, 0.04% w/v bromophenol blue.

Prior to staining with Coomassie blue, protein bands were examined under a hand-held UV transilluminator. No fluorescent bands were visible under UV light in any of the samples. However, under natural light a blue band running at the same position as the 28 kDa protein standard was visible in the concentrated protein sample from the D2 supernatant. Blue smears that extended between the 28 kDa and 51 kDa protein standards were visible under natural light in the non-denatured concentrated protein samples from T1 and S1 supernatants. A purple smear which extended between the 28 kDa and 51 kDa protein standards was visible under natural light in the non-denatured concentrated protein samples from the S3 supernatant. There were no bands observed under natural light in samples that were denatured by boiling. Staining the gel with Coomassie blue showed that the proteins produced co-migrated with a 25 kDa protein marker (Biorad Precision Broadrange Prestained Marker).

Cultures of (E. coli XL1-blue) coral cDNA clones from Discosoma sp. (D1 in pCGP2919), Sinularia sp. (S1 in pCGP2923) and Tubastrea sp. (T1 in pCGP2921 and T3 in pCGP2922) that had grown at 37° C. overnight with shaking were used to inoculate 100 mL LB media containing 100 μg/mL ampicillin and fisher incubated with shaking at 37° C. until the OD₆₀₀ was ˜0.5. IPTG was added to a final concentration of 1 mM and the cultures were grown overnight with shaking at 37° C. Proteins expressed by Tubastrea sp. clones (T1 and T3) were purified under native conditions using Ni-NTA Superflow resin (Qiagen; QIAexpressionist 03/97) as recommended by the manufacturer. The elution buffer was exchanged with 20 mM Tris-HCl pH 8.0 using Sephadex G-25 columns (NAP10; Pharmacia) as per the manufacturer's instructions. Proteins expressed by the Discosoma sp. clone D1 and the Sinularia sp. clone S1 were purified under native conditions using the Ni-NTA method (Qiagen; QIAexpressionist 03/97) except that protein was precipitated from cleared bacterial lysate using 65% isopropanol and centrifuged at 10,000 rpm, 4° C., 10 min. The colored pellet was resuspended in 20 mM Tris-HCl pH 8.0.

The proteins encoded by the Acropora sp. A8 clone in pCGP2918, the Discosoma sp. D10 clone in pCGP2920, the Sinularia sp S3 clone in pCGP2924 and the Tubastrea sp. T3 clone in pCGP2922 were a purple color (Royal Horticultural Society Color Chart (RHSCC) 88A) when concentrated. The proteins from Tubastrea sp. T3 clone and the Sinularia sp. 53 clone had absorbance peaks at approximately 580 mn.

The proteins encoded by the Discosoma sp. D1 clone in pCGP2919 and the Tubastrea sp. T1 clone in pCGP2921 were a blue color (RHSCC 102A) when concentrated and absorbance peaks at approximately 595 nm. The protein encoded by Sinularia sp. S1 clone in pCGP2923 was a blue-purple color (RHSCC 90A) when concentrated and had an absorbance peak at approximately 590 nm.

Amino Acid Sequence Alignment

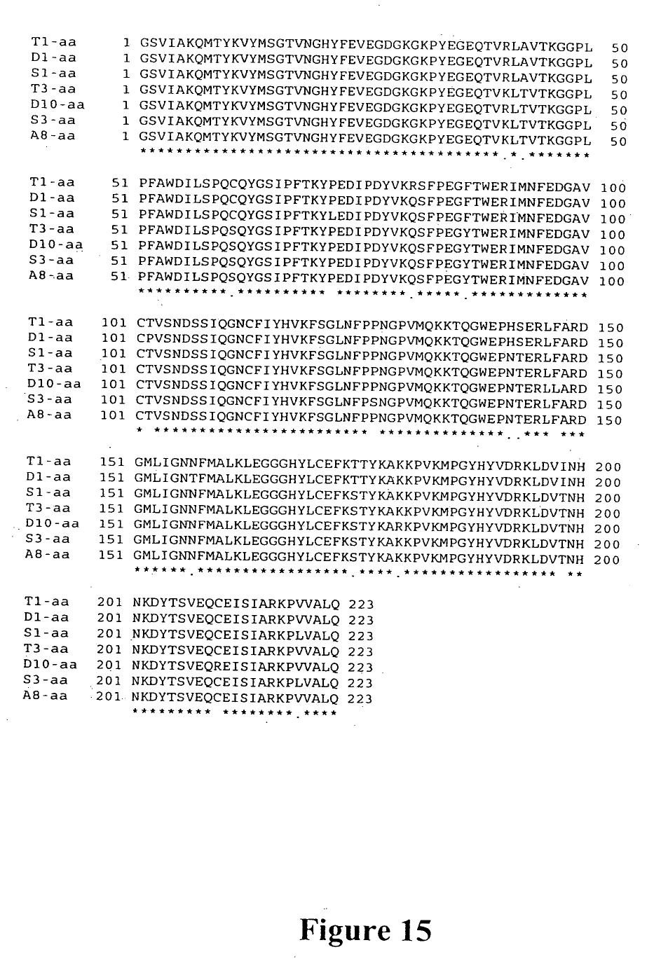

A multiple alignment of the encoded amino acid sequence of T1 (SEQ ID NO:202), D1 (SEQ ID NO:198), S1 (SEQ ID NO:200), A8 (SEQ ID NO:190), T3 (SEQ ID NO:196), D10 (SEQ ID NO:192) and S3 (SEQ ID NO:194) was produced using the Clustal W (1.4) program in MacVector (6.5.3; Oxford Molecular Group Plc, 1999) (FIG. 15). The multiple alignment of encoded amino acids showed that there are only 16 amino acid positions that differed between proteins exhibiting blue, blue-purple and purple color. From this alignment there appear to be eight amino acid positions that may influence the color of the protein (Table 15).

The protein encoded by S1 (SEQ ID NO:200) has a color that is intermediate of the blue and purple proteins. The amino acid sequence alignment (FIG. 15) showed that the S1 amino acid sequence contained four amino acid identities characteristic of blue proteins towards the amino-terminal end and four amino acid identities characteristic to purple proteins towards the carboxy-terminal end (Table 15). The substitution of one or more amino acids listed in Table 15 may influence the visible color characteristics of the protein.

Alignment of Melbourne and Heron Island Coral protein Sequences

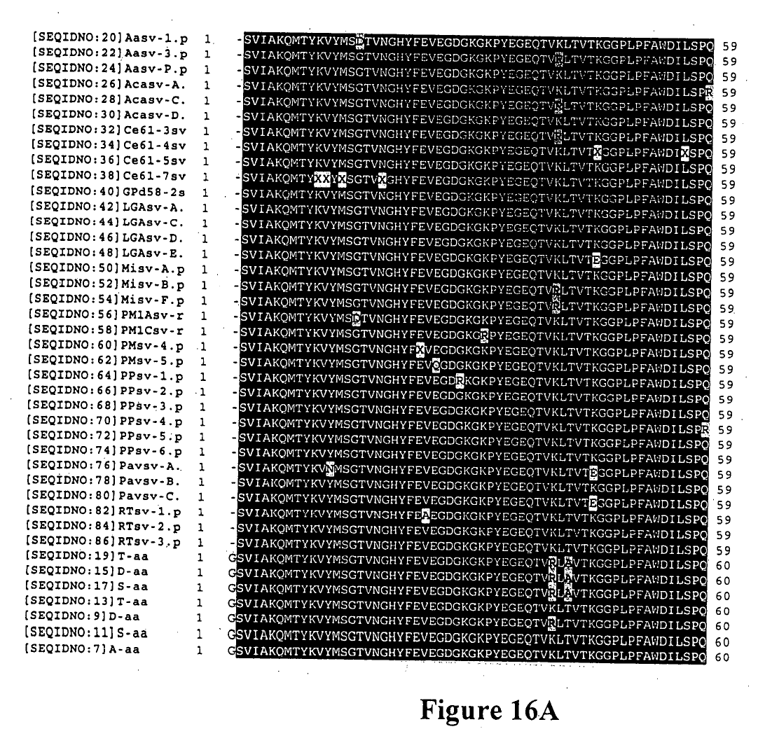

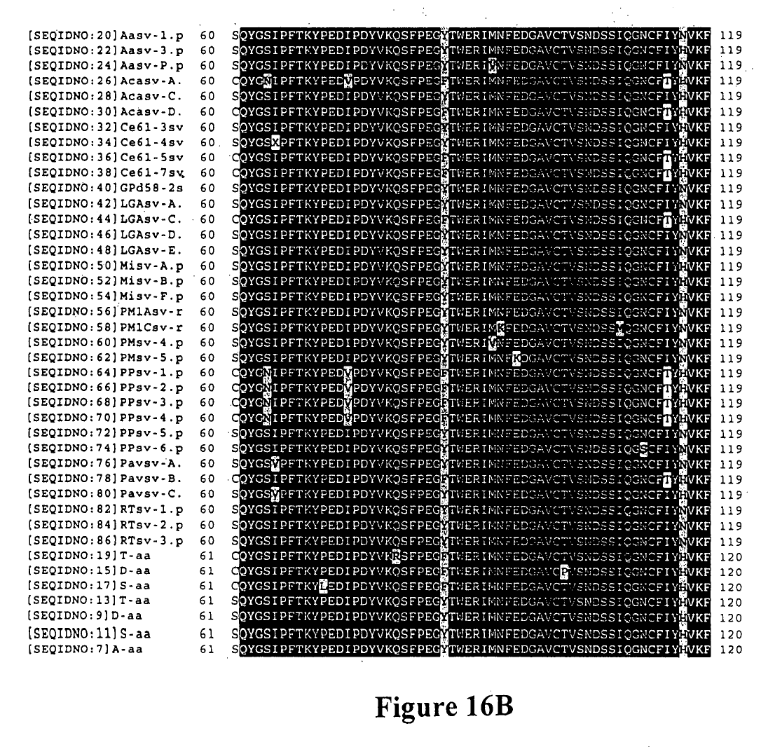

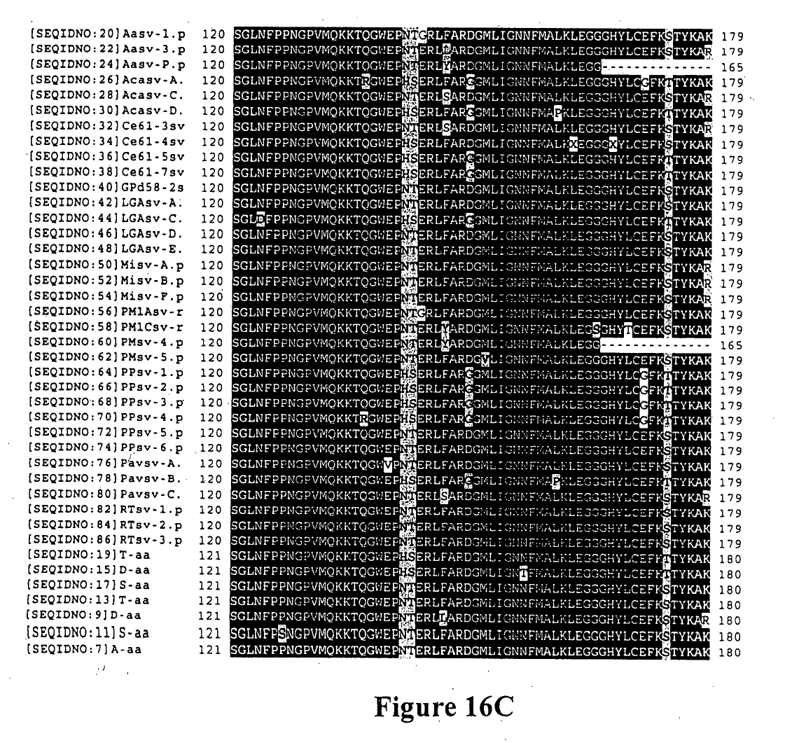

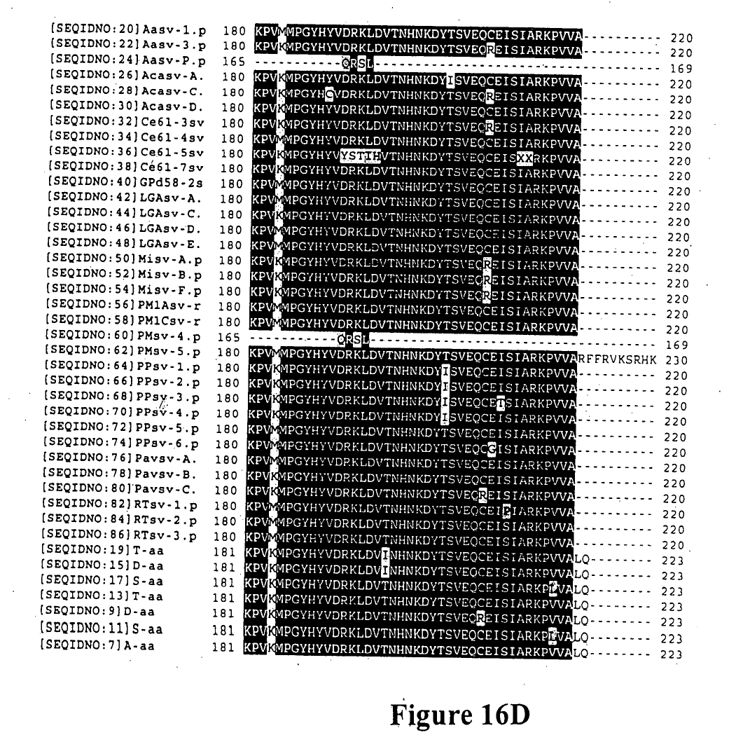

The amino acid sequences of the above seven polypeptides (SEQ ID NOs 190, 192, 194, 196, 198, 200 and 202) were compared with other SVIAK (SEQ ID NO:5)-containing polypeptides, as set forth in FIG. 1. The resulting alignment is shown in FIG. 16.

### EXAMPLE 7

### Expression of Colored Proteins in an Eukaryotic Organism Saccharomyces Cerevisiae

In order to observe whether the colored protein sequences were able to produce color in a eukaryotic cell, the colored protein cDNA clones T1 (SEQ ID NO:201) and A8 (SEQ ID NO:189) were introduced into a yeast expression vector (pYE22m) (Tanaka et al., 1988, supra) and transformed into Saccharomyces cerevisiae strain G1315.

Construction of pCGP3269 and pCGP3270 (T1 or A8 in pYE22m)

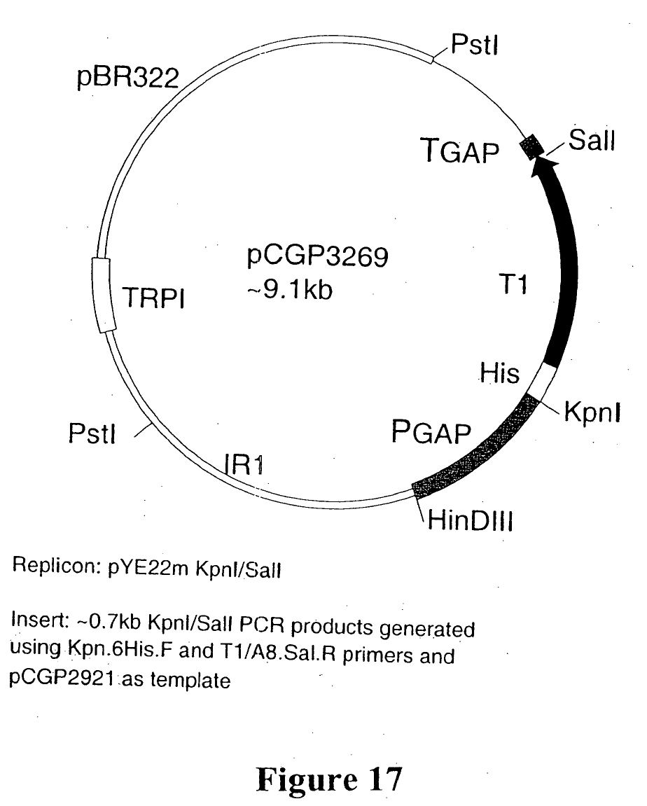

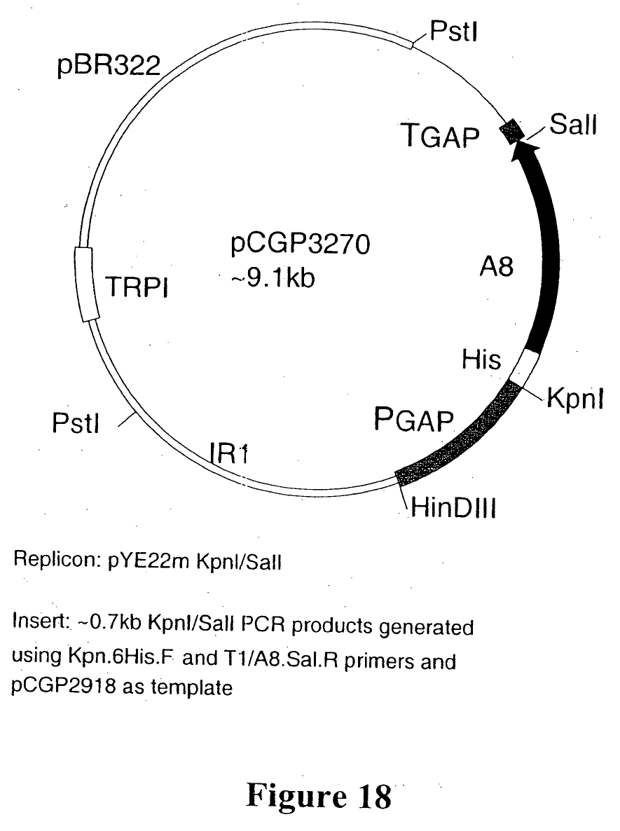

The plasmids pCGP3269 (FIG. 17) and pCGP3270 (FIG. 18) were constructed by cloning the Ti or A8 cDNA clones, respectively, in a sense orientation behind the yeast glyceraldehyde 3-phosphate dehydrogenase promoter of pYE22m (Tanaka et al., 1988, supra).

A forward primer (Kpn.6His.F; SEQ ID NO:203) was designed to amplify the colored protein sequences that would result in 6 x Histidine tag fused in-frame with the colored protein at the N-terminus and a KpnI restriction endonuclease recognition site at the 5′ end. A reverse primer (T1/A8.SaI.R; SEQ ID NO:204) included a SalI restriction endonucdease recognition site at the 3′ end

```
SEQ ID NO:203 Kpn.6His.F
            KpnI
GCAT GGT ACC ATG AGA GGA TCG CAT CAC CAT CAC CAT CAC
                            M R G S H H H H H H
SEQ ID NO:204 T1/A8.Sa1.R
            SalI
CTGA GTC GAC TCA CTG CAG GGC GAC CAC AGG TTT
                            * Q L A V V P K
```

The coding regions of T1 (SEQ ID NO:201) and A8 (SEQ ID NO:189) were amplified by PCR using the primers Kpn.6His.F (SEQ ID NO:203) and T1/A8.Sal.R (SEQ ID NO:204) and the plasmid DNA pCGP2921 (T1) (FIG. 10) and pCGP2918 (A8) (FIG. 10) as template. The ˜700 bp PCR products were purified using a QIAquick. PCR purification kit (Qiagen) and then digested with the restriction endonucleases KpnI and SalI. The KpnI/SalI digested products were finally purified using a QIAquick PCR purification kit (Qiagen) and subsequently ligated with the KpnI/SalI ends of the pYE22m yeast expression vector (Tanaka et al., 1988 supra) using a DNA Ligation Kit (Amersham) according to the manufacturer's recommendations. Correct insertion of the T1 or A8 cDNA clones into the yeast expression vector was confirmed by visualisation of colour of transfonnants that were selected by their ability to restore G-1315 to tryptophan prototrophy. The T1 clone in the yeast expression vector pYE22m (designated as pCGP3269) produced blue coloured colonies (RHSCC 101C) when introduced into the yeast strain G1315. The A8 clone in the yeast expression vector pYE22m (designated as pCGP3270) produced purple coloured colonies (RHSCC 82B) when introduced into the yeast strain G1315.

### EXAMPLE 8

### Estimation of Colored Protein Amounts Produced by Bacterial and Yeast Cultures

Quantitation of Colored Protein Expression in Saccaryomyces cerevisiae

Pure cultures of yeast cells harbouring pCGP3269 (FIG. 17) or pCGP3270 (FIG. 18) were grown at 29° C. for 48 hours in 100 mL of YEPD liquid broth (1% yeast extract, 2% bacto-peptone, 2% w/v glucose, pH5.0). The cultures were centrifuged at 2000 rpm for 15 min. The resulting pellets were blue (pCGP3270) and purple (pCGP3269). The His-tagged colored proteins were extracted under native conditions by first resuspending the pellets in 4 mL lysis buffer (50 mM NaH₂PO₄, pH 8.0, 300 mM NaCl, 10 mM imidazole, 5 mg/nL Yeast Lytic enzyme (IBN)) and incubated at 30° C. for 1 hour. The solutions were sonicated on ice 10 times for 10 sec with 15 sec cooling between treatments. The lysates were then centrifiged at 10 000 rpm for 10 min and the supernatants (crude extract) collected. The His-tagged colored proteins were purified by nickel-nitrilotriacetic acid metal-affinity chromatography (Qiagen) as recommended by the manufacturer.

The protein content of the crude extracts and purified His-tagged colored proteins were measured using a Bio-Rad Protein Assay using 1, 3 and 5 μL aliquots of extracts as per the manufacturer's instructions (Bio-Rad Microassay Procedure). The absorbances at 595 nm were compared with bovine serum albumin (BSA) standard curves (0-10 μg/mL) to obtain estimations of protein concentrations.

Samples of crude extracts and a dilution series of known amounts of purified His-tagged colored protein were electrophoresed through precast SDS PAGE gels (12% w/v resolving, 4% w/v stacking gel) (Ready Gels, Biorad) as described in Example 3. The gels were then stained with Coomasie blue (0.25% (w/v) Coomassie Brilliant Blue, 45% (v/v) methanol, 10% (v/v) acetic acid) and the amounts of colored protein in the crude extracts were estimated by comparing the intensities of the stained bands with those of the purified His-tagged colored protein dilution series. This allowed the estimation of expression of colored protein in yeast as a percentage of total soluble protein (Table 16).

Quantitation of Colored Protein Expression in Escherichia coli

One mL of an overnight Escherichia coli XL1blue culuture harbouring the plasmid pCGP2921 (T1) (FIG. 10) (Example 3) was used to inoculate 100 mL LB broth (containing 50 μg/mL ampicillin) and incubated 37° C. with shaking at 200 rpm until the OD600 was between 0.5 - 0.7. Protein production was induced with the addition of IPTG to 1 mM and incubation overnight at 29° C. with shaking at 200 rpm. The cells were pelleted by centrifugation at 2000 rpm for 15 min. The resulting pellet was blue. The pellet was resuspended in 4 mL lysis buffer (50 mM NaH₂PO₄, pH 8.0, 300 mM NaCl, 10 mM imidazole) and sonicated on ice 6 times for 10 sec with 15 sec cooling between treatments. The solution was centrifuged at 10 000 rpm for 10 min and the (crude extract) supernatant collected. The His-tagged colored protein (T1) was extracted under native conditions by nickel-nitrilotriacetic acid metal-affinity chromatography (Qiagen) as recommended by the manufacturer.

The protein content of the crude extract and purified His-tagged colored protein was measured using a Bio-Rad Protein Assay using 1, 3 and 5 μL of extracts as per the manufacturers instructions (Bio-Rad Microassay Procedure). The absorbances at 595 nm were compared with BSA standard curves (0-10 μg/mL) to obtain estimations of protein concentrations.

Samples of crude extract and a dilution series of known amounts of purified His-tagged colored protein were electrophoresed through SDS PAGE gels as per the crude extract from yeast cultures (as described above). The amounts of colored protein in the crude extracts were estimated by comparing the intensites of the stained bands with those of the purified His-tagged colored protein dilution series. This allowed the estimation of expression of colored protein in E. coli as a percentage of total soluble protein (Table 16).

### EXAMPLE 9

### Expression of Colored Proteins in Plants Under the Control of a Constitutive Promoter

Construction of pCGP2756 (35S: MCS: 35S Expression Cassette)

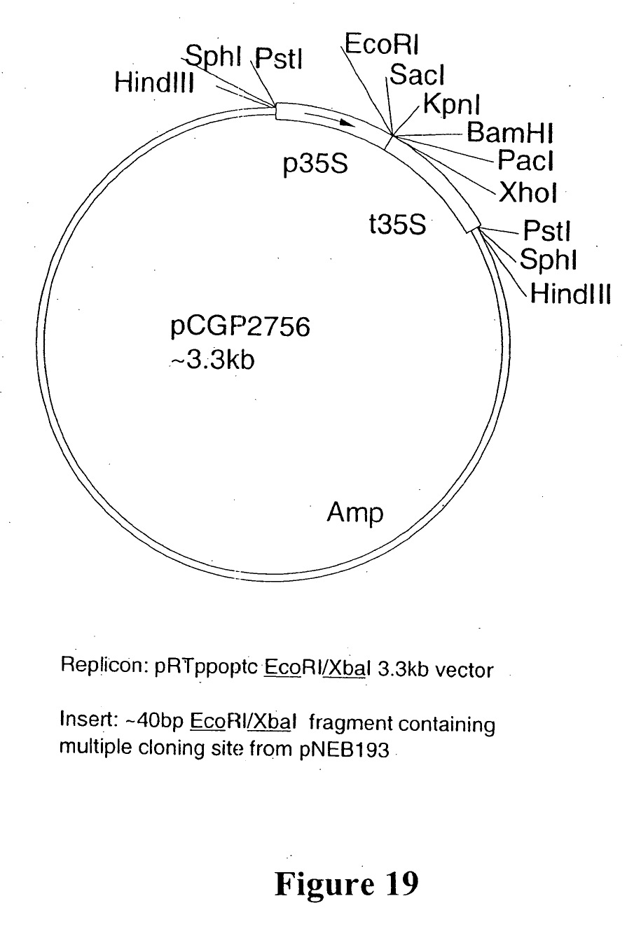

Plasmid pCGP2756 (FIG. 19) was constructed by cloning the multicloning site (MCS) (containing the rare restriction endonuclease sites PacI and AscI) from pNEB193 (New England Biolabs) into the CaMV35S expression cassette of pRTppoptcAFP (Wnendt et al., Curr Genet 25:510-523, 1994). The plasmid pRTppoptcAFP was digested with EcoRI and XbaI to release 300 bp AFP (antifungal protein) insert and the 3.3 kb vector containing the CaMV 35S expression cassette. The plasmid pNEB 193 was digested with EcoRI and XbaI to release the 40 bp fragment containing the multicloning site. The 40 bp EcoRI(XbaI fragment from pNEB193 and the 3.3 kb vector containing the CaMV35 expression cassette from pRTppoptcAFP were isolated and purified using the QIABX II Gel Extraction kit (Qiagen) and ligated together. The ligation was carried out using the Amersham ligation kit. Correct insertion of the fragment in pCGP2756 was established by restriction enzyme analysis (SalI, KpnI, BamHI, XbaI, AscI, PacI, HindIII/BamHI) of DNA isolated from ampicillin-resistant transfonnants.

Construction of pCGP2757 (35S: MCS: 35S Binary Vector)

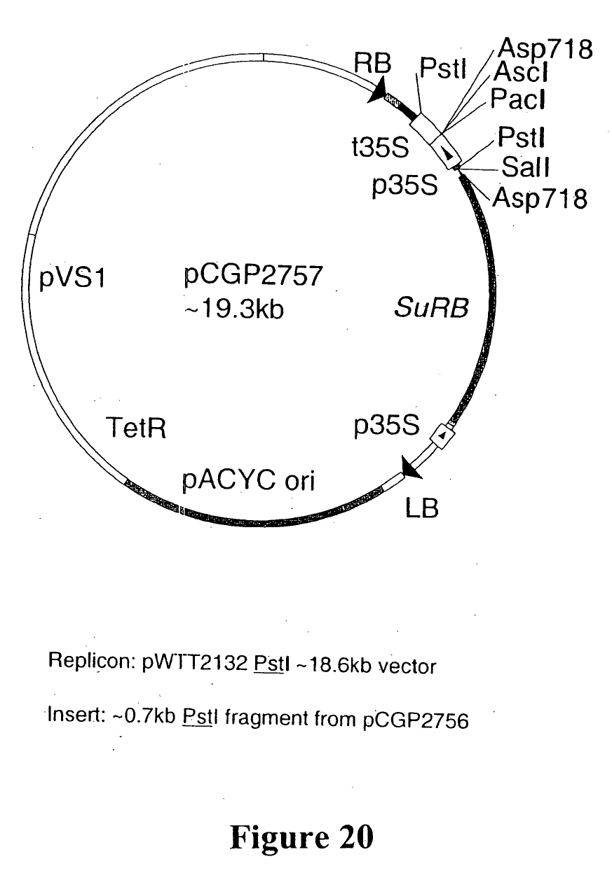

Plasmid pCGP2757 (FIG. 20) was constructed by cloning the CaMV35S expression cassette of pCGP2756 (described above) into the binary vector pWTT2132 (DNAP). The plasmid pCGP2756 was digested with PstI to release the 0.7 kb CaMV35S expression cassette containing the multicloning site from pNEB193. The 0.7 kb fragment was isolated and purified using the QIAEX II Gel Extraction kit (Qiagen) and ligated with PstI ends of pWTT2132 binary vector. Correct insertion of the fragment in a tandem orientation to the CaMV35S: surB cassette in pWTT2132 was established by restriction enzyme analysis (KpnI, PacI/AscI, EcoRI, XbaI, PstI) of DNA isolated from tetracycline-resistant transformants.

PCR products of CFMs or colored proteins derived using the primers vispro-F1 (SEQ ID NO:184) and vispro-R1 (SEQ ID NO:185) or using any primers containing AscI and PacI restriction endonuclease recognition sites, can be digested with AscI and PacI and ligated with AscI/PacI ends of pCGP2757.

Construction of pCGP2765 (35S: A8: 35S Binary)

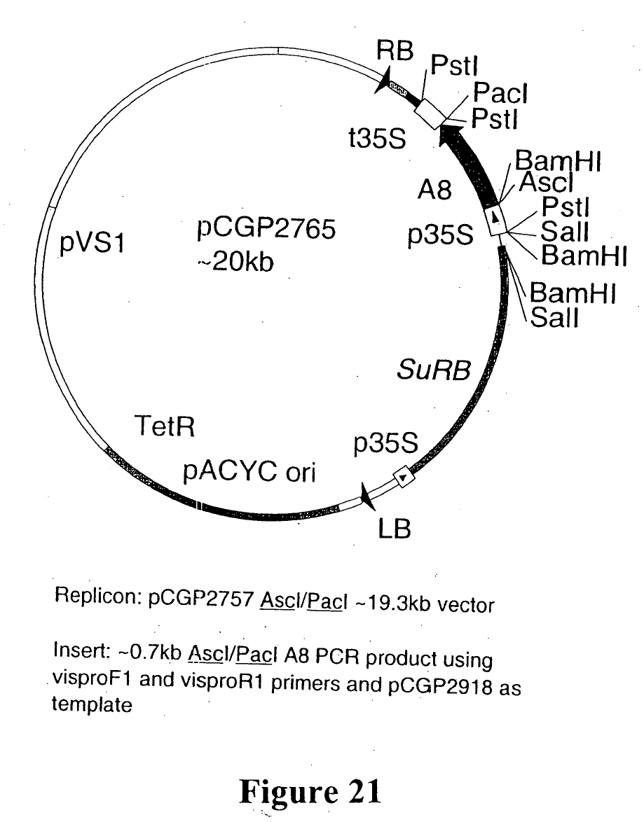

Plasmid pCGP2765 (FIG. 21) was constructed by cloning the A8 PCR clone amplified from Acropora sp. into the CaMV35S expression cassette contained in the binary vector of pCGP2757 (described above). The A8 PCR product generated using the vispro-F1 (SEQ ID NO:184) and vispro-R1 (SEQ ID NO:185) primers and cDNA synthesized from Acropora sp. total RNA as template (see Example 1), was digested with AscI and PacI. The ˜0.7 kb fragment was isolated and purified using the QIAEX II Gel Extraction kit (Qiagen) and ligated with AscI/PacI ends of pCGP2757 binary vector. Correct insertion of the fragment in a sense orientation behind the CaMV35S promoter was established by restriction enzyme analysis (EcoRI, PstI, BstXI) of DNA isolated from tetracycline-resistant transformants.

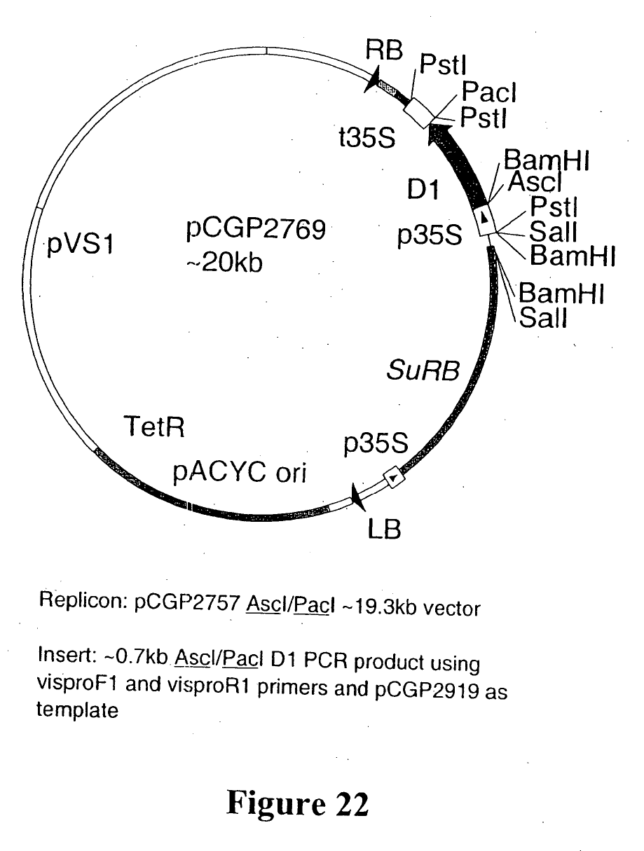

Construction of pCGP2769 (35S: D1: 35S Binary) (FIG. 22)

Plasmid pCGP2769 (FIG. 22) was constructed by cloning the D1 PCR clone amplified from Discosoma sp. into the CaMV35S expression cassette contained in the binary vector of pCGP2757 (described above). The PCR product generated using the primers vispro-F1 (SEQ ID NO:184) and vispro-R1 (SEQ ID NO:185) and the template pCGP2919 (containing the D1 cDNA clone) was digested with AscI and PacI. PCR was carried out in 50 μL reactions with 200 μM dNTPs, 20 pmol vispro-F1 (SEQ ID NO:184), 20 pmol visproR1 (SEQ ID NO:185), 1×Pfu buffer (Stratagene), 2.5 units Pfu trubo DNA Polymerase (Stratagene) and ˜2 ng pCGP2919 plasmid DNA as template. The ˜0.7 kb fragment was isolated and purified using the QIAEX II Gel Extraction kit (Qiagen) and ligated with AscI/PacI ends of pCGP2757 binary vector. Correct insertion of the fragment in a sense orientation behind the CaMV35S promoter was established by restriction enzyme analysis (EcoRI, PstI, BstXI, BamHI) of DNA isolated from tetracycline-resistant transformants.

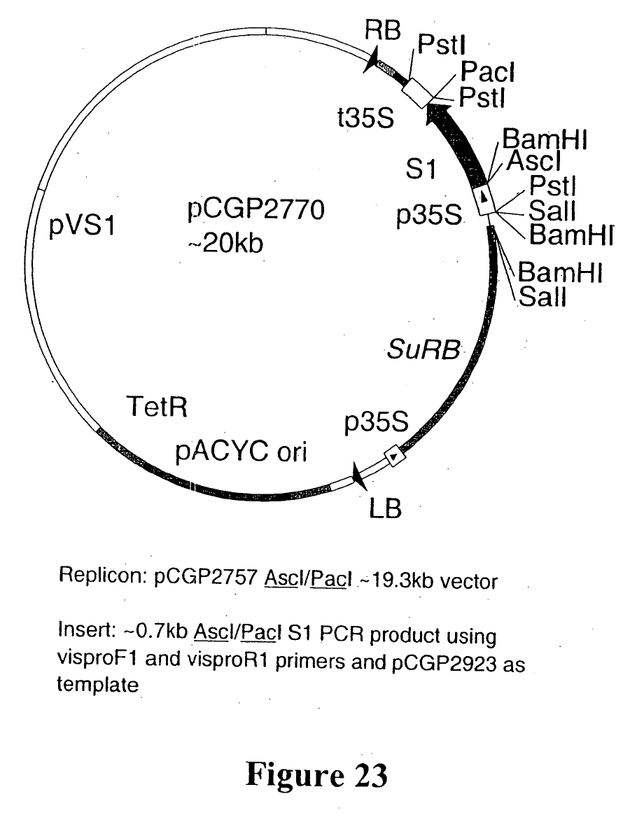

Construction of pCGP2770 (35S: S1: 35S Binary) (FIG. 23)

Plasmid pCGP2770 (FIG. 23) was constructed by cloning the S1 PCR clone amplified from Sinularia sp. into the CaMV35S expression cassette contained in the binary vector of pCGP2757 (described above). The PCR product generated using the primers vispro-F1 (SEQ ID NO:184) and vispro-R1 (SEQ ID NO:185) and the template pCGP2923 (containing the S1 cDNA clone) was digested with AscI and PacI. PCR was carried out in 50 μL reactions with 200 μM dNTPs, 20 pmol vispro-F1 (SEQ ID NO:184), 20 pmol vispro-R1 (SEQ ID NO:185), 1×Pfu buffer (Stratagene), 2.5 units Pfu trubo DNA Polymerase (Stratagene) and ˜2 ng pCGP2923 plasmid DNA as template. The ˜0.7 kb fragment was isolated and purified using the QIABX II Gel Extraction kit (Qiagen) and ligated with AscYPaci ends of pCGP2757 binary vector. Correct insertion of the fragment in a sense orientation behind the CaMV35S promoter was established by restriction enzyme analysis (EcoRI, PstI, BstXI, BamHI) of DNA isolated from tetracycline-resistant transformants.

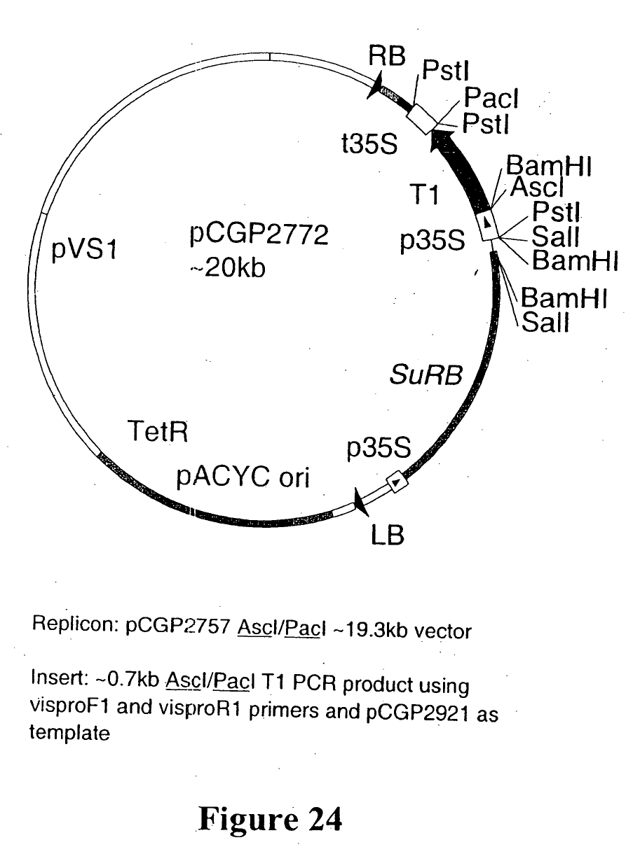

Construction of pCGP2772 (35S: T1: 35S Binary) (FIG. 24)

Plasmid pCGP2772 FIG. 24) was constructed by cloning the T1 PCR clone amplified from Tubastrea sp. into the CaMV35S expression cassette contained in the binary vector of pCGP2757 (described above). The PCR product generated using the primers vispro-F1 (SEQ ID NO:184) and vispro-R1 (SEQ ID NO:185) and the template pCGP2921 (containing the T1 CDNA clone) was digested with AscI and PacI. PCR was carried out in 50 μL reactions with 200 μM dNTPs, 20 pmol vispro-F1 (SEQ ID NO:184), 20 pmol vispro-R1 (SEQ ID NO:185), 1×Pfu buffer (Stratagent), 2.5 units Pfu trubo DNA Polymerase (Stratagene) and ˜2 ng pCGP2921 plasmid DNA as template. The ˜0.7 kb fragment was isolated and purified using the QIAEX II Gel Extraction kit (Qiagen) and ligated with AscI/PacI ends of pCGP2757 binary vector. Correct insertion of the fragment in a sense orientation behind the CaMV35S promoter was established by restriction enzyme analysis (EcoRI, PstI, BstXI, BamHI) of DNA isolated from tetracycline-resistant transformants.

Construction of PCGP2926 (35S:His T1: 35S Binary)

A histidine-tagged version of T1 was also produced for expression in the CaMV 35S gene expression cassette. The expression of this modified version of T1 will allow for a way of easily concentrating the expressed T1 protein to calculate the amount being produced in plants.

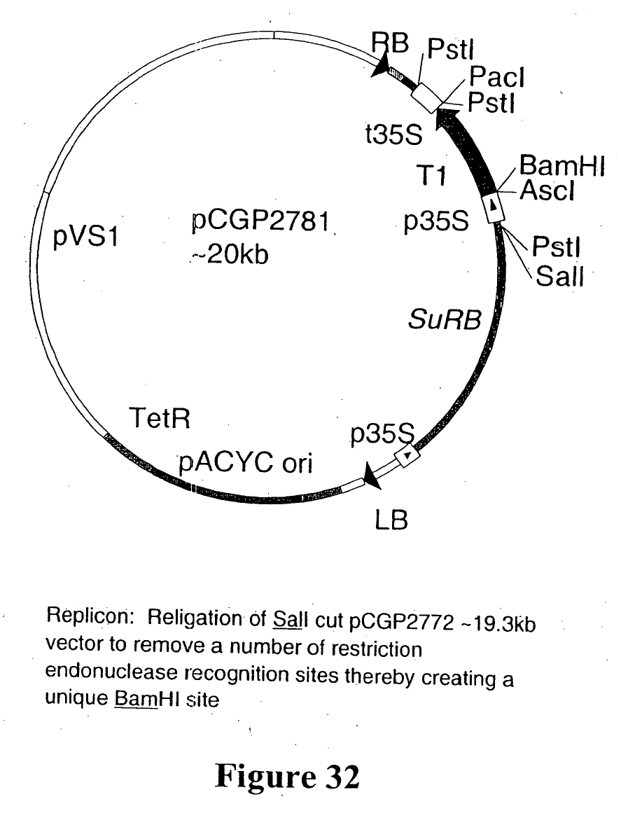

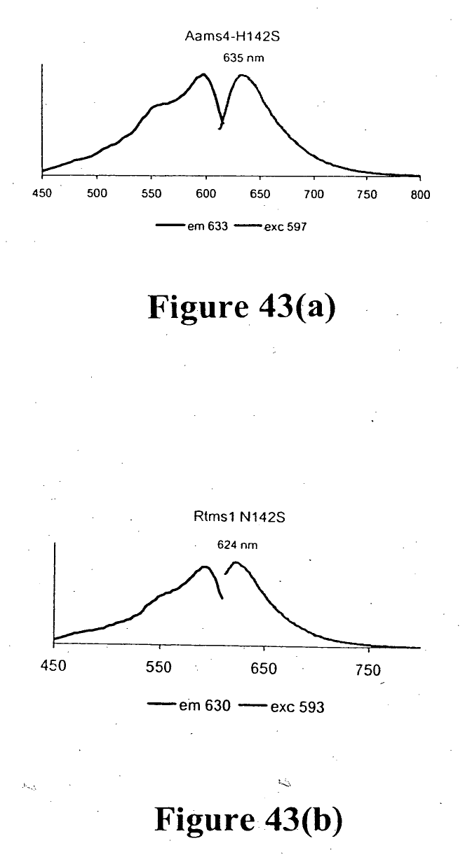

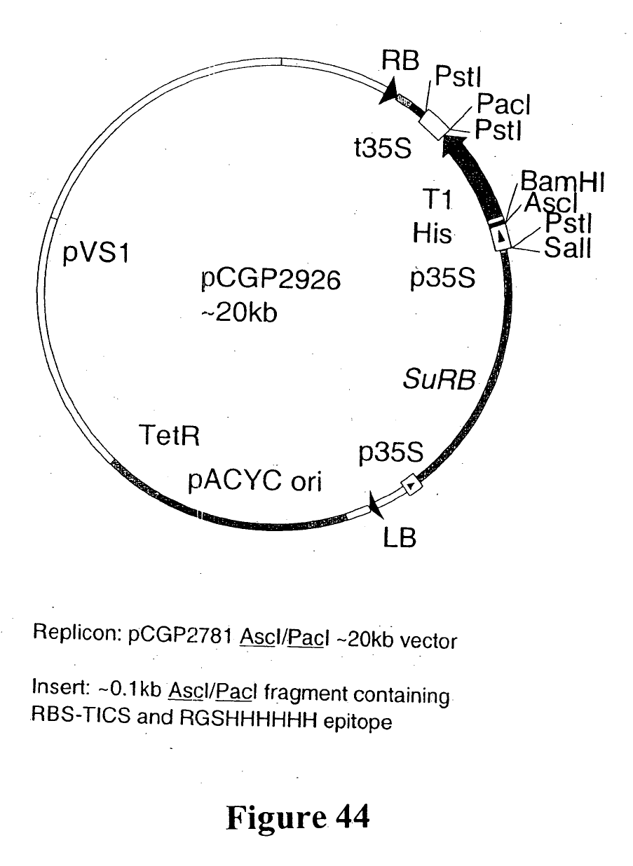

The RGS-His epitope was created by ligation of the 2 complementary primers TICS-His-FWD (SEQ ID NO:227) and TICS-His-REV (SEQ ID NO:228). This ligation resulted in a fragment containing the sequences to a prokaryotic ribosome binding site (RBS), a translational initiation consensus sequence (TICS) (for optimal translation in plants), the RGS-His epitope (consisting of sequences that encode the amino acids RGSHHHHHH and overhanging AscI (at 5′ end) and BamHI (at 3′ end). This AscI/BamHI fragment was ligated with AscI/BamHI ends of plasmid pCGP2781 (FIG. 32). Correct ligation of the insert into pCGP2781 was established by restriction enzyme analysis of DNA isolated from tetracycline-resistant transfonnants. The plasmid was designated as pCGP2926 (FIG. 44).

```
SEQ ID NO:227 TICS-His-FWD (5′ to 3′)
CGCGCC AAGGAGATAT AACA ATG AGA GGA TCG CAT CAC CAT CAC CAT CAC G
                        RBS TICS M R G S H H H H H H
                                                            RGS-His epitope
SEQ ID NO:228 TICS-His-REV (5′ to 3′)
GATCC GTG ATG GTG ATG GTG ATG CGA TCC TCT CAT TGTT ATATCTCCTT GG
                                      RGS-His epitope TICS RBS
```

A. Tumefaciens Transformations

The plasmids pCGP2772 and pCGP2765 were introduced into the Agrobacterium tumefaciens strain AGL0 by adding 5 μg of plasmid DNA to 100 μL of competent AGL0 cells prepared by inoculating a 50 mL LB culture and growing for 16 hours with shaking at 28° C. The cells were then pelleted and resuspended in 0.5 mL of 85% v/v 100 mM CaCl₂/15% v/v) glycerol. The DNA-Agrobacterium mixture was frozen by incubation in liquid N₂ for 2 minutes and then allowed to thaw by incubation at 37° C. for 5 minutes. The DNA/bacterial mix was then placed on ice for a further 10 minutes. The cells were then mixed with 1 mL of LB (Sambrook et al., 1989, supra) media and incubated with shaking for 16 hours at 28° C. Cells of A. tumefaciens carrying pCGP2772 and pCGP2765 were selected on LB agar plates containing 50 μg/mL tetracycline. The presence of pCGP2772 and pCGP2765 were confirmed by restriction enzyme analysis of DNA isolated from the tetracycline-resistant transformants.

### EXAMPLE 10

### Spatial and Temporal Expression of Colored Proteins in Plants

The use of constitutive promoters such as CaMV35S can be used to direct expression of CFM or colored proteins throughout the whole plant and may be useful in cases where a novel phenotype is sought with respect to the whole plant. However in some cases novel color is sought in specific tissues such as floral, seeds, leaves, fibre (e.g. cotton fibre), stems, roots, pollen, etc. In these cases tissue-specific promoters can be used to target expression of CFM or colored proteins to specific tissues. There are many cases in the literature, which describe the use of promoters to direct spatial and temporal expression. These promoters include, but are not limited to, the examples of a seed specific promoters (Song et al., Journal of Cotton Science 4:217-223, 2000), leaf and chlorophyll containing tissue specific promoters (Song et al., 2000, supra), and tuber specific promoters (Rocha-Sosa et al., EMBO J8:23-29, 1989).

Isolation of Rose CHS Promoter

A rose genomic DNA library was prepared from Rosa hybrida cv. Kardinal.

The rose library was screened with rose CHS cDNA clone.

A 6.6 kb fragment upstream from the translational initiation site was cloned into pBluescript KS (−) (Stratagene) and the plasmid designated pCGP1114.

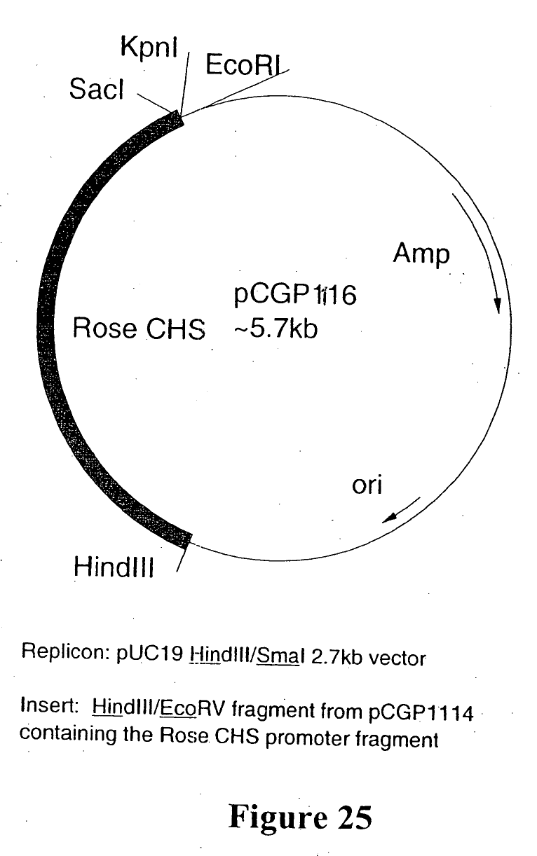

The plasmid pCGP1114 was digested with HindIII and EcoRV to release a ˜2.7-3.0 kb fragment which was purified using a Bresaclean kit (Geneworks) and ligated with HindIII/SmaI ends of pUC19 (New England Biolabs). Correct insertion of the Rose CHS promoter fragment was established by restriction enzyme analysis of DNA isolated from ampicillin-resistant transformants. The resulting plasmid was designated as pCGP1116 (FIG. 25).

Construction of pCGP3255 (Rose CHS 5′: 35S 3 ′ Pre-binary)

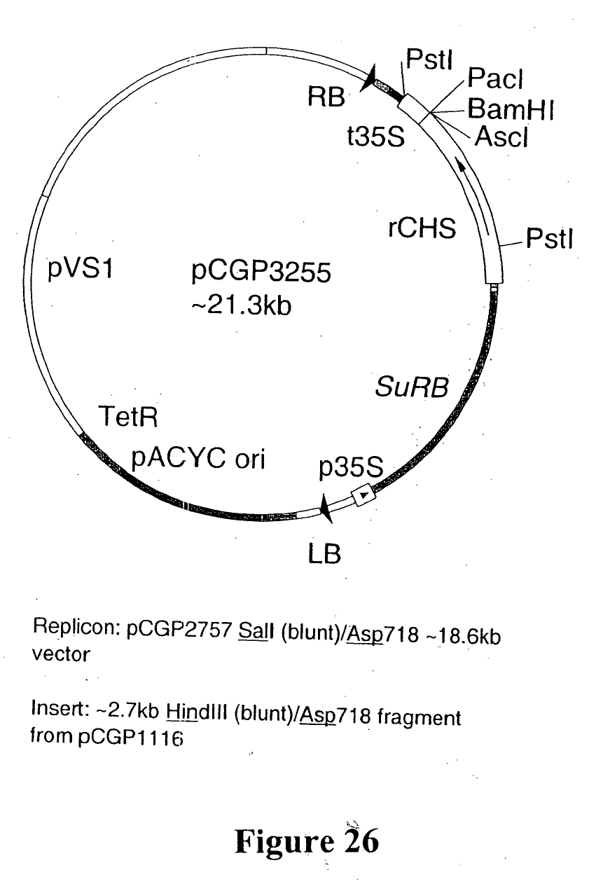

The plasmid pCGP3255 (FIG. 26) was constructed by replacing the CaMV 35S promoter in the binary vector pCGP2757 with the Rose CHS promoter fragment from pCGP1116. Plasmid pCGP1116 was initially digested with HindIII. The overhanging 5′ ends were filled-in using DNA polymerase (Klenow fragment) (Promega) according to the manufacturer's recommendation. The linearized vector was then digested with Asp718 to release a ˜2.7 kb rose CHS promoter fragment. The plasmid pCGP2757 was initially digested with SalI. The overhanging 5′ ends were filled-in using DNA polymerase (Klenow fragment) (Promega) according to the manufacturer's recommendation. The Sall digested pCGP2757 was then digested with Asp718 to release the ˜19 kb binary vector fragment and the CaMV 35S promoter fragment. The SalI (filled-in)/Asp718 ˜19 kb vector fragment was purified using QIAEX II Gel Extraction kit (Qiagen) and ligated with the HindIII (filled-in)/Asp718 ends of the rose CHS promoter fragment. Correct insertion of the rose CHS promoter was established by restriction enzyme analysis (BglII, PstI, EcoRI, HindIII, XbaI, EcoRV) of DNA isolated from tetracycline-resistant transformants.

PCR products of CFMs or colored proteins derived using the primers vispro-F1 (SEQ ID NO:184) and vispro-R1 (SEQ ID NO:185) or using any primers containing AscI and PacI restriction endonuclease recognition sites, can be digested with AscI and PacI and ligated with AscI/PacI ends of pCGP3255.

Construction of pCGP2782 (Rose CHS: T: 35S 3′ Binary)

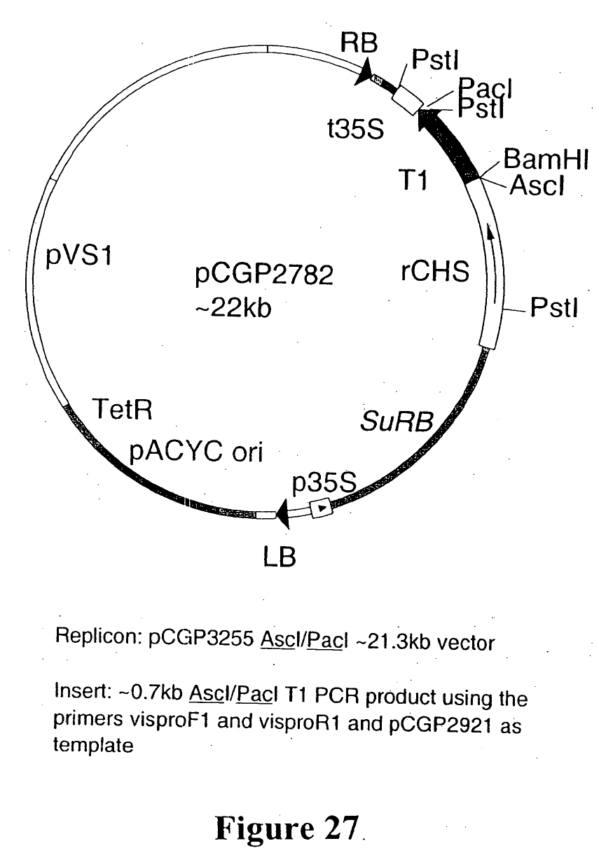

The plasmid pCGP2782 (FIG. 27) was constructed by inserting the cDNA of the T1 coral protein contained in pCGP2921 (Example 1) behind the Rose CHS promoter contained in pCGP3255.

The PCR product generated using the primers vispro-F1 (SEQ ID NO:184) and vispro-R1 (SEQ ID NO:185) and the template pCGP2921 (containing the T1 cDNA clone) was digested with AscI and PacI. PCR was carried out in 50 μL reactions with 200 μM dNTPs, 20 pmol vispro-F1 (SEQ ID NO:184), 20 pmol vispro-R1 (SEQ ID NO:185), 1×Pfu buffer (Stratagene), 2.5 units Pfu trubo DNA Polymerase (Stratagene) and ˜2 ng pCGP2921 plasmid DNA as template. The resulting product was purified using QIAquick Gel Extraction (Qiagen) and ligated with AscI/PacI ends of pCGP3255. Correct insertion of the T1 coding region behind the Rose CHS promoter was established by restriction endonuclease digestion (HindIII, EcoRI, PstI, XbaI, BstX1) of tetracycline-resistant transfonnants.

Construction of PCGP2773 (Rose CHS: D1: 35S 3′ Binary)

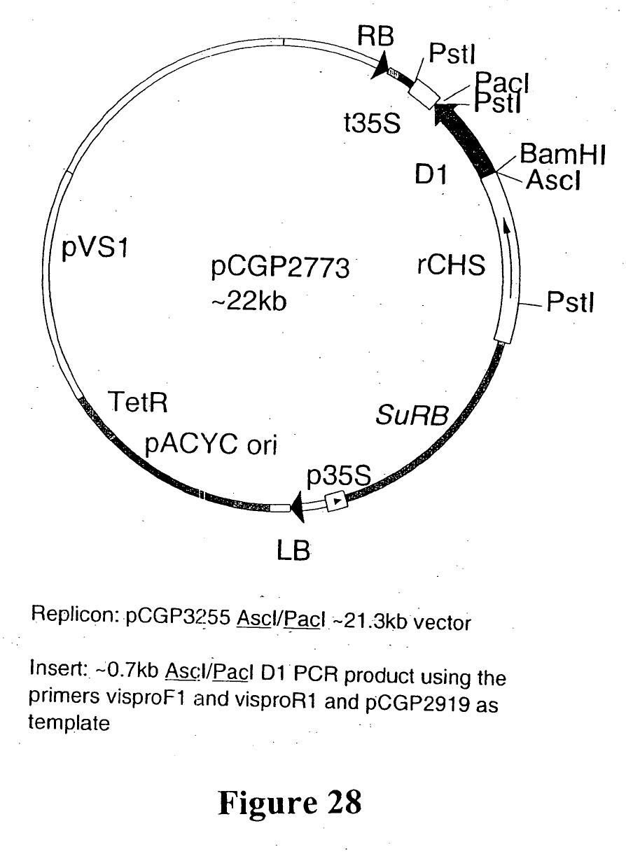

The plasmid pCGP2773 (FIG. 28) was constructed by inserting the cDNA of the D1 coral protein (Example 1) contained in pCGP2919 behind the Rose CHS promoter contained in pCGP3255.

The PCR product generated using the primers vispro-F1 (SEQ ID NO:184) and vispro-R1 (SEQ ID NO:185) and the template pCGP2919 (containing the D1 cDNA clone) was digested with AscI and PacI. The PCR product generated using the primers vispro-F1 (SEQ ID NO:184) and vispro-R1 (SEQ ID NO:185) and the template pCGP2919 (containing the D1 cDNA clone) was digested with AscI and PacI. PCR was carried out in 50 μL reactions with 200 μM dNTPs, 20 pmol vispro-F1 (SEQ ID NO:184), 20 pmol vispro-R1 (SEQ ID NO:185), 1×Pfu buffer (Stratagene), 2.5 units Pfu trubo DNA Polymerase (Stratagene) and ˜2 ng pCGP2919 plasmid DNA as template. The resulting fragment was purified using QIAquick Gel Extraction (Qiagen) and ligated with AscI/PacI ends of pCGP3255. Correct insertion of the D1 coding region behind the Rose CHS promoter was established by restriction endonuclease digestion (HindIII, EcoRI, PstI, XbaI) of tetracycline-resistant transformants.

Construction of pCGP2774 (Rose CHS: S1: 35S 3′ Binary)

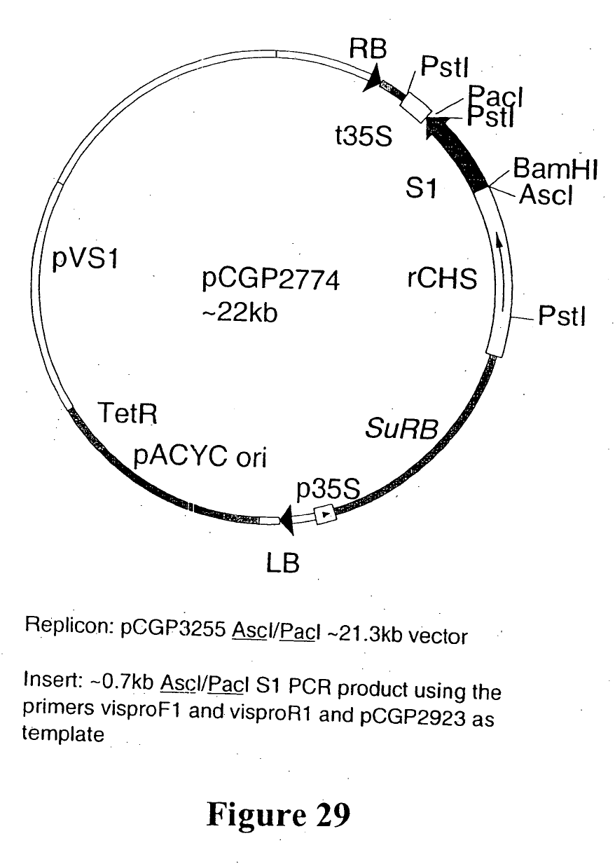

The plasmid pCGP2774 (FIG. 29) was constructed by inserting the cDNA of the S1 coral protein (Example 1) contained in pCGP2923 behind the Rose CHS promoter contained in pCGP3255.

The PCR product generated using the primers vispro-F1 (SEQ ID NO:184) and vispro-R1 (SEQ ID NO:185) and the template pCGP2923 (containing the S1 cDNA clone) was digested with AscI and PacI. The PCR product generated using the primers vispro-F1 (SEQ ID NO:184) and vispro-R1 (SEQ ID NO:185) and the template pCGP2923 (containing the S1 cDNA clone) was digested with AscI and PacI. PCR was carried out in 50 μL reactions with 200 μM dNTPs, 20 pmol vispro-F1 (SEQ DD NO:184), 20 pmol vispro-R1 (SEQ ID NO:185), 1×Pfu buffer (Stratagene), 2.5 units Pfu trubo DNA Polymerase (Stratagene) and ˜2ng pCGP2923 plasmid DNA as template. The resulting fragment was purified using QIAquick Gel Extraction (Qiagen) and ligated with AscI/PacI ends of pCGP3255. Correct insertion of the S1 coding region behind the Rose CHS promoter was established by restriction endonuclease digestion (HindIII, EcoRI, PstI, XbaI) of tetracycline-resistant transformants.

### EXAMPLE 11

### Targeting of Colored Proteins to Increase Expression in Plants

The levels of some CFMs or colored proteins produced in the cytosol of cells may have to be elevated in order to impart a visible color or a phenotype with commercial value. It is expected that targeting the CFM or colored proteins to different organelles within transgenic cells will significantly increase CFM or colored protein levels. The increased accumulation of transgene products by targeting to organelles has been demonstrated previously. For example, see Table 17.

It is also expected that plasmid transformation of Arabidopsis, carnation, rose or other plant species will significantly increase CFM or colored protein levels. Increased accumulation of transgene products by plastid transformation has been demonstrated previously. For example, see Table 18.

Cloning of the Chloroplast/plastid Transit Peptide Sequence from Tobacco

CFMs or colored proteins may be targeted to plastids with the inclusion of N-terminal plastid or chloroplast targeting peptides.

The 57 amino acid transit peptide of small subunit (SSU) of ribulose biphosphate carboxylase from Nicotiana sylvestris (Pinck et al., Biochimie 66:539-545, 1984) was selected to target coral colored proteins to plastids of transgenic Arabidopsis, carnation, rose or other plant species.

The primers TSSU-Fnew (SEQ ID NO:205) and TSSU-R (SEQ ID NO:206) were used to amplify the tobacco chloroplast transit-peptide coding region using the plasmid pCGN5075 (Calgene) as template.

```
SEQ ID NO:205 TSSU-Fnew
CAG GGCGCGCC AAGGAGATAT AACA ATG GCT TCC TCA GTT CTT TCC
            AsaI RBS TICS M A S S V L S
SEQ ID NO:206 TSSU-R
CACT GGATCC GCA TTG CAC TCT TCC GCC GTT GC
          BamHI C Q V R G G N
```

TSSU-Fnew (SEQ ED NO:205) contains all AscI site for cloning into 35S and Rose CHS expression vectors, a prokaryotic ribosomal binding site (RBS) for bacterial expression and a plant translational initiation context sequence (TICS) for improved translation in plants. TSSU-R (SEQ ID NO:206) contains a BamHI site to allow the cloning of the transit peptide in frame with coral colored protein sequences produced using vispro-F1 (SEQ ID NO:184) and vispro-R1 (SEQ ID NO:185) primers.

PCR conditions included 1 μL TSSU-Fnew (20 pmol/μL) (SEQ ID NO:205), 1 μL TSSU-R (20 pmol/μL) (SEQ ID NO:206), 5 μL 10×pfu buffer (Stratagene), ˜20 ng pCGN5075 plasmid DNA as template, 1 μL 10 mM dNTP mix, 0.5 μL Pfu turbo DNA polymerase (2.5 U/μL) (Stratagene) in a 50 μL reaction. The cycling conditions were 94° C. for 5 minutes, followed by 35 cycles of 94° C. for 30 min, 50° C. for 30 min and 72° C. for 60 min, and a final incubation at 72° C. for 10 min. After completion of the PCR the products were stored at 4° C. PCR products were purified using a QIAquick PCR purification Kit (Qiagen) and cloned into pUC18 SmaI vector (Pharmacia/Amersham). The resulting plasmid was designated pCGP2783. The sequence of the transit peptide (TSSU) was confirmed by sequencing across both strands.

Construction of pCGP2780 (35 Expression Binary with Unique BamHI site)

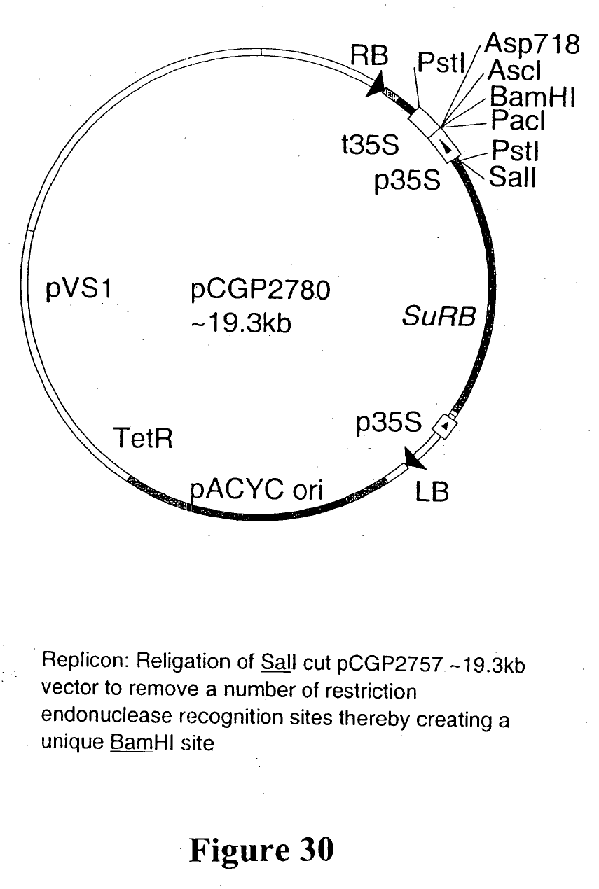

Plasmid pCGP2780 (FIG. 30) was constructed by removing a ˜290 bp SalI fragment from pCGP2757. The plasmid pCGP2757 was digested with SalI to release a ˜290 bp fragment and ˜19 kb binary vector. The ˜19 kb binary vector was isolated and purified using the QIAEX II Gel Extraction kit (Qiagen) and self-ligated using the Amersham Ligation Kit. Correct religation of the SalI ends was established by restriction enzyme analysis (PvuII, BamHI, SalI) of DNA isolated from tetracycline-resistant transformants.

Construction of pCGP2784 (35S Expression Pre-binary Containing Plastid Transit Peptide)

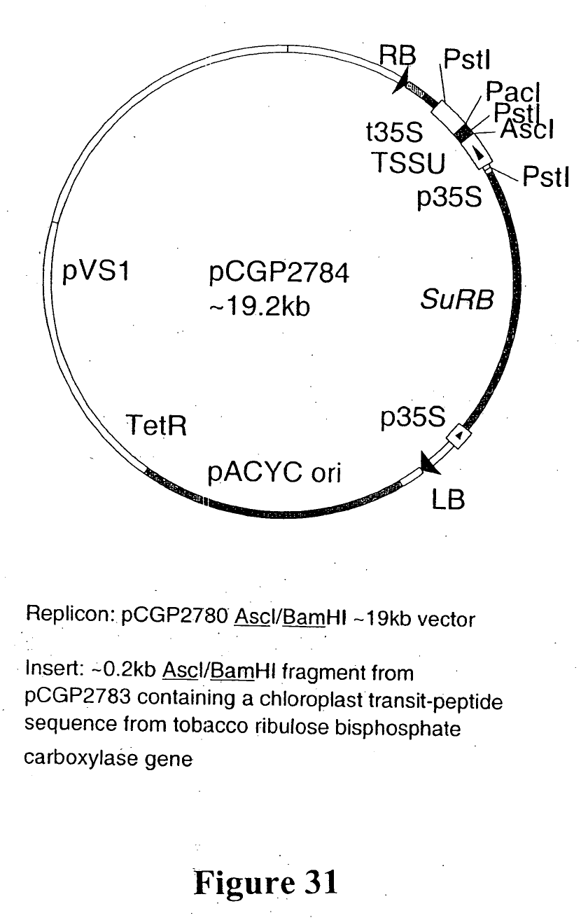

The plasmid pCGP2784 (FIG. 31) was constructed by inserting the chloroplast transit peptide from tobacco contained in pCGP2783 into the binary vector pCGP2781.

Plasmid pCGP2783 was digested with AscI and BamHI to release the ˜0.2 kb TSSU fragment. The 0.2 kb TSSU fragment was isolated and purified using the QIAEX II Gel Extraction kit (Qiagen) and ligated with AscI/BamHI ends of pCGP2781 binary vector. Correct insertion of the transit peptide in frame and upstream of the T1 coding sequence was established by restriction enzyme analysis (EcoRI, PstI, XbaI, AscI/PacI) of DNA isolated from tetracycline-resistant transformants.

PCR products of CFMs or colored proteins derived using the primers vispro-F1 (SEQ ID NO:184) and vispro-R1 (SEQ ID NO:185) or using any primers containing BamHI and PacI restriction endonuclease recognition sites, can be digested with BamHI and PacI and ligated with BamHI/PacI ends of pCGP2784. The coding region of the CFMs or colored proteins will then be in-frame with the plastid targeting peptide to allow expression of the proteins in the plastids or chloroplasts.

Construction of pCGP2781 (35S: T1: 35S Binary with Unique BamHI Site)

Plasmid pCGP2781 (FIG. 32) was constructed by removing a ˜290 bp SalI fragment from pCGP2772. The plasmid pCGP2772 was digested with SalI to release a ˜290 bp fragment and ˜19 kb binary vector. The ˜19 kb binary vector was isolated and purified using the QIAEX II Gel Extraction kit (Qiagen) and self-ligated using the Amersham Ligation Kit. Correct religation of the SalI ends was established by restriction enzyme analysis (PvuII, BamHI, SalI, XbaI) of DNA isolated from tetracycline-resistant transformants.

Construction of pCGP2785 (35S: TSSU.: T1: 355 Binary)

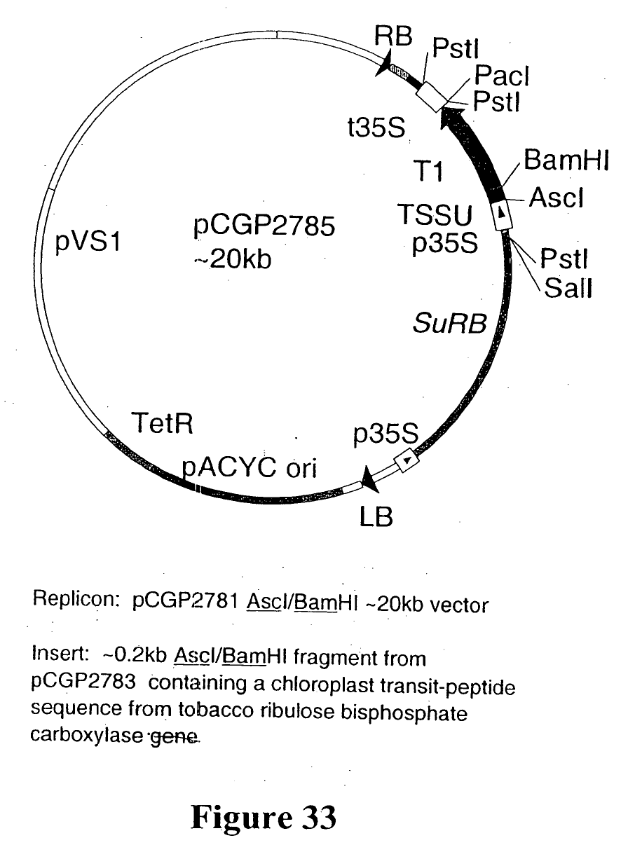

The plasmid pCGP2785 (FIG. 33) was constructed by inserting the chloroplast transit peptide from tobacco contained in pCGP2783 into the binary vector pCGP2781.

Plasmid pCGP2783 was digested with AscI and BamHI to release the ˜0.2 kb TSSU fragment. The 0.2 kb TSSU fragment was isolated and purified using the QIAEX II Gel Extraction kit (Qiagen) and ligated with AscI/BamHI ends of pCGP2781 binary vector. Correct insertion of the transit peptide in frame and upstream of the T1 coding sequence was established by restriction enzyme analysis (EcoRI, PstI, XbaI, AscI/PacI) of DNA isolated from tetracycline-resistant transformants.

Construction of pCGP2787 (Rose CHS: TSSU: T1: 35S Binary)

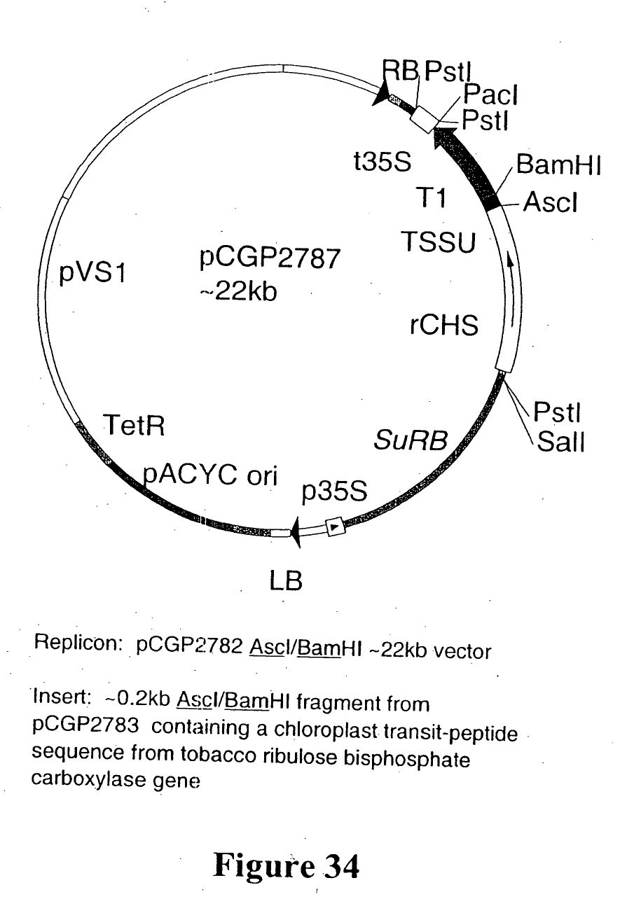

The plasmid pCGP2787 FIG. 34) was constructed by inserting the chloroplast transit peptide from tobacco contained in pCGP2783 (Example 11) into the binary vector pCGP2782 (FIG. 27).

Plasmid pCGP2783 was digested with AscI and BamHI to release the ˜0.2 kb TSSU fragment. The 0.2 kb TSSU fragment was isolated and purified using the QIAEX II Gel Extraction kit (Qiagen) and ligated with AscI/BamHI ends of pCGP2782 binary vector. Correct insertion of the transit peptide in frame and upstream of the T1 coding sequence was established by restriction enzyme analysis of DNA isolated from tetracycline-resistant transformants (FIG. 34)

Targeting of CFMs or Colored Proteins to Endoplasmic Reticulum

CFMs or colored proteins are targeted to endoplasmic reticulum with the inclusion of N-terminal endoplasmic reticulum (ER) targeting peptides and C-terminal ER retaining signals.

The Arabidopsis thaliana basic chitinase N-terminal signal sequence was isolated to target CFMs and colored proteins to the ER (Haseloff et al., 1997, supra). To retain the proteins in the ER an HDEL peptide sequence was generated to be cloned in at the 3′ end of the coding region (Haseloff et al., 1997, supra). These ER-targeting and ER-retention signals are used to increase levels of CFMs and colored protein in transgenic Arabidopsis, carnation, rose or other plant species.

The plasmid pBIN35Sm-GFP4ER Haseloff et al., 1997, supra) (http:www.plantsci.cam.ac.uk/Haseloff/GFP/mgfp4.html) was used as the source of Arabidopsis thaliana basic chitinase N-terminal signal sequence and HDEL ER-retention signal.

A PCR based approach was used to generate AscI and BamHI sites flanking the N-terminal ER transit peptide sequence. The primers AscI-ER.F (SEQ ID NO:207) and ER-BamHI.R (SEQ ID NO:208) were used to amplify the N-terminal ER sequence contained in pBIN35Sm-GFP4-ER.

Primer AscI-ER.F (SEQ ID NO:207) contains an AscI site for cloning into 35S and Rose CHS expression binaries (see Examples 9 and 10), a prokaryotic ribosome binding site (RBS) to allow for bacterial expression and a plant translational initiation context sequence (TICS).

```
SEQ ID NO:207 AscI-ER.F (5′ to 3′)
GCAT GGCGCGCC AAGGAGATAT AACA ATG AAG ACT AAT CTT TTT C
              AscI RBS TICS M K T N L F
SEQ ID NO:208 ER-BamHI.R (5′ to 3′)
            BamHI EcoRI
GCAT GGA TCC GAA TTC GGC CGA GGA TAA TGA TAG
            S G F E A S S L S L
```

PCR conditions included using 1 ng plasmid pBIN35Sm-GFP4-ER template, 100 ng each of primers AscI-ER.F (SEQ ID NO:207) and ER-BamHI.R (SEQ ID NO:208), 2.5 μL 10×pfu turbo buffer (Stratagene), 1 μL pfu turbo (Stratagene) in a total volume of 25 μL. Cycling conditions were an initial denaturation step of 5 min at 94° C., followed by 35 cycles of 94° C. for 30 sec, 50° C. for 30 sec and 72° C. for 1 min with a last treatment of 72° C. for 5 min and then finally storage at 4° C.

An expected product of ˜100 bp was amplified and purified using the QIAEX II Gel Extraction kit (Qiagen) according to procedures recommended by the manufacturer. The 100 bp fragment was then cloned into pCR2.1 (Invitrogen) and the plasmid was designated pCGP3256. The sequence of the N-terminal ER transit peptide fragment was confirmed by sequence analysis using the M13 reverse and M13-20 primers.

Construction of pCGP3257 (35S:ER.MCS:35S Pre-binary)

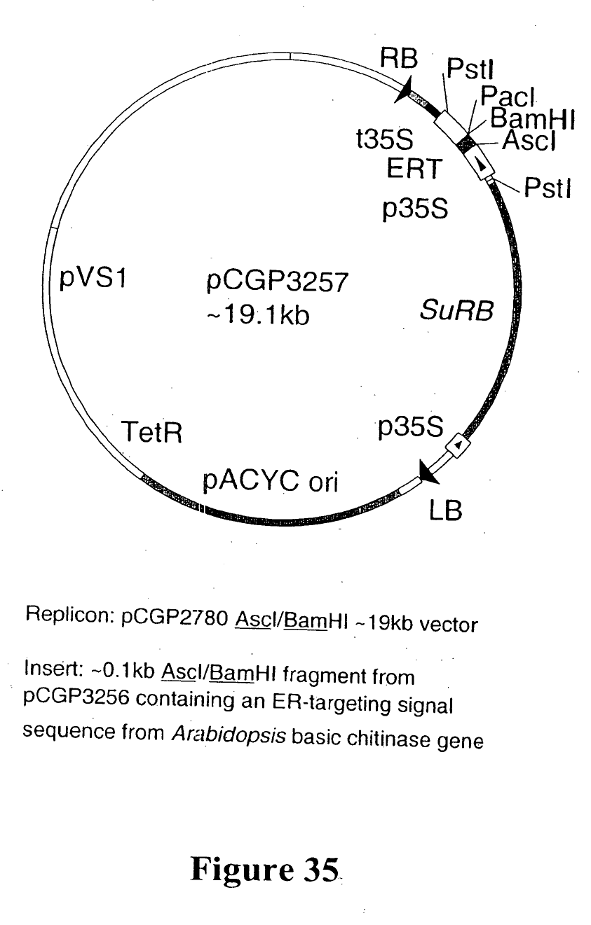

The N-terminal ER transit peptide fragment was cloned downstream of the 35S promoter contained in the pre-binary pCGP2780 (FIG. 30) to produce pCGP3257 (FIG. 35). Plasmid pCGP3256 was digested with AscI and BamHI to release the ˜100 bp N-terminal ER transit peptide fragment. The fragment was isolated and purified using QIAEX II Gel Extraction kit (Qiagen) and ligated with AscI/BamHI ends of pCGP2780. Correct insertion of the N-terminal ER transit peptide fragment was established by restriction endonuclease analysis of DNA isolated from tetracycline-resistant transfonnants.

PCR products of CFMs or colored proteins derived using the primers vispro F1 (SEQ ID NO:185) and CP-HDEL-PacI.R (described in this Example below) can be digested with BamHI and PacI and ligated with BamHI/PacI ends of pCGP3257. The coding region of the CFMs or colored proteins will be under the control of the CaMV 35S promoter and in-frame with the ER transit targeting peptide to allow targeting of the proteins to the ER. The coding region of the CFMs or colored proteins will also contain the HDEL sequence at the C-terminal end to allow retention of the proteins in the ER.

Construction of pCGP3259 (35S: ER: T1.HDEL: 35S binary)

The coding sequence of the colored protein T1 was amplified by PCR using the primers vispro-F1 (SEQ ID NO:184) and CP-HDEL-PacI.R (SEQ ID NO:209) and the plasmid pCGP2779 as template. The primer CP-HDEL-PacI.R was designed to include a PacI site with a translational termination codon for cloning into the binary vectors described in this specification, a HDEL peptide sequence in-frame with the colored protein sequence and a PstI site for cloning into the bacterial expression vector pQE-30 (Qiagen).

```
SEQ ID NO:209 CP-HDEL-PacI.R (5′ to 3′)
              PacI PstI
GATCTTAAT°TAA°AGC TCA TCA TGC TGC°AGG GCG ACC ACA GGT TTG C
                      * L E D H Q L A V V P K
```

PCR conditions included using 2 ng plasmid pCGP2779 as template, 100 ng each of primers vispro-F1 (SEQ ID NO:184) and CP-HDEL-PacI.R (SEQ ID NO:209), 2 μL 10 mM dNTP mix, 5 μL 10×PfuTurbo (registered trademark) DNA polymerase buffer (Stratagene), 0.5 μL PfuTurbo (registered trademark) DNA polymerase (2.5 units/μL) (Stratagene) in a total volume of 50 μL. Cycling conditions were an initial denaturation step of 5 min at 94° C., followed by 35 cycles of 94° C. for 20 sec, 50° C. for 30 sec and 72° C. for 1 min with a last treatment of 72° C. for 10 min and then finally storage at 4° C.

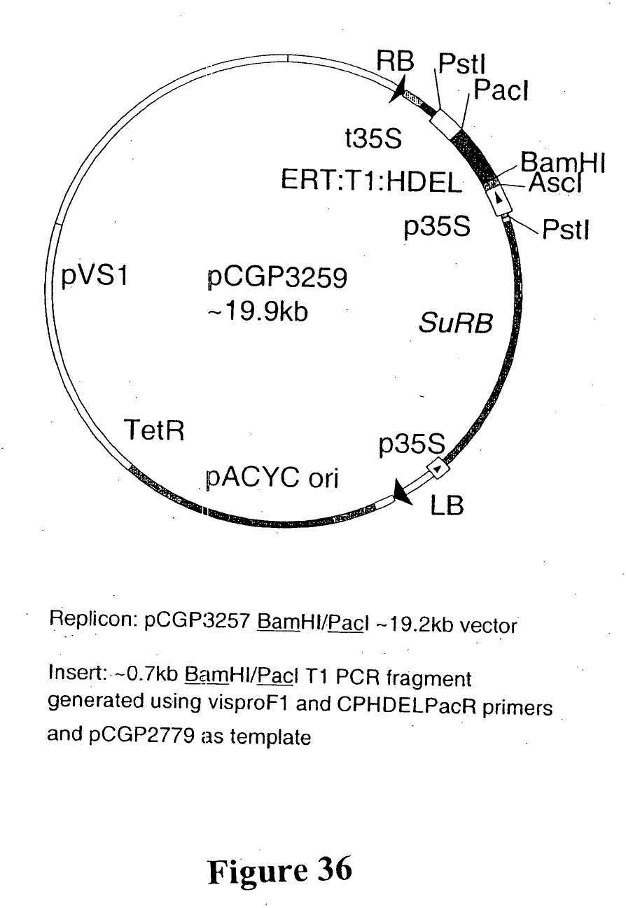

The resulting ˜700 bp product was digested with BamHI and PacI, isolated and purified using QIAEXII Gel Extraction kit (Qiagen) and ligated with BamHI/PacI ends of pCGP3257. Correct insertion of the T1 coding region and HDEL sequence in-frame with the ER transit peptide sequence under the control of the 35S promoter was established by restriction endonuclease analysis (BamHI, EcoRI, AscI, PacI) of DNA isolated from tetracycline-resistant transformants. The resulting plasmid was designated pCGP3259 (FIG. 36).

Construction of pCGP3262 (RoseCHS:ER:MCS:35S Pre-binary)

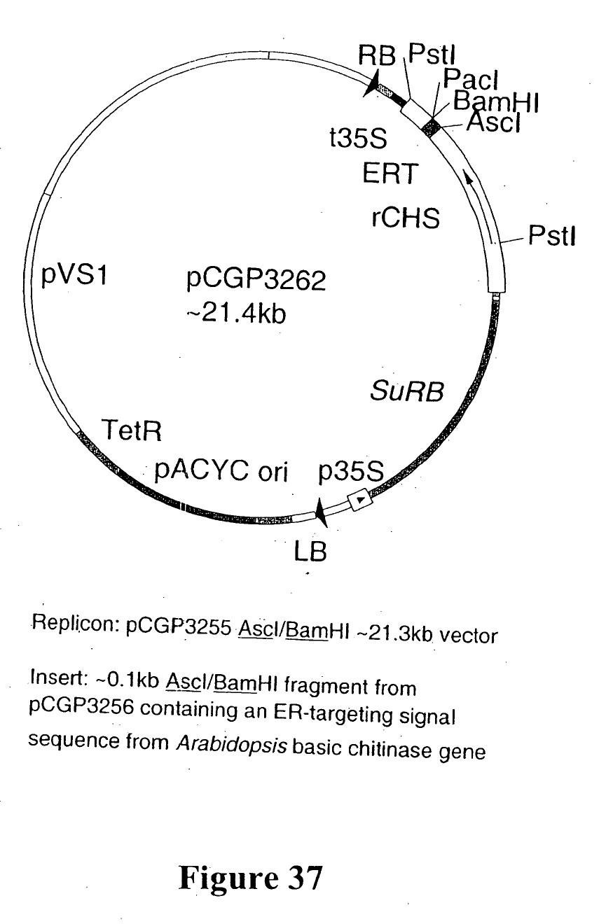

The N-terminal ER transit peptide fragment was cloned downstream of the Rose CHS promoter contained in the pre-binary pCGP3255 to produce pCGP3262 (FIG. 37). Plasmid pCGP3256 was digested with AscI and BamHI to release the ˜100 bp N-terminal ER transit peptide fragment. The fragment was isolated and purified using QIAEX II Gel Extraction kit (Qiagen) and ligated with AscI/BamHI ends of pCGP3255. Correct insertion of the N-terminal ER transit peptide fragment was established by restriction endonuclease analysis of DNA isolated from tetracycline-resistant transformants.

PCR products of CFMs or colored proteins derived using the primers vispro-F1 (SEQ ID NO:184) and CP-HDEL-PacI.R (SEQ ID NO:209) can be digested with BamHI and PacI and ligated with BamHI/PacI ends of pCGP3262. The coding region of the CFMs or colored proteins will be under the control of the Rose CHS promoter and in-frame with the ER transit targeting peptide to allow targeting of the proteins to the ER. The coding region of the CFMs or colored proteins will also contain the HDEL sequence at the C-terminal to allow retention of the proteins in the ER of floral tissues.

Construction of pCGP3263 (Rose CHS:ER: T1-HDEL:35S Binary)

The coding sequence of the colored protein T1 was amplified by PCR using the primers vispro-F1 (SEQ ID NO:184) and CP-HDEL-PacI.R (SEQ ID NO:209) and the plasmid pCGP2779 as template.

PCR conditions were as described above for construction of pCGP3259.

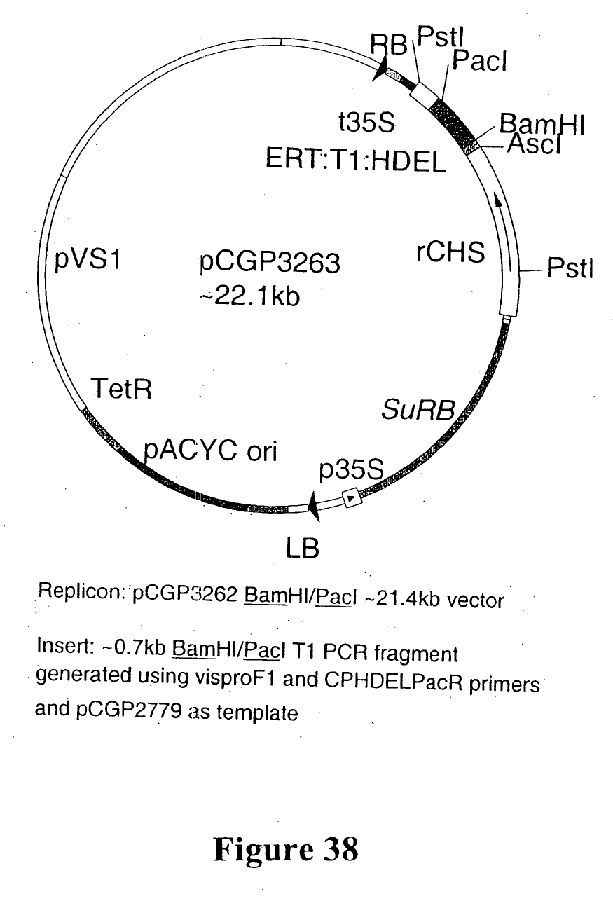

The resulting ˜700bp product was digested with BamHI and PacI, isolated and purified using QIABX II Gel Extraction kit (Qiagen) and ligated with BamHI/PacI ends of pCGP3262. Correct insertion of the T1 coding region and HDEL sequence in-frame with the ER transit peptide sequence under the control of the Rose CHS promoter was established by restriction endonuclease analysis (BamHI, EcoRI, AscI, PacI) of DNA isolated from tetracycline-resistant transformants. The resulting plasmid was designated pCGP3263 (FIG. 38).

A site predicting N-glycosylation was identified within the coloured protein T1 (‘NDS’-surrounding amino acid 107) (SEQ ID NO:202). This site is conserved among the colored protein clones D1, D10, T1, T3, S3 and A8 and these include both purple and blue varieties. Comparison of this region in sequences of other coloured and fluorescent varieties in the GenBank database (e.g., asCP562, asFP499, Clavularia FP484, Discosoma FP483 etc) indicate the presence of two alternative sequences in this position—QDS or NDI. The first converts an asparagine residue (N) to a glutamine (Q) (a conservative change given both residues are polar) and the second changes the serine (S) to an isoluecine (I) (a non conservative change from a polar to a non polar residue). Both naturally occurring sequence alternatives for this region of the protein were be performed separately. That is, mutation of the T1 sequence from NDS to QDS and a separate mutation from NDS to NDI.

The plasmid pCGP2921 (FIG. 10) was used as a source of the coding sequence for T1 blue protein. A BamHI/HindII fragment was isolated from pCGP2921 and cloned with BamHI/HindIII ends of pBluescript to produce pCGP3268. The GeneEditor in vitro Site Directed Mutagenesis Kit (Promega) was used following the manufacturer's instructions along with the following oligonucleotides (T1.N-Q N(AAT)>Q(CAG) SEQ ID NO:230) and T1.S-I S(C)>I(ATC) SEQ ID NO:231) to introduce the mutations in pCGP3268.

SEQ ID NO:230 T1.N-Q N(AAT) > Q(CAG)
GTG TGT ACT GTC AGC CAG GAT TCC AGC ATC CAA C
V C T V S Q D S S I Q
SEQ ID NO:231 T1.S-I S(TCC) > I(ATC)
CT GTC AGC AAT GAT ATC AGC ATC CAA GGC AAC

The resultant plasmids pCGP3271 and pCGP3272 containing the N107Q and S109I mutated forms of T1 blue protein in pBluescript were sequenced thoroughly to confirm the presence of the mutated sequence.

Construction of pCGP3273 (pQE30:T1(N107Q) and pCGP3274 (QE30:T1(S109I)

E. coli expression of the mutated forms of T1 in pCGP3271 and pCGP3272 was necessary to determine if the mutations had any effect on the colour of the expressed protein. Thus, BamHI/HindIII fragments pCGP3271 and pCGP3272 were subcloned with BamHI/HindIII ends of pQE30. The resultant plasmids were designated pCGP3273 (T1-N107Q) and and 6) to determine the colour of the expressed protein. The protein expressed by the sequence encoded in pCGP3273 was found to retain the original colour of T1 as expressed by pCGP2921, while the protein expressed by pCGP3274 was not coloured. This suggested that the S109I mutation may have had a deleterious effect on the color of the protein. Investigation of this protein will provide information on the amino acids that are critical to color formation of colored proteins.

Construction of pCGP3275 (35S: ER:T1(N107 Q).HDEL:35S Binary) and pCGP3276 (35S: ER:T1(S109I).HDEL:35S Binary)

The coding sequence of the coloured protein T1(N107Q) was amplified by PCR using the primers vispro-F1 (SEQ ID NO:184) and CP-HDEL-PacI.R (SEQ ID NO:207) and the plasmids pCGP3271 (described above) and pCGP3272 (described above) as template essentially as described in the construction of pCGP3259 (Example 11).

The resulting ˜700 bp products were digested with BamHI and PacI, isolated and purified using QLAEXII Gel Extraction kit (Qiagen) and ligated with BamHI/PacI ends of pCGP3257 (FIG. 35). Correct insertion of the coding regions of T1(N107Q) and T1(S109I) and HDEL sequence in-frame with the ER transit peptide sequence under the control of the CaMV 35S promoter was established by restriction endonuclease analysis (BamHI, EcoRI, AscI, PacI, EcoRV) of DNA isolated from tetracycline resistant transformants. The resulting plasmids were designated pCGP3275 and pCGP3276.

Construction of pCGP3277 (RoseCHS: ER:T1(N170Q).HDEL:35S Binary) and pCGP3276 (Rose CHS: ER:T1(S109I).HDEL:35S Binary

The coding sequence of the coloured protein T1 (N107Q) was amplified by PCR using the primers vispro F1 (SEQ ID NO:184) and CP-HDEL-PacI.R (SEQ ID NO:207) and the plasmids pCGP3271 and pCGP3272 as template essentially as described in the construction of pCGP3259 (Example 11).

The resulting ˜700 bp products were digested with BamHI and PacI, isolated and purified using QIAEXII Gel Extraction kit (Qiagen) and ligated with BamHI/PacI ends of pCGP3262 (FIG. 37). Correct insertion of the coding regions of T1(N107Q) and T1(S109I) and BDEL sequence in-frame with the ER transit peptide sequence under the control of the Rose CHS promoter was established by restriction endonuclease analysis (BamHI, EcoRI, AscI, PacI, EcoRV) of DNA isolated from tetracycline resistant transformants. The resulting plasmids were designated pCGP3277 and pCGP3278.

### EXAMPLE 12

### Fusion Proteins with GFP

Construction of pCGP3258 (35S: T1/m GFP4:35S Binary)

As a way of tracking the expression and localisation of the T1 coloured protein the T1 coding region was fused with the N-terminus of mGFP4 (Haseloff et al., PNAS 94:2122-2127, 1997).

The mGFP4 coding sequence was amplified using the primers PstI-mGFP4F (SEQ ED NO:210) and mGFP4-PacIR (SEQ ID NO:211) and pBIN35SmGFP4ER (Haseloff et al., 1997) as template. A ˜700 bp product was gel purified and then digested with the restriction endonucleases PstI and PacI. The T1 coding sequence was amplified using the primers visproF1-new (SEQ ID NO:212) and visproR1 (SEQ ID NO:185) and pCGP2779 as template.

```
SEQ ID NO:210 Pst-mGFP4F (5′ to 3′)
              PstI linker sequences
GCAT CTG CAG GTC GCC ACC AGT AAA GGA GAA GAA CTT TTC AC
            L Q V A T S K G E E L F
SEQ ID NO:211 mGFP4-PacIR
            PacI
CTGA TTAATTAA TTA TTT GTA TAG TTC ATC CAT GCC ATG
                              * K Y L E D M G H
SEQ ID NO:212 visproF1-new
            AscI RBS TICS BamHI
CAG GGCGCGCC AAGGAGATAT AACA ATG GGA TCC GTT ATC GCT AAA CAG ATG ACC
                                                            M G S V I A K Q M T
```

A ˜700 bp product was gel purified and then digested with the restriction endonucleases AscI and PstI.

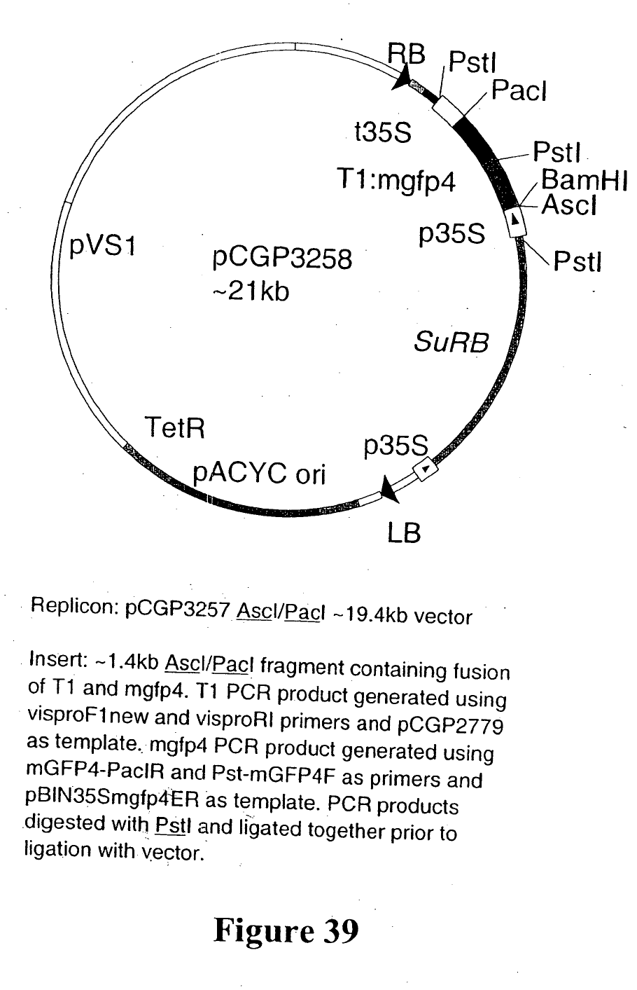

The PstI/PacI mGFP4 fragment was ligated with the AscI/PstI T1 fragment. The resulting ligated fragment was then ligated with the AscI/PacI ends of the binary vector pCGP3257 (FIG. 35) to produce pCGP3258 (FIG. 39). Correct insertion of the T1:mGFP4 fusion was established by restriction endonuclease analysis (BstXI, EcoRI, NcoI, PstI) of DNA isolated from tetracycline-resistant transformants. The resulting plasmid was designated pCGP3258 (FIG. 39).

Construction of pCGP3261 (35S.ER:T1:GFP: 35S Binary)

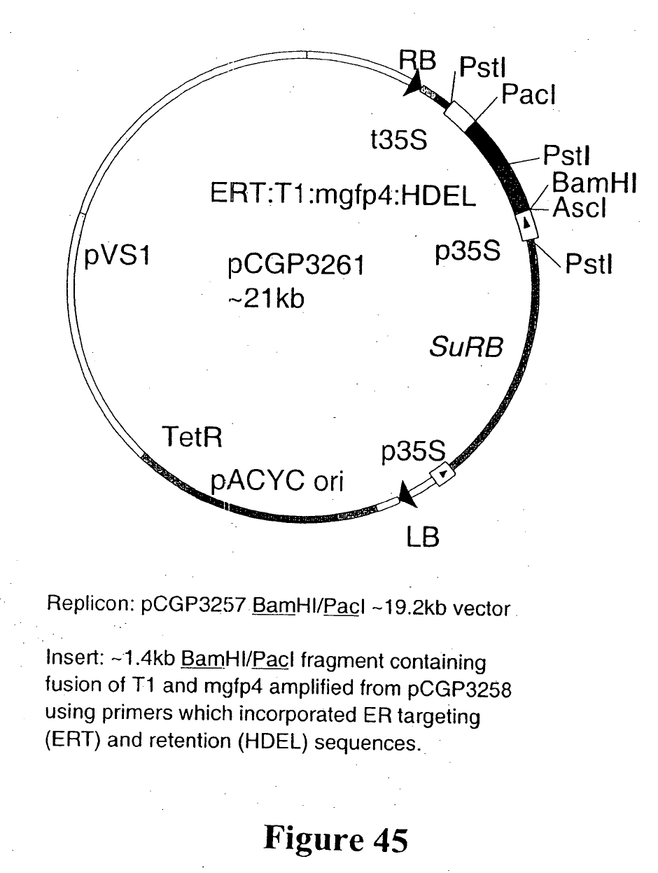

An ER targeted version of the T1:mGFP4 fusion in pCGP3258 under the control of the CaMV 35S promoter was also prepared. This plasmid was designated pCGP3261 (FIG. 45).

The T1:mGFP4 fusion was amplified using the primers vispro-F1 (SEQ ID NO:184) and mGFP4-HDEL-PacR (SEQ ID NO:229) and pCGP3258 (FIG. 39) as template. A ˜1.4 kb product was gel purified and then digested with the restriction endonucleases BamHI and PacI. The resulting fragment was then ligated with BamHI/PacI ends of the binary vector pCGP3257 (FIG. 35) to produce pCGP3261 (FIG. 45). Correct insertion of the T1:mGFP4 fusion was established by restriction endonuclease analysis (BstXI, EcoRI, NcoI, PstI, AscI/PacI, XbaI) of DNA isolated from tetracycline-resistant transformants. The resulting plasmid was designated pCGP3261 (FIG. 45).

| | |
|---|---|
| mGFP4-HDEL-PacR (5′ TO 3′) | |
| CTG ATT AAT TAA AGC TCA TCA TGT TTG TAT AGT TCA TCC ATG CCA TG | SEQ ID NO:229 |

Construction of pCGP3260 (35S.ER:GFP: 35S Binary

An ER targeted version of the mGFP4 in pBIN35SmGFP4ER (Haseloff et al., 1997 supra) under the control of the CaMV 35S promoter and CaMV 35S terminator was prepared to use as a control for the binaries pCGP3258 (FIG. 39) and pCGP3261 (FIG. 45).

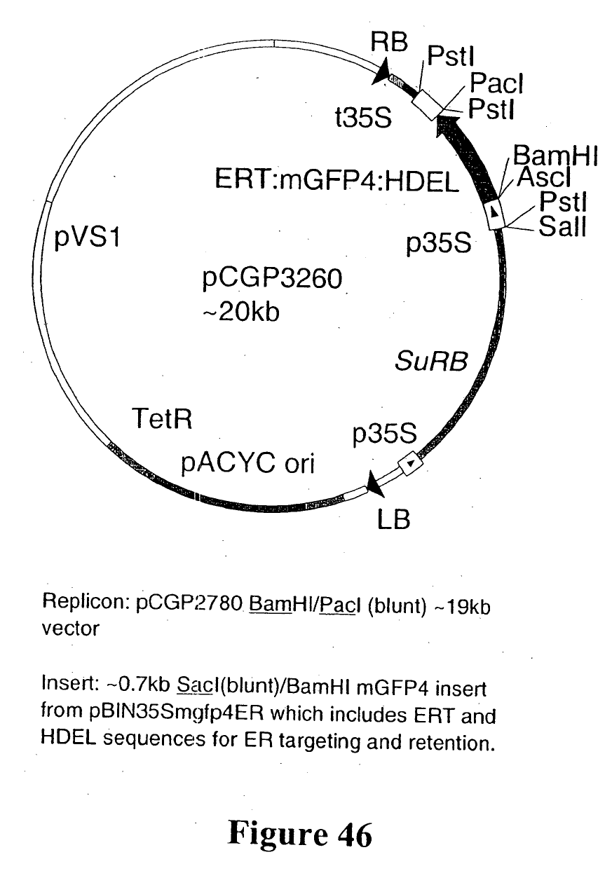

The plasmid pBIN35SmGFP4ER (Haseloff et al., 1997 supra) was initially digested with the restriction endonuclease SacI. The resulting overhang was repaired and the linearized vector was then digested with BamHI to release a ˜0.7 kb fragment containing the mGFP4 coding sequence. The resulting SacI(blunt)/BamHI mGFP4 fragment was gel purified and then ligated with BamHI/PacI (blunt) ends of the binary vector pCGP2780 (FIG. 30). Correct insertion of the mGFP4 coding sequence was established by restriction endonuclease analysis (EcoRI, NcoI, PstI, BamHI, XbaI) of DNA isolated from tetracycline-resistant transformants. The resulting plasmid was designated pCGP3260 (FIG. 46).

### EXAMPLE 13

### Reconstruction of Color

In order to determine whether rose petals or plant material in general conatin proteases that may degrade colored proteins reconstructions of rose petal extracts with the T1 colored protein were set up.

Petals of Rosa hybrids cultivar Medeo are generally white to pale apricot. Expression of colored proteins in a white flower should allow visualisation of color when colored proteins are expressed in flowers.

One gram amounts of Medeo rose-petals were ground in 500 μL water using a mortar and pestle. The resultant slurries were centrifuged at 14 000 rpm for 5 min in 1.5 mL centrifuge tubes. The supernatants were collected and 100 tμL of the extracts were aliquoted into the wells of a microtitre tray. Ten microlitres aliquots containing ˜30 μg of His-tag purified T1 protein (purifed as described in Example 8) were added to the Medeo extracts. In order to determine whether the color of the colored protein is affected by pH, the pH of some of the reconstructions was modified by addition of NaOH so that the final pH was 7.0, 8.5 or 10.0. The pH of Medeo petal extract alone was pH 4.5 and 4.6. The pH of Medeo petal extract mixed with T1 protein was pH 5.2, 5.8 and 6.1. The color of reconstructions of Medeo petal extract mixed with T1 protein at pH 5.2, 5.8 and 6.1 was light blue (RHSCC 101C/RHSCC 115B). However the color at pH 7.0 and 8.5 was a pale blue-green (RHSCC 122C) and that at pH 10.0 was yellow. The colors were still evident after 5 hours incubation at room temperature as well as 48 hours at room temperature indicating that the colored protein was stable in petal extract.

An interesting and unexpected observation was that the color of the T1 protein changed to yellow when in a high pH solution. Analysis of the conformation of the protein at this high pH provides information that allows for the design of targeted mutations to T1 or other colored protein sequence and thus allows for the production of a yellow color in a low to neutral pH environment such is found in plant cells. Alternatively random shuffling (U.S. Pat. No. 6,132,970) using selections of the vast number of colored protein sequences isolated and then expressing these mutated versions in E. coli or yeast as described in Examples 3, 4, 6 and 7 will provide a means of selecting for altered or improved colors and/or brightness of the proteins expressed.

Incubation of Petunia Petals with T1 Protein

The flowers of Petunia hybrida cultivar Mitchell are white. Mitchell petal sections were incubated with the Ti protein to determine the color that would be produced in white petals upon production of the colored proteins. Petal sections (including part of the tube and limb) were incubated in 200 μL His-tag purified T1 protein (from E. coli cultures as described in Example 8) (6 mg/mL in 20 mM Tris HCl pH 8.0) and His-tag purified A8 protein (from yeast cultures as described in Example 8) (1 mg/mL in 20 mM Tris HCl pH 8.0). In both cases the colored proteins were taking up by the petal fragments within a few minutes as visualised by coloration of the cut surface of the petal. Incubation of white petals in the T1 protein solution resulted in petals of a pale blue (RHSCC 112D) color whereas incubation of white petals in the A8 protein solution resulted in a pale purple color in the petal tissue. This experiment showed that the protein is stable in petal tissue and that the color produced will not be masked or quenched by other plant compounds.

### EXAMPLE 14

### Expression of Colored Proteins in Arabidopsis

Transformation of Arabidopsis

Construction of pCGP960 (35S:gus:ocs Binary)

The binary vector pCGP960 was prepared to use as a control in plant transformation experiments. A CaMV35S:GUS:ocs3′ expression cassette was isolated from pKIWI101 (Klee et al., Bio/Technology 3:637-642, 1985) and inserted into the pWTT2132 (DNAP) binary vector backbone which contains a CaMV 35S:SuRB selectable marker gene.

The binary vectors pCGP2772 (FIG. 24), pCGP2765 (FIG. 21), pCGP3259 (FIG. 36), pCGP2785 FIG. 33), pCGP3258 (FIG. 39), pCGP2926 (FIG. 44), pCGP3263 (FIG. 38), pCGP2787 (FIG. 34), pCGP2782 (FIG. 27), pCGP960 (see above), pCGP3261 (FIG. 45), pCGP3260 (FIG. 46), pBINmGFP4ER (Haseloff et al., 1997, supra) were introduced into Agrobacterium tumefaciens strain AGL0 as described in Example 1.

Arabidopsis thaliana

ecotype WS-2 was transformed with the above constructs using the floral dip method as mentioned in Example 1. Seeds from dipped plants were plated on selection and transgenic plants were allowed to grow until flowering. Plants can be allowed to self-fertilize to produce seed. The T2 seed can then be germinated on selection (e.g. 100 μg/mL chlorsulfuron selection for those transformed with a CaMV 35S: SuRB selectable marker gene) and allowed to grow to flowering. A number of the T2 generation would be expected to be homozygous for the introduced transgenes with the expectation that these plants would have increased coloured protein gene expression and protein production than the heterozygous parental lines.

Northern Analysis

Leaves from a random selection of 2 events per construct (pCGP2772, pCGP2765, pCGP3259, pCGP2785, pCGP3258, pCGP3261, pCGP960, pBIN35Smgfp4ER, pCGP3260) were analysed for the presence of transcripts of the introduced T1 or A8 colored protein genes. Total RNA was isolated from these events using a Plant RNAeasy kit (QIAGEN) following procedures recommended by the manufacturer.

RNA samples (5 μg) were electrophoresed through 2.2 M formaldehyde/1.2% w/v agarose gels using running buffer containing 40 mM morpholinopropanesulphonic acid (pH 7.0), 5 mM sodium acetate, 0.1 mM EDTA (pH 8.0). The RNA was transferred to Hybond-N filters (Amersham) as described by the manufacturer.

The RNA blot was initially probed with ³²P-labelled fragments of a BamHI/HindIII fragment isolated from pCGP2921 (T1) (FIG. 10) (10⁸ cpm/μg, 2×10⁶ cpm/mL). Prehybridization (1 hour at 42° C.) and hybridization (16 hours at 42° C.) of the membrane were carried out in 50% v/v formamide, 1 M NaCl, 1% w/v SDS, 10% w/v dextran sulphate. The filter was washed in 2 x SSC, 1% w/v SDS at 65° C. for between 1 to 2 hours and then 0.2×SSC, 1% w/v SDS at 65° C. for between 0.5 to 1 hour. The filter was exposed to Kodak XAR film with an intensifying screen at −70° C. for 22 hours.

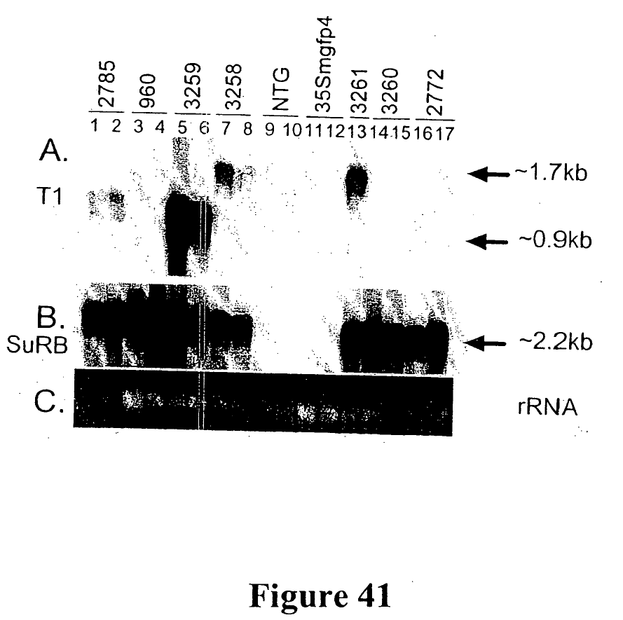

The T1 probe hybridized with transcripts of expected sizes (see Table 20) in RNA of transgenic plants that had been transformed with constructs carrying the T1 or A8 clones (lanes 1, 2, 5, 6, 7, 8, 13, 16 and 17) (eg. pCGP2772, pCGP2765, pCGP3259, pCGP2785, pCGP3258, pCGP3261) (FIG. 41A) (Table 20). Under the conditions used, no hybridizing transcript was detected by Northern analysis of total RNA isolated from non transgenic control plants (lanes 9 and 10) or transgenic plants transformed with non-TI carrying constructs (lanes 3, 4, 11, 12, 14 and 15) (e.g. pCGP960 (GUS), pBIN35Smgfp4, pCGP3260 (ER:mGFP4).

The ³²P-labelled T1 DNA probe was then stripped from the RNA blot by soaking the membrane in 0.1% SDS at 100° C. and incubating it in a 65° C. oven for 30 minutes with a final incubation step at room temperature for around 30 minutes.

The RNA blot was then probed with ³²P-labelled fragments of a ˜0.8 kb HindIII fragment from pCGP1651 (SuRB) (10⁸ cpm/μg, 2×10⁶ cpm/mL). Prehybridization and hybridization were carried out as described above. The plasmid pCGP1651 contains a 0.8 kb HindIII fragment from the SuRB coding region contained in the binary plasmid vector pWTT2132 (DNAP).

The SuRB probe hybridized with a 2.2 kb transcript in transgenic plants that had been transformed with the constructs carrying the CaMV 35S: SuRB transgene (FIG. 41B) (lanes 1 to 8, 13 to 17) (eg. pCGP2772, pCGP2765, pCGP3259, pCGP2785, pCGP3258, pCGP3261) (Table 20). Under the conditions used, no hybridizing transcript was detected by Northern analysis of total RNA isolated from non transgenic control plants (lanes 9 and 10) or transgenic plants transformed with non-SuRB constructs (lanes 11 and 12) (e.g. pBIN35Smgfp4ER).

Detection of Colored Proteins in Transgenic Arabidopsis

Polyclonal Rabbit Antibodies to T1 Protein

T1 protein was extracted from cultures of E. coli harbouring pCGP2921 (FIG. 10) as described previously in Example 6.

Polyclonal rabbit antibodies against the T1 protein were produced by Institute of Medical and Veterinary Sciences, Veterinary Services Division, 101 Blacks Rd. Gilles Plains, South Australia 5086, Australia. An amount of 300 μg of Ti protein (with Freunds complete adjuvent) was initially administered. Serial doses of 300 μg T1 protein (with Freunds incomplete adjuvent) were subsequently administered 22 days and 36 days after the initial dose. Antibodies collected in the first bleed (which was taken at 45 days after the initial dose) were used to probe Western blots in the first instance.

Protein Extraction from Plants

Leaf material (20-120 mg) was collected from Arabidopsis plants, snap frozen in liquid nitrogen and then ground to a fine powder using a mortar and pestle. An equal volume (w/v) of extraction buffer (100 mM Na₂PO₄pH 6.8, 150 mM NaCl, 10 nM EDTA, 10 mM DTT, 0.3% Tween 20, 0.05% Triton X) was then added to the fine powder and the mixture was further ground using the mortar and pestle. The resultant slurry was centrifiged at 10 000 rpm for 10 min and the supernatant was collected.

Western Blot Analysis of Proteins Extracted from Transgenic Arabidopsis

Aliquots (8 μL) of the protein extracts were mixed with 2 μL of 5×SDS loading buffer 10% v/v glycerol, 3% w/v SDS, 3% β-mercaptoethanol, 0.025% w/v bromophenol blue) electrophoresed through precast SDS PAGE gels (12% w/v resolving, 4% w/v stacking gel) Ready Gels, Biorad) at 100 V for 1 h 15 min in a Min-Protean System (Bio-Rad) using conditions as described previously in Example 6. The proteins were then transferred to Immun-Blot PVDF membrane (Bio-Rad) using a Mini Trans-Blot Electrophoretic Transfer Cell (Bio-Rad) in Towbin buffer (25 mM Tris, 20% methanol, 192 mM glycine) at 100 V for 1 h. PVDF membranes were incubated in blocking buffer (5% non-fat dry milk, 0.2% Tween-20, 75 mM NaPi pH 7.4, 68 mM NaCl) at room temperature for 1 h. Membranes were then further incubated with Rabbit anti-T1 antibody (diluted 1/200 in blocking buffer) for 2 h at room temperature then washed twice for 5 min in wash buffer (0.2% Tween, NaPi pH 7.4, 68 mM NaCl). The membranes were finally incubated with goat anti-rabbit-IgG-alkaline phosphatase congugate (Bio-Rad) (diluted 1/300 in blocking buffer) for 1 h at room temperature followed by 4 washes for 10 min each in wash buffer. Colorimetric detection was carried out with Western Blue Stabilized Substrate for Alkaline Phosphatase (Promega).

The polyclonal T1 antibody detected a protein band running at the same position as T1 protein extracted from E.coli cultures harbouring pCGP2921 in extracts from Arabidopsis/2772 event 1.2, Arabidopsis/3259 event 1.5. The same T1 protein band was not detected in extracts from the non-transgenic controls.

The protein content in a 2 μL sample of the protein extracts was estimated using a Bio-Rad Protein Assay as per the manufacturers instructions (Microassay Procedure). The absorbance of each extract at 595 nm was compared with BSA standard curves (0-10 μg/mL) to estimate protein concentrations.

Samples of protein extract and a dilution series of known amounts of purified His-tagged colored protein (T1) were electrophoresed through SDS PAGE gels as described previously. The proteins were transferred to PVDF membranes (as described above) and probed with rabbit anti-T1 antibodies. The amounts of T1 colored protein in the protein extracts was estimated by comparison with the purified His-tagged colored protein dilution series. This allowed an estimation of expression of colored protein in Arabidopsis leaf as a percentage of total soluble protein (Table 21).

### EXAMPLE 15

### Expression of Colored Proteins in Petunia

Transformation of Petunia

Petunia hybrida cultivar Mitchell produces white flowers. Mitchell was transformed with the binary constructs pCGP2772 (FIG. 24), pCGP2765 (FIG. 21), pCGP3259 FIG. 36) pCGP2785 (FIG. 33) and pCGP2926 (FIG. 44) via Agrobacterium-mediated transformation as described in Example 1.

Northern Analysis

Flowers from a random selection of events transformed with the T-DNAs of pCGP2772 and pCGP2765 were analysed for the presence of transcripts of the introduced T1 or A8 colored protein. Total RNA was isolated using a Plant RNAeasy kit (Qiagen) following procedures recommended by the manufacturer. Northern analysis was performed as described above for analysis of the Arabidopsis transgenic plants.

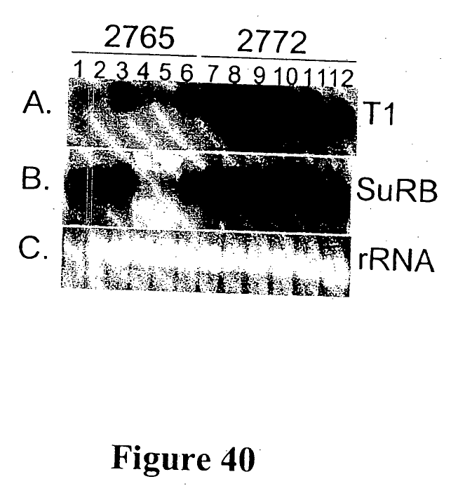

The T1 probe hybridized with transcripts of around 0.9 kb in petal RNA of transgenic Mitchell plants that had been transformed with constructs carrying the T1 or A8 clones (FIG. 40A) (pCGP2772 (lanes 7 to 12) and pCGP2765 (lanes 1 to 6), respectively). Under the conditions used no hybridising transcript was detected in RNA isolated from petals of a non transgenic control (data not shown).

The SuRB probe hybridized with a 2.2 kb transcript in transgenic plants that had been transformed with the constructs carrying the CaMV 35S: SuRB transgene (FIG. 40B).

Under the conditions used no hybridizing transcript was detected in RNA isolated from petals of a non transgenic control (data not shown).

Detection of Colored Proteins in transgenic P. hybrida

Western Blot Analysis of Proteins Extracted from Transgenic Petunia

Proteins were extracted from leaf and flower material (petal tube, petal limb, anthers, pistil, stigma and style) (100-300 mg) of transgenic and non-transgenic P. hybrida cv, Mitchell plants as described for Arabidopsis.

Western blot analysis of these protein extracts was performed as described for Arabidopsis.

The polyclonal T1 antibody detected a protein band running at the same position as T1 protein extracted from E. coli cultures harbouring pCGP2921 in extracts from Petunia accession 24534 (pCGP2765) and Petunia accession 24444 (pCGP2772). The same T1 protein band was not detected in the non-transgenic controls.

An estimation of expression of colored protein in Petunia leaf and petal as a percentage of total soluble protein was made as described above for Arabidopsis extracts (Table 22).

The T1 protein was produced in Arabidopsis leaf (Example 14) and Petunia leaf and flower tissue (Example 15). It is expected that an increase in protein accumulation will produce stronger colours in flower and leaf tissue. The first generation of transformed plants are selfed to give homozygous second generation transformants with higher T1 protein or other CFM accumulation and stronger colour.

Alternatively, different transgenic events are crossed to produce second generation transformants with higher T1 protein or other CFM accumulation and stronger colour. Methods envisaged to increase total T1 protein or other CFM accumulating in transformed plants include targeting T1 or other CFM to the chloroplast using a chloroplast transit peptide such as that from the small subunit of ribulose-bisphosphate from tobacco (see Example 11 or Table 17). These chloroplast transit peptides will facilitate the movement and accumulation of CFMs into chioroplasts which are abundant in leaves and chromoplasts which are abundant in flowers petals. Another method envisaged to produce higher levels of CFMs in plant tissues is the use of chloroplast/plastid transformation techniques which have been used in the past to generate plants expressing recombinant proteins at levels of up to 46 % of total soluble protein (De Cosa et al., Nat. Biotechnol. 19, 71-74, 2001; Daniell et al., Trends in Plant Sci. 7:84-91, 2002, see Example 11, Table 18). It is also envisaged that the co-expression of a suitable chaperonin in conjunction with one or more CFMs allows the efficient folding and packaging of CFMs into stable structures which are accumulated in higher amounts than would normally be expected. It is also envisaged that producing a fusion of CFM with ubiquitin in plants will increase levels of accumulated CFMs in transgenic plants as has been demonstrated in yeast (Baker, Curr. Opinions in Biotech, 7:541-546, 1996 and references within). It is also envisaged that targeting T1 or other CFM to the endoplasmic reticulum (see Example 11) will increase the levels of accumulated recombinant protein in plant tissues (Haseloff et al., 1997, supra).

Detection of Correctly Folded CFMs in Plant Extracts.

CFMs that are folded correctly in heterologous systems (such as when expressed in flowers or other plant tissues) are expected to retain characteristic absorbance and corresponding colour (see Example 13). The level of CFM production or accumulation may initially be too low for significant color change in plant tissue. A method for detecting low levels of correctly folded CFMs in plant extracts is described for leaf material from Petunia transformed with pCGP2772 and pCGP2765, however, this method can be used with other plant tissues such as but not limited to Petunia or rose or gerbera.

Total soluble proteins were extracted from transgenic leaves of Mitchell/pCGP2772 and Mitchell/pCGP2765) (see Example 15). These samples were frozen in liquid nitrogen and ground using a mortar and pestle. An equal volume (wlv) of extraction buffer (100 mM NaPO4 pH 6.8, 150 mM NaCl, 10 mM EDTA, 10 mM DTT, 0.3% Tween 20, 0.05% Triton X) was added to the sample and further ground. The resultant slurry was centrifuged at 10000 rpm for 10 min and the supernatant collected.

The extracts were used undiluted or diluted 1:2 in water and their absorbance characteristics determined between 400 nm and 700 nm using a Varian Cary 50 Bio UV-Visible Spectrophotometer. The absorbance spectra were compared to those of extracts of non-transgenic control tissue and non-transgenic control tissue spiked with either T1 or T3 His-tagged purified protein (see Example 8). Detectable color was observed through the detection of peaks at approximately 580-590 mn in the extracts from transgenic plant tissue that were not evident in non-transgenic control tissue.

Methods envisaged to increase protein levels are as described above or by Bailey-Serres and Gallie (American Society of Plant Physiologists, Look beyond transcription, UCLA, USA, 1998) or by modification of mRNA sequence to optimize 5′ and 3′ untranslated sequences thereby improving message stability and/or translation efficiency, optimisation of codon usage in the introduced gene to more closely match that found in highly expressed genes (that is genes which give rise to high levels or encoded protein synthesis) in particular those of target crops, augmentation of protein stability via the attachment for example of stabilising sequences such as ubiquitin, changes to specific N-terminal amino acid residues to promote altered aggregation of monomeric forms of the protein, more effective targeting of the synthesized polypeptide to intracellular organelles or compartments, duplication and there for amplification of introduced genes leading to increased levels of protein biosynthesis for example using Gene Amplification Technology' (Boisjuk et al., Nature Biotechnology 18:1303-1306, 2000).

### EXAMPLE 16

### Expression of Colored Proteins in other Plants

The horticultural industry relies on the production of novel traits such as new colors, fragrances, productivity and disease resistance introduction of colored protein sequences (via genetic engineering) into commercially important plant lines such as, for example, but not limited to roses, carnations and gerberas provides a means to produce novel colors in flowers or plants that lack such colors.

Introduction of colored protein genes into roses is achieved using methods such as those described, for example, in International Patent Application Number PCT/US91/04412, or by Robinson and Firoozabady (Scientia Horticulturae, 55:83-99, 1993), Rout et al. (Scientia Horticulturae, 81:201-238, 1999) or Marchant et al. (Molecular Breeding 4:187-194, 1998) or by any other method well known in the art.

Introduction of colored protein genes into carnations is achieved using methods such as those described, for example, in International Patent Application Number PCT/US92/02612 or by Lu et al. (Bio/Technology 9:864-868, 1991), Robinson and Firoozabady (1993, spra) or by any other method known in the art.

Introduction of colored protein genes into carnations is achieved using methods such as those described, for example, by Robinson and Firoozabady (1993, supra).

The cotton industry relies on the production of dyed cotton, using dyes that can have concomitant detrimental effects on the environment. Introduction of colored protein sequences (via genetic engineering) into commercially important cotton lines, or other plant lines that allow for production of fabrics (such as, but not limited to, hemp), and also relies on use of colored dyes to dye said fabrics, is achieved using methods such as those described, for example, in an International Patent Application having Publication Number WO 00/77230.

### EXAMPLE 17

### Generation of Transformed Animals

The use of the CFMs of the present invention are employed to produce transgenic animals which exhibit novel color, for example, sheep with blue or red colored fleece, cows with red colored hide inter alia. The transgenic animals of the present invention can be produced by any number of method know in the art. Such as, but not limited to transgenic animals are produced by any number of methods, for example, microinjection of constructs comprising a CFM nucleotide sequence into the pronucleus of a fertilized ovum, or injection of embryonic stem (ES) cells into embryos.

Microinjection

Following fertilization a single celled embryo is removed from the animal (e.g. sheep, cow, pig, goat). Micromanipulators on a specially equipped microscope are used to grasp each i embryo. A glass pipette drawn to a fine point immobilizes the embryo on one side. On the opposite side, a construct containing a CFM nucleotide sequence is injected into the embryo's pronucleus with a second finely drawn injection needle. Following the injection, the embryos are transferred back into the hormonally prepared or pseudopregnant recipient females or foster mothers. The recipients follow normal pregnancy and deliver full-term young.

Injection of Embryonic Stem Cells

ES cells are isolated from the inner cell mass of blastocyst-stage embryos (about 7 days postfertilization), ES cells are grown in the lab for many generations to produce an unlimited number of identical cells capable of developing into fully formed adults. These ES cells are altered genetically by injection of a construct containing a CFM nucleotide sequence.

Transgenic individuals are produced by microinjection of embryonic stem (ES) cells containing the CFM construct into embryos to produce “hybrid” embryos of two or more distinct cell types. Following the injection, the embryos are transferred back into the hormonally prepared or pseudopregnant recipient females or foster mothers. The recipients follow normal pregnancy and deliver full-term young.

### EXAMPLE 18

### Generation of a Far Red Fluorescent Monomeric Protein

Cloning and Expression

cDNA encoding the colored protein Rtms-5 (SEQ ID NO:166) was isolated from Montipora efflorescens (Scleractina Acropodiae). Under daylight. illumination, Montipora efflorescens was a purply-red colour, but fluoresced yellow under blue illumination and red under green illumination.

To further characterise the protein, the cDNA was tagged with hexahistidine at its C-terminus and expressed at high levels in Escherichia coli. For expression in bacteria, the nucleotide sequence encoding Rtms-5.pep (SEQ ID NO:166) was retrieved from pGEM-T cloning vector (Promega) using forward oligonucleotide primers consisting of the NotI restriction binding site, a ribosomal binding site, a spacer and 15 bases encoding the N-terminus of the protein (MSV-RBS, SEQ ID NO:213; SVIAK-RBS, SEQ ID NO:214) and a reverse oligonucleotide primer encoding H6-tag (POC220-H6, SEQ ID NO:215).

| | |
|---|---|
| MSV-RBS | |
| GGC TCT AGA AAG GAG ATA TAC AAG TGT GAT CGC TAC ACA AAT GA | SEQ ID NO:213 |
| SVIAK-RBS | |
| GGC TCT AGA AAG GAG ATA TAC AAT GTC CGT TAT CGC TAA ACA GAT | SEQ ID NO:214 |
| POC220-H6 | |
| GGC AAG CTT TCA GTG GTG GTG GTG GTG GTG GGC GAC CAC AGG TTT GCG TG | SEQ ID NO:215 |

PCR product was gel purified and diluted (×10) prior to cloning into pCRII-TOPO (Invitrogen) and transforming into Top 10 cells (Invitogen). Cells were induced with 0.5 mM IPTG, and protein was purified on Ni-columns Pro-Bond, Invitrogen) eluting with 50 mM, 200 mM, 350 mM and 500 mM Imidazole in PBS pH 6.0, prior to overnight dialysis against 50 mM Potassium phosphate pH 6.65.

Fluorescence Characteristics of Rtms-5

E. coli colonies were blue in colour in daylight, and weakly red fluorescent when excited with light of wavelength 595 nm.

An alignment of the amino acid sequence of Rtms-5 (SEQ ID NO:166) with other fluorescent proteins was constructed (Table 19). Rtms-5 (SEQ ID NO:166) contains the key amino acids (Tyr-66 and Gly-67) that correspond to those that form the fluorophore in other well-characterised proteins, dsRed583 (also known herein as drFP583, SEQ ID NO:221) and GFP (SEQ ID NO:222). Overall, 67% and 20% of the Rtms-5 (SEQ ID NO:166) sequence is identical to dsRed583 (SEQ ID NO:221) and GFP (SEQ ID NO:222), respectively. The protein shares a high degree of identity with a number of chromoproteins recently isolated from the Anthozoa species (Gurskaya et al., FEDS Lett. 507: 16-20, 2001).

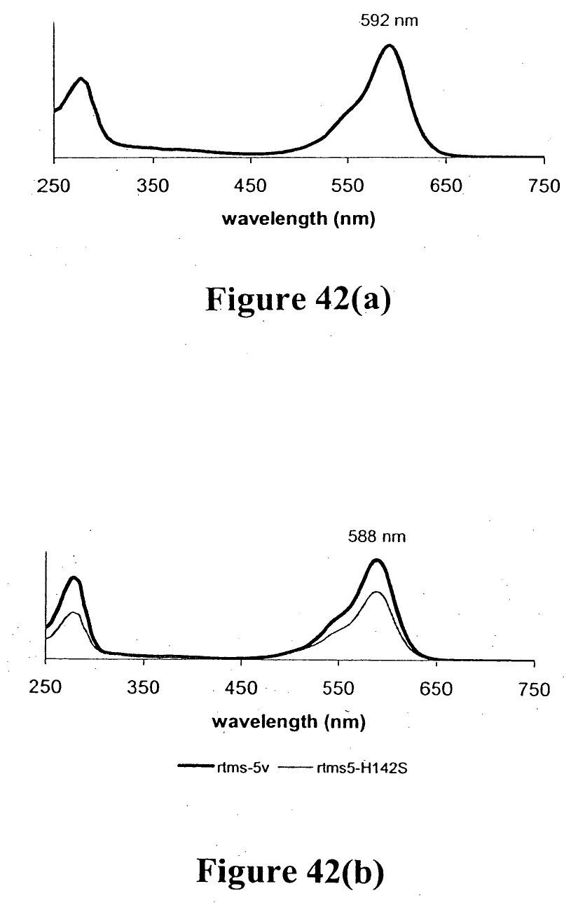

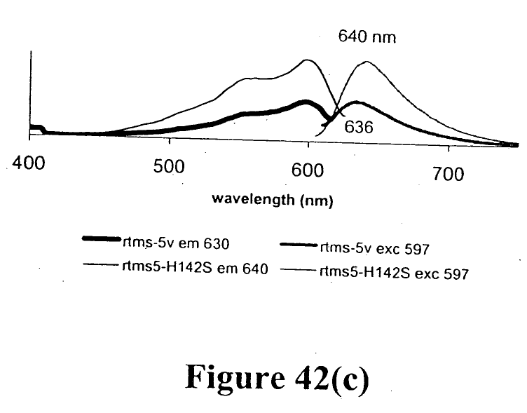

The absorption and excitation emission spectra were measured for the purified “wild-type” Rtms-5 (SEQ ID NO: 166). The protein displays a major absorption peak at 592 nm, with an extinction that is highly variable (ε₅₉₂=53,000 M⁻¹ cm⁻¹-111,000 M⁻¹ cm⁻¹) and a shoulder peak at 454 nm (FIG. 42. The variability in the extinction coefficient is similar to that observed for drFP583 (SEQ ID NO:221) and, similarly, it is dependent on the state of maturity, as well as the conditions under which the protein is expressed (Baird et al., 2000, supra).

Site Directed Mutagenesis

Rtms-5 (SEQ ID NO:166) was only weakly fluorescent. To enhance this, site-directed mutagenesis was carried out, The alignment of the Rtms-5 sequence (SEQ ID NO:166) with other sequences (Table 19) indicated that position 142 was occupied by the residue histine. A variant Rtms-5-H142S, containing the substitution H142S, was engineered by mutagenesis of pCRII-TOPO::RTms5 to produce pCRII-TOPO::RTms5-H142S. This single substitution increased the quantum yield of far-red fluorescence by 170-fold to a quantum yield of less than 0.02. Minor effects on the excitation and emission spectra and the absorption spectra were observed (4 nm shift towards the blue end of the spectrum, refer to FIG. 42A,B,C).

Analysis of Oligomeric Structure

dsRed583 (SEQ ID NO:221) is known to be an obligate tetramer. The formation of oligomers by fluorescent proteins can present a serious problem when expressed fused to other proteins of interest. Consequently, it was important to establish the degree of oligomerisation of Rtms-5 (SEQ ID NO:166). The protein has a predicted size of 25,820 Da (with H6). When subjected to SDS-PAGE under reducing conditions, purified Rtms-5 (SEQ ID NO:166) migrated with an Mᵣ of 26,900. However, under non-reducing conditions the majority of the protein migrated with an Mᵣ of 114,000. These results indicated that native Rtms-5 (SEQ ED NO: 166) was predominantly a tetramer.

Further Site Directed Mutagenesis and Analysis of Structure

A second round of site-directed mutagenesis was carried out, to mutagenise CRII-TOPO::RTms5-H142S to produce the variant pCRII-TOP-RTms5-H142S-F158H (pCRII::Rtms-5v). This colored peptide contained the additional substitutions F158H and R145H, and is designated Rtms-5v (SEQ ID NO:216).

Rtms-5v (SEQ ID NO:216) was expressed in E. coli and the purified six His-tagged protein was subjected to analytical ultracentrifugation. The results indicated that the mutagenised variant sedimented predominantly as a monomer (82%, 30,700 Da) with the remaining proportion sedimenting as a dimer (18%, 50,800 Da). This colored protein fluoresced in the far-red range (see FIG. 42C), and can be used effectively in yeast cells and mammalian cells.

Effect of Site Directed Mutagenesis of Other Colored Proteins

Site directed mutagenesis of residue H or N 142 to S, in other colored protein sequences, also leads to the generation of far-red fluorescence. Examples of the excitation and emission spectra for two other colored proteins, Aams-4 (SEQ ID NO:90)-H142S, and Rtms-1 (SEQ ID NO:162)-N142S are shown in FIG. 43.

### EXAMPLE 19

### Expression in Yeast, Mammals and as a Fusion Protein

The subject inventors sought to demonstrate that the instant CFMs can be expressed in yeast and mammalian cells and can be used as fusion proteins for genetic marking of cells.

(a) Expression in Yeast

For expression in yeast cells a BamHI(Not1 DNA cassette encoding dsRed or YGFP3 (an enhanced variant for expression in yeast) or a BglII/Not1 cassette encoding the novel fluorescent protein, Rtms-5v (SEQ ID NO:216), were retrieved using the pair of oligonucleotide primers RFPUP1 (SEQ ID NO:234), /RFPDO1 (SEQ ID NO:235), YGFP3UP (SEQ ID NO:232), /YGFP3DO (SEQ ID NO:233), or MSVIATUP (SEQ ID NO:236)/COFPDO (SEQ ID NO:237), respectively, using as templates the vectors pYGFP3 (Cormack et al.,. Microbiology 143:303-11, 1977), pDsRed-1 [Clontech Industries] or cDNA for pCRII-TOPO::RTms-5v. In the case of YGFP3UP, the Not1 site was retrieved after digesting the PCR product from pGEM-T (Promega). The PCR product was cloned into the BamHI(NotI site of the multi-copy yeast expression vector pAS1NB to produce pAS1NB::dsRedL, pAS1NB::YEGFP3L or pAS1NB::Rtms-5v from which the DNA cassette encoding wild-type GFP had been removed but retaining the multiple cloning sites of that vector and linker sequence of that vector [Prescott et al., FEBS Letts. 411:97-101, 1997]. pASN1B is a derivative of pAS1N (Prescott et al., 1997, supra) in which a BamHI restriction site has been removed from the PGK promoter region. This series of vectors allows the expression of fluorescent proteins not fused to a partner protein and provides.

| | |
|---|---|
| YGFP3UP | |
| 5′-GGATCCATCGCCACCATGTCTAAAGGTGAAGAATTATTCACTGG | SEQ ID NO:232 |
| YGFP3DO | |
| 5′-CAGCTGTTATTTGTACAATTCATCCATACCATGG | SEQ ID NO:233 |
| RFPUP1 | |
| 5′-CGGGATCCATCGCCACCATGAGGTCTTCCAAGAATGTTATC | SEQ ID NO:234 |
| RFPDO1 | |
| 5′-GAGGATCCGCGGCCGCTAAAGGAACAGATGG | SEQ ID NO:235 |
| MSVIATUP | |
| 5′-GAAGATCTAAAACAATGAGTGTGATCGCTACACAAATG | SEQ ID NO:236 |
| COFPDO | |
| 5′-TATCAAATCGCCGGCGTCAGGCGACCACAGGTTTG | SEQ ID NO:237 |

(b) Expression as a Fusion Protein

Two DNA cassettes encompassing segments of the yeast genes ATP4 and ATP7 for subunit b and d of ATP synthase, respectively, were recovered by PCR from YRD15 genomic DNA using the oligonucleotide primer pairs ATP4PROMUP2 (SEQ ID NO:238)/ATP4DO2 (SEQ ID NO:239), or ATP7TUP (SEQ ID NO:240)/ATP3TDO (SEQ ID NO:241), respectively. The first, ATP4PO, encompasses the open reading frame for ATP4 and 500 bp of sequence upstream of the initiation codon flanked by BglII and BamHI restriction sites at the 5′ and 3′, respectively. The BamHI restriction site allows for an in frame-fusion between the C-temunus of subunit b and each of the three fluorescent protein cassettes. The second, ATP7T, encompasses the. transcription terminator cassette representing the terminator region of the ATP7 gene flanked at the 5′ and 3′ ends by restriction sites for NotI and SacII, respectively. These restriction sites were obtained on cloning the PCR product into GEM-T. The ATP4PO & ATP7T DNA cassettes were cloned sequentially into the BamHI and NotI/SacII sites, respectively of the yeast expression vector pRS413 to produce the expression vector construction denoted pRS413::ATP4PO:ATP7T. A BglIIHI/NotI DNA fragment encoding YGFP3L was excised from pAS1NB::YEGFP3L and then cloned into the BglII/NotI site of pRS413::ATP4PO:ATP7T to produce a vector (pRS306::ATP4PO:YEGFP3L:ATP7T) encoding subunit b fuised to YEGFP3 with a polypeptide linker of 25 amino acids. A vector (pRS413::ATP4PO:RTms-5:ATP7T or pRS413::ATP4PO:dsRed:ATP7T) encoding subunit b fused to RTms-5B or dsRed with a polypeptide linker of 27 amino acids was derived from pRS306::ATP4PO:YGFP3L:ATP7T by replacing the BamHI/NotI fragment encoding YEGFP3 with an equivalent fragment encoding Rtms-5v or dsRed.

| | |
|---|---|
| ATP4PROMUP2 | |
| 5′-AGATCTGTGTTGTGACGCAACTGCAACTCC | SEQ ID NO:238 |
| ATP4DO2 | |
| 5′-GTGATCAGCGGATCCCTTCAATTTAGAAAGCAATTGTTC | SEQ ID NO:239 |
| ATP7TUP | |
| 5′-CCTCTATATATTACGCACCATATTC | SEQ ID NO:240 |
| ATP7TDO | |
| 5′-ATACGTGACGACATTGGTAGTC | SEQ ID NO:241 |

(c) Results were Visualised using Clear Native Gels.

These were run essentially as described hereinafter. Briefly, 200 μg of mitochondrial protein was pelleted for 5 min at 100,000 g. Yeast mitochondria were isolated from spheropblasts (Law et al., Methods in Enzymol. 260:122-163, 1995). The pellet was solubilized in buffer (40 μl) containing in dodceyl β-maltoside to isolate the monomer form or digitonin (20 g/g protein) to isolate the dimer form and incubated on ice for 20 min and centrifuged 100,000 g for 30 min. Supernatants (30 μl) were loaded into wells of 4-16% gradient gels (13 cm×10 cm×0.075 cm). After running and while still between the glass plates, gels were imaged for fluorescence using a Perkin-Elmer multi-wavelength imager in ‘edge-illumination mode’ using appropriate filters for excitation (GFP, 480±20 nm; dsRed and Rtms-5v, 540±25 nm) and emission (GFP, 535±20 nm; dsRed, 590±35 nm; Rtms-5v, 620±30 nm).

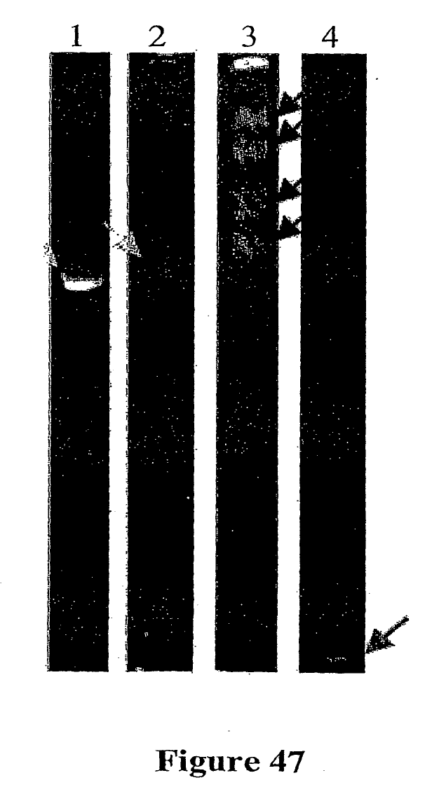

DNA cassettes encoding subunit b fused to the N-terminus of each of the three fluorescent proteins were expressed in a yeast strain lacking expression of endogenous subunit b. The ATP synthase in each of these strains was established to be assembled and functional as cells of each strain were able to grow using the non-fermentable substrate ethanol as carbon source. Yeast cells lacking endogenous subunit b do not assemble finctional mtATPase and cannot grow using ethanol as the sole carbon source. Yeast cells of each strain expressing the individual fuision proteins were visualized using fluorescence microscopy. For cells of each strain the distribution of fluorescence in the cell was similar and consistent with localisation to the mitochondrion. Mitochondria were isolated from cells of each of the strains and, after extraction, AT? synthase complexes were subjected to analysis by clear native gel electrophoresis (CNGE). ATP synthase isolated from yeast is a large membrane bound complex (˜800 kDa for the monomeric form) made up of 20 different types of subunits some of which are present in the complex as more than one copy. The complex can be isolated as a monomer or a dimner depending on the detergent, dodceyl β-maltoside or digitonin, respectively, used to extract the complex from mitochondrial membranes. Subunit b is present in a single copy in the monomer. ATP synthase in this experiment was extracted from each preparation of mitochondria under conditions that favour the isolation of the monomer. Subunit b is present in a single copy in the monomer. Samples were subjected to analysis by CNGE and the gel imaged for fluorescence using conditions of illumination and light detection specific for each fluorescent protein (FIG. 47). A single fluorescent band corresponding to the position of assembled monomeric ATP synthase was observed for complexes containing the b-GFP fusion protein (FIG. 47, lane 1). The position of GFP not fused to another protein is shown (FIG. 47, lane 4). A single fluorescent band was seen for complexes containing the fusion protein b-Rtms-5v (FIG. 47, lane 2). However, multiple bands were observed for samples containing b-dsRed FIG. 47, lane 3). It is possible that, in order of decreasing mobility, each fluorescent band corresponds to a monomer, dimer, trimer and tetramer.

(d) For Expression in Mammals

For expression in mammalian cells, a SmaI/NotI fragment encoding Rtms-5v (SEQ ID NO:216) was excised from pAS1NB::RTms-5v and cloned into the expression site of the mammalian expression vector pCI-Neo (Promega Corporation, Madison USA). This vector allows the expression of Rtms-5v not fuised to a partner protein.

A major benefit of fluorescent protein technology is the ability to simultaneously monitor using spectrally distinct variants more than one event in the living cell. The spectral properties of Rtms-5v suggest that should be feasible to image both dsRed and Rtms-5v expressed in the same cell. This would allow Rtms-5 to be used in combination with dsRed rather than substitute for dsRed. The emission maxima for dsRed and Rtms-5v are separated by 50 nm. We tested the possibility of imaging dsRed, RTms-5v and EGFP expressed in the same cell. Three individual DNA cassettes were constructed encoding dsRed fused at its N-terminus to the 16 amino acid mitochondrial targeting sequence of human 3-oxoacyl-CoA thiolase, EGFP fused to the C-terminus of Rab6 and Rtms-5v not fused to any other protein. Cells were imaged using a Zeiss 510 Meta confocal laser scanning microscopy (Zeiss). The distribution of fluorescence arising from each of the Rtms-5v, dsRed and EGFP fusions was consistent with the locations expected (cytosol/nucleus, mitochondrion and golgi, respectively). These results show that Rtms-5v is able to fluorescently label other compartments of the cell such as the mitochondrion in addition to the cytoplasm. The position of a non-transfected and, therefore, non-fluorescent cell is shown in the transmitted light image by the white arrow Rtms-5v showed no evidence of aggregation. Similar results were observed for the expression of Rtms-5v not targeted in yeast cells. Multiple fluorescent proteins are commonly (eg. GFP, dsRed, CFP) imaged in the same cell.

### EXAMPLE 20

### Additional Color Proteins from Coral

The inventors sought additional color proteins from two corals, Montipora efforescens and Pavona decussaca.

(a) Montipora Efforescens

Standard purification techniques (Dove et al., 2001, supra) were adopted for the purification of a red fluorescent protein from phosphate buffer extract of M. efforescens. A protein was purified using gel filtration and subject to N-terminal amino acid sequencing. A polymorphism was identified, comprising F and R residues. The N-terminal amino acid sequences are represented as follows:

| | |
|---|---|
| SPPDY TLE [symbol] P KKXVA | SEQ ID NO:242 |
| SPPDY TLE [symbol] P KKGVA | SEQ ID NO:243 |

The polymorphism is indicated in bold larger type.

(b) Pavona decussaca

Similar techniques as those described in (a), above, were used to identify and purify a green fluorescent protein from P. decussaca. Gel electrophoresis showed that the proteins ran as two bands and N-terminal amino acid sequencing identified polymorphic variants, shown in bold larger type, below:

| | |
|---|---|
| Top band: | |
| (D)SS(P)E SYL [symbol] N GIAEE MKTDV MEGI | SEQ ID NO:244 |
| Lower band: | |
| S [symbol] N GIAEE MKTDL MEGIV NG | SEQ ID NO:245 |
| S [symbol] N GIAEE MKTDL MEGIV NG | SEQ ID NO:246 |

The protein fraction was generating these N-terminal sequences had absorbed maximally at 440 nm with maximal excitation at 440 nm and emission at 488 nm.

Oligonucleotide probes were designed in both forward and reverse directions for PCR amplification from a ZAP express cDNA library of Acropora millepora (Scleractinian coral). The oligonucleotide primers used were as follows:

Forward

| | | |
|---|---|---|
| MEGIVNG-A | ATG GAA GGG ATA GTC GAT GG | SEQ ID NO:247 |
| MEGIVNG-T | ATG GAA GGG ATT GTC GAT GG | SEQ ID NO:248 |
| MEGIVNG-C | ATG GAA GGG ATC GTC GAT GG | SEQ ID NO:249 |
| Reverse | | |
| REV-MEG-T | CCT CCA CAA TCC CTT CCA T | SEQ ID NO:250 |
| REV-MEG-C | CCT CGA CGA TCC CTT CCA T | SEQ ID NO:251 |

DNA was amplified and separated using gel electrophoresis. Bands were purified and cloned into pCRII-TOPO and transfected into TOP 10 cells (Invitrogen). Plasmids were then purified and subjected to nucleotide sequencing. The complete sequence is shown in Table 23.

In this experiment, therefore, a protein identified from P. decussaca was used to identify a clone from Acropora millepora.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 2

| Class | SubClass | Order | GFP | Initial studies | Sequence information | % identity^{(length)} * |
|---|---|---|---|---|---|---|
| Hydrozoa | | Hydroida | yes | Shimomura et al., J. Cell. | Prasher et al. (1992) Gene 111: | 23⁽²³⁸⁾ |
| | | | | Comp. Physiol. 59: 223-239, | 229-33; Chalfie et al. (1994) Science | |
| | | | | 1962; Morise et al., | 263: 802-5; Inouye & Tsuji (1994) | |
| | | | | Biochemistry 13: 2656-2662, | FEBS Lett. 341: 277-80. | |
| | | | | 1974; Morin & Hastings, J. | | |
| | | | | Cell Physiol. 77: 313-318, 1971 | | |
| | | Milliporina | yes | | Invention | 95.0⁽²²⁰⁾-97.3⁽²²¹⁾ |
| | | Stylasterine | | | | |
| | | Trachylina | | | | |
| | | Siphonophora | | | | |
| | | Chondrophora | | | | |
| | | Actinulida | | | | |
| Anthozoa | Octocorallia | Gorgonacea | | | | |
| | (=Alcyonaria) | | | | | |
| | | Telestacea | | | | |
| | | Pennatulacea | yes | Morin & Hastings, J. Cell | WO 99/49019 | 43.5⁽²²⁵⁾ & 43.5⁽²²⁵⁾ |
| | | (see pens | | Physiol. 77: 313-318, 1977 | | |
| | | & sea pansies) | | | | |
| | | Alcyonacea | | | | |
| | | Helioporacea | | | | |
| | | Stolonifera | yes | | Matz et al., Nature Biotechnology | 52.7⁽²²⁶⁾ |
| | | | | | 17: 969-973, 1999 | |
| | Hexacorallia | Actiniaria (sea | yes | | Matz et al., Nature Biotechnology | 46.2⁽²²⁷⁾ & 47.2⁽²²³⁾ |
| | (=Zoantharia) | anemones) | | | 17: 969-973; 1999; Lukyanov et | |
| | | | | | al., JBC 275: 25879-25882, 2000 | |
| | | Scleractinia | yes | Kawaguti, Paloa Trop. Boil. | WO 00/46233; Dove et al., Coral | 93.2⁽²²⁰⁾-100⁽²⁵⁵⁾ |
| | | (true or | | Stn. Stud. 2: 617-673, 1994; | Reefs 19: 197-204, 2001; Invention. | |
| | | stony corals) | | Dove et al. Biol. Bull. 189: | | |
| | | | | 288-297, 1995 | | |
| | | Zoanthidea | yes | | Matz et al., Nature Biotechnology | 44.1⁽²³¹⁾ & 45.5⁽²³⁶⁾ |
| | | | | | 17: 969-973, 1999 | |
| | | Corallimorpharia | yes | | Matz et al., Nature Biotechnology | 57.8⁽²²⁵⁾ & 62.5⁽²²⁴⁾ |
| | | (coral-like | | | 17: 969-973, 1999 | |
| | | anemones) | | | | |
| | Ceriantipatharia | Antipatharia | | | | |
| | | Ceriantharia | | | | |
| Cubozoa | | | | | | |
| Scyphozoa | | Stauromedusae | no | | | |
| (jellyfish) | | | | | | |
| | | Coronatae | | | | |
| | | Semaeostomae | | | | |
| | | Rhizostaomae | | | | |

* Best fit in relation to Aams2-pep (SEQ ID NO: 88) over 220-238 amino acids as indicated in length

TABLE 3
Fluorescent properties

| | | | Additional barrier |
|---|---|---|---|
| Excitation region | Exciter filter | Dichroic mirror | filter |
| Ultra-violet | UG-1 | DM400 + L420 | L435 |
| Violet | BP 405 | DM455 + Y455 | Y475 |
| Blue | BP 490 | DM500 + O515 | O515 |
| Green | BP 545 | DM580 + O590 | R610 |

TABLE 4
Class: Anthozoa; Order: Scleractinia

| | | Color | |
|---|---|---|---|
| Family | Genus, Species | morph | Fluorescent properties |
| Pocilloporidae | Pocillopora damicormis | Pink | Faintgreen fluorescence under blue light |
| | Pocillopora damicormis | Green | Fluoresce blue-green, green, green and red |
| | | | under UV, violet, blue and green light, respectively |
| Acroporidae | Acropora aspera | Blue tipped | Tentacles and calices fluoresce violet, blue, green, and |
| | | | faint red under UV, violet, blue and green light, respectively |
| | Acropora aspera | Blue light | Fluoresces green under UV and violet light |
| | | fluorescent | |
| | Acropora nobilis | Green | Calices and tentacles fluorescent violet, blue, green and |
| | | | red under UV, violet, blue and green light, respectively |
| | Montipora sp. (plating) | Red/red | Yellow fluorescence under blue light, red |
| | | fluorescent | fluorescence under green light |
| Poritidae | Porites murrayensis | Purple | Calices fluoresce faint green under violet and blue light |
| Agariciidae | Pavona decussaca | Green | Blue under UV light; green under violet light; |
| | | | and blue and moderate red under green light |
| Mussidae | Acanthasthastrea | Green | Calices and polyps fluorescent violet, blue, green and |
| | | | faint under UV, violet, blue and green light, respectively |
| Faviidae | Platygyra sp. | Green | Blue under UV and violet light; green under blue light |
| | Caulastrea sp. | Green | Blue under UV light; green under violet and blue light |

TABLE 5
Class: Hydrozoa; Order: Milleporina

| Genus | Color | Fluorescent properties |
|---|---|---|
| Millepora | Green | Blue under UV and |
| | | violet light; green under blue light |

TABLE 6

| | | | | |
|---|---|---|---|---|
| N-terminus | Genus species | Name | Type | Amino acids within 5 Å of fluorophore |

| | | | | |
|---|---|---|---|---|
| sgiat | Acropora aspera | Aams-5.pep | 1 | QVLSPQCQYGSIFWRNSYEHENMERLQCE |
| sviat | Acropora aspera | Aams-2.pep | 1 | QVLSPQCQYGSIFWRNSYEHENMERLQCE |
| sviat | Acropora aspera | Aams-4.pep | 1 | QVLSPQCQYGSIFWRNSYEHENMERLQCE |
| sviat | Acropora aspera | Aams-6.pep | 1 | QVLSPQCQYGSIFWRNSYEHENMERLQCE |
| sviat | Acanthastrea sp. | Acams-2.pep | 6* | QVLSPQYQYGSIFWRNSYENENMERLQCE |
| sviat | Acanthastrea sp. | Acams-3.pep | 6 | QVLSPQYQYGSIFWRNSYENENMERLQCE |
| sviat | Acanthastrea sp. | Acams-4.pep | 3 | QVLSPQYQYGSIFWRNSHENENMERLQCE |
| sviat | Acanthastrea sp. | Acams-5.pep | * | |
| sviat | Caulastrea sp. | Cems-F.pep | 5 | QVLSPQCQYGNIFWRNSYEHENMGRLQCE |
| sviat | Caulastrea sp. | Cems-G.pep | 5 | QVLSPQCQYGNIFWRNSYEHENMGRLQCE |
| sviat | Caulastrea sp. | Cems-H.pep | 5 | QVLSPQCQYGNIFWRNSYEHENMGRLQCE |
| sviat | Caulastrea sp. | Cems-I.pep | 16* | QVLSPQCQYGNIFWRNSYEHENMERLQCE |
| sviat | Acropora nobilis | LGAms-5.pep | 6 | QVLSPQCYYGSIYWRNSYENENMERLQCE |
| sviat | Acropora nobilis | LGAms-6.pep | 18* | QVLSPQYQYGSIFWRNSYENENMERLQCE |
| sviat | Millepora sp. | Mi68Dms.pep | 1 | QVLSPQCQYGSIFWRNSYEHENMERLQCE |
| sviat | Millepora sp. | Mims-A.pep | 1 | QVLSPQCQYGSIFWRNSYEHENMERLQCE |
| sviat | Millepora sp. | Mims-B.pep | 1 | QVLSPQCQYGSIFWRNSYEHENMERLQCE |
| sviat | Millepora sp. | Mims-C.pep | 1 | QVLSPQCQYGSIFWRNSYEHENMERLQCE |
| sviat | Pavona decussata | Pav5ms.pep | 6 | QVLSPQYQYGSIYWRNSYENENMERLQCE |
| sviat | Pavona decussata | Pavms-2.pep | 6* | QVLSPQYQYGSIYWRNSYENENMERLQCE |
| sviat | Pavona decussata | Pavms-3.pep | 6 | QVLSPQYQYGSIYWRNSYENENMERLQCE |
| sviat | Pavona decussata | Pavms-4.pep | 11 | QVLSPQYQYGSIYWGNSYENENMERLQCE |
| sviat | Porites murrayensis | PMms-A.pep | 2 | QVLSPQSQYGSIYWRNSYENENMERLQCE |
| sviat | Porites murrayensis | PMms-B.pep | 2 | QVLSPQSQYGSIYWRNSYENENMERLQCE |
| sviat | Porites murrayensis | PMms-C.pep | 9 | QVLSPQTQYGSIYWRNSYENGNMERLQCE |
| sviat | Porites murrayensis | PMms-D.pep | 2 | QVLSPQSQYGSIYWRNSYENENMERLQCE |
| sviat | Porites murrayensis | PMms-E.pep | 2 | QVLSPQSQYGSIYWRNSYENENMERLQCE |
| sviat | Pink Pocillopora | PPd57-1ms.pep | 12 | QVLSPQTQYGSIYWRNSYENENMERLQCE |
| sviat | Pink Pocillopora | PPd57-2ms.pep | 1 | QVLSPQCQYGSIFWRNSYEHENMERLQCE |
| sviat | Pink Pocillopora | PPd57-3.pep | 1 | QVLSPQCQYGSIFWRNSYEHENMERLQCE |
| sviat | Pink Pocillopora | PPd57-4ms.pep | 2 | QVLSPQSQYGSIYWRNSYENENMERLQCE |
| sviat | Platygyra sp. | PPms-1.pep | 8 | RVLSPQCQYGNIFWRNSYEHENMGRLQCE |
| sviat | Platygyra sp. | PPms-2.pep | 19* | QVLSPQYQYGSIFWRNSYENENMERLRCE |
| sviat | Platygyra sp. | PPms-E.pep | 5 | QVLSPQCQYGNIFWRNSYEHENMGRLQCE |
| sviat | Platygyra sp. | PPms-G.pep | 13 | QVLSPQCQYGNIFWGNSYEHENMGRLQCE |
| sviat | Montipora sp. | RTms-1.pep | 6 | QVLSPQYQYGSIYWRNSYENENMERLQCE |
| sviat | Montipora sp. | RTms-5.pep | 1 | QVLSPQCQYGSIFWRNSYEHENMERLQCE |
| svivt | Montipora sp. | RTms-6.pep | 6 | QVLSPQYQYGSIYWRNSYENENMERLQCE |
| svsat | Montipora sp. | RTms-2.pep | 6 | QVLSPQYQYGSIYWRNSYENENMERLQCE |

TABLE 7

| | | | | |
|---|---|---|---|---|
| N-terminus | Species | Name | Type | Amino acids within 5 Å of fluorophore |

| | | | | |
|---|---|---|---|---|
| sviak | Acropora aspera | Aasv-I.pep | 15 | QVLSPQSQYGSIYWRNSYENGNMERLQCE |
| sviak | Acropora aspera | Aasv-3.pep | 14 | QVLSPQSQYGSIYWRNSYENENMERLQRE |
| sviak | Acropora aspera | Aasv-P.pep | 2 | QVLSPQSQYGSIYWRNSYENENMERLQCE |
| sviak | Acanthastria sp. | Acasv-A.pep | 4 | QVLSPRCQYGNIFWRNSYEHENMGRLQCE |
| sviak | Acanthastria sp. | Acasv-C.pep | 14 | QVLSPQSQYGSIYWRNSYENENMERLQRE |
| sviak | Acanthastria sp. | Acasv-D.pep | 1 | QVLSPQCQYGSIFWRNSYEHENMERLQCE |
| sviak | Caulaserea ap. | Ce61-3sv.pep | 14 | QVLSPQSQYGSIYWRNSYENEMERLQRE |
| sviak | Caulastrea ap. | Ce61-4sv.pep | 20 | QVXSPQSQYGSXYWRNSYEHENMERLQCE |
| sviak | Caulastrea ap. | Ce61-5sv-rep.pep | 18 | QVLSPQCQYGSIFWRNSYEHENMESIQCE |
| sviak | Caulastrea ap. | Ce61-7sv-rep.pep | 1 | QVLSPQCQYGSIFWRNSYEHENMERLQCE |
| sviak | Green Pocillopora | GPd58-2sv.pep | 2 | QVLSPQSQYGSIYWRNSYENENMERLQCE |
| sviak | Acropora nobilis | LGAsv-A.pep | 2 | QVLSPQSQYGSIYWRNSYENENMERLQCE |
| sviak | Acropora nobills | LGAsv-C.pep | 1 | QVLSPQCQYGSIFWRNSYEHENMERLQCE |
| sviak | Acropora nobilis | LGAsv-D.pep | 2 | QVLSPQSQYGSIYWRNSYENENMERLQCE |
| sviak | Acropora nobilis | LGAsv-E.pep | 2 | QVLSPQSQYGSIYWRNSYENENMERLQCE |
| sviak | Millepora sp. (Hydrozoan) | Misv-A.pep | 14 | QVLSPQSQYGSIYWRNSYENENMERLQRE |
| sviak | Millepora sp. (Hydrozoan) | Misv-B.pep | 14 | QVLSPQSQYGSIYWRNSYENENMERLQRE |
| sviak | Millepora sp. (Hydrozoan) | Misv-F.pep | 14 | QVLSPQSQYGSIYWRNSYENENMERLQRE |
| sviak | Pavona decussaca | Pavsv-A.pep | 7 | QVLSPQSQYGSVYWRNSYVNENMERLQCE |
| sviak | Pavona decussaca | Pavsv-B.pep | 1 | QVLSPQCQYGSIFWRNSYEHENMERLQCE |
| sviak | Pavona decussaca | Pavsv-C.pep | 17 | QVLSPQSQYGSVYWRNSYENENMERLQRE |
| sviak | Porites Murrayensis | PM1Asv-rep.pep | 15 | QVLSPQSQYGSIYWRNSYENGNMERLQCE |
| sviak | Porites Murrayensis | PM1Csv-rep.pep | 2 | QVLSPQSQYGSIYWRNSYENENMERLQCE |
| sviak | Porites Murrayensis | PMsv-4.pep | 2* | QVLSPQSQYGSIYWRNSYENENM* |
| sviak | Porites Murrayensis | PMsv-5.pep | 2 | QVLSPQSQYGSIYWRNSYENENMERLQCE |
| sviak | Platygyra sp. | PPsv-1.pep | 5 | QVLSPQCQYGNIFWRNSYEHENMGRLQCE |
| sviak | Platygyra sp. | PPsv-2.pep | 5 | QVLSPQCQYGNIFWRNSYEHENMGRLQCE |
| sviak | Platygyra sp. | PPsv-3.pep | 5 | QVLSPQCQYGNIFWRNSYEHENMGRLQCE |
| sviak | Platygyra sp. | PPsv-4.pep | 4 | QVLSPRCQYGNIFWRNSYEHENMGRLQCE |
| sviak | Platygyra sp. | PPsv-5.pep | 2 | QVLSPQSQYGSIYWRNSYENENMERLQCE |
| sviak | Platygyra sp. | PPsv-6.pep | 10 | QVLSPQSQYGSIYWRNSYENENMERLQCG |
| sviak | Montipora sp. | RTsv-1.pep | 2 | QVLSPQSQYGSIYWRNSYENENMERLQCE |
| sviak | Montipora sp. | RTsv-2.pep | 2 | QVLSPQSQYGSIYWRNSYENENMERLQCE |
| sviak | Montipora sp. | RTsv-3.pep | 2 | QVLSPQSQYGSIYWRNSYENENMERLQCE |

TABLE 8
Percentage DNA sequence similarities generated using LALIGN

| | A8 | D1 | D10 | S1 | S3 | T1 | T3 |
|---|---|---|---|---|---|---|---|
| A8 | | 97.3 | 98.7 | 97.7 | 99.6 | 97.5 | 99.9 |
| D1 | | | 97.5 | 98.1 | 96.9 | 99.6 | 97.2 |
| D10 | | | | 98.2 | 98.2 | 97.6 | 98.5 |
| S1 | | | | | 97.9 | 98.2 | 97.5 |
| S3 | | | | | | 97.0 | 99.4 |
| T1 | | | | | | | 97.3 |
| T3 | | | | | | | |

TABLE 9
Percentage amino acid sequence similarities
generated using LALIGN

| | A8 | D1 | D10 | S1 | S3 | T1 | T3 |
|---|---|---|---|---|---|---|---|
| A8 | | 95.5 | 98.2 | 97.3 | 99.1 | 96.0 | 100.0 |
| D1 | | | 94.6 | 96.4 | 94.6 | 98.7 | 95.5 |
| D10 | | | | 96.4 | 97.3 | 95.1 | 98.2 |
| S1 | | | | | 97.3 | 96.9 | 97.3 |
| S3 | | | | | | 95.1 | 99.1 |
| T1 | | | | | | | 96.0 |
| T3 | | | | | | | |

[image] [image] [image] [image] [image] TABLE 15
Conserved amino acid differences between
blue and purple colored proteins

| | | | Amino acid |
|---|---|---|---|
| | | | in blue- |
| | | | purple |
| Posi- | Amino acid in blue | Amino acid in purple | protein |
| tion | proteins (n = 2) | proteins (n = 4) | (n = 1) |
| 41 | Arg (charged, polar) | Lys (charged, polar)* | Arg |
| 43 | Ala (nonpolar) | Thr (uncharged, polar) | Ala |
| 61 | Cys (uncharged, polar) | Ser (uncharged, polar) | Cys |
| 87 | Phe (nonpolar) | Tyr (uncharged, polar) | Phe |
| 142 | His (charged, polar) | Asn (uncharged, polar) | Asn |
| 143 | Ser (uncharged, polar) | Thr (uncharged, polar) | Thr |
| 175 | Thr (uncharged, polar) | Ser (uncharged, polar) | Ser |
| 198 | Ile (nonpolar) | Thr (uncharged, polar) | Thr |

*Amino acid position 41 of the purple protein encoded by D10 (SEQ ID NO: 192) is Arg.

TABLE 16
Amount of colored protein (expressed as a percentage of total soluble
protein) produced in cultures of E. coli and S. cerevisiae.

| Species | Plasmid | CP clone | Colour | RHSCC | % CP |
|---|---|---|---|---|---|
| E. coli | pCGP2921 | T1 | blue | 102A | 50% |
| S. cerevisiae | pCGP3269 | A8 | purple | 82B | 8% |
| S. cerevisiae | pCGP3270 | T1 | blue | 101C | 6% |

RHSCC = Royal Horticultural Society Colour Chart (Kew, UK)

TABLE 17
Summary of recombinant protein accumulation levels
in plants after nuclear DNA transformation.

| Protein | Plant | Targeting | Accumulation | Reference |
|---|---|---|---|---|
| Endoglucanase | Tobacco | Chloroplast (Tomato- | Up to 1.35% TSP in | Transgenic Res 9: |
| | | Rubisco small | leaves. | 43-54, 2000 |
| | | subunit protein) | | |
| PEPC | Rice | cytosol | Up to 12% TSP in | Nat Biotechnol. 17: |
| | | | leaves. | 22-23, 1999 |
| Cystatin | Rice | cytosol | Up to 2% TSP. | Plant Mol Biol 30: |
| | | | | 149-157, 1996 |
| Antibody | Arabid. | ER-retained | Up to 6% TSP | Eur J Biochem |
| | | (DIKDEL), ER- | | |
| | | secreted | | |
| Spider Silk | Tobacco | ER-retained | 2% + TSP in leaves and | Nat Biotechnol 19: |
| | Potato | | potato tubers | 573-577. 2000 |
| Cry9Aa | Tobacco | cytosol | 0.3% TSP in Tobacco | Plant Sci 160: |
| | Potato | | leaves. Expression in | 341-353, 2001 |
| | Cauliflower | | other plants 0.1-0.03%. | |
| | Turnip | | | |
| Xylanase | Tobacco | ER-excreted in | 3% TSP (alk. phos.) | Plant Physiol 124: |
| GFP | | guttation fluid | | 927-934, 2000 |
| Alkaline | | | | |
| phosphatase | | | | |
| GUS- | ? | cytosol | Up to 3% TSP | FEBS Lett 488: |
| Peptide vaccine | | | | 13-17, 2001 |
| Bt, NPTII | Tobacco | cytosol | Bt: 0.02% TSP | Nature 328: 33-37, |
| | | | NPTII: 0-07-0.27% TSP | 1987 |
| AlMV-CP | Tobacco | cytosol | 0.1-0.4% TSP Tobacco | EMBO 6: |
| | Tomato | | 0.1-0.8% TSP Tomato | 1181-11188, 1987 |
| sGFP | Rice | Chloroplast (rbsS-Tp) | 10% of TSP. Much | Plant & Animal |
| | | | higher than cytoplasmic | Genome VII |
| | | | control (0.5% TSP) | Conference 1999 |
| | | | | abstract |

TSP = total soluble protein

TABLE 18
Summary of recombinant protein accumulation levels
in plants after Plastid DNA transformation.

| Protein | Plant | Targeting | Accumulation | Reference |
|---|---|---|---|---|
| GFP | tobacco | Chloroplast expression | 5% TSP in leaves | Plant Journal 27: |
| | | | | 257-265 |
| Bt (cry2Aa2) | tobacco | Chloroplast expression | 2-3% TSP in leaves | Proc Natl Acad Sci |
| | | | | 1840-1845, 1999 |
| Bt (cry2Aa2) | ? | Chloroplast expression | 45.3% TSP | Nat Biotechnol 19: |
| | | | | 71-74, 2001 |
| Somatotropin | Tobacco | Chloroplast expression | 7% + “more than 300- | Nat Biotechnol 18: |
| | | | fold higher than a similar | 333-338, 2000 |
| | | | gene expressed using a | |
| | | | nuclear transgenic | |
| | | | approach” | |
| Bt (cry1Ac) | Tobacco | Chloroplast expression | 3-5% TSP in leaves | Biotechnology 13: |
| | | | | 362-365, 1995 |
| EPSPS | Tobacco | Chloroplast expression | 10% + TSP in leaves | Plant J 25: |
| | | | | 261-270, 2001 |

TSP = total soluble protein

[image] TABLE 20
Summary of Northern analysis of Arabidopsis transgenic plants

| Construct | | Selectable | | |
|---|---|---|---|---|
| number | CP Cassette | marker | T1 | SuRB |
| pCGP2772 | 35S:T1:35S | 35S:SuRB | ˜0.9 kb | ˜2.2 kb |
| pCGP2765 | 35S:A8:35S | 35S:SuRB | ˜0.9 kb | ˜2.2 kb |
| pCGP3259 | 35S:ER:T1:35S | 35S:SuRB | ˜1.0 kb | ˜2.2 kb |
| pCGP2785 | 35S:SSU:T1:35S | 35S:SuRB | ˜1.1 kb | ˜2.2 kb |
| pCGP3258 | 35S:T1:GFP:35S | 35S:SuRB | ˜1.6 kb | ˜2.2 kb |
| pCGP3261 | 35S:ER:T1:GFP:35S | 35S:SuRB | ˜1.7 kb | ˜2.2 kb |
| pCGP960 | 35S:GUS | 35S:SuRB | none | ˜2.2 kb |
| pBINmgfp4 | 35S:mGFP4:nos | 35S:nptII | none | none |
| non | NA | NA | none | none |
| transgenic | | | | |

CP cassette = Colored protein cassette contained in construct;
SM cassette = the selectable marker gene contained in construct;
NA = not applicable;
none = no transcripts detected

TABLE 21
Estimations of T1 protein in leaf samples from 2 transgenic Arabidopsis
events (expressed as a percentage of total protein)

| Construct | Cassette | Acc # | Sample | % T1 |
|---|---|---|---|---|
| pCGP3259 | 35S:ERT:T1:35S | 1.5 | leaf | 0.005% |
| pCGP2772 | 35S:T1:35S | 1.2b | leaf | 0.005% |

Construct = Binary vector used in transformation;
Cassette refers to the chimaeric T1 transgene contained in the T-DNA;
Acc# refers to the accession number of the transgenic plant.

TABLE 22
Estimations of T1 protein in petal and/or leaf
samples from 2 transgenic P. hybrida events (expressed
as a percentage of total protein)

| Construct | Cassette | Acc # | Sample | % T1 |
|---|---|---|---|---|
| pCGP3259 | 35S:ERT:T1:35S | 24444 | leaf | 0.009% |
| pCGP2765 | 35S:A8:35S | 24534 | petal | 0.02% |
| pCGP2765 | 35S:A8:35S | 24534 | leaf | 0.006% |

[image]

1. An isolated nucleic acid molecule comprising a nucleotide sequence encoding a color-facilitating molecule (CFM) which in a cell, alone or together with one or more other molecules imparts an altered visual characteristic to said cell when visualized by a human eye in the absence of excitation by extraneous non-white light or particle emission, wherein the CFM is derived from Anemonia majano, Anemonia sulcata, Clavularia sp, Zoanthus sp, Discosoma sp (e. g. Discosoma striata), Aequorea sp (e. g. Aequorea victoria), Anthozoa sp, Cassiopea sp, (e. g. Cassiopea xamachana), Millepora sp, Acropora sp (e. g. Acropora aspera and Acropora nobilis), Montipora sp, Poritesmurrayensis, Pocillopora damiconnis, Pavona descussaca, Acanthastrea sp, Platvgyra sp or Caulastrea sp, and wherein the CFM comprises an amino acid sequence in its N-terninal end selected from the group consisting of SVIAK (SEQ ID NO: 5), (M)SVIAT (SEQ ID NO: 6), SGIAT (SEQ ID NO: 7), SVIVT (SEQ ID NO: 8) or SVSAT (SEQ ID NO :9), SVIAK QMTY KVNM SGT (SEQ ID NO: 16), and SVIAK QMTY KVYM SDT (SEQ ID NO: 17).

2-3. (canceled)

4. The isolated nucleic acid molecule of claim 1 wherein the CFM comprises an amino acid sequence selected from the list comprising SVIAT QMTY KVYM SGT (SEQ ID NO: 10), SVIAT QMTY KVYM PEG (SEQ ID NO :11), SVIAT QVTY KVYM
SGT (SEQ ID NO: 12), SGIAT QMTY KVYM SGT (SEQ ID NO: 13), SVIVT QMTY
KVYM SGT (SEQ ID NO: 14), SVSAT QMTY KVYM SGT (SEQ ID NO: 15), SVIAK
QMTY KVNM SGT (SEQ ID NO: 16), SVIAK QMTY KVYM SDT (SEQ ID NO: 17) and
SVIAK QMTYXIX2YX3SGT (SEQ ID NO: 18) wherein_XI, Xz and X3 may be any amino acid provided that_Xl is not K;_Xz is not_V ; X3 is not M.

5. The isolated nucleic acid molecule of claim 4 wherein the CFM comprises an amino acid sequence selected from the list comprising SEQ ID NOs: 20,22, 24,26,28,30,32,34,36,38,40,42,44,46,48,50,52,54,56,58,60,62,64,66,68 70,72, 74, 76, 78, 80,82,84,86,88,90,92,94,96,98,100,102,104,106,108,110,112, 114,116,118,120,122,124,126,128,130,132,134,136,138,140,142,144,146,148, 150,152,154,156,158,160,162,164,166,168,170,172,174,176,178,180,190,192, 194,196,198,200 and 202 provided that, where the said amino acid sequence comprises the sequence SVIAK QMTYXIX2yX3 SGT, Xi is not lysine,X2 is not valine, and X3 is not methionine or an amino acid sequence having at least 60% similarity to any one or more of the above referenced sequences.

6. The isolated nucleic acid molecule of claim 5 comprising a nucleotide sequence encoding a color-facilitating molecule (CFM), wherein the nucleotide sequence is selected from the list comprising SEQ ID NOs :19, 21,23,25,27,29,31,33,35,37,39, 41,43,45,47,49,51,53,55,57,59,61,63,65,67,69,71,73,75,77,79,81,83,85,87,89, 91,93,95,97,99,101,103,105,107,109,111,113,115,117,119,121,123,125, 127,129,131,133,135,137,139,141,143,145,147,149,151,153,155,157,159,161, 163,165,167,169,171,173,175,177,179,189,191,193,195,197,199 and 201 or a nucleotide sequence having at least 60% similarity to one or more of the above referenced sequences or a nucleotide sequence capable of hybridizing to one of the above referenced sequences ora complementary form thereof under low stringency conditions.

7. The isolated nucleic acid molecule of claim 4 wherein the cell is a prokaryotic cell.

8. The isolated nucleic acid molecule of claim 4 wherein the cell is a eukaryotic cell.

9. The isolated nucleic acid molecule of claim 8 wherein the eukaryotic cell is a mammalian animal cell.

10. The isolated nucleic acid molecule of claim 8 wherein the eukaryotic cell is a non-mammalian animal cell.

11. The isolated nucleic acid molecule of claim 10 wherein the eukaryotic cell is a plant cell.

12. The isolated nucleic acid molecule of claim 11 wherein the plant cell is part of a plant callus or a whole plant.

13. The isolated nucleic acid molecule of claim 12 wherein the whole plant is an ornamental or flowering plant or a part thereof.

14. The isolated nucleic acid molecule of claim 13 wherein the plant part is a flower, root, leaf, stem, seed, fruit or fiber.

15. The isolated nucleic acid molecule of claim 13 wherein the plant is selected from a rose, carnation, lisianthus, petunia, lily, tulip, pansy, gerbera or chrysanthemum.

16. The isolated nucleic acid molecule of claim 4 wherein the CFM is a GFP or derivative or homolog thereof.

17. The isolated nucleic acid molecule of claim 16 wherein the homolog of GFP is a non-fluorescent GFP.

18. An isolated color-facilitating molecule (CM) comprising a polypeptide which, in a cell, alone or together with one or more other molecules imparts an altered visual characteristic to said cell when visualized by a human eye in the absence of excitation by extraneous non-white light or particle emission, wherein the CFM is derived from Anemonia majano, Aneznonia sulcata, Clavularia sp, Zoanthus sp, Diycosoma sp (e. g.Discosoma striata) Aequorea sp (e.g. Aequorea victoria), Anthozoa sp, Cassiopea sp, (e. g. Cassiopea xamachana), Millepora sp, Acropora sp (e. R. Acropora aspera and Acropora nobilis), Montipora sp, Poritesmurravensis, Pocillopora damiconrcis, Pavona descussaca, Acanthastrea sp, Platvgyra sp or Caulastrea sp, and wherein the CFM comprises an amino acid sequence in its N-terminal end selected from SVIAK (SEQ ID NO: 5), (M)SVIAT (SEQ ID NO: 6), SGIAT (SEQ ID NO: 7), SVIVT (SEQ ID NO: 8) or SVSAT (SEQ ID NO: 9), SVIAK QMTY KVNM SGT (SEQ ID NO: 16), and SVIAK QMTY KVYM SDT (SEQ ID NO: 17).

19-20. (canceled)

21. The isolated CFM of claim 18 wherein the CFM comprises an amino acid sequence selected from the list comprising SVIAT QMTY KVYM SGT (SEQ ID NO: 10),SVIAT QMTY KVYM PEG (SEQ ID NO:11), SVIAT QVTY KVYM SGT (SEQ ID NO: 12), SGIAT QMTY KVYM SGT (SEQ ID NO:13), SVIVT QMTY KVYM SGT (SEQ ID NO: 14), SVSAT QMTY KVYM SGT (SEQ ID NO: 15), SVIAK QMTY KVNM SGT (SEQ ID NO: 16), SVIAK QMTY KVYM SDT (SEQ ID NO: 17) and SVIAK QMTYXIX2yX3 SGT (SEQ ID NO: 18) wherein Xi, X2 and X3 may be any amino acid provided that Xi is not K;X2 is not V; X3 is not M.

22. The isolated CFM of claim 21 wherein the CFM comprises a polypeptide having an amino acid sequence selected from the list comprising SEQ ID NOs : 20,22,24, 26,28,30,32,34,36,38,40,42,44,46,48,50,52,54,56,58,60,62,64,66,68 70,72, 74,76,78,80,82,84,86,88,90,92,94,96,98,100,102,104,106,108,110,112,114, 116, 118,120, 122, 124, 126,128,130,132,134,136,138,140,142,144,146,148,150, 152,154,156,158,160,162,164,166,168,170,172,174,176,178,180,190,192,194, 196,198,200 and 202 provided that, where the said amino acid sequence comprises the sequence SVIAK QMTYX, X2YX3 SGT, Xi is not lysine, X2 is not valine, and X3 is not methionine or an amino acid sequence having at least 60% similarity to any one or more of the above referenced sequences.

23. The isolated CFM of claim 18 wherein the cell is a prokaryotic cell.

24. The isolated CFM of claim 18 wherein the cell is a eukaryotic cell.

25. The isolated CFM of claim 24 wherein the eukaryotic cell is a mammalian animal cell.

26. The isolated CFM of claim 24 wherein the eukaryotic cell is a nonmammalian animal cell.

27. The isolated CFM of claim 26 wherein the non-mammalian animal cell is a plant cell.

28. The isolated CFM of claim 27 wherein the plant cell is part of a plant callus or a whole plant.

29. The isolated CFM of claim 28 wherein the whole plant is an ornamental or flowering plant or a part thereof

30. The isolated CFM of claim 29 wherein the plant part is a flower, root, leaf, stem, seed, fruit or fiber.

31. The isolated CFM of claim 29 wherein the plant is selected from a rose, carnation, lisianthus, petunia, lily, tulip, pansy, gerbera or chrysanthemum.

32. The isolated CFM of claim 21 wherein the CFM is a GFP or derivative or homolog thereof.

33. The isolated CFM of claim 32 wherein the homolog of GFP is a nonfluorescent GFP.

34. An isolated cell wherein said cell or a parent cell is genetically modified to enable the production of a color-facilitating molecule (CFM) of claims 18 or 21 which alone or together with one or more other molecules imparts an altered visual characteristic to said cell when visualized by a human eye in the absence of excitation by extraneous non-white light or particle emission.

35-38. (canceled)

39. The isolated cell of claim 34 wherein the cell is a prokaryotic cell.

40. The isolated cell of claim 34 wherein the cell is a eukaryotic cell.

41-42. (canceled)

43. The isolated cell of claim 40 wherein the eukaryotic cell is a plant cell.

44. The isolated plant cell of claim 43 wherein the cell is part of a plant callus or a whole plant.

45-76. (canceled)

77. An isolated antibody specific for a CFM, of any one of claims 18 or 21.

78-114. (canceled)


SEQUENCE LISTING (251 sequences)

SEQ ID NO: 1 - DNA, 30 bp, Artificial Sequence
  Primer
tccgttatcgctaaacagatgacctacaaa

SEQ ID NO: 2 - DNA, 21 bp, Artificial Sequence
  Primer
ggcgaccacaggtttgcgtgt

SEQ ID NO: 3 - DNA, 21 bp, Artificial Sequence
  Primer
atgagtgtgatcgctacacaa

SEQ ID NO: 4 - DNA, 21 bp, Artificial Sequence
  Primer
tttgtgccttgatttgactct

SEQ ID NO: 5 - protein, 5 aa, Artificial Sequence
  N-terminal amino acid sequence derived from Acropora sp.
SVIAK

SEQ ID NO: 6 - protein, 6 aa, Artificial Sequence
  N-terminal amino acid sequence derived from Acropora sp.
MSVIAT

SEQ ID NO: 7 - protein, 5 aa, Artificial Sequence
  N-terminal amino acid sequence derived from Montipora sp.
SGIAT

SEQ ID NO: 8 - protein, 5 aa, Artificial Sequence
  N-terminal amino acid sequence derived from Montipora sp.
SVIVT

SEQ ID NO: 9 - protein, 5 aa, Artificial Sequence
  N-terminal amino acid sequence derived from Montipora sp.
SVSAT

SEQ ID NO: 10 - protein, 16 aa, Artificial Sequence
  N-terminal amino acid sequence derived from Montipora sp.
SVIATQMTYKVYMSGT

SEQ ID NO: 11 - protein, 16 aa, Artificial Sequence
  N-terminal amino acid sequence derived from Montipora sp.
SVIATQMTYKVYMPGT

SEQ ID NO: 12 - protein, 16 aa, Artificial Sequence
  N-terminal amino acid sequence derived from Montipora sp.
SVIATQVTYKVYMSGT

SEQ ID NO: 13 - protein, 16 aa, Artificial Sequence
  N-terminal amino acid sequence derived from Montipora sp.
SGIATQMTYKVYMSGT

SEQ ID NO: 14 - protein, 16 aa, Artificial Sequence
  N-terminal amino acid sequence derived from Montipora sp.
SVIVTQMTYKVYMSGT

SEQ ID NO: 15 - protein, 16 aa, Artificial Sequence
  N-terminal amino acid sequence derived from Montipora sp.
SVSATQMTYKVYMSGT

SEQ ID NO: 16 - protein, 16 aa, Artificial Sequence
  N-terminal amino acid sequence derived from Acropora sp.
SVIAKQMTYKVNMSGT

SEQ ID NO: 17 - protein, 16 aa, Artificial Sequence
  N-terminal amino acid sequence derived from Acropora sp.
SVIAKQMTYKVYMSDT

SEQ ID NO: 18 - protein, 16 aa, Artificial Sequence
  N-terminal amino acid sequence derived from Acropora sp.
SVIAKQMTYXXYXSGT

SEQ ID NO: 19 - DNA, 660 bp, Acropora aspera
  CDS (1)...(660)
tccgttatcgctaaacagatgacctacaaggtttatatgtcagacacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggtaactgtttcatctacaatgtcaagttctctggtttgaactttcctcccaatggacctgttatgcaaaagaagacacagggctgggaacccaacactgggcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaagttggatgtaaccaatcacaacaaggattacacttccgttgagcagtgtgaaatttccattgcacgcaaacctgtggtcgcc

SEQ ID NO: 20 - protein, 220 aa, Acropora aspera
SVIAKQMTYKVYMSDTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTGRLFARDGMLIGNNFMALKLEGGGHYLCEFKSTYKAKKPVMMPGYHYVDRKLDVTNHNKDYTSVEQCEISIARKPVVA

SEQ ID NO: 21 - DNA, 660 bp, Acropora aspera
  CDS (1)...(660)
tccgttatcgctaaacagatgacctacaaagtttatatgtcaggcacggtcaatggacactactttgaggtcgaaggcgatggaaaaggaaagccttacgagggggagcagacggtaaggctgactgtcaccaagggcggacctctgccatttgcttgggatattttatcaccacagtcacagtacggaagcataccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctcttagcacgagatggaatgctgataggaaacaactttatggctctgaagttagaaggaggtggtcactatttgtgtgaattcaaatctacttacaaggcaaggaagcctgtgaagatgccagggtatcactatgttgaccgcaaactggatgtaaccaatcacaacaaggattacacttccgttgagcagcgtgaaatttccattgcacgcaaacctgtggtcgcc

SEQ ID NO: 22 - protein, 220 aa, Acropora aspera
SVIAKQMTYKVYMSGTVNGHYFEVEGDGKGKPYEGEQTVRLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLLARDGMLIGNNFMALKLEGGGHYLCEFKSTYKARKPVKMPGYHYVDRKLDVTNHNKDYTSVEQREISIARKPVVA

SEQ ID NO: 23 - DNA, 663 bp, Acropora aspera
  CDS (1)...(663)
tccgttatcgctaaacagatgacctacaaagtttatatgtcaggcacggtcaatggacactactttgaggtcgaaggcgatggaaaaggaaagccttacgagggggagcagacggtaaggctgactgtcaccaagggcggacctctgccatttgcttgggatattttatcaccacagtcacagtacggaagcataccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctttgcacgagatggaatgctgataggaaacaactttatggctctgaagttagaaggaggtggtcactatttgtgtgaattcaaatctacttacaaggcaaagaagcctgtgaggatgccagggtatcactatgttgaccgcaaactggatgtaaccaatcacaacaaggattacacttccgttgagcagtgtgaaatttccattgcacgcaaacctgtggtcgcctga

SEQ ID NO: 24 - protein, 220 aa, Acropora aspera
SVIAKQMTYKVYMSGTVNGHYFEVEGDGKGKPYEGEQTVRLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVRMPGYHYVDRKLDVTNHNKDYTSVEQCEISIARKPVVA

SEQ ID NO: 25 - DNA, 660 bp, Aranthastria sp.
  CDS (1)...(660)
tccgttatcgctaaacagatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacggtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacggggctgggaaccccactctgagcgtctctttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 26 - protein, 220 aa, Aranthastria sp.
SVIAKQMTYKVYMSGTVNGHYFEVEGDGKGKPYEGEQTVKLTVTKGGPLPFAWDILSPRCQYGNIPFTKYPEDVPDYVKQSFPEGFTWERIMNFEDGAVCTVSNDSSIQGNCFTYHVKFSGLNFPPNGPVMQKKTRGWEPHSERLFARGGMLIGNNFMALKLEGGGHYLCGFKTTYKAKKPVKMPGYHYVDRKLDVTNHNKDYISVEQCEISIARKPVVA

SEQ ID NO: 27 - DNA, 660 bp, Aranthastria sp.
  CDS (1)...(660)
tccgttatcgctaaacagatgacctacaaagtttatatgtcaggcacggtcaatggacactactttgaggtcgaaggcgatggaaaaggaaagccttacgagggggagcagacggtaaggctgactgtcaccaagggcggacctctgccatttgcttgggatattttatcaccacagtcacagtacggaagcataccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctctgcacgagatggaatgctgataggaaacaactttatggctctgaagttagaaggaggtggtcactatttgtgtgaattcaaatctacttacaaggcaaggaagcctgtgaagatgccagggtatcactgtgttgaccgcaaactggatgtaaccaatcacaacaaggattacacttccgttgagcagcgtgaaatttccattgcacgcaaacctgtggtcgcc

SEQ ID NO: 28 - protein, 220 aa, Aranthastria sp.
SVIAKQMTYKVYMSGTVNGHYFEVEGDGKGKPYEGEQTVRLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLSARDGMLIGNNFMALKLEGGGHYLCEFKSTYKARKPVKMPGYHCVDRKLDVTNHNKDYTSVEQREISIARKPVVA

SEQ ID NO: 29 - DNA, 660 bp, Aranthastria sp.
  CDS (1)...(660)
tccgttatcgctaaacagatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgactccagcatccaaggcaactgtttcacctaccatgtcaagttctctggtttgaactttcctcccaatgggcctgtgatgcagaagaagacacagggctgggaaccccactctgagcgtctctttgcacggggtggaatgctgataggaaacaactttatggctccgaagttagaaggaggcggtcactatttgtgtgaattcaaaactacttacaaggcaaagaagcctgtgaagatgccggggtatcattatgttgaccgcaaactggatgtaaccaatcacaacaaggattacacttccgttgagcagtgtgaaatctccattgcacgcaaacctgtggtcgcc

SEQ ID NO: 30 - protein, 220 aa, Aranthastria sp.
SVIAKQMTYKVYMSGTVNGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLNFPPNGPVMQKKTQGWEPHSERLFARGGMLIGNNFMAPKLEGGGHYLCEFKTTYKAKKPVKMPGYHYVDRKLDVTNHNKDYTSVEQCEISIARKPVVA

SEQ ID NO: 31 - DNA, 660 bp, Caulastrea sp.
  CDS (1)...(660)
tccgttatcgctaaacagatgacctacaaagtttatatgtcaggcacggtcaatggacactactttgaggtcgaaggcgatggaaaaggaaagccttacgagggggagcagacggtaaggctgactgtcaccaagggcggacctctgccatttgcttgggatattttatcaccacagtcacagtacggaagcataccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctctgcacgagatggaatgctgataggaaacaactttatggctctgaagttagaaggaggtggtcactatttgtgtgaattcaaatctacttacaaggcaaggaagcctgtgaagatgccagggtatcactatgttgaccgcaaactggatgtaaccaatcacaacaaggattacacttccgttgagcagcgtgaaatttccattgcacgcaaacctgtggtcgcc

SEQ ID NO: 32 - protein, 220 aa, Caulastrea sp.
SVIAKQMTYKVYMSGTVNGHYFEVEGDGKGKPYEGEQTVRLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLSARDGMLIGNNFMALKLEGGGHYLCEFKSTYKARKPVKMPGYHYVDRKLDVTNHNKDYTSVEQREISIARKPVVA

SEQ ID NO: 33 - DNA, 660 bp, Caulastrea sp.
  CDS (1)...(660)
  misc_feature 72,133,166,193,486,495,501: n = any nucleotide
tccgttatcgctaaacagatgacctacaaggtttatatgtcaggcacggtcaatggacactactttgaggtngaaggcgatggaaaaggaaagccttacgagggggagcagacggtaaagctcactgtcaccnagggcggacctctgccatttgcttgggatattntatcaccacagagtcagtacggaagcntaccattcaccaagtaccctgaagacatccctgactatgtaaagcagtcattccctgagggatatacatgggagaggatcatgaactttgaagatggtgcagtgtgtactgtcagcaatgattccagcatccaaggtaactgtttcatctaccatgtcaagttctctggtttgaactttcctcccaatgggcctgttatgcaaaagaagacacagggctgggaaccccactctgagcgtctctttgcacgagatggaatgctgataggaaacaactttatggctctgaagttngaaggaggnggtcantatttgtgtgaattcaaatctacttacaaggcaaagaagcctgtgatgatgccagggtatcactatgttgaccgcaaattggatgtaaccaatcacaacaaggattacacttccgttgagcagtgtgaaatatccattgcacgcaaacctgtggtcgcc

SEQ ID NO: 34 - protein, 220 aa, Caulastrea sp.
  MOD_RES (45)...(45): Xaa = Lys, Glu, Gln or stop codon
SVIAKQMTYKVYMSGTVNGHYFEVEGDGKGKPYEGEQTVKLTVTXGGPLPFAWDIXSPQSQYGSXPFTKYPEDIPDYVKQSFPEGYTWERIMNFEDGAVCTVSNDSSIQGNCFIYHVKFSGLNFPPNGPVMQKKTQGWEPHSERLFARDGMLIGNNFMALKXEGGGXYLCEFKSTYKAKKPVMMPGYHYVDRKLDVTNHNKDYTSVEQCEISIARKPVVA

SEQ ID NO: 35 - DNA, 660 bp, Caulastrea sp.
  CDS (1)...(660)
  misc_feature 637,638,640: n = any nucleotide
tccgttatcgctaaacagatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatgggcctgtgatgcagaagaagacacagggctgggaaccccactctgagcgtctctttgcacggggtggaatgctgataggaaacaactttatggctctgaagttagaagggggcggtcactatttgtgtgaattcaaaactacttacaaggcaaagaagcctgtgaagatgccagggtatcattatgtttacagcaccattcatgtaaccaatcacaacaaggattacacttccgttgagcagtgtgaaatttccnntncacgcaaacctgtggtcgcc

SEQ ID NO: 36 - protein, 220 aa, Caulastrea sp.
  MOD_RES (213)...(213): Xaa = Asn, Ser, Thr, Ile, Asp, Gly, Ala, Val, His, Arg, Pro, Leu, Tyr, Cys or Phe
SVIAKQMTYKVYMSGTVNGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCEFKTTYKAKKPVKMPGYHYVYSTIHVTNHNKDYTSVEQCEISXXRKPVVA

SEQ ID NO: 37 - DNA, 660 bp, Caulastrea sp.
  CDS (1)...(660)
  misc_feature 30,32,37,48,52: n = any nucleotide
tccgttatcgctaaacagatgacctacatngnttatntgtcaggcacngtcnatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatgggcctgtgatgcagaagaagacacagggctgggaaccccactctgagcgtctctttgcacggggtggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattgcacgcaaacctgtggtcgcc

SEQ ID NO: 38 - protein, 220 aa, Caulastrea sp.
  MOD_RES (10)...(10): Xaa = Ile or Met
SVIAKQMTYXXYXSGTVXGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCEFKTTYKAKKPVKMPGYHYVDRKLDVTNHNKDYTSVEQCEISIARKPVVA

SEQ ID NO: 39 - DNA, 660 bp, Green Pcillopora
  CDS (1)...(660)
tccgttatcgctaaacagatgacctacaaggtttatatgtcaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggtaactgtttcatctacaatgtcaagttctctggtttgaactttcctcccaatggacctgttatgcaaaagaagacacagggctgggaacccaacactgagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgaaatttccattgcacgcaaacctgtggtcgcc

SEQ ID NO: 40 - protein, 220 aa, Green Pcillopora
SVIAKQMTYKVYMSGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLFARDGMLIGNNFMALKLEGGGHYLCEFKSTYKAKKPVMMPGYHYVDRKLDVTNHNKDYTSVEQCEISIARKPVVA

SEQ ID NO: 41 - DNA, 660 bp, Acropora nobilis; identical to SEQ ID NO: 39
  CDS (1)...(660)

SEQ ID NO: 42 - protein, 220 aa, Acropora nobilis; identical to SEQ ID NO: 40

SEQ ID NO: 43 - DNA, 660 bp, Acropora nobilis
  CDS (1)...(660)
tccgttatcgctaaacagatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcacctaccacgtcaagttctctggtttggactttcctcccaatgggcctgtgatgcagaagaagacacagggctgggaaccccactctgagcgtctctttgcacggggtggaatgctgataggaaacaactttatggctctgaagttagaaggaggcggtcactatttgtgtgaattcaaaactacttacaaggcaaagaagcctgtgaagatgccaggatatcattatgttgaccgcaaactggatgtaaccaatcacaacaaggattacacttccgttgagcagtgtgaaatttccattgcacgcaaacctgtggtcgcc

SEQ ID NO: 44 - protein, 220 aa, Acropora nobilis
SVIAKQMTYKVYMSGTVNGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLDFPPNGPVMQKKTQGWEPHSERLFARGGMLIGNNFMALKLEGGGHYLCEFKTTYKAKKPVKMPGYHYVDRKLDVTNHNKDYTSVEQCEISIARKPVVA

SEQ ID NO: 45 - DNA, 660 bp, Acropora nobilis; identical to SEQ ID NO: 39
  CDS (1)...(660)

SEQ ID NO: 46 - protein, 220 aa, Acropora nobilis; identical to SEQ ID NO: 40

SEQ ID NO: 47 - DNA, 660 bp, Acropora nobilis
  CDS (1)...(660)
tccgttatcgctaaacagatgacctacaaggtttatatgtcaggcacggtcaatggacactactttgaggttgaaggcgatggaaaaggaaagccttacgagggggagcagacggtaaagctcactgtcaccgagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggtaactgtttcatctacaatgtcaagttctctggtttgaactttcctcccaatggacctgttatgcaaaagaagacacagggctgggaacccaacactgagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgaaatttccattgcacgcaaacctgtggtcgcc

SEQ ID NO: 48 - protein, 220 aa, Acropora nobilis
SVIAKQMTYKVYMSGTVNGHYFEVEGDGKGKPYEGEQTVKLTVTEGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLFARDGMLIGNNFMALKLEGGGHYLCEFKSTYKAKKPVMMPGYHYVDRKLDVTNHNKDYTSVEQCEISIARKPVVA

SEQ ID NO: 49 - DNA, 660 bp, Millepora sp. (Hydrozoan)
  CDS (1)...(660)
tccgttatcgctaaacagatgacctacaaggtttatatgtcaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctttgcacgagatggaatgctgataggaaacaactttatggctctgaagttagaaggaggtggtcactatttatgtgaattcaaatctacttacaaggcaaggaagcctgtgaagatgccagggtatcactatgttgaccgcaaactggatgtaaccaatcacaacaaggattacacttccgttgagcagcgtgaaatttccattgcacgcaaacctgtggtcgcc

SEQ ID NO: 50 - protein, 220 aa, Millepora sp. (Hydrozoan)
SVIAKQMTYKVYMSGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKARKPVKMPGYHYVDRKLDVTNHNKDYTSVEQREISIARKPVVA

SEQ ID NO: 51 - DNA, 660 bp, Millepora sp. (Hydrozoan)
  CDS (1)...(660)
tccgttatcgctaaacagatgacctacaaagtttatatgtcaggcacggtcaatggacactactttgaggtcgaaggcgatggaaaaggaaagccttacgagggggagcagacggtaaggctgactgtcaccaagggcggacctctgccatttgcttgggatattttatcaccacagtcacagtacggaagcataccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctttgcacgagatggaatgctgataggaaacaactttatggctctgaagttagaaggaggtggtcactatttgtgtgaattcaaatctacttacaaggcaaggaagcctgtgaagatgccagggtatcactatgttgaccgcaaactggatgtaaccaatcacaacaaggattacacttccgttgagcagcgtgaaatttccattgcacgcaaacctgtggtcgcc

SEQ ID NO: 52 - protein, 220 aa, Millepora sp. (Hydrozoan)
SVIAKQMTYKVYMSGTVNGHYFEVEGDGKGKPYEGEQTVRLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKARKPVKMPGYHYVDRKLDVTNHNKDYTSVEQREISIARKPVVA

SEQ ID NO: 53 - DNA, 660 bp, Millepora sp. (Hydrazoan)
  CDS (1)...(660)
tccgttatcgctaaacagatgacctacaaagtttatatgtcaggcacggtcaatggacactactttgaggtcgaaggcgatggaaaaggaaagccttacgagggggagcagacggtaaggctgactgtcaccaagggcggacctctgccatttgcttgggatattttatcaccacagtcacagtacggaagcataccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctttgcacgagatggaatgctgataggaaacaactttatggctctgaagttagaaggaggtggccactatttgtgtgaattcaaatctacttacaaggcaaggaagcctgtgaagatgccagggtatcactatgttgaccgcaaactggatgtaaccaatcacaacaaggattacacttccgttgagcagcgtgaaatttccattgcacgcaaacctgtggtcgcc

SEQ ID NO: 54 - protein, 220 aa, Millepora sp. (Hydrazoan); identical to SEQ ID NO: 52

SEQ ID NO: 55 - DNA, 660 bp, Porites Murrayensis; identical to SEQ ID NO: 19
  CDS (1)...(660)

SEQ ID NO: 56 - protein, 220 aa, Porites Murrayensis; identical to SEQ ID NO: 20

SEQ ID NO: 57 - DNA, 660 bp, Porites Murrayensis
  CDS (1)...(660)
tccgttatcgctaaacagatgacctacaaggtttatatgtcaggcacggtcaatggacactactttgaggttgaaggcgatggaaaaggaaggccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaagtttgaagatggtgcagtatgtactgtcagcaatgattccagcatgcaaggcaactgtttcatctacaatgtcaagttctctggtttgaactttcctcccaatggacctgttatgcagaagaagacacagggctgggaacccaacactgagcgactttatgcacgagatggaatgctgataggaaacaactttatggctctgaagttggaaggaagtggtcattatacctgtgaattcaaatctacttacaaggcaaagaagcctgtgatgatgcctggatatcactatgttgaccgcaaattggatgtaaccaatcacaacaaggattacacttccgttgagcagtgtgaaatttccattgcacgcaaacctgtggtcgcc

SEQ ID NO: 58 - protein, 220 aa, Porites Murrayensis
SVIAKQMTYKVYMSGTVNGHYFEVEGDGKGRPYEGEQTVKLTVTKGGPLPFAWDILSPQSQYGSIPFTKYPEDIPDYVKQSFPEGYTWERIMKFEDGAVCTVSNDSSMQGNCFIYNVKFSGLNFPPNGPVMQKKTQGWEPNTERLYARDGMLIGNNFMALKLEGSGHYTCEFKSTYKAKKPVMMPGYHYVDRKLDVTNHNKDYTSVEQCEISIARKPVVA

SEQ ID NO: 59 - DNA, 656 bp, Porites Murrayensis
  CDS (1)...(507)
tccgttatcgctaaacagatgacctacaaggtttatatgtcaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaggacatccctgactatgtaaagcagtcattccctgagggatatacatgggagaggatcgtgaacttcgaagatggtgcagtgtgtactgtcagcaatgattccagcatccaaggcaactgtttcatctacaatgtcaagttctctggtttgaactttcctcccaatggacctgttatgcagaagaagacacagggctgggaacccaacactgagcgtctttatgcacgggatggaatgctgataggaaacaactttatggctctgaagttggaaggagggcaaagaagcctgtgatgatgccagggtatcactatgttgaccgcgaattggatgtaaccaatcacaacaaggattacacttccgttgagcagtgtgagatttccatcgcacgcaaacctgtggtcgcctgacgttttttcagagtcaaatcaaggcacaaa

SEQ ID NO: 60 - protein, 169 aa, Porites Murrayensis
SVIAKQMTYKVYMSGTVNGHYFEVEGDGKGKPYEGEQTVKLTVTKGGPLPFAWDILSPQSQYGSIPFTKYPEDIPDYVKQSFPEGYTWERIVNFEDGAVCTVSNDSSIQGNCFIYNVKFSGLNFPPNGPVMQKKTQGWEPNTERLYARDGMLIGNNFMALKLEGGQRSL

SEQ ID NO: 61 - DNA, 693 bp, Porites Murrayensis
tccgttatcgctaaacagatgacctacaaggtttatatgtcaggcacggtcaatggacactactttgaggtccaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcaaagatggtgcagtgtgtactgtcagcaatgattccagcatccaaggcaactgtttcatctacaatgtcaagttctctggtttgaactttcctcccaatggacctgttatgcaaaagaagacacagggctgggaacccaacactgagcgtctctttgcacgagatggagtgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgagatttccatcgcacgcaaacctgtggtcgcctgacgttttttcagagtcaaatcaaggcacaaa

SEQ ID NO: 62 - protein, 220 aa, Porites Murrayensis
SVIAKQMTYKVYMSGTVNGHYFEVQGDGKGKPYEGEQTVKLTVTKGGPLPFAWDILSPQSQYGSIPFTKYPEDIPDYVKQSFPEGYTWERIMNFKDGAVCTVSNDSSIQGNCFIYNVKFSGLNFPPNGPVMQKKTQGWEPNTERLFARDGVLIGNNFMALKLEGGGHYLCEFKSTYKAKKPVMMPGYHYVDRKLDVTNHNKDYTSVEQCEISIARKPVVA

SEQ ID NO: 63 - DNA, 660 bp, Platygyra sp.
  CDS (1)...(660)
tccgttatcgctaaacagatgacctacaaggtttatatgtcaggcacggtcaatggacactactttgaggtcgaaggcgatagaaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 64 - protein, 220 aa, Platygyra sp.
SVIAKQMTYKVYMSGTVNGHYFEVEGDRKGKPYEGEQTVKLTVTKGGPLPFAWDILSPQCQYGNIPFTKYPEDVPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCGFKTTYKAKKPVKMPGYHYVDRKLDVTNHNKDYISVEQCEISIARKPVVA

SEQ ID NO: 65 - DNA, 660 bp, Platygyra sp.
  CDS (1)...(660)
tccgttatcgctaaacagatgacctacaaggtttatatgtcaggcacggtcaatggacactactttgaggtcgaaggcgatgggaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 66 - protein, 220 aa, Platygyra sp.
SVIAKQMTYKVYMSGTVNGHYFEVEGDGKGKPYEGEQTVKLTVTKGGPLPFAWDILSPQCQYGNIPFTKYPEDVPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCGFKTTYKAKKPVKMPGYHYVDRKLDVTNHNKDYISVEQCEISIARKPVVA

SEQ ID NO: 67 - DNA, 660 bp, Platygyra sp.
  CDS (1)...(660)
tccgttatcgctaaacagatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaacttccattgcacgcaaacctgtggtcgcc

SEQ ID NO: 68 - protein, 220 aa, Platygyra sp.
SVIAKQMTYKVYMSGTVNGHYFEVEGDGKGKPYEGEQTVKLTVTKGGPLPFAWDILSPQCQYGNIPFTKYPEDVPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCGFKTTYKAKKPVKMPGYHYVDRKLDVTNHNKDYISVEQCETSIARKPVVA

SEQ ID NO: 69 - DNA, 660 bp, Platygyra sp.; identical to SEQ ID NO: 25
  CDS (1)...(660)

SEQ ID NO: 70 - protein, 220 aa, Platygyra sp.; identical to SEQ ID NO: 26

SEQ ID NO: 71 - DNA, 660 bp, Platygyra sp.; identical to SEQ ID NO: 39
  CDS (1)...(660)

SEQ ID NO: 72 - protein, 220 aa, Platygyra sp.; identical to SEQ ID NO: 40

SEQ ID NO: 73 - DNA, 660 bp, Platygyra sp.
  CDS (1)...(660)
tccgttatcgctaaacagatgacctacaaggtttatatgtcaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggtagctgtttcatctacaatgtcaagttctctggtttgaactttcctcccaatggacctgttatgcaaaagaagacacagggctgggaacccaacactgagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtggaatttccattgcacgcaaacctgtggtcgcc

SEQ ID NO: 74 - protein, 220 aa, Platygyra sp.
SVIAKQMTYKVYMSGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGSCFIYNVKFSGLNFPPNGPVMQKKTQGWEPNTERLFARDGMLIGNNFMALKLEGGGHYLCEFKSTYKAKKPVMMPGYHYVDRKLDVTNHNKDYTSVEQCGISIARKPVVA

SEQ ID NO: 75 - DNA, 660 bp, Pavona decussaca
  CDS (1)...(660)
tccgttatcgctaaacagatgacctacaaggttaatatgtcaggcacggtcaatggacactactttgaggttgaaggcgatggaaaaggaaagccttacgagggggagcagacggtaaagctcactgtcaccgagggcggacctctgccatttgcttgggatattctatcaccacagagtcagtacggaagcgtaccattcaccaagtaccctgaagacatccctgactatgtaaagcagtcattccctgagggatatacatgggagaggatcatgaacttcgaagatggtgcagtgtgtactgtcagcaatgattccagcatccaaggtaactgtttcatctacaatgtcaagttctctggtttgaactttcctcccaatggacctgttatgcaaaagaagacacagggctgggtacccaacactgagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgaaatttccattgcacgcaaacctgtggtcgcc

SEQ ID NO: 76 - protein, 220 aa, Pavona decussaca
SVIAKQMTYKVNMSGTVNGHYFEVEGDGKGKPYEGEQTVKLTVTEGGPLPFAWDILSPQSQYGSVPFTKYPEDIPDYVKQSFPEGYTWERIMNFEDGAVCTVSNDSSIQGNCFIYNVKFSGLNFPPNGPVMQKKTQGWVPNTERLFARDGMLIGNNFMALKLEGGGHYLCEFKSTYKAKKPVMMPGYHYVDRKLDVTNHNKDYTSVEQCEISIARKPVVA

SEQ ID NO: 77 - DNA, 660 bp, Pavona decussaca
  CDS (1)...(660)
tccgttatcgctaaacagatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatgggcctgtgatgcagaagaagacacagggctgggaaccccactctgagcgtctctttgcacggggtggaatgctgataggaaacaactttatggctccgaagttagaaggaggcggtcactatttgtgtgaattcaaaactacttacaaggcaaagaagcctgtgaagatgccggggtatcattatgttgaccgcaaactggatgtaaccaatcacaacaaggattacacttccgttgagcagtgtgaaatctccattgcacgcaaacctgtggtcgcc

SEQ ID NO: 78 - protein, 220 aa, Pavona decussaca; identical to SEQ ID NO: 30

SEQ ID NO: 79 - DNA, 660 bp, Pavona decussaca
  CDS (1)...(660)
tccgttatcgctaaacagatgacctacaaggtttatatgtcaggcacggtcaatggacactactttgaggttgaaggcgatggaaaaggaaagccttacgagggggagcagacggtaaagctcactgtcaccgagggcggacctctgccatttgcttgggatattctatcaccacagagtcagtacggaagcgtaccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctctgcacgagatggaatgctaataggaaacaactttatggctctgaagttagaaggaggtggtcactatttgtgtgaattcaaatctacttacaaggcaaggaagcctgtgaagatgccagggtatcactatgttgaccgcaaactggatgtaaccaatcacaacaaggattacacttccgttgagcagcgtgaaatttccattgcacgcaaacctgtggtcgcc

SEQ ID NO: 80 - protein, 220 aa, Pavona decussaca
SVIAKQMTYKVYMSGTVNGHYFEVEGDGKGKPYEGEQTVKLTVTEGGPLPFAWDILSPQSQYGSVPFTKYPEDIPDYVKQSFPEGYTWERIMNFEDGAVCTVSNDSSIQGNCFIYHVKFSGLNFPPNGPVMQKKTQGWEPNTERLSARDGMLIGNNFMALKLEGGGHYLCEFKSTYKARKPVKMPGYHYVDRKLDVTNHNKDYTSVEQREISIARKPVVA

SEQ ID NO: 81 - DNA, 660 bp, Montipora sp.
  CDS (1)...(660)
tccgttatcgctaaacagatgacctacaaggtttatatgtcaggcacggtcaatggacactactttgaggctgaaggcgatggaaaaggaaagccttacgagggggaccagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggtaactgtttcatctacaatgtcaagttctctggtttgaactttcctcccaatggacctgttatgcaaaagaagacacagggctgggaacccaacactgagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgaaattcccattgcacgcaaacctgtggtcgcc

SEQ ID NO: 82 - protein, 220 aa, Montipora sp.
SVIAKQMTYKVYMSGTVNGHYFEAEGDGKGKPYEGDQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLFARDGMLIGNNFMALKLEGGGHYLCEFKSTYKAKKPVMMPGYHYVDRKLDVTNHNKDYTSVEQCEIPIARKPVVA

SEQ ID NO: 83 - DNA, 660 bp, Montipora sp.; identical to SEQ ID NO: 39
  CDS (1)...(660)

SEQ ID NO: 84 - protein, 220 aa, Montipora sp.; identical to SEQ ID NO: 40

SEQ ID NO: 85 - DNA, 660 bp, Montipora sp.; identical to SEQ ID NO: 39
  CDS (1)...(660)

SEQ ID NO: 86 - protein, 220 aa, Montipora sp.; identical to SEQ ID NO: 40

SEQ ID NO: 87 - DNA, 765 bp, Acropora aspera
gcgaccacaggtttgcgtgtaatggacatcagcatctctttcacggaaggagctacttaagaaacgtttgcgaatcgttgttctgcgctacttattctcaatatgagtgtgatcgctacacaaatgacctacaaggtttatatgtcaggcacggtcaatggacactactttgaggtcgaaggcgatggaaaaggaaagccttacgagggggagcagacggtaaggctggctgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaaccccactctgagcgtctctttgcacgagatggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattacacgcaaacctgtggtcgcc

SEQ ID NO: 88 - protein, 254 aa, Acropora aspera
ATTGLRVMDISISFTEGATETFANRCSALLILNMSVIATQMTYKVYMSGTVNGHYFEVEGDGKGKPYEGEQTVRLAVTKGGPLPFAWDILSPQCQYGSIPFTKYPEDIPDYVKQSFPEGFTWERIMNFEDGAVCTVSNDSSIQGNCFIYHVKFSGLNFPPNGPVMQKKTQGWEPHSERLFARDGMLIGNNFMALKLEGGGHYLCEFKTTYKAKKPVKMPGYHYVDRKLDVTNHNKDYTSVEQCEISITRKPVVA

SEQ ID NO: 89 - DNA, 765 bp, Acropora aspera; identical to SEQ ID NO: 87

SEQ ID NO: 90 - protein, 254 aa, Acropora aspera; identical to SEQ ID NO: 88

SEQ ID NO: 91 - DNA, 660 bp, Acrospora aspera
  CDS (1)...(660)
agtgggatcgctacacaaatgacctacaaggtttatatgtcaggcacggtcaatggacactactttgaggtcgaaggcgatggaaaaggaaagccttacgagggggagcagacggtaaggctggctgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaaccccactctgagcgtctctttgcacgagacggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaatcaatcacaacaaggattacacttccgttgagcagtgtgaaatttccattgcacgcaaacctgtggtcgcc

SEQ ID NO: 92 - protein, 220 aa, Acrospora aspera
SGIATQMTYKVYMSGTVNGHYFEVEGDGKGKPYEGEQTVRLAVTKGGPLPFAWDILSPQCQYGSIPFTKYPEDIPDYVKQSFPEGFTWERIMNFEDGAVCTVSNDSSIQGNCFIYHVKFSGLNFPPNGPVMQKKTQGWEPHSERLFARDGMLIGNNFMALKLEGGGHYLCEFKTTYKAKKPVKMPGYHYVDRKLDVINHNKDYTSVEQCEISIARKPVVA

SEQ ID NO: 93 - DNA, 660 bp, Acropora aspera
  CDS (1)...(660)
agtgtgatcgctacacaaatgacctacaaggtttatatgtcaggcacggtcaatggacactactttgaggtcgaaggcgatggaaaaggaaagccttacgagggggagcagacggtaaggctggctgtcaccaagggcggacctctgccatttgcctgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaaccccactctgagcgtctctttgcacgagacggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaatcaatcacaacaaggattacacttccgttgagcagtgtgaaatttccattgcacgcaaccctgtggtcgcc

SEQ ID NO: 94 - protein, 220 aa, Acropora aspera
SVIATQMTYKVYMSGTVNGHYFEVEGDGKGKPYEGEQTVRLAVTKGGPLPFAWDILSPQCQYGSIPFTKYPEDIPDYVKQSFPEGFTWERIMNFEDGAVCTVSNDSSIQGNCFIYHVKFSGLNFPPNGPVMQKKTQGWEPHSERLFARDGMLIGNNFMALKLEGGGHYLCEFKTTYKAKKPVKMPGYHYVDRKLDVINHNKDYTSVEQCEISIARNPVVA

SEQ ID NO: 95 - DNA, 663 bp, Acrospora aspera
  CDS (1)...(663)
atgagtgtgatcgctacacaaatgacctacaaggtttatatgtcaggcacggtcaatggacactactttgaggtcgaaggcgatggaaaaggaaagccttacgagggggagcagacggtaaggctggctgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaaccccactctgagcgtctctttgcacgagatggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattacacgcaaacctgtggtcgcc

SEQ ID NO: 96 - protein, 221 aa, Acrospora aspera
MSVIATQMTYKVYMSGTVNGHYFEVEGDGKGKPYEGEQTVRLAVTKGGPLPFAWDILSPQCQYGSIPFTKYPEDIPDYVKQSFPEGFTWERIMNFEDGAVCTVSNDSSIQGNCFIYHVKFSGLNFPPNGPVMQKKTQGWEPHSERLFARDGMLIGNNFMALKLEGGGHYLCEFKTTYKAKKPVKMPGYHYVDRKLDVTNHNKDYTSVEQCEISITRKPVVA

SEQ ID NO: 97 - DNA, 663 bp, Acropora aspera; identical to SEQ ID NO: 95
  CDS (1)...(663)

SEQ ID NO: 98 - protein, 221 aa, Acropora aspera; identical to SEQ ID NO: 96

SEQ ID NO: 99 - DNA, 663 bp, Acanthastrea sp.
atgagtgtgatcgctacacaaatgacctacaaggtttatatgtcaggcacggccaatggacactactttgaggttgaaggcgatggaaaaggaaagccttacgaaggggagcagacggtaaggctcattgtcacaaagggcggacctctgccatttgcttgagatattttatcaccacagtatcagtacggaagcataccattcaccaagtaccctgaagacatccctgactatgtaaagcagtcattcccggaaggatatacatgggagaggatcatgaactttgaagatggtgcagtgtgtactgtcagcaatgattccagcatccaaggcaactgtttcatctaccatgtcaagttctctggtttgaactttcctcccaatggacctgtgatgcagaagaagacacagggctgggaacccaacactgagcgtctctttgcacgagatggaatgctgataggaaacaactttatggctctgaagttagaaggaggcggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcacgcaaacctgtggtcgcc

SEQ ID NO: 100 - protein, 220 aa, Acanthastrea sp.
MSVIATQMTYKVYMSGTANGHYFEVEGDGKGKPYEGEQTVRLIVTKGGPLPFADILSPQYQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPVVA

SEQ ID NO: 101 - DNA, 663 bp, Acanthastrea sp.; identical to SEQ ID NO: 99

SEQ ID NO: 102 - protein, 220 aa, Acanthastrea sp.; identical to SEQ ID NO: 100

SEQ ID NO: 103 - DNA, 663 bp, Acanthastrea sp.
  CDS (1)...(663)
atgagtgtgatcgctacacaaatgacctacaaggtttatatgtcaggcacggccaatggacactactttgaggttgaaggcgatggaaaaggaaagccttacgaaggggagcagacggtaaggctcactgtcacaaagggcggacctctgccatttgcttgggatattttatcaccacagtatcagtacggaagcataccattcaccaagtaccctgaagacatccctgactatgtaaagcagtcattcccggaaggatatacatgggagaggatcatgaactttgaagatggtgcagtgtgtactgtcagcaatgattccagcatccaaggcaactgtttcatccaccatgtcaagttctctggtttgaactttcctcccaatggacctgtgatgcagaagaagacacagggctgggaacccaacactgagcgtctctttgcacgagatggaatgctgataggaaacaactttatggctctgaagttagaaggaggcggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcacgcaaacctgtggtcgcc

SEQ ID NO: 104 - protein, 221 aa, Acanthastrea sp.
MSVIATQMTYKVYMSGTANGHYFEVEGDGKGKPYEGEQTVRLTVTKGGPLPFAWDILSPQYQYGSIPFTKYPEDIPDYVKQSFPEGYTWERIMNFEDGAVCTVSNDSSIQGNCFIHHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPVVA

SEQ ID NO: 105 - DNA, 659 bp, Acanthastrea sp.
agtgtgatcgctacacaaatgacctacaaggtttatatgtcaggcacggtcaatggacactactttgaggtcgaaggcgatggaaaaggaaagcctacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaaggagacacagggctgggaaccccactctgagcgtctctttgcacggggtggaatgctgataggaaacaactttgtggctctgaagttagaaggaggcggtcactatttgtgtggattcaaaactacttacaaggcaaagaaacctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 106 - protein, 208 aa, Acanthastrea sp.
SVIATQMTYKVYMSGTVNGHYFEVEGDGKGKPTRGSRRSSLSPRADLCHLLGIFYHHSVSTETYHSPSTLKTSLTMSSHSRRDLHGRGSTLKMVQCVLSAMIPASKATVSPTMSSSLVTFLPMDLCRRRHRAGNPTLSVSLHGVECETTLWLSKEAVTICVDSKLLTRQRNLRCQGIIMLTANWMPITTRITFPLSSVKFPLHANLWS

SEQ ID NO: 107 - DNA, 663 bp, Caulastrea sp.
  CDS (1)...(663)
atgagtgtgatcgctacacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaaatataccattcaccaagtaccctgaagacgtccctgactatgtaaagcggtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 108 - protein, 221 aa, Caulastrea sp.
MSVIATQMTYKVYMSGTVNGHYFEVEGDGKGKPYEGEQTVKLTVTKGGPLPFAWDILSPQCQYGNIPFTKYPEDVPDYVKRSFPEGFTWERIMNFEDGAVCTVSNDSSIQGNCFTYHVKFSGLNFPPNGPVMQKKTQGWEPHSERLFARGGMLIGNNFMALKLEGGGHYLCGFKTTYKAKKPVKMPGYHYVDRKLDVTNHNKDYISVEQCEISIARKPVVA

SEQ ID NO: 109 - DNA, 663 bp, Caulastrea sp.
  CDS (1)...(663)
atgagtgtgatcgctacacaaatgacctacaaggtttatatgtcgggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctccttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 110 - protein, 221 aa, Caulastrea sp.
MSVIATQMTYKVYMSGTVNGHYFEVEGDGKGKPYEGEQTVKLTVTKGGPLPFAWDILSPQCQYGNIPFTKYPEDVPDYVKQSFPEGFTWERIMNFEDGAVCTVSNDSSIQGNCFTYHVKFSGLNFPPNGPVMQKKTQGWEPHSERLLARGGMLIGNNFMALKLEGGGHYLCGFKTTYKAKKPVKMPGYHYVDRKLDVTNHNKDYISVEQCEISIARKPVVA

SEQ ID NO: 111 - DNA, 660 bp, Caulastrea sp.
  CDS (1)...(660)
agtgtgatcgctacacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcttgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 112 - protein, 220 aa, Caulastrea sp.
SVIATQMTYKVYMSGTVNGHYFEVEGDGKGKPYEGEQTVKLTVTKGGPLPFAWDILSPQCQYGNIPFTKYPEDVPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCGFKTTYKAKKLVKMPGYHYVDRKLDVTNHNKDYISVEQCEISIARKPVVA

SEQ ID NO: 113 - DNA, 660 bp, Caulastrea sp.
agtgtgatcgctacacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgtagaagaagacacagggctgggaaccccactctgagcgtctctttgcacggggtggaatgctgataggaaacaactttatggctctgaagttagaaggaggtggtcactatttgtgtgaattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 114 - protein, 219 aa, Caulastrea sp.
SVIATQMTYKVYMSGTVNGHYFEVEGDGKGKPYEGEQTVKLTVTKGGPLPFAWDILSPQCQYGNIPFTKYPEDVPDYVKQSFPEGFTWERIMNFEDGAVCTVSNDSSIQGNCFTYHVKFSGLNFPPNGPVMKKTQGWEPHSERLFARGGMLIGNNFMALKLEGGGHYLCEFKTTYKAKKPVKMPGYHYVDRKLDVTNHNKDYISVEQCEISIARKPVVA

SEQ ID NO: 115 - DNA, 663 bp, Acropora nobilis
  CDS (1)...(663)
atgagtgtgatcgctacacaaatgacctacaaggtttatatgtcaggcacggtcaatggacactactttgaggttgaaggcgatggaaaaggaaagccttacgaaggggagcagacggtaaggctcactgtcacaaagggcggacctctgccatttgcttgggatattttatcaccacagtatcagtacggaagcataccattcaccaagtaccctgaagacatccctgactatgtaaagcagtcattcccggaaggatatacatgggagaggatcatgaactttgaagatggtgcagtgtgtactgtcagcaatgattccagcatccaaggcaactgtttcatctaccatgtcaagttctctggtttgaactttcctcccaacggacctgtgatgcagaagaagacacagggctgggaacccaacactgagcgtctcttagcacgagatggaatgctgataggaaacaactttatggctctgaagttagaaggaggcggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcacgcaaacctgtggtcgcc

SEQ ID NO: 116 - protein, 221 aa, Acropora nobilis
MSVIATQMTYKVYMSGTVNGHYFEVEGDGKGKPYEGEQTVRLTVTKGGPLPFAWDILSPQYQYGSIPFTKYPEDIPDYVKQSFPEGYTWERIMNFEDGAVCTVSNDSSIQGNCFIYHVKFSGLNFPPNGPVMQKKTQGWEPNTERLLARDGMLIGNNFMALKLEGGGHYLCEFKSTYKAKKPVKMPGYHYVDRKLDVTNHNKDYTSVEQCEISIARKPVVA

SEQ ID NO: 117 - DNA, 663 bp, Acropora nobilis
atgagtgtgatcgctacacaaatgacctacaaggtttatatgtcaggcacagtcaatggacactaccttgaggtcgaaggcgatggaaaaggaaagccttacgagggggagcagacggtaaggctcactgtcaccaagggcggacctctgccatttgcttgggatattttgtcaccacagtatcagtacggaagcataccattcaccaagtaccctgaagacatccctgactatgtaaagtagtcattcccggagggatttacatgggacaggatcatggactttgaagatggtgcagtgtgtaccgtcagcaatgattccagcatccaaggcaactgtttcatctaccatgtcaagttctctggtttgaactttcctcccaatggacctgttatgcagaagaagacacagggctgggaacccaacactgagcgtctctttgcacgagatggaatgctgctaggaaacaactttatggctctgaagttagaaggaggtggtcactatttgtgtgaattcaaaactacttacaaggcaaagaagcctgtgaagatgccagggtatcactatgttgaccgcaaactggatgtaaccaatcacaacaaggattacacttccgttgagcagtgtgaaatttccattgcacgcaaacctgtggtcgcc

SEQ ID NO: 118 - protein, 220 aa, Acropora nobilis
MSVIATQMTYKVYMSGTVNGHYLEVEGDGKGKPYEGEQTVRLTVTKGGPLPFAWDILSPQYQYGSIPFTKYPEDIPDYVKSFPEGFTWDRIMDFEDGAVCTVSNDSSIQGNCFIYHVKFSGLNFPPNGPVMQKKTQGWEPNTERLFARDGMLLGNNFMALKLEGGGHYLCEFKTTYKAKKPVKMPGYHYVDRKLDVTNHNKDYTSVEQCEISIARKPVVA

SEQ ID NO: 119 - DNA, 660 bp, Millepora sp.
  CDS (1)...(660)
agtgtgatcgctacacaaatgacctacaaggtttatatgtcaggcacggtcgatggacactactttgaggtcgaaggcgatggaaaaggaaagccttacgagggcgagcagactgtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaaaatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattgcacgcaaacctgtggtcgcc

SEQ ID NO: 120 - protein, 220 aa, Millepora sp.
SVIATQMTYKVYMSGTVDGHYFEVEGDGKGKPYEGEQTVKLTVTKGGPLPFAWDILSPQCQYGSIPFTKYPEDIPDYVKQSFPEGFTWERIMNFENGAVCTVSNDSSIQGNCFTYHVKFSGLNFPPNGPVMQKKTQGWEPHSERLFARGGMLIGNNFMALKLEGGGHYLCEFKTTYKAKKPVKMPGYHYVDRKLDVTNHNKDYTSVEQCEISIARKPVVA

SEQ ID NO: 121 - DNA, 663 bp, Millepora sp.
  CDS (1)...(663)
atgagtgtgatcgctacacaaatgacctacaaggtttatatgtcaggcacggtcgatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattgcacgcaaacctgtggtcgcc

SEQ ID NO: 122 - protein, 221 aa, Millepora sp.
MSVIATQMTYKVYMSGTVDGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCEFKTTYKAKKPVKMPGYHYVDRKLDVTNHNKDYTSVEQCEISIARKPVVA

SEQ ID NO: 123 - DNA, 663 bp, Millepora sp.
  CDS (1)...(663)
atgagtgtgatcgctacacaaatgacctacaaggtttatatgtcaggcacggtcgatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatggttccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttaggaggaggcggtcactatttgtgtgaattcaaaactacttacagggcaaagaagcctgtgaagatgccagggtatcattatgttgaccgcaaactggatgtaaccaatcacaacaaggattacacttccgttgagcagtgtgaaatttccattgcacgcaaacctgtggtcgcc

SEQ ID NO: 124 - protein, 221 aa, Millepora sp.
MSVIATQMTYKVYMSGTVDGHYFEVEGDGKGKPYEGEQTVKLTVTKGGPLPFAWDILSPQCQYGSIPFTKYPEDIPDYVKQSFPEGFTWERIMNFEDGAVCTVSNGSSIQGNCFTYHVKFSGLNFPPNGPVMQKKTQGWEPHSERLFARGGMLIGNNFMALKLGGGGHYLCEFKTTYRAKKPVKMPGYHYVDRKLDVTNHNKDYTSVEQCEISIARKPVVA

SEQ ID NO: 125 - DNA, 660 bp, Millepora sp.
  CDS (1)...(660)
agtgtgatcgctacacaaatgacctacaaggtttatatgtcaggcacggtcgatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattgcacgcaaacctgtggtcgcc

SEQ ID NO: 126 - protein, 220 aa, Millepora sp.
SVIATQMTYKVYMSGTVDGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCEFKTTYKAKKPVKMPGYHYVDRKLDVTNHNKDYTSVEQCEISIARKPVVA

SEQ ID NO: 127 - DNA, 663 bp, Porites murrayensis
  CDS (1)...(663)
atgagtgtgatcgctacacaaatgacctacaaggtttatatgccaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggtaactgtttcatctacaatgtcaagttctctggtttgaactttcctcccaatggacctgttatgcaaaagaagacacagggctgggaacccaacactgagcgtctttatgcacgagatggaatgctgataggaaacaactttatggctctgaagttggaaggaggtggtcattatttgtgtgaattcaaatctacttacaaggcaaagaagcctgtgatgatgcctggatatcactatgttgaccgcaaattggatgtaaccaatcacaacaaggattacacttccgttgagcagtgtgaaatttccattgcacgcaaacctgtggtcgcc

SEQ ID NO: 128 - protein, 221 aa, Porites murrayensis
MSVIATQMTYKVYMPGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLYARDGMLIGNNFMALKLEGGGHYLCEFKSTYKAKKPVMMPGYHYVDRKLDVTNHNKDYTSVEQCEISIARKPVVA

SEQ ID NO: 129 - DNA, 663 bp, Porites murrayensis
  CDS (1)...(663)
atgagtgtgatcgctacacaaatgacctacaaggtttatatgtcaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggtaactgtttcatctacaatgtcaagttctctggtttgaactttcctcccaatggacctgttatgcaaaagaagacacagggttgggaacccaacactgagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgaaatttccattgcacgcaaacctgtggtcgcc

SEQ ID NO: 130 - protein, 221 aa, Porites murrayensis
MSVIATQMTYKVYMSGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLFARDGMLIGNNFMALKLEGGGHYLCEFKSTYKAKKPVMMPGYHYVDRKLDVTNHNKDYTSVEQCEISIARKPVVA

SEQ ID NO: 131 - DNA, 660 bp, Porites murrayensis
  CDS (1)...(660)
agtgtgatcgctacacaaatgacctacaaggtttatatgtcaggcacggtcaatggacactactttgaggtccaaggcgatggaaaaggaaagccttacgagggcgagcagactgtaaagctcactgtcaccaagggcggacctctgccctttgcttgggatattttatcacctcagactcagtacggaagcataccattcaccaagtaccctgacgacatccctgactatgtaaaacagtcattccctgagggatatacatgggagaggatcatgaagtttgaagatggtgcagtgtgtactgtcaccaatgactccagcatgcaaggcaactgtttcatctacaatgtcaagttctctggtttgaactttcctcccaatggacctgttatgcagaagaagacacagggctgggaacccaacactgggcgtctttatgcacgagatggaatgctgataggaaacaactttatggctctgaagttggaaggaggtggtcattatacctgtgaattcaaatctacttacaaggcaaagaagcctgtgatgatgcctggatatcactatgttgaccgcaaattggatgtaaccaatcacaacaaggattacacttccgttgagcagtgtgaaatttccattgcacgcaaacctgtggtcgcc

SEQ ID NO: 132 - protein, 220 aa, Porites murrayensis
SVIATQMTYKVYMSGTVNGHYFEVQGDGKGKPYEGEQTVKLTVTKGGPLPFAWDILSPQTQYGSIPFTKYPDDIPDYVKQSFPEGYTWERIMKFEDGAVCTVTNDSSMQGNCFIYNVKFSGLNFPPNGPVMQKKTQGWEPNTGRLYARDGMLIGNNFMALKLEGGGHYTCEFKSTYKAKKPVMMPGYHYVDRKLDVTNHNKDYTSVEQCEISIARKPVVA

SEQ ID NO: 133 - DNA, 660 bp, Porites murrayensis
  CDS (1)...(660)
agtgtgatcgctacacaaatgacctacaaggtttatatgtcaggcacggtcaatggacactactttgaggttgaaggcgatggacaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggcaactgtttcatctacaatgtcaagttctctggtttgaactttcctcccaatggacctgttatgcaaaagaagacacagggctgggaacccaacactgagcgtctctttgcacgagatggaatgctgataggaaacaactttatggctctgaagtcggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgaaatttccattgcacgcaaacctgtggtcgcc

SEQ ID NO: 134 - protein, 220 aa, Porites murrayensis
SVIATQMTYKVYMSGTVNGHYFEVEGDGQGKPYEGEQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLFARDGMLIGNNFMALKSEGGGHYLCEFKSTYKAKKPVMMPGYHYVDRKLDVTNHNKDYTSVEQCEISIARKPVVA

SEQ ID NO: 135 - DNA, 660 bp, Porites murrayensis
agtgtgatcgctacacaaatgacctacaaggtttatatgtcaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggcaactgtttcatctacaatgtcaagttctctggtttgaactttcctcccaatggacctgttatgcaaaagaagacacagggctgggaacccaacacagagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgaaatttccattgcacgcaaacctgtggtcgcc

SEQ ID NO: 136 - protein, 220 aa, Porites murrayensis
SVIATQMTYKVYMSGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLFARDGMLIGNNFMALKLEGGGHYLCEFKSTYKAKKPVMMPGYHYVDRKLDVTNHNKDYTSVEQCEISIARKPVVA

SEQ ID NO: 137 - DNA, 660 bp, Pink Pocillopora
agtgtgatcgctacacaaatgacctacaaggtttatatgtcaggcacggtcaatggacactactttgaggttgaaggcgatggaaaaggaaagccttacgagggcgagcagactgtaaagctcactgtcaccaagggcggacctctgccgtttgcttgggatattttatcaccacagactcagtacggaagcataccattcaccaagtaccctgaagacattcctgactatgtaaaacagtcattccctgagggatatacatgggagaggatcatgaagtttgaagatggtgcagtatgtactgtcagcaatgattccagcatgcaaggcaactgtttcatctacaatgtcaagttctctggtttgaactttcctcccaatggacctgttatgcagaagaagacacagggctgggaacccaacactgagcgtctttatgcacgagatggaatgctgataggaaacaactttatggctctgaagttggaaggaggtggtcattatacctgtgaattcaaatctacttacaaggcaaagaagcctgtgatgatgcctggatatcactatgttgaccgcaaattggatgtaaccaatcacaacaaggattacacttccgttgagcagtgtgaaatttccattgcacgcaaacctgtggtcgcc

SEQ ID NO: 138 - protein, 220 aa, Pink Pocillapora
SVIATQMTYKVYMSGTVNGHYFEVEGDGKGKPYEGEQTVKLTVTKGGPLPFAWDILSPQTQYGSIPFTKYPEDIPDYVKQSFPEGYTWERIMKFEDGAVCTVSNDSSMQGNCFIYNVKFSGLNFPPNGPVMQKKTQGWEPNTERLYARDGMLIGNNFMALKLEGGGHYTCEFKSTYKAKKPVMMPGYHYVDRKLDVTNHNKDYTSVEQCEISIARKPVVA

SEQ ID NO: 139 - DNA, 663 bp, Pink Pocillapora
atgagtgtgatcgctacacaaatgacctacaaggtttatatgtcaggcacggtcaatggacactactttgaggtcgaaggcgatggaaaaggaaagccttacgagggggagcagacggtaaggctggctgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaaccccactctgagcgtctctttgcacgagatggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattgcacgcaaacctgtggtcgcc

SEQ ID NO: 140 - protein, 220 aa, Pink pocillapora
SVIATQMTYKVYMSGTVNGHYFEVEGDGKGKPYEGEQTVRLAVTKGGPLPFAWDILSPQCQYGSIPFTKYPEDIPDYVKQSFPEGFTWERIMNFEDGAVCTVSNDSSIQGNCFIYHVKFSGLNFPPNGPVMQKKTQGWEPHSERLFARDGMLIGNNFMALKLEGGGHYLCEFKTTYKAKKPVKMPGYHYVDRKLDVTNHNKDYTSVEQCEISIARKPVVA

SEQ ID NO: 141 - DNA, 663 bp, Pink Pocillapora; identical to SEQ ID NO: 139

SEQ ID NO: 142 - protein, 220 aa, Pink Pocillapora; identical to SEQ ID NO: 140

SEQ ID NO: 143 - DNA, 660 bp, Pink Pocillapora
agtgtgatcgctacacaaatgacctacaaggtttatatgtcaggcacggtcaatggacactactttgaggttgaaggcgatggaaaaggaaagccttacgagggggagcagacggtaaagctcactgtcaccaagggcggacctctgccatttgcttgggatattctatcaccacagagtcagtacggaagcataccattcaccaagtaccctgaagacatccctgactatgtaaagcagtcattccctgagggatatacatgggagaggatcatgaacttcgaagatggtgcagtgtgtactgtcagcaatgattccagcatccaaggcaactgtttcatctacaatgtcaagttctctggtttgaactttcctcccaatggacctgttatgcaaaagaagacacagggctgggaacccaacactgagcgtctctttgcacgagatggaatgctgataggaaacaactttatggctctgaagttggaaggaggtggtcattatttgtgtgaattcaaatctacttacaaggcaaagaagcctgtgatgatgccagggtatcactatgttgaccgcaaattggatgtaaccaatcacaacaaggattacacttccgttgagcagtgtgagatttccattgcacgcaaacctgtggtcgcc

SEQ ID NO: 144 - protein, 220 aa, Pink Pocillapora; identical to SEQ ID NO: 136

SEQ ID NO: 145 - DNA, 663 bp, Platygyra sp.
atgagtgtgatcgctacacaaatgacctacaaggtttatatgtcaggcacggtcaatggacactactttgaggtcgaaggcgatggaaaaggaaagccttacgagggggagcggacggtaaagctcactgtcaccaagggcggacctctgccgtttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 146 - protein, 221 aa, Platygyra sp.
MSVIATQMTYKVYMSGTVNGHYFEVEGDGKGKPYEGERTVKLTVTKGGPLPFAWDILSPQCQYGNIPFTKYPEDVPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCGFKTTYKAKKPVKMPGYHYVDRKLDVTNHNKDYISVEQCEISIARKPVVA

SEQ ID NO: 147 - DNA, 663 bp, Platygyra sp.
atgagtgtgatcgctacacaaatgacctacaaggtttatatgtcaggcacggtcaatggacactactttgaggtcgaaggcgatggaaaaggaaagccttacgagggggagcagacggtaaggctcactgtcaccaagggcggacctctgccatttgcttgggatattttgtcaccacagtatcagtacggaagcataccattcaccaagtaccctgaagacatccctgactatgtaaagtagtcattcccggagggatttacatgggacaggatcatgaactttgaagatggtgcagtgtgtaccgtcagcaatgattccagcatccaaggcaactgtttcatctaccatgtcaagttctctggtttgaactttcctcccaatggacctgttatgcagaagaagacacagggctgggaacccaacactgagcgtctccttgcacgagatggaatgctgctaggaaacaactttatggctctgaagttagaaggaggtggtcactatttgtgtgaattcaaaactacttacaaggcaaagaagcctgtgaagatgccagggtatcactatgttgaccgcaaactggatgtaaccaatcacaacaaggattacacttccgttgagcggtgtgaaatttccattgcacgcaaacctgtggtcgcc

SEQ ID NO: 148 - protein, 220 aa, Platygyra sp.
MSVIATQMTYKVYMSGTVNGHYFEVEGDGKGKPYEGEQTVRLTVTKGGPLPFAWDILSPQYQYGSIPFTKYPEDIPDYVKSFPEGFTWDRIMNFEDGAVCTVSNDSSIQGNCFIYHVKFSGLNFPPNGPVMQKKTQGWEPNTERLLARDGMLIGNNFMALKLEGGGHYLCEFKTTYKAKKPVKMPGYHYVDRKLDVTNHNKDYTSVERCEISIARKPVVA

SEQ ID NO: 149 - DNA, 660 bp, Platygyra sp.
agtgtgatcgctacacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcccgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 150 - protein, 220 aa, Platygyra sp.
SVIATQMTYKVYMSGTVNGHYFEVEGDGKGKPYEGEQTVKLTVTKGGPLPFAWDILSPQSQYGSIPFTKYPEDIPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCGFKTTYKAKKPVKMPGYHYVDRKLDVTNHNKDYISVEQCEISIARKPVVA

SEQ ID NO: 151 - DNA, 660 bp, Platygyra sp.
agtgtgatcgctacacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaaacataccattcaccaagtaccctgaagacgtccctgactatgtaaagcagtcattcccggagggatttacatgggaggggatcatgaactttgaagatggtgcagtgtgtactgtcagcaatgattccagcatccaaggcaactgtttcacctaccatgtcaagttctctggtttgaactttcctcccaatggacctgtgatgcagaagaagacacagggctgggaaccccactctgagcgtctctttgcacggggtggaatgctgataggaaacaactttatggctctgaagttagaaggaggcggtcactatttgtgtggattcaaaactacttacaaggcaaagaagcctgtgaagatgccagggtatcattatgttgaccgcaaactggatgtaaccaatcacaacaaggattacatttccgttgagcagtgtgaaatttccattgcacgcaaacctgtggtcgcc

SEQ ID NO: 152 - protein, 220 aa, Platygyra sp.
SVIATQMTYKVYMSGTVNGHYFEVEGDGKGKPYEGEQTVKLTVTKGGPLPFAWDILSPQCQYGNIPFTKYPEDVPDYVKQSFPEGFTWEGIMNFEDGAVCTVSNDSSIQGNCFTYHVKFSGLNFPPNGPVMQKKTQGWEPHSERLFARGGMLIGNNFMALKLEGGGHYLCGFKTTYKAKKPVKMPGYHYVDRKLDVTNHNKDYISVEQCEISIARKPVVA

SEQ ID NO: 153 - DNA, 660 bp, Pavona decussata
agtgtgatcgctacacaaatgacctacaaggtttatatgtcaggcacggtcaatggacactactttgaggttgaaggcgatggaaaaggagagccttacgagggggagcagacggtaaggctcactgtcacaaagggcggacctctgccatttgcttgggatattttatcaccacagtatcagtacggaagcataccattcaccaagtaccctgaagacatccctgactatgtaaagcagtcattcccggaaggatatacatgggagaggatcatgaactttgaagatggtgctgtgtgtactgtcagcaatgattccagcatccaaggcaactgtttcatctaccatgtcaagttctctggtttgaactttcctcccaatggacctgtgacgcagaagaagacacagggctgggaacccaacactgagcgtctctttgcacgagatggaatgctgataggaaacaactttatggctctgaagttagaaggaggcggtcactatttgtgtgaattcaaatcgacttacaaggcaaagaagactgtgaagatgccagggtatcactatgttgaccgcaaactggatgtaaccaatcacaacaaggattacacttccgttgagcagtgtgaaatttccattgcacgcaaacctgtggtcgcc

SEQ ID NO: 154 - protein, 220 aa, Pavona decussata
SVIATQMTYKVYMSGTVNGHYFEVEGDGKGKPYEGEQTVRLTVTKGGPLPFAWDILSPQYQYGSIPFTKYPEDIPDYVKQSFPEGYTWERIMNFEDGAVCTVSNDSSIQGNCFIYHVKFSGLNFPPNGPVTQKKTQGWEPNTERLFARDGMLIGNNFMALKLEGGGHYLCEFKSTYKAKKTVKMPGYHYVDRKLDVTNHNKDYTSVEQCEISIARKPVVA

SEQ ID NO: 155 - DNA, 663 bp, Pavona decussata
atgagtgtgatcgctacacaaatgacctacaaggtttatatgtcaggcacggtcaatggacactactttgaggttgaaggcgatggaaaaggaaagccttacgagggggagcagacggtaaggctcactgtcacaaagggcggacctctgccatttgcttgggatattttatcaccacagtatcagtacggaagcataccattcaccaagtaccctgaagacatccctgactatgtatagcagtcattcccggaaggatatacatgggagaggatcatgaactttgaagatggtgctgtgtgtactgtcagcaatgattccagcatccaaggcaactgtttcatctaccatgtcaagttttctggtttgaactttcctcccaatggacctgtgatgcagaagaagacacagggctgggaacccaacactgagcgtctctttgcacgagatggattgctgataggaaacaactttatggctctgaagttagaagaaggcggtcactatttgtgtgaattcaaatcgacttacaaggcaaagaagactgcgaagatgccagggtatcactatgttgaccgcaaactggatgtaaccaatcacaacaaggattacacttccgttgagcagtgtgaaatttccattgcacgcaaacctgtggtcgcc

SEQ ID NO: 156 - protein, 220 aa, Pavona decussata
MSVIATQMTYKVYMSGTVNGHYFEVEGDGKGKPYEGEQTVRLTVTKGGPLPFAWDILSPQYQYGSIPFTKYPEDIPDYVQSFPEGYTWERIMNFEDGAVCTVSNDSSIQGNCFIYHVKFSGLNFPPNGPVMQKKTQGWEPNTERLFARDGLLIGNNFMALKLEGGGHYLCEFKSTYKAKKTAKMPGYHYVDRKLDVTNHNKDYTSVEQCEISIARKPVVA

SEQ ID NO: 157 - DNA, 663 bp, Pavona decussata
atgagtgtgatcgctacacaagtgacctacaaggtttatatgtcaggcacggtcaatggacactactttgaggttgaaggcgatggaaaaggaaagccttacgagggggagcaaacggtaaggctcactgtcacaaagggcggacctctgccatttgcttgggatattttatcaccacagtatcagtacggaagcataccattcaccaagtaccctgaagacatccctgactatgtaaagcagtcattcccggaaggatatacatgggagaggatcatgaactttgaagatggtgctgtgtgtactgtcagcaatgattccagcatccaaggcaactgtttcatctaccatgtcaagttttctggtttgaactttcctcccaatggacctgtgatgcagaagaagacacagggctgggaacccaacactgagcgtctctttgcacgagatggaatgctgataggaaacaactttatggctctgaagttagaaggaggcggtcactatttgtgtgaattcaaatcgacttacaaggcaaagaagactgtgaagatgccagggtatcactatgttgaccgcaaactggatgtaaccaatcacaacaaggattacacttccgttgagcagtgtgaaatttccattgcacgcaaacctgtggtcgcc

SEQ ID NO: 158 - protein, 221 aa, Pavona decussata
MSVIATQVTYKVYMSGTVNGHYFEVEGDGKGKPYEGEQTVRLTVTKGGPLPFAWDILSPQYQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKTVKMPGYHYVDRKLDVTNHNKDYTSVEQCEISIARKPVVA

SEQ ID NO: 159 - DNA, 660 bp, Pavona decussata
agtgtgatcgctacacaaatgacctacaaggtttatatgtcaggcacggtcaatggacactactttgaggttgaaggcgatggaaaaggaaagccttacgagggggagcagacggtaaggctcactgtcacaaagggcggacctctgccatttgcttgggatattttatcaccacagtatcagtacggaagcataccattcaccaagtaccctgaagacatccctgactatgtaaagcagtcattcccggaaggatatacatgggaggggatcatgaactttgaagatggtgctgtgtgtactgtcagcaatgattccagcatccaaggcaactgtttcatctaccatgtcaagttctctggtttgaactttcctcccaatggacctgtgatgcagaagaagacacagggctgggaacccaacactgagcgtctctttgcacgagatggaatgctgataggaaacaactttatggctctgaagttagaaggaggcggtcactatttgtgtgaattcaaatcgacttacaaggcaaagaagactgtgaagatgccagggtatcactatgttgaccgcaaactggttgtaaccaatcacaacaaggattacacttccgttgagcagtgtgaaatttccattgcacgcaaacctgtggtcgcc

SEQ ID NO: 160 - protein, 220 aa, Pavona decussata
SVIATQMTYKVYMSGTVNGHYFEVEGDGKGKPYEGEQTVRLTVTKGGPLPFAWDILSPQYQYGSIPFTKYPEDIPDYVKQSFPEGYTWEGIMNFEDGAVCTVSNDSSIQGNCFIYHVKFSGLNFPPNGPVMQKKTQGWEPNTERLFARDGMLIGNNFMALKLEGGGHYLCEFKSTYKAKKTVKMPGYHYVDRKLVVTNHNKDYTSVEQCEISIARKPVVA

SEQ ID NO: 161 - DNA, 663 bp, Montipora sp.
atgagtgtgatcgctacacaaatgacctacaaggtttatatgtcaggcacggtcaatggacactactttgaggttgaaggcgatggaaaaggaaagccttacgaaggggagcagacggtaaggctcactgtcacaaagggcggacctctgccatttgcttgggatattttatcaccacagtatcagtacggaagcataccattcaccaagtaccctgaagacatccctgactatgtaaagcagtcattcccggaaggatatacatgggagaggatcatgaactttgaagatggtgcagtgtgtgctgtcagcaatgattccagcatccaaggcaactgtttcatctaccatgtcaagttctctggtttgaactttcctcccaatggacctgtgatgcaaaagaagacacagggctgggaacccaacactgagcgtctctttgcacgagatggaatgctgataggaaacaactttatggctctgaagttagaaggaggcggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcacgcaaacctgtggtcgcc

SEQ ID NO: 162 - protein, 221 aa, Montipora sp.
MSVIATQMTYKVYMSGTVNGHYFEVEGDGKGKPYEGEQTVRLTVTKGGPLPFAWDILSPQYQYGSIPFTKYPEDIPDYVKQSFPEGYTWERIMNFEDGAVCAVSNDSSIQGNCFIYHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPVVA

SEQ ID NO: 163 - DNA, 663 bp, Montipora sp.
atgagtgtgagcgctacacaaatgacctacaaggtttatatgtcaggcacggtcaatggacactactttgaggttgaaggcgatggaaaaggaaagccttacgaaggggagcagacggtaaggctcactgtcacaaagggcggacctctgccatttgcttgggatattttatcaccacagtatcagtacggaagcataccattcaccaagtaccctgaagacatccctggctatgtaaagcagtcattcccggaaggatatacatgggagaggatcatgaactttgaagatggtgcagtgtgtactgtcagcaatgattccagcatccaaggcaactgtttcatctaccatgtcaagttctctggtttgaactttcctcccaatggacctgtgatgcaaaaaaagacacaaggctgggaacccaacactgagcgtctctttgcacgagatggaatgctgataggaaacaactttatggctctgaagttagaaggaggcggtcactatttgtgtgaattcaaatctacttacaaggcaaagaagcctgtgaagatgccagggtatcactatgttgaccgcaaactggatgtaaccaatcacaacaaggattacacttccgttgggcagtgtgaaatttccattgcccccaaacctgtggtcgcc

SEQ ID NO: 164 - protein, 221 aa, Montipora sp.
MSVSATQMTYKVYMSGTVNGHYFEVEGDGKGKPYEGEQTVRLTVTKGGPLPFAWDILSPQYQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPVVA

SEQ ID NO: 165 - DNA, 660 bp, Montipora sp.
agtgtgatcgctacacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtactgtcagcaatgattccagcatccaaggcaactgtttcacctaccatgtcaagttctctggtttgaactttcctcctaatggacctgtgatgcagaagaagacacagggctgggaaccccactctgagcgtctctttgcacggggtggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaaccaatcacaacaaggattacacttccgttgagcagtgtgaaatttccattgcacgcaaacctgtggtcgcc

SEQ ID NO: 166 - protein, 220 aa, Montipora sp.
SVIATQMTYKVYMSGTVNGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLNFPPNGPVMQKKTQGWEPHSERLFARGGMLIGNNFMALKLEGGGHYLCEFKTTYKAKKPVKMPGYHYVDRKLDVTNHNKDYTSVEQCEISIARKPVVA

SEQ ID NO: 167 - DNA, 660 bp, Montipora sp.
agtgtgatcgttacacaaatgacctacaaggtttatatgtcaggcacggtcaatggacactactttgaggttgaaggcgatggaaaaggaaagccttacgaaggggagcagacggtaaggctcactgtcacaaagggcggacccctgccatttgcttgggatattttatcaccacagtatcagtacggaagcataccattcaccaagtaccctgaagacatccctgactatgtaaagcagtcattcccggaaggatatacatgggagaggatcatgaactttgaagatggtgcagtgtgtactgtcagcaatgattccagcatccaaggcaactgtttcatctaccatgtcaagttctctggtttgaactttcctcccaatggacctgtgatgcagaagaagacacagggctgggaacccaacactgagcgtctctttgcacgagatggaatgctgataggaaacaactttatggctctgaagttagaaggaggcggtcactatttgtgtgaattcaaatctacttacaaggcaaagaagcctgtgaagatgccagggtatcactatgttgaccgcaaactggatgtaaccaatcacaacaaggattacacctccgttgagcagtgtgaaatttccattgcacgcaaacctgtggtcgcc

SEQ ID NO: 168 - protein, 220 aa, Montipora sp.
SVIVTQMTYKVYMSGTVNGHYFEVEGDGKGKPYEGEQTVRLTVTKGGPLPFAWDILSPQYQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPVVA

SEQ ID NO: 169 - DNA, 862 bp, Acanthastria sp.
tccgttatcgctaaacagatgaccgcttcaacgttaagttgacaacaggaagcacgacggagactgcagtcccgtacgcgcgaacgggatacctgggatttatcaagagaacagatttcacgcagacagatggagcccggcatgacgcgttatttgtggttggccctcttgaagaaaccatgatattgcgtggtatgaggtatcacccggtagatatcgagaacacagtgacgagatgtcatcgatcaatctgtgaaagtgcggtcttcacgatgacaaacctacttgtggtagcagtggagcttgatgcagatgaacgcgaggcacttgacgtggttccgctggtgacgacatccgtactgaatgaacagcaacttgtcgtaggggtggtggtagtggttgaccctggcgtagtcccgatcaattctcgcggagagaaacaacggatgcatctgagggacgggttcctgggggaccagttggatcctatctacgtggcgtataatatgtagacacctcactgcttagtttcgtaattgaattgtgtcgtagtttttttaaatgacaattaatagacaagtttgaaattgactgtagcgctaggtttaggtataaactagcgtttggtaaggcaattatgacaggaactactgtcacgcgtgacgcgagaccgtcactttacacgcaaacctgtggtcgccattgtgtcgtagtttttttaaatgacaattaatagacaagtttgaaattgactgtagcgctaggtttaggtataaactagcgtttggtaaggcaattatgacaggaactactgtcacgcgtgacgcgagaccgtcactttacacgcaaacctgtggtcgcc

SEQ ID NO: 170 - protein, 208 aa, Acanthastrea sp.
SVIAKQMTASTLSQQEARRRLQSRTRERDTWDLSREQISRRQMEPGMTRYLWLALLKKPYCVVGITRISRTQRDVIDQSVKVRSSRQTYLWQWSLMQMNARHLTWFRWRHPYMNSNLSGWWWLTLASRSILAERNNGCIGTGSWGTSWILSTWRIICRHLTAFRNIVSFFMTINRQVNLRVVTSVWGNYDRNYCHARETVTLHANLWS

SEQ ID NO: 171 - DNA, 701 bp, Green Pocillopora
tccgttatcgctaaacagatgaccgcttcaacgttaagttgacaacaggaagcacgacggagactgcagtcccgtacgcgcgaacgggatacctgggatttatcaagagaacagatttcacgcagacagatggagcccggcatgacgcgttatttgtggttggccctcttgaagaaaccatgatattgcgtggtatgaggtatcacccggtagatatcgagaacacagtgacgagatgtcatcgatcaatctgtgaaagtgcggtcttcacgatgacaaacctacttgtggtagcagtggagcttgatgcagatgaacgcgaggcacttgacgtggttccgctggtgacgacatccgtactgaatgaacagcaacttgtcgtaggggtggtggtagtggttgaccctggtgtagtcccgatcaattctcgcggagagaaacaacggatgcatctgagggacgggttcctgggggaccagttggatcctatctacgtggcgtataatatgtagacacctcactgcttaattttcgtaattgaattgtgtcgtagtttttttaaatgacaactaatagacagtttgaaattgactgtagcgctaggtttaggtataaactagcgtttggtaaggcaattatgacaggaattactgtcacgcgtgacgcgagaccgtcactttacacgcaaacctgtggtcgcc

SEQ ID NO: 172 - protein, 208 aa, Green Pocillopora
SVIAKQMTASTLSQQEARRRLQSRTRERDTWDLSREQISRRQMEPGMTRYLWLALLKKPYCVVGITRISRTQRDVIDQSVKVRSSRQTYLWQWSLMQMNARHLTWFRWRHPYMNSNLSGWWWLTLVSRSILAERNNGCIGTGSWGTSWILSTWRIICRHLTAFSLNCVVVFLNDNTVNLRVVTSVWGNYDRNYCHARETVTLHANLWS

SEQ ID NO: 173 - DNA, 700 bp, Green Pocillopora
  misc_feature 633, 639: n = any nucleotide
tccgttatcgctaaacagatgaccgcttcaacgttaagttgacaacaggaagcacgacggagactgcagtcccgtacgcgcgaacgggatacctgggatttatcaagagaacagatttcacgcagacagatggagcccggcatgacgcgttatttgtggttggccctcttgaagaaaccatgatattgcgtggtatgaggtatcacccggtagatatcgagaacacagtgacgagatgtcatcgatcaatctgtgaaagtgcggtcttcacgatgacaaacctacttgtggtagcagtggagcttgatgcagatgaacgcgaggcacttgacgtggttccgctggtgacgacatccgtactgaatgaacagcaacttgtcgtaggggtggtggtagtggttgaccctggcgtagtcccgatcaattctcgcggagagaaacaacggatgcatctgagggacgggttcctgggggaccagttggatcctatctacgtggcgtataatatgtagacacctcactgcttaatttcgtaattgaattgtgtcgtagtttttttaaatgacaattaatagacagtttgaaattgactgtagcgctaggtttaggtataaactagcgtttggtaaggcnattatnacaggaactactgtcacgcgtgacgcgagaccgtcactttacacgcaaacctgtggtcgcc

SEQ ID NO: 174 - protein, 194 aa, Green Pocillopora sp.
  MOD_RES 188, 190: Xaa = any amino acid
SVIAKQMTASTLSQQEARRRLQSRTRERDTWDLSREQISRRQMEPGMTRYLWLALLKKPYCVVGITRISRTQRDVIDQSVKVRSSRQTYLWQWSLMQMNARHLTWFRWRHPYMNSNLSGWWWLTLASRSILAERNNGCIGTGSWGTSWILSTWRIICRHLTAFRNIVSFFMTINRQVNLRVVTSVWGXYXRNYC

SEQ ID NO: 175 - DNA, 700 bp, Green Pocillopora
tccgttatcgctaaacagatgaccgcttcaccgttaagttgacaacaggaagcacgacggagactgcagtcccgtacgcgcgaacgggatacctgggatttatcaagagaacagatttcacgcagacaggtggagcccggcatgacgcgttatttgtggttggccctcttgaagaaaccatgatattgcgtggtatgaggtatcacccggtagatatcgagaacacagtgacgagatgtcatcgatcaatctgtgaaagtgcggtcttcacgatgacaaacctacttgtggtagcagtggagcttgatgcagatgaacgcgaggcacttgacgtggttccgctggtgacgacatccgtactgaatgaacagcaacttgtcgtaggggtggtggtagtggttgaccctggtgtagtcccgatcaattctcgcggagagaaacaacggatgcatctgagggacgggttcctgggggaccagttggatcctatctacgtggcgtataatatgtagacacctcactgcttaatttcgtaattgaattgtgtcgtagtttttttaaatgacaattaatagacagtttgaaattgactgtagcgctaggtttaggtataaactagcgtttggtaaggcaattatgacaggaattactgtcacgcgtgacgcgagaccgtcacttcacacgcaaacctgtggtcgcc

SEQ ID NO: 176 - protein, 208 aa, Green Pocillopora sp.
SVIAKQMTASPLSQQEARRRLQSRTRERDTWDLSREQISRRQVEPGMTRYLWLALLKKPYCVVGITRISRTQRDVIDQSVKVRSSRQTYLWQWSLMQMNARHLTWFRWRHPYMNSNLSGWWWLTLVSRSILAERNNGCIGTGSWGTSWILSTWRIICRHLTAFRNIVSFFMTINRQVNLRVVTSVWGNYDRNYCHARETVTSHANLWS

SEQ ID NO: 177 - DNA, 700 bp, Millepora sp. (Hydrozoan)
tccgttatcgctaaacagatgaccgcttcaacgttaagttgacaacaggaagcacgacggagactgcagtcccgtacgcgcgaacgggatacctgggatttatcaagagaacagatttcacgcagacaggtggagcccggcatgacgcgttatttgtggttggccctcttgaagaaaccatgatattgcgtggtatgaggtatcacccggtagatatcgagaacacagtgacgagatgtcatcgatcaatctgtgaaagtgcggtcttcacgatgacaaacctacttgtggtagcagtggagcttgatgcagatgaacgcgaggcacttgacgtggttccgctggtgacgacatccgtactgtatgaacagcaacttgtcgtaggggtggtggtagtggttgaccctggtgtagtcccgatcaattctcgcggagagaaacaacggatgcatctgagggacgggttcctgggggaccagttggatcctatctacgtggcgtataatatgtagacacctcactgcttaatttcgtaattgaattgtgtcgtagtttttttaaatgacaattaatagacagtttgaaattgactgtagcgctaggtttaggtataaactagcgtttggtaaggcaattatgacaggaattactgtcacgcgtgacgcgagaccgtcacttcacacgcaaacctgtggtcgcc

SEQ ID NO: 178 - protein, 209 aa, Millepora sp.
SVIAKQMTASTLSQQEARRRLQSRTRERDTWDLSREQISRRQVEPGMTRYLWLALLKKPYCVVGITRISRTQRDVIDQSVKVRSSRQTYLWQWSLMQMNARHLTWFRWRHPYCMNSNLSGWWWLTLVSRSILAERNNGCIGTGSWGTSWILSTWRIICRHLTAFRNIVSFFMTINRQVNLRVVTSVWGNYDRNYCHARETVTSHANLWS

SEQ ID NO: 179 - DNA, 640 bp, Pavona decussaca
agactgcagtcccgtacgcgcgaacgggatacctgggatttatcaagagaacagatttcacgcagacagatggagcccggcatgacgcgttatttgtggttggccctcttgaagaaaccatgatattgcgtggtatgaggtatcacccggtagatatcgagaacacagtgacgagatgtcatcgatcaatctgtgaaagtgcggtcttcacgatgacaaacctacttgtggtagcagtggagcttgatgcagatgaacgcgaggcacttgacgtggttccgctggtgacgacatccgtactgaatgaacagcaacttgtcgtaggggtggtggtagtggttgaccctggtgtagtcccgatcaattctcgcggagagaaacaacggatgcatctgagggacgggttcctgggggaccagttggatcctatctacgtggcgtataatatgtagacacctcactgcttaatttcgtaattgaattgtgtcgtagtttttttaaatgacaattaatagacagtttgaaattgactgtagcgctaggtttaggtataaactagcgtttggtaaggcaattatgacaggaactactgtcacgcgtgacgcgagaccgtcactttacacgcaaacctgtggtcgcc

SEQ ID NO: 180 - protein, 208 aa, Pavona decussaca; identical to SEQ ID NO: 170

SEQ ID NO: 181 - protein, 231 aa, Coral
SVIAKQMTYKVYMSGTVNGHYFEVEGDGKGKPYEGEQTVRLAVTKGGPLPFAWDILSPQCQYGSIPFTKYPEDIPDYVKQSFPGRYTWERIMNFEDGAVCTVSNDSSIQGNCFIYHVKFSGLNFPPNGPVMQKKTQGWEPNTERLFARDGMLIGNNFMALKLEGGGHYLCEFKSTYKARKPVKMPGYHYVDRKLDVTNHNKDYTSVEQREISIARKPLVACCFFRVKSRHK

SEQ ID NO: 182 - protein, 235 aa, Coral
SVIAKQMTYKVYMSGTVNGHYFEVEGDGKGLPYEGGQTVRLAVTKGGPLPFAWDILSPQCQYGSIPFTKYPEDIPDYVKQSFPGRYTWERIMNFEDGAVCTVSNDSSIQGNCFIYHVKRSGLNFPPNGPVMQKKTQGWEPNTERLFARDGMLIGNNFMALKLEGGGHYLCEFKSTYKAKKPVKMPGYHYVDRKLDVTNHNLDYTSVEQCEISIARKPVVACRFFRVKSRHKYAVA

SEQ ID NO: 183 - DNA, 49 bp, Artificial Sequence
  Primer
tgagagaactagtctcgagctctagaacaagcttttttttttttttttt

SEQ ID NO: 184 - DNA, 41 bp, Artificial Sequence
  Primer
cagggcgcgccatgggatccgttatcgctaaacagatgacc

SEQ ID NO: 185 - DNA, 38 bp, Artificial Sequence
  Primer
gggttaattaagctgcagggcgaccacaggtttgcgtg

SEQ ID NO: 186 - DNA, 18 bp, Artificial Sequence
  Primer
cccgaaaagtgccacctg

SEQ ID NO: 187 - DNA, 19 bp, Artificial Sequence
  Primer
gttctgaggtcattactgg

SEQ ID NO: 188 - DNA, 20 bp, Artificial Sequence
  Primer
tcagggtacttggtgaatgg

SEQ ID NO: 189 - DNA, 669 bp, Acropora sp.
  CDS (1)...(669)
ggatccgttatcgctaaacagatgacctacaaggtttatatgtcaggcacggtcaatggacactactttgaggtcgaaggcgatggaaaaggaaagccttacgagggggagcagacggtaaagctcactgtcaccaagggtggacctctgccatttgcttgggatattttatcaccacagtcacagtacggaagcataccattcaccaagtaccctgaagacatcccggactatgtaaagcagtcattcccggagggatatacatgggagaggatcatgaactttgaagatggtgcagtgtgtactgtcagcaatgactccagcatccaaggcaactgtttcatctaccatgtcaagttctctggtttgaactttcctcccaatggacctgttatgcagaagaagacacagggctgggaacccaacactgagcgtctctttgcacgagatggaatgctgataggaaacaactttatggctctgaagttagaaggaggtggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcacgcaaacctgtggtcgccctgcag

SEQ ID NO: 190 - protein, 223 aa, Acropora sp.
GSVIAKQMTYKVYMSGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPVVALQ

SEQ ID NO: 191 - DNA, 669 bp, Discosoma sp.
  CDS (1)...(669)
ggatccgttatcgctaaacagatgacctacaaagtttatatgtcaggcacggtcaatggacactactttgaggtcgaaggcgatggaaaaggaaagccttacgagggggagcagacggtaaggctgactgtcaccaagggcggacctctgccatttgcttgggatattttatcaccacagtcacagtacggaagcataccattcaccaagtaccctgaagacatccctgactatgtaaagcagtcattcccggagggatatacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctcttagcacgagatggaatgctgataggaaacaactttatggctctgaagttagaaggaggtggtcactatttgtgtgaattcaaatctacttacaaggcaaggaagcctgtgaagatgccagggtatcactatgttgaccgcaaactggatgtaaccaatcacaacaaggattacacttccgttgagcagcgtgaaatttccattgcacgcaaacctgtggtcgccctgcag

SEQ ID NO: 192 - protein, 223 aa, Discosoma sp.
GSVIAKQMTYKVYMSGTVNGHYFEVEGDGKGKPYEGEQTVRLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLLARDGMLIGNNFMALKLEGGGHYLCEFKSTYKARKPVKMPGYHYVDRKLDVTNHNKDYTSVEQREISIARKPVVALQ

SEQ ID NO: 193 - DNA, 669 bp, Sinularia sp.
  CDS (1)...(669)
ggatccgttatcgctaaacagatgacctacaaggtttatatgtcaggcacggtcaatggacactactttgaggtcgaaggcgatggaaaaggaaagccttacgagggggagcagacggtaaagctcactgtcaccaagggtggacctctgccatttgcttgggatattttatcaccacagtcacagtacggaagcataccattcaccaagtaccctgaagacatcccggactatgtaaagcagtcattcccggaggggtatacatgggagaggatcatgaactttgaagatggtgcagtgtgtactgtcagcaatgactccagcatccaaggcaactgtttcatctaccatgtcaagttctctggtttgaactttccttccaatggacctgttatgcagaagaagacacagggctgggaacccaacactgagcgtctctttgcacgagatggaatgctgataggaaacaactttatggctctgaagttagaaggaggtggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcacgcaaacctttggtcgccctgcag

SEQ ID NO: 194 - protein, 223 aa, Sinularia sp.
GSVIAKQMTYKVYMSGTVNGHYFEVEGDGKGKPYEGEQTVKLTVTKGGPLPFAWDILSPQSQYGSIPFTKYPEDIPDYVKQSFPEGYTWERIMNFEDGAVCTVSNDSSIQGNCFIYHVKFSGLNFPSNGPVMQKKTQGWEPNTERLFARDGMLIGNNFMALKLEGGGHYLCEFKSTYKAKKPVKMPGYHYVDRKLDVTNHNKDYTSVEQCEISIARKPLVALQ

SEQ ID NO: 195 - DNA, 669 bp, Tubastrea sp.
  CDS (1)...(669)
ggatccgttatcgctaaacagatgacctacaaggtttatatgtcaggcacggtcaatggacactactttgaggtcgaaggcgatggaaaaggaaagccttacgagggggagcagacggtaaagctcactgtcaccaagggtggacctctgccatttgcttgggatattttatcaccacagtcacagtacggaagcataccattcaccaagtaccctgaagacatcccggactatgtaaagcagtcattcccggagggatatacatgggagaggatcatgaactttgaagatggtgcagtgtgtactgtcagcaatgactccagcatccaaggcaactgtttcatctaccatgtcaagttctctggtttgaactttcctcccaatggacctgttatgcagaagaagacacagggctgggaacccaacactgagcgtctctttgcacgagatggaatgctgataggaaacaactttatggctctgaagttagaaggaggtggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcgcgcaaacctgtggtcgccctgcag

SEQ ID NO: 196 - protein, 223 aa, Tubastrea sp.; identical to SEQ ID NO: 190

SEQ ID NO: 197 - DNA, 669 bp, Discosoma sp.
  CDS (1)...(669)
ggatccgttatcgctaaacagatgacctacaaggtttatatgtcaggcacggtcaatggacactactttgaggtcgaaggcgatggaaaaggaaagccttacgagggggagcagacggtaaggctggctgtcaccaagggcggacctctgccatttgcttgggatattttatcaccacagtgtcagtacggaagcataccattcaccaagtaccctgaagacatccctgactatgtaaagcagtcattcccggagggatttacatgggagaggatcatgaactttgaagatggtgcagtgtgtcctgtcagcaatgattccagcatccaaggcaactgtttcatctaccatgtcaagttctctggtttgaactttcctcccaatggacctgttatgcagaagaagacacagggctgggaaccccactctgagcgtctctttgcacgagacggaatgctgataggaaacacctttatggctctgaagttagaaggaggcggtcactatttgtgtgaattcaaaactacttacaaggcaaagaagcctgtgaagatgccagggtatcattatgttgaccgcaaactggatgtaatcaatcacaacaaggattacacttccgttgagcagtgtgaaatttccattgcacgcaaacctgtggtcgccctgcag

SEQ ID NO: 198 - protein, 223 aa, Discosoma sp.
GSVIAKQMTYKVYMSGTVNGHYFEVEGDGKGKPYEGEQTVRLAVTKGGPLPFAWDILSPQCQYGSIPFTKYPEDIPDYVKQSFPEGFTWERIMNFEDGAVCPVSNDSSIQGNCFIYHVKFSGLNFPPNGPVMQKKTQGWEPHSERLFARDGMLIGNTFMALKLEGGGHYLCEFKTTYKAKKPVKMPGYHYVDRKLDVINHNKDYTSVEQCEISIARKPVVALQ

SEQ ID NO: 199 - DNA, 555 bp, Sinularia sp.
  CDS (1)...(555)
acggtaaggctggctgtcaccaagggcggacctctgccatttgcttgggatattttatcaccacagtgtcagtacggaagcataccattcaccaagtaccttgaagacatccctgactatgtaaagcagtcattcccggagggatttacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaacccaacactgagcgtctctttgcacgagatggaatgctgataggaaacaactttatggctctaaagttagagggaggtggtcactatttgtgtgaattcaaatctacttacaaggcaaagaagcctgtgaagatgccagggtatcactatgttgaccgcaaactggatgtaaccaatcacaacaaggattacacttccgttgagcagtgtgaaatttccattgcacgcaaacctttggtcgccctgcag

SEQ ID NO: 200 - protein, 185 aa, Sinularia sp.
TVRLAVTKGGPLPFAWDILSPQCQYGSIPFTKYLEDIPDYVKQSFPEGFTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPLVALQ

SEQ ID NO: 201 - DNA, 669 bp, Tubastrea sp.
  CDS (1)...(669)
ggatccgttatcgctaaacagatgacctacaaggtttatatgtcaggcacggtcaatggacactactttgaggtcgaaggcgatggaaaaggaaagccttacgagggggagcagacggtaaggctggctgtcaccaagggcggacctctgccatttgcttgggatattttatcaccacagtgtcagtacggaagcataccattcaccaagtaccctgaagacatccctgactatgtaaagcggtcattcccggagggatttacatgggagaggatcatgaactttgaagatggtgcagtgtgtactgtcagcaatgattccagcatccaaggcaactgtttcatctaccatgtcaagttctctggtttgaactttcctcccaatggacctgttatgcagaagaagacacagggctgggaaccccactctgagcgtctctttgcacgagacggaatgctgataggaaacaactttatggctctgaagttagaaggaggcggtcactatttgtgtgaattcaaaactacttacaaggcaaagaagcctgtgaagatgccagggtatcattatgttgaccgcaaactggatgtaatcaatcacaacaaggattacacttccgttgagcagtgtgaaatttccattgcacgcaaacctgtggtcgccctgcag

SEQ ID NO: 202 - protein, 223 aa, Tubastrea sp.
GSVIAKQMTYKVYMSGTVNGHYFEVEGDGKGKPYEGEQTVRLAVTKGGPLPFAWDILSPQCQYGSIPFTKYPEDIPDYVKRSFPEGFTWERIMNFEDGAVCTVSNDSSIQGNCFIYHVKFSGLNFPPNGPVMQKKTQGWEPHSERLFARDGMLIGNNFMALKLEGGGHYLCEFKTTYKAKKPVKMPGYHYVDRKLDVINHNKDYTSVEQCEISIARKPVVALQ

SEQ ID NO: 203 - DNA, 40 bp, Artificial Sequence
  Primer
gcatggtaccatgagaggatcgcatcaccatcaccatcac

SEQ ID NO: 204 - DNA, 34 bp, Artificial Sequence
  Primer
ctgagtcgactcactgcagggcgaccacaggttt

SEQ ID NO: 205 - DNA, 46 bp, Artificial Sequence
  Primer
cagggcgcgccaaggagatataacaatggcttcctcagttctttcc

SEQ ID NO: 206 - DNA, 33 bp, Artificial Sequence
  Primer
cactggatccgcattgcactcttccgccgttgc

SEQ ID NO: 207 - DNA, 45 bp, Artificial Sequence
  Primer
gcatggcgcgccaaggagatataacaatgaagactaatctttttc

SEQ ID NO: 208 - DNA, 34 bp, Artificial Sequence
  Primer
gcatggatccgaattcggccgaggataatgatag

SEQ ID NO: 209 - DNA, 46 bp, Artificial Sequence
  Primer
gatcttaattaaagctcatcatgctgcagggcgaccacaggtttgc

SEQ ID NO: 210 - DNA, 42 bp, Artificial Sequence
  Primer
gcatctgcaggtcgccaccagtaaaggagaagaacttttcac

SEQ ID NO: 211 - DNA, 39 bp, Artificial Sequence
  Primer
ctgattaattaattatttgtatagttcatccatgccatg

SEQ ID NO: 212 - DNA, 55 bp, Artificial Sequence
  Primer
cagggcgcgccaaggagatataacaatgggatccgttatcgctaaacagatgacc

SEQ ID NO: 213 - DNA, 44 bp, Artificial Sequence
  Primer
ggctctagaaaggagatatacaagtgtgatcgctacacaaatga

SEQ ID NO: 214 - DNA, 45 bp, Artificial Sequence
  Primer
ggctctagaaaggagatatacaatgtccgttatcgctaaacagat

SEQ ID NO: 215 - DNA, 50 bp, Artificial Sequence
  Primer
ggcaagctttcagtggtggtggtggtggtgggcgaccacaggtttgcgtg

SEQ ID NO: 216 - protein, 221 aa, Coral
MSVIATQMTYKVYMSGTVNGHYFEVEGDGKGKPYEGEQTVKLTVTKGGPLPFAWDILSPQCQYGSIPFTKYPEDIPDYVKQSFPEGFTWERIMNFEDGAVCTVSNDSSIQGNCFTYHVKFSGLNFPPNGPVMQKKTQGWEPSSEHLFARGGMLIGNNHMALKLEGGGHYLCEFKTTYKAKKPVKMPGYHYVDRKLDVTNHNKDYTSVEQCEISIARKPVVA

SEQ ID NO: 217 - protein, 221 aa, Coral
MSVIAKQMTYKVYMSGTVNGHYFEVQGDGKGKPYEEEQTVKLTVTKGGPLPFAWDILSPQSQYGSIPFTKYPEDIPDYVKQSFPEGYTWERIMNFEDGAVCTVSNDSSIQGNCFIYNVKFSGLNFPPNGPVMQKKTQGWEPNTERLFARDGMLIGNNFMALKLEGGGHYLCEFKSTYKAKKPVKMPGYHYVDRKLDVTNHNIDYTSVEQCEISIARKPVVA

SEQ ID NO: 218 - protein, 226 aa, Coral
SVIAKQMTYKVYMSGTVNGHYFEVEGDGKGKPYEGEQTVRLTVTKGGPLPFAWDILSPQSQYGSIPFTKYPEDIPDYVKQSFPEGYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVRMPGYHYVDRKLDVTNHNKDYTSVEQCEISIARKPVVAWCFFRV

SEQ ID NO: 219 - protein, 220 aa, Coral
SVIAKQMTYKVYMSGTVNGHYFEVEGDGKGKPYEGEQTVRLAVTKGGPLPFAWDILSPQCQYGSIPFTKYPEDIPDYVKQSFPGRYTWERIMNFEDGAVCTVSNDSSIQGNCFIYHVKFSGLNFPPNGPVMQKKTQGWEPNTERLFARDGMLIGNNFMALKLEGGGHYLCEFKSTYKARKPVKMPGYHYVDRKLDVTNHNKDYTSVEQREISIARKPLVA

SEQ ID NO: 220 - protein, 230 aa, Coral
MSCSKNVIKEFMRFKVRMEGTVNGHEFEIKGEGEGRPYEGHCSVKLMVTKGGPLPFAFDILSPQFQYGSKVYVKHPADIPDYKKLSFPEGFKWERVMNFEDGGVVTVSQDSSLKDGCFIYEVKFIGVNFPSDGPVMQRRTRGWEASSERLYPRDGVLKGDIHMALRLEGGGHYLVEFKSIYMVKKPSVQLPGYYYVDSKLDMTSHNEDYTVVEQYEKTQGRHHPFIKPLQ

SEQ ID NO: 221 - protein, 225 aa, Coral
MRSSKNVIKEFMRFKVRMEGTVNGHEFEIEGEGEGRPYEGHNTVKLKVTKGGPLPFAWDILSPQFQYGNKVYVKHPADIPDYKKLSFPEGFKWERWMNFEDGGVVTVTQDSSLQDGCFIYKVKFIGVNFPSDGPVMQKKTMGWEASTKRLYPRDGVLKGEIHKALKLKDGGHYLVEFKSIYMAKKPVQLPGYYYVDSKLDITSHNEDYTIVEQYERTEGRHHLFL

SEQ ID NO: 222 - protein, 230 aa, Coral
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGIT

SEQ ID NO: 223 - DNA, 818 bp, Aequorea victoria
ggatccaaggagatataacaatgaagactaatctttttctctttctcatcttttcacttctcctatcattatcctcggccgaattcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatgatgagctttaagagctc

SEQ ID NO: 224 - protein, 265 aa, Aequorea victoria
GSKEIRLIFFSFSSFHFSYHYPRPNSVKEKNFSLELSQFLLNMVMLMGTNFLSVERVKVMQHTENLPLNLFALLENYLFHGQHLSLLSLMVFNAFQDTQIISGTTSSRAPCLRDTCRRGPSSSRTTGTTRHVLKSSLRETPSSTGSSLRESISRRTETSSATSWNTTTTPTTYTSWQTNKRMESKLTSKLDTTLKMEAFNQTIINKILQLAMALSFYQTTITCPHNLPFRKIPTKRETTWSFLSLQLLGLHMAWMNYTNMMSFKS

SEQ ID NO: 225 - protein, 235 aa, Acropora aspera
SVIAKQMTYKVYMSGTVNGHYFEVEGDGKGKPYEGEQTVRLAVTKGGPLPFAWDILSPQCQYGSIPFTKYPEDIPDYVKQSFPGRYTWERIMNFEDGAVCTVSNDSSIQGNCFIYHVKFSGLNFPPNGPVMQKKTQGWEPNTERLFARDGMLIGNNFMALKLEGGGHYLCEFKSTYKAKKPVKMPGYHYVDRKLDVTNHNKDYTSVEQCEISIARKPVVACRFFRVKSRHKVAVA

SEQ ID NO: 226 - protein, 232 aa, Acropora aspera
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPVMQNKAGRWEPATEIVYEVDGVLRGQSLMALKCPGGRHLTCHLHTTYRSKKPASALKMPGFHFEDHRIEIMEEVEKGKCYKQYEAAVGRYCDAAPSKLGHN

SEQ ID NO: 227 - DNA, 51 bp, Artificial Sequence
  Primer
cgcgccaaggagatataacaatgagaggatcgcatcaccatcaccatcacg

SEQ ID NO: 228 - DNA, 51 bp, Artificial Sequence
  Primer
gatccgtgatggtgatggtgatgcgatcctctcattgttatatctccttgg

SEQ ID NO: 229 - DNA, 47 bp, Artificial Sequence
  Primer
ctgattaattaaagctcatcatgtttgtatagttcatccatgccatg

SEQ ID NO: 230 - DNA, 34 bp, Artificial Sequence
  Primer
gtgtgtactgtcagccaggattccagcatccaag

SEQ ID NO: 231 - DNA, 32 bp, Artificial Sequence
  Primer
ctgtcagcaatgatatcagcatccaaggcaac

SEQ ID NO: 232 - DNA, 44 bp, Artificial Sequence
  Primer
ggatccatcgccaccatgtctaaaggtgaagaattattcactgg

SEQ ID NO: 233 - DNA, 34 bp, Artificial Sequence
  Primer
cagctgttatttgtacaattcatccataccatgg

SEQ ID NO: 234 - DNA, 41 bp, Artificial Sequence
  Primer
cgggatccatcgccaccatgaggtcttccaagaatgttatc

SEQ ID NO: 235 - DNA, 31 bp, Artificial Sequence
  Primer
gaggatccgcggccgctaaaggaacagatgg

SEQ ID NO: 236 - DNA, 38 bp, Artificial Sequence
  Primer
gaagatctaaaacaatgagtgtgatcgctacacaaatg

SEQ ID NO: 237 - DNA, 35 bp, Artificial Sequence
  Primer
tatcaaatcgccggcgtcaggcgaccacaggtttg

SEQ ID NO: 238 - DNA, 30 bp, Artificial Sequence
  Primer
agatctgtgttgtgacgcaactgcaactcc

SEQ ID NO: 239 - DNA, 39 bp, Artificial Sequence
  Primer
gtgatcagcggatcccttcaatttagaaagcaattgttc

SEQ ID NO: 240 - DNA, 25 bp, Artificial Sequence
  Primer
cctctatatattacgcaccatattc

SEQ ID NO: 241 - DNA, 22 bp, Artificial Sequence
  Primer
atacgtgacgacattggtagtc

SEQ ID NO: 242 - protein, 15 aa, Artificial Sequence
  N-terminal amino acid sequence derived from Montipora sp.
SPPDYTLEFPKKXVA

SEQ ID NO: 243 - protein, 15 aa, Artificial Sequence
  N-terminal amino acid sequence derived from Montipora sp.
SPPDYTLERPKKGVA

SEQ ID NO: 244 - protein, 24 aa, Artificial Sequence
  N-terminal amino acid sequence derived from Pavona sp.
DSSPESYLKNGIAEEMKTDVMEGI

SEQ ID NO: 245 - protein, 22 aa, Artificial Sequence
  N-terminal amino acid sequence derived from Pavona sp.
SYLPNGIAEEMKTDLMEGIVNG

SEQ ID NO: 246 - protein, 22 aa, Artificial Sequence
  N-terminal amino acid sequence derived from Pavona sp.
SLYQNGIAEEMKTDLMEGIVNG

SEQ ID NO: 247 - DNA, 20 bp, Artificial Sequence
  Primer
atggaagggatagtcgatgg

SEQ ID NO: 248 - DNA, 20 bp, Artificial Sequence
  Primer
atggaagggattgtcgatgg

SEQ ID NO: 249 - DNA, 20 bp, Artificial Sequence
  Primer
atggaagggatcgtcgatgg

SEQ ID NO: 250 - DNA, 19 bp, Artificial Sequence
  Primer
cctcgacaatcccttccat

SEQ ID NO: 251 - DNA, 19 bp, Artificial Sequence
  Primer
cctcgacgatcccttccat